SEQUENCE LISTING (199 sequences)

SEQ ID NO: 1 - DNA, 2802 bp, Aspergillus fumigatus
atgcctcaccgcgcggcatccccagcggtgtcggaaaatgagttcgacatcacaggcgctctcttccagaacgacagcgactccgacaacgaacagccctcggccaagtccaaacgacaacctccgaagaaggttccctcgcaggcgctcgacttcctcggcgatgtgaatgaagacgacaatgacgacgaggcttttattgccgagcagcaaacttctgccaaccgcaaggcctcgaatctcaaaggtcgcactgttaagaagggtggtggtttccaagccatgggcttgagtgccaatctgttaaaggcaatcgctcggaaaggcttctcggtaccgactcccattcagcgcaagaccattcccgttattatggacgaccaggatgtagttggtatggcacggactggttcaggaaagacggccgctttcgttatcccaatgatcgagaaattgaagagccatagcaccaaggttggagcccgcggtctggtcttgtccccatcgagagagctggcactgcagacattgaaagtcgtcaaggaactgggtagaggcactgacctgaagtcggttcttcttgttggtggagacagcctggaggagcaattcgcgatgatcgccggcaatccagatattattattgcaacacctggtcgattcctgcatttgaaggtggaaatgaacctggacttgtccagtatccgctatgtcgttttcgacgaggctgatcgactgtttgagatgggtttcgccgcgcaactaacagagattttgcacggtcttcctgcgaatcggcagactctcctgttctctgccactcttccgaagtcccttgtcgagtttgcccgcgccggcttgcaggaacctacactagtccgtctggataccgagagcaagatctcgcccgatcttcagaatgctttcttctctgtgaaatcctcagagaaggaaggggccttgctctacatccttcatgaggtcatcaagatgccgactgggccaaccgaggtttcgcaacaaaggaaagaagaagatgcaagcgccaagaacttgaagaacaagaagaggaagagagcggaaatggagaaggctgtcaatacgagagagtctccaaccaagcattcgacaatcgtttttgccgcgacgaagcaccatgtcgattatctttactcactactctgcgaggcgggattcgccgtctcctacgtttacggctctttagatcaaaccgcccgaaagatccaggttcaaaactttagaacgggcatgaccaatatcctcgttgtcaccgacgttgcggctagaggtattgatattcctatcctggcaaatgtcattaattacgatttcccctcccagccaaagatttttgttcaccgtgtcggccgaactgctcgtgccgggcgcaagggctggagttacagtctggtccgcgatgcagacgctccttatttacttgatttgcaactattcctaggaagaaggctggttgttggccgcgaattcggggatcaagtgaacttcgccgaagacgttgtaacgggcagtctacctagggatgggctctctcaaagctgtgagtgggtaaccaaggtcctggacgataatgcagaccttgcagcccaacgcacggtcgctgctaagggtgagaagctctatatgcgaacccggaacgcggcatctcttgagagtgcgaagcgatcgaaacaggtggtttcttccgacaattggacaagcgtacacccgctcttccaagatgaaactagtaatctggaggccgagcgggagaagatgctcgcccgtattggtggttaccggccgccagagacaatctttgaggtcaacaaccggcggatgggcaaacatgagaacgtcgacgctctcgatacgattaagagagttcgtagcactctagagtccaagaagaagcgtgcacaagcgaatgaaaagtccgagttcctcgaagacggtcccgacgacggaaaggcagtcaacgaagccaaagaaaccgagagcgagggagcattttctgatgaggacgacgacgttcccaccggcgtggcagataacatgtcgatggcatccgattcagagctggaagtcaccttctcgtcatattcaaaatcaaaggacaacaaggcgaagaaagcgagcgcggcatctttccagaaccctgaatacttcatgtcctataccccgaataacacctccctggcggaggaccgagcttatggtgtgcattccggtaccaactccaacttcgcccaggcctcacgcagcgcgaccatggatctggcaggcgacgatggaggccgcgggttcggcgaggctcgtacgctgatgcgctgggacaagcgacacaagaagtacgtggctcgacagaatgacgaggacggctccaagggcacacgcctcgtccgcggtgagagcggggcgaagatcgcagccagcttccggagcggacggttcgacgcttggaagagagaaaatcgtctcggccgcttgcctcgggtgggtgaggccgaagctgctaatctcgctgctggtctcaacgcagccatctcaggcaagcggttcaggcatcgcaaggagcaggcgcccaagaaggctgatcctctccgtggtgattacgagaagatgaagaagaaggctgaactcgccaaggagcgagcaatgtctaaggctggcggcgctgcaccacgtggcaagagcgagctgaagagtacggacgatatccggattgcgcgcaaattgaagcagaagagacgagagaagaatgctcgtccctcgaggaagaag

SEQ ID NO: 2 - DNA, 2802 bp, Aspergillus fumigatus; identical to SEQ ID NO: 1

SEQ ID NO: 3 - protein, 934 aa, Aspergillus fumigatus
MPHRAASPAVSENEFDITGALFQNDSDSDNEQPSAKSKRQPPKKVPSQALDFLGDVNEDDNDDEAFIAEQQTSANRKASNLKGRTVKKGGGFQAMGLSANLLKAIARKGFSVPTPIQRKTIPVIMDDQDVVGMARTGSGKTAAFVIPMIEKLKSHSTKVGARGLVLSPSRELALQTLKVVKELGRGTDLKSVLLVGGDSLEEQFAMIAGNPDIIIATPGRFLHLKVEMNLDLSSIRYVVFDEADRLFEMGFAAQLTEILHGLPANRQTLLFSATLPKSLVEFARAGLQEPTLVRLDTESKISPDLQNAFFSVKSSEKEGALLYILHEVIKMPTGPTEVSQQRKEEDASAKNLKNKKRKRAEMEKAVNTRESPTKHSTIVFAATKHHVDYLYSLLCEAGFAVSYVYGSLDQTARKIQVQNFRTGMTNILVVTDVAARGIDIPILANVINYDFPSQPKIFVHRVGRTARAGRKGWSYSLVRDADAPYLLDLQLFLGRRLVVGREFGDQVNFAEDVVTGSLPRDGLSQSCEWVTKVLDDNADLAAQRTVAAKGEKLYMRTRNAASLESAKRSKQVVSSDNWTSVHPLFQDETSNLEAEREKMLARIGGYRPPETIFEVNNRRMGKHENVDALDTIKRVRSTLESKKKRAQANEKSEFLEDGPDDGKAVNEAKETESEGAFSDEDDDVPTGVADNMSMASDSELEVTFSSYSKSKDNKAKKASAASFQNPEYFMSYTPNNTSLAEDRAYGVHSGTNSNFAQASRSATMDLAGDDGGRGFGEARTLMRWDKRHKKYVARQNDEDGSKGTRLVRGESGAKIAASFRSGRFDAWKRENRLGRLPRVGEAEAANLAAGLNAAISGKRFRHRKEQAPKKADPLRGDYEKMKKKAELAKERAMSKAGGAAPRGKSELKSTDDIRIARKLKQKRREKNARPSRKK

SEQ ID NO: 4 - DNA, 1735 bp, Aspergillus fumigatus
atgagtgcaatcctttctgcagacgatttgaacgatttcatttctcccggggttgcttgcatcaagcccgttgagagtctaccacaaaaagaatcccagtcggaggtatctttcctgtcttaccagtcatctgttgatatcagccaataggctaacgctcatttccaattcaatagaatccctatgaggtgacaaaggaagacaaagttcaaccggaaaaccttcccccggctcagatttcattgactgattgccttgcatgctccggatgtgtcacgtctgcggaagcagtgttgatatccttgcaatcacatacggaggttctcaatactcttgattcgtaccccgaattgccgcttggttctacaagctaccaaagaggcacacaaaaagttggatcagcagacagcgatggtcgcatctttgttgctagcgtcagccctcaagtcagggcgagcttggcagccacatacggaatcaccgagcgggaggcgaaatatatgattgaccaatttcttatgggccctcacggtctcagagctggtggaaaacatggcaatgggtttacatgggttgtggacacgaacgttatgcgtgaagcagtgttggctctgacagcggacgaggtcacgagctctttattatcaactggatcgggcagccttcccaagagtccaattctttcgtccgcttgccccggctggatatgttatgctgaaaaaacacacccttttatccttccgcatttatctcgcctcaagtctcctcaggcgttgagcggcacatttctgaagtcagtgctaagcaaggcacttggggtcccgccttctcagatatggcatttagctatcatgccatgcttcgacaagaagctggaagctagccgggaagagctgacagacattgcatgggcttcaaccttcacccagtcacagacaacacccgtccgcgacgttgactgtgtcataaccacccgtgagctactaactttagccactgctagggggctttctctacccaatttgccgctcaaaccattgcccgcgtcatgtttaactccatttccagatcaagccctagaatcatttttgttctctaagagctcgtcgggccaaacagtcgaatcagggacatctggaggctatcttcatcacgtcctccaaatcttccaagccagaaaccccggcagcaagattgtcacccagcgtgggcgcaacgccgatgttgtggaatatgtgctcatgtcgtctggggatgagcctctttttagggcggctcggtattatggcttcaggaatatacaaaatctcgtcagaaaacttaaacccgcacgcgtgtcaagactgccaggcgccaagccgcaagcggtctcttcaagtgcaaatcgacgacagcccatgtcaaggaacgcagctccggctggaacaggcgctgattatgcatatgttgaagtcatggcttgtcctggcggctgtaccaatggtggtgggcaaataaggattgaagatgcccgggaggctgttccgaacgcactaaaagagacatcgactgaaactcctgtggctgcaccgaaacccacgccgcatgagcagcgtgcctggctagcccgggtagatgaagcgtactactctgcggactcggatagcgagggatctgtcacgacggagccggtttctgtcctgtcaagggataaccagattcatgagtttttgaactattggtcagagaaggttga

SEQ ID NO: 5 - DNA, 1592 bp, Aspergillus fumigatus
atgagtgcaatcctttctgcagacgatttgaacgatttcatttctcccggggttgcttgcatcaagcccgttgagagtctaccacaaaaagaatcccagtcggagaatccctatgaggtgacaaaggaagacaaagttcaaccggaaaaccttcccccggctcagatttcattgactgattgccttgcatgctccggatgtgtcacgtctgcggaagcagtgttgatatccttgcaatcacatacggaggttctcaatactcttgattccgatggtcgcatctttgttgctagcgtcagccctcaagtcagggcgagcttggcagccacatacggaatcaccgagcgggaggcgaaatatatgattgaccaatttcttatgggccctcacggtctcagagctggtggaaaacatggcaatgggtttacatgggttgtggacacgaacgttatgcgtgaagcagtgttggctctgacagcggacgaggtcacgagctctttattatcaactggatcgggcagccttcccaagagtccaattctttcgtccgcttgccccggctggatatgttatgctgaaaaaacacacccttttatccttccgcatttatctcgcctcaagtctcctcaggcgttgagcggcacatttctgaagtcagtgctaagcaaggcacttggggtcccgccttctcagatatggcatttagctatcatgccatgcttcgacaagaagctggaagctagccgggaagagctgacagacattgcatgggcttcaaccttcacccagtcacagacaacacccgtccgcgacgttgactgtgtcataaccacccgtgagctactaactttagccactgctagggggctttctctacccaatttgccgctcaaaccattgcccgcgtcatgtttaactccatttccagatcaagccctagaatcatttttgttctctaagagctcgtcgggccaaacagtcgaatcagggacatctggaggctatcttcatcacgtcctccaaatcttccaagccagaaaccccggcagcaagattgtcacccagcgtgggcgcaacgccgatgttgtggaatatgtgctcatgtcgtctggggatgagcctctttttagggcggctcggtattatggcttcaggaatatacaaaatctcgtcagaaaacttaaacccgcacgcgtgtcaagactgccaggcgccaagccgcaagcggtctcttcaagtgcaaatcgacgacagcccatgtcaaggaacgcagctccggctggaacaggcgctgattatgcatatgttgaagtcatggcttgtcctggcggctgtaccaatggtggtgggcaaataaggattgaagatgcccgggaggctgttccgaacgcactaaaagagacatcgactgaaactcctgtggctgcaccgaaacccacgccgcatgagcagcgtgcctggctagcccgggtagatgaagcgtactactctgcggactcggatagcgagggatctgtcacgacggagccggtttctgtcctgtcaagggataaccagattcatgagtttttgaactattggtcagagaaggt

SEQ ID NO: 6 - protein, 530 aa, Aspergillus fumigatus
MSAILSADDLNDFISPGVACIKPVESLPQKESQSENPYEVTKEDKVQPENLPPAQISLTDCLACSGCVTSAEAVLISLQSHTEVLNTLDSDGRIFVASVSPQVRASLAATYGITEREAKYMIDQFLMGPHGLRAGGKHGNGFTWVVDTNVMREAVLALTADEVTSSLLSTGSGSLPKSPILSSACPGWICYAEKTHPFILPHLSRLKSPQALSGTFLKSVLSKALGVPPSQIWHLAIMPCFDKKLEASREELTDIAWASTFTQSQTTPVRDVDCVITTRELLTLATARGLSLPNLPLKPLPASCLTPFPDQALESFLFSKSSSGQTVESGTSGGYLHHVLQIFQARNPGSKIVTQRGRNADVVEYVLMSSGDEPLFRAARYYGFRNIQNLVRKLKPARVSRLPGAKPQAVSSSANRRQPMSRNAAPAGTGADYAYVEVMACPGGCTNGGGQIRIEDAREAVPNALKETSTETPVAAPKPTPHEQRAWLARVDEAYYSADSDSEGSVTTEPVSVLSRDNQIHEFLNYWSEK

SEQ ID NO: 7 - DNA, 942 bp, Aspergillus fumigatus
atgactaccggggctggtacgatctctcattccaacacctatcatcgtattcctcgccgttaactgaccaatccaccagtgcaaaggttccgtccagtggtggtatcgggtccctctgggactgggaagtcgaccttgctcaagagactcttcgctgaataccccgatactttcgatttatccgtgtctcgtacgtctaaccccttgccaaccctcattgactatgcctgcgaattgtttcttttggtggaattgcgctgaacggtgtttgttatatttagataccactcgagctccccgtcccggggaagaaaatggacgtgagtattacttcacaactaaagaagatttcctggatcttgtgagcaagaatgcctttatcgagcatgcgcagtttggtggcaattactacggtactactgtgcaggcagtgaaggatgttgcgcagaagggcaagatctgcgttctcgacattgagatgaggtaataatagtcctgcaacgtgaactgatatgaccggagaagcagaggaaatccatcatcaaatggattgtagttcaacccaaacaacagctgacgactgaattgcaatagggcgtgaaacaagtcaagcgcaccgatcttgatgctcgattcttatttttagcacccccgtcccttgaagaactagagaaaagactgcgtgggagagcaaccgagactgaggagagcttgacggtatggctgtcctccacattccttcacttccccaactcgccagactgtcccgctggaattctaactttgcgtcagaaacgccttgcccaagctaaaaatgaattggaatatgcggcgcagcctggctctcatgataagattgtcgtgaacgatgacctggagaaggcttataaggaactgcgggattggattgtcgacggtggtaactttggagcgcgtcaatga

SEQ ID NO: 8 - DNA, 600 bp, Aspergillus fumigatus
atgactaccggggctgtgcaaaggttccgtccagtggtggtatcgggtccctctgggactgggaagtcgaccttgctcaagagactcttcgctgaataccccgatactttcgatttatccgtgtctcataccactcgagctccccgtcccggggaagaaaatggacgtgagtattacttcacaactaaagaagatttcctggatcttgtgagcaagaatgcctttatcgagcatgcgcagtttggtggcaattactacggtactactgtgcaggcagtgaaggatgttgcgcagaagggcaagatctgcgttctcgacattgagatgaggggcgtgaaacaagtcaagcgcaccgatcttgatgctcgattcttatttttagcacccccgtcccttgaagaactagagaaaagactgcgtgggagagcaaccgagactgaggagagcttgacgaaacgccttgcccaagctaaaaatgaattggaatatgcggcgcagcctggctctcatgataagattgtcgtgaacgatgacctggagaaggcttataaggaactgcgggattggattgtcgacggtggtaactttggagcgcgtcaa

SEQ ID NO: 9 - protein, 200 aa, Aspergillus fumigatus
MTTGAVQRFRPVVVSGPSGTGKSTLLKRLFAEYPDTFDLSVSHTTRAPRPGEENGREYYFTTKEDFLDLVSKNAFIEHAQFGGNYYGTTVQAVKDVAQKGKICVLDIEMRGVKQVKRTDLDARFLFLAPPSLEELEKRLRGRATETEESLTKRLAQAKNELEYAAQPGSHDKIVVNDDLEKAYKELRDWIVDGGNFGARQ

SEQ ID NO: 10 - DNA, 2059 bp, Aspergillus fumigatus
atgttagaagccttcgaagtcttgacaacatctggggtggtgctgtggtcgaagtcgtatgcgccggtcggagcgcatgttgtcaacagcctaatcaacgatgtcttcattgaggagaaggttcgagcgcagaatcaggcagcgagcagtgcagctcctatctacaagaaggaaaagtatactctgaaatggaagcaagtaaaggatttcaatctgatatttgtggtatgttcacgccgctcgttgattcaatggcgccactgaccgattccataggctgtatatcaatctctgctacatcttggttggatcgacaaactcttggataatgtttcgaccatattcatcgacttatataaggatgagctaaggagcacacgggctaggattattgagtacccattcgataagtacttcgaccagcaggtgcgagagcttgaggacaatgctggggctcctacatcagaatctctcgtagtagagatcaacgagagaaaggaccctcttgtctcatcagataacggcgggccacctccgccacccgtgcctggtctgctgaaaggtatctgacgtcgataatttttctctgctagtgatcatattgctaactacctccgaagcgcaacgtccagttgcgcagggcgtggcgacctcggacgagggttcgccaccccaaaccccagatctttctcgatcgtcaacgcccatttcaggtcatctattgaccgcgaaaggagggcctgctggccgcgcctctcgtcgcgcacgcaaagcggccaacgcgagcgctaccgcttcttctggagatgaaagcattcggaaggggaaaacattgaaaagtggaaaaaagatgcgcaagtgggatgctgatggctttgcggatgaggacgacggcaaggtcctcgattactccgcccccgcagatggtgaggacgcaccggctcctgtagtcgaggctgttgcgcaggaatcctggggacgccgaacaggcaagggccaatttgtgctgaaagatctaggggatgaagtccattccattcttgagaatgctgatcatgaaaagacaaagtcttcctcgtccacgggctttgttgggtctggagtcaacgcacttggtggattcttccgtaatattgtcggcggcaaggtccttactgaggctgacttggagaaacccttgaaagccatggaagaccatttgctgaagaagaacgttgcgcgcgaagcggccgtccgtctatgtcaaggcgtccagcgcgaattagttggcaagaagacaggcaactttcaaagtgttgatgcagcactgcgctccgcaatggagtcctcgttgcgcaaaatattgacgccaacgtcatctctcgatctactgcgtgagatcgatgctgttagatctccgacgagcaaaggacaggctcctcgcccatatgtcatttccatcgtgggcgtgaacggtgttgggaagtcgacaaatctgggcaaaatttgttacttccttctccagaataactatcgtgttctgattgcagcctgtgacaccttccgctctggagccgtggagcagttacgagtccatgctcgcaatttgaaggaacttagtacccgggagaatgctggagaggttgaactctacgagaagggatatggaaaggatgcagcgaatgtagcgaaggatgcagtggagtacggtgcggcgaatcatttcgacgttgtgttgattgatactgccggtcgccgtcataacgaccaacgccttatgtcttcgctcgagaagttcgccaagttcgccaaaccagataagatcttcatggtcggtgaagctctggtcggtacggacagcgtgatgcaggctcgcaacttcaaccaagctttcggcactgggagaaacctcgatgggttcatcatcagtaaatgtgataccgttggtgacatggtaggtacgcttgtcagcatggtgcatgctacaggcattcctattgtttttctgggtgtaggccagcactatggtgatttgaggggcctaagtgttccttgggctgtcaatctgctgatgaagtga

SEQ ID NO: 11 - DNA, 1923 bp, Aspergillus fumigatus
atgttagaagccttcgaagtcttgacaacatctggggtggtgctgtggtcgaagtcgtatgcgccggtcggagcgcatgttgtcaacagcctaatcaacgatgtcttcattgaggagaaggttcgagcgcagaatcaggcagcgagcagtgcagctcctatctacaagaaggaaaagtatactctgaaatggaagcaagtaaaggatttcaatctgatatttgtggctgtatatcaatctctgctacatcttggttggatcgacaaactcttggataatgtttcgaccatattcatcgacttatataaggatgagctaaggagcacacgggctaggattattgagtacccattcgataagtacttcgaccagcaggtgcgagagcttgaggacaatgctggggctcctacatcagaatctctcgtagtagagatcaacgagagaaaggaccctcttgtctcatcagataacggcgggccacctccgccacccgtgcctgttgcgcagggcgtggcgacctcggacgagggttcgccaccccaaaccccagatctttctcgatcgtcaacgcccatttcaggtcatctattgaccgcgaaaggagggcctgctggccgcgcctctcgtcgcgcacgcaaagcggccaacgcgagcgctaccgcttcttctggagatgaaagcattcggaaggggaaaacattgaaaagtggaaaaaagatgcgcaagtgggatgctgatggctttgcggatgaggacgacggcaaggtcctcgattactccgcccccgcagatggtgaggacgcaccggctcctgtagtcgaggctgttgcgcaggaatcctggggacgccgaacaggcaagggccaatttgtgctgaaagatctaggggatgaagtccattccattcttgagaatgctgatcatgaaaagacaaagtcttcctcgtccacgggctttgttgggtctggagtcaacgcacttggtggattcttccgtaatattgtcggcggcaaggtccttactgaggctgacttggagaaacccttgaaagccatggaagaccatttgctgaagaagaacgttgcgcgcgaagcggccgtccgtctatgtcaaggcgtccagcgcgaattagttggcaagaagacaggcaactttcaaagtgttgatgcagcactgcgctccgcaatggagtcctcgttgcgcaaaatattgacgccaacgtcatctctcgatctactgcgtgagatcgatgctgttagatctccgacgagcaaaggacaggctcctcgcccatatgtcatttccatcgtgggcgtgaacggtgttgggaagtcgacaaatctgggcaaaatttgttacttccttctccagaataactatcgtgttctgattgcagcctgtgacaccttccgctctggagccgtggagcagttacgagtccatgctcgcaatttgaaggaacttagtacccgggagaatgctggagaggttgaactctacgagaagggatatggaaaggatgcagcgaatgtagcgaaggatgcagtggagtacggtgcggcgaatcatttcgacgttgtgttgattgatactgccggtcgccgtcataacgaccaacgccttatgtcttcgctcgagaagttcgccaagttcgccaaaccagataagatcttcatggtcggtgaagctctggtcggtacggacagcgtgatgcaggctcgcaacttcaaccaagctttcggcactgggagaaacctcgatgggttcatcatcagtaaatgtgataccgttggtgacatggtaggtacgcttgtcagcatggtgcatgctacaggcattcctattgtttttctgggtgtaggccagcactatggtgatttgaggggcctaagtgttccttgggctgtcaatctgctgatgaag

SEQ ID NO: 12 - protein, 641 aa, Aspergillus fumigatus
MLEAFEVLTTSGVVLWSKSYAPVGAHVVNSLINDVFIEEKVRAQNQAASSAAPIYKKEKYTLKWKQVKDFNLIFVAVYQSLLHLGWIDKLLDNVSTIFIDLYKDELRSTRARIIEYPFDKYFDQQVRELEDNAGAPTSESLVVEINERKDPLVSSDNGGPPPPPVPVAQGVATSDEGSPPQTPDLSRSSTPISGHLLTAKGGPAGRASRRARKAANASATASSGDESIRKGKTLKSGKKMRKWDADGFADEDDGKVLDYSAPADGEDAPAPVVEAVAQESWGRRTGKGQFVLKDLGDEVHSILENADHEKTKSSSSTGFVGSGVNALGGFFRNIVGGKVLTEADLEKPLKAMEDHLLKKNVAREAAVRLCQGVQRELVGKKTGNFQSVDAALRSAMESSLRKILTPTSSLDLLREIDAVRSPTSKGQAPRPYVISIVGVNGVGKSTNLGKICYFLLQNNYRVLIAACDTFRSGAVEQLRVHARNLKELSTRENAGEVELYEKGYGKDAANVAKDAVEYGAANHFDVVLIDTAGRRHNDQRLMSSLEKFAKFAKPDKIFMVGEALVGTDSVMQARNFNQAFGTGRNLDGFIISKCDTVGDMVGTLVSMVHATGIPIVFLGVGQHYGDLRGLSVPWAVNLLMK

SEQ ID NO: 13 - DNA, 1564 bp, Aspergillus fumigatus
atgcggtggtgcctcactcttctggcattctgcttcttggcagttgtacgtgcattaagtagctccggcagtcgtctgttggttgttttggaagatgccacagaaaaggaattatactcgaaattatgggctgacctagaaggtgctctaacctactgaacttctacgttaatatgctaatattaattggtagctcgaggatataacctcgacttcgaatcccccaagaatgacaagctcagcctgttcgaactcggagaccgagtctacgaccacatgcttctcctgcctcccaagtcaaagggttagcgttacccttagacatgtccatatgctctgctttgtacatctcaattgacctcttggccaggctatggaccctcccttacccccaagaatatcattgatttcatgaacaaggacggtaacgtcctcctcgccttgtcgggcaagtccacaaccgccagcgctatcagctcgctgctattggagctcgatctccatctccctgtcgatcgttcctctgtcaccgtcgatcacttcaactacgatacactttctgcctccgataagcatgatgttctgctactccaccgaccaggcaagttgaggtccgataccaaggctttctttgatggcgagggcgttgtagcatttcccagagccgtcccccacaccctgggcgatgcaaaccctctcattgcgcctattctgcgagcgcccgccactgcgtatagttacaaccccaaggaggacgcgtcgtcagttgaggatgttgcagctacgggttcgcagttggctctggtctcggccatgcaggctagaaactccgctcggttcactctactgggatccgtggagagtctgcaggatcagtggttttctgcgactgtcaaggctcctggtgatgggaagcagatgaagacggtcaaccaggaattcgccaagcagcttactgcgtggacattcaaggaaaccggagtcctcaaggtcggaaagatcgagcatcatctggctgaagatggtgaaatcactcccgagaagctgaaccctaagatctatcgaataaagaatgaaactgtaagtgacagccatctgaggttccattgcctatttgcatgctcacccttctcaacaggtctttagcattgaactttccgaatacaactatgatcgttacgcgcccttcgaggttccaactggcgatgccgtccagctcgagtttaccatgctgtctcccttccatcgcctgaacttggaacccgtccgtcgaacagataacagtacagtttacagcacacgattcaccacccccgatcagcatggaatcttctccttccgagtgaactacaagcgcccgttcctcacgaacatcgaagaaaaacttgaggtgaccgttcgtcatttcgctcataacgagtacccccgaagctggaaaatcagcggtggatgggtctggattgcgggtctgtggtccgtcatcgctggcttcttagtattcgttgttgcatggctttactcagcgccttctgccgccgcactgaacacaaagaagacacaataa

SEQ ID NO: 14 - DNA, 1380 bp, Aspergillus fumigatus
atgcggtggtgcctcactcttctggcattctgcttcttggcagttgtacgtgcattaagtagctccggcagtcgtctgttggttgttttggaagatgccacagaaaaggaattatactcgaaattatgggctgacctagaaggatataacctcgacttcgaatcccccaagaatgacaagctcagcctgttcgaactcggagaccgagtctacgaccacatgcttctcctgcctcccaagtcaaagggctatggaccctcccttacccccaagaatatcattgatttcatgaacaaggacggtaacgtcctcctcgccttgtcgggcaagtccacaaccgccagcgctatcagctcgctgctattggagctcgatctccatctccctgtcgatcgttcctctgtcaccgtcgatcacttcaactacgatacactttctgcctccgataagcatgatgttctgctactccaccgaccaggcaagttgaggtccgataccaaggctttctttgatggcgagggcgttgtagcatttcccagagccgtcccccacaccctgggcgatgcaaaccctctcattgcgcctattctgcgagcgcccgccactgcgtatagttacaaccccaaggaggacgcgtcgtcagttgaggatgttgcagctacgggttcgcagttggctctggtctcggccatgcaggctagaaactccgctcggttcactctactgggatccgtggagagtctgcaggatcagtggttttctgcgactgtcaaggctcctggtgatgggaagcagatgaagacggtcaaccaggaattcgccaagcagcttactgcgtggacattcaaggaaaccggagtcctcaaggtcggaaagatcgagcatcatctggctgaagatggtgaaatcactcccgagaagctgaaccctaagatctatcgaataaagaatgaaactgtctttagcattgaactttccgaatacaactatgatcgttacgcgcccttcgaggttccaactggcgatgccgtccagctcgagtttaccatgctgtctcccttccatcgcctgaacttggaacccgtccgtcgaacagataacagtacagtttacagcacacgattcaccacccccgatcagcatggaatcttctccttccgagtgaactacaagcgcccgttcctcacgaacatcgaagaaaaacttgaggtgaccgttcgtcatttcgctcataacgagtacccccgaagctggaaaatcagcggtggatgggtctggattgcgggtctgtggtccgtcatcgctggcttcttagtattcgttgttgcatggctttactcagcgccttctgccgccgcactgaacacaaagaagacacaa

SEQ ID NO: 15 - protein, 460 aa, Aspergillus fumigatus
MRWCLTLLAFCFLAVVRALSSSGSRLLVVLEDATEKELYSKLWADLEGYNLDFESPKNDKLSLFELGDRVYDHMLLLPPKSKGYGPSLTPKNIIDFMNKDGNVLLALSGKSTTASAISSLLLELDLHLPVDRSSVTVDHFNYDTLSASDKHDVLLLHRPGKLRSDTKAFFDGEGVVAFPRAVPHTLGDANPLIAPILRAPATAYSYNPKEDASSVEDVAATGSQLALVSAMQARNSARFTLLGSVESLQDQWFSATVKAPGDGKQMKTVNQEFAKQLTAWTFKETGVLKVGKIEHHLAEDGEITPEKLNPKIYRIKNETVFSIELSEYNYDRYAPFEVPTGDAVQLEFTMLSPFHRLNLEPVRRTDNSTVYSTRFTTPDQHGIFSFRVNYKRPFLTNIEEKLEVTVRHFAHNEYPRSWKISGGWVWIAGLWSVIAGFLVFVVAWLYSAPSAAALNTKKTQ

SEQ ID NO: 16 - DNA, 2376 bp, Aspergillus fumigatus
atgtctcagtatcagcttactgtggccaccagggccaatcagccctatgtacttcctgtcctactggtcgcaacttccatcaacgaggcacgaccaagcccagtgatatcgatcacctatgaggatactgcggttcttcgtgaaggagacaaggccgtcgtgcaatacactggagctagcggtaatcctatctttggccttatcaatgctgttcaggaactccgcaaagacttccccttccttaacagcaaggatgagaagctggtaagaggcgccatggagccttactgctgatgagcactgataagtgatactaaccctccttttataggagaatgaatggctgtctcagttggaagcatttgctcctctagatttcaaggcccttgaccctgaattgcagcgcctcgatacccacctcctgctgagatctttcgtcgtcggttacgctctctcgacggccgacattgccctttggggtgccatccgaggcaaccgtgtcgcagttgccgcgatcaagaagggctcacttgtcaatgtgactcgttggttctatttcttggaggatctgtgcccgtgggccacatctacactggaggtcttgaaccaggctgtgcgagagaagaaggccgccaaggcgaaggagggagctagctacgacatcgctcttctcaacactgaaaaaggcgtggtgacaaggtttcctcccgagccttcaggttatcttcacatcggtcacgcaaaagctgcgctgctcaacgactactttgcccacgagaagtataatggcacccttcttgtccgctttgacgacacaaatccttcgaacgagaagctcgagttccaggacgcgatcattgaagatcttgctctcatgggcatcaagcccgacaagatgagctacaccagtgactactttgacgagctttaccagtacgcccttcaaatcatcaaggacggtaacgcctacgccgacgataccgagaaggaggtcatggctgagcagagaatgaatggaaaacccagcaagcgtcgtgatgcatccgtcgaggagaaccttgcccgcttcgaggagatgaagaagggtacccctgagggtctccgttggtgtatccgagccaagatgtctgtcgataaccccaacaaggccatgcgtgatcctgtcatttaccgctgcaaccctgcccctcaccaccgcactgggacgaagtggaagatctatcctacctatgacttcgcctgccctatcgtcgattcaattgagggtgtgactcatgccctcagaaccattgaataccgcgatcgcaaccctcagtaccagtggttcttggacacgctcaagcttcgccatgtccaaatctgggattttgctcgcatgaacttcattcgcaccttgctgtctaagagaaaacttaccaagctcgttaaccaaggtgtcgtctggggatgggatgagtaagtttacctttgcttgcaaacggattcttgtcttactaacgatgtcagtcctcgtttccccaccatccgagtaagtaacatgcctagtcatgcctgcgaatccccttattcatccggcattttttaccatctcacctacttcccatgtactgttaacttcccacgctaatcctttcatagggcatccgacgaaggggaatgactatccctgctctgagagaattcattcttaagcagggacccagcaagaacatcaccaaccttgactggaccctgatctgggcgaccaacaagaagtacattgatcctgtcgcacctcgtcacactgccattctcaagaaggatatggtcaaggcgatcgtcaagggaggcccggctacaccttacacggaagagaaacctaagcacggcaagaaccctgcagttggtatgaagaaggtggtttttggtaacacggtcattttcgaccagaaagatgccaagagcttcaagcaagatgaagagatcaccttgatgagctggggtaatgccattgtccgtaagatcgagaccgatcctacctcaggcatcgtcaaggagctggagctggagctccacctggaaggtgacttcaaaaagaccgagaagaaggtcacgtggctctctactgagggacaggacctaattcccgttgaattggtcgatttcgactatctcctcaacaaggacaccctgcaggaggacgacgtccttgaggatgtcctgaacaagaacaccgagttcagagaggacgctgttgctgactgcaacgtcgctgaactgaaagaaggtgacatcatccagtttgagcgcaagggctattaccgtgttgaccgggcctatgtaccgggcaagccggctgttttgttcaacattcccacgggcaagacgggcaaatag

SEQ ID NO: 17 - DNA, 2145 bp, Aspergillus fumigatus
atgtctcagtatcagcttactgtggccaccagggccaatcagccctatgtacttcctgtcctactggtcgcaacttccatcaacgaggcacgaccaagcccagtgatatcgatcacctatgaggatactgcggttcttcgtgaaggagacaaggccgtcgtgcaatacactggagctagcggtaatcctatctttggccttatcaatgctgttcaggaactccgcaaagacttccccttccttaacagcaaggatgagaagctggagaatgaatggctgtctcagttggaagcatttgctcctctagatttcaaggcccttgaccctgaattgcagcgcctcgatacccacctcctgctgagatctttcgtcgtcggttacgctctctcgacggccgacattgccctttggggtgccatccgaggcaaccgtgtcgcagttgccgcgatcaagaagggctcacttgtcaatgtgactcgttggttctatttcttggaggatctgtgcccgtgggccacatctacactggaggtcttgaaccaggctgtgcgagagaagaaggccgccaaggcgaaggagggagctagctacgacatcgctcttctcaacactgaaaaaggcgtggtgacaaggtttcctcccgagccttcaggttatcttcacatcggtcacgcaaaagctgcgctgctcaacgactactttgcccacgagaagtataatggcacccttcttgtccgctttgacgacacaaatccttcgaacgagaagctcgagttccaggacgcgatcattgaagatcttgctctcatgggcatcaagcccgacaagatgagctacaccagtgactactttgacgagctttaccagtacgcccttcaaatcatcaaggacggtaacgcctacgccgacgataccgagaaggaggtcatggctgagcagagaatgaatggaaaacccagcaagcgtcgtgatgcatccgtcgaggagaaccttgcccgcttcgaggagatgaagaagggtacccctgagggtctccgttggtgtatccgagccaagatgtctgtcgataaccccaacaaggccatgcgtgatcctgtcatttaccgctgcaaccctgcccctcaccaccgcactgggacgaagtggaagatctatcctacctatgacttcgcctgccctatcgtcgattcaattgagggtgtgactcatgccctcagaaccattgaataccgcgatcgcaaccctcagtaccagtggttcttggacacgctcaagcttcgccatgtccaaatctgggattttgctcgcatgaacttcattcgcaccttgctgtctaagagaaaacttaccaagctcgttaaccaaggtgtcgtctggggatgggatgatcctcgtttccccaccatccgaggcatccgacgaaggggaatgactatccctgctctgagagaattcattcttaagcagggacccagcaagaacatcaccaaccttgactggaccctgatctgggcgaccaacaagaagtacattgatcctgtcgcacctcgtcacactgccattctcaagaaggatatggtcaaggcgatcgtcaagggaggcccggctacaccttacacggaagagaaacctaagcacggcaagaaccctgcagttggtatgaagaaggtggtttttggtaacacggtcattttcgaccagaaagatgccaagagcttcaagcaagatgaagagatcaccttgatgagctggggtaatgccattgtccgtaagatcgagaccgatcctacctcaggcatcgtcaaggagctggagctggagctccacctggaaggtgacttcaaaaagaccgagaagaaggtcacgtggctctctactgagggacaggacctaattcccgttgaattggtcgatttcgactatctcctcaacaaggacaccctgcaggaggacgacgtccttgaggatgtcctgaacaagaacaccgagttcagagaggacgctgttgctgactgcaacgtcgctgaactgaaagaaggtgacatcatccagtttgagcgcaagggctattaccgtgttgaccgggcctatgtaccgggcaagccggctgttttgttcaacattcccacgggcaagacgggcaaa

SEQ ID NO: 18 - protein, 715 aa, Aspergillus fumigatus
MSQYQLTVATRANQPYVLPVLLVATSINEARPSPVISITYEDTAVLREGDKAVVQYTGASGNPIFGLINAVQELRKDFPFLNSKDEKLENEWLSQLEAFAPLDFKALDPELQRLDTHLLLRSFVVGYALSTADIALWGAIRGNRVAVAAIKKGSLVNVTRWFYFLEDLCPWATSTLEVLNQAVREKKAAKAKEGASYDIALLNTEKGVVTRFPPEPSGYLHIGHAKAALLNDYFAHEKYNGTLLVRFDDTNPSNEKLEFQDAIIEDLALMGIKPDKMSYTSDYFDELYQYALQIIKDGNAYADDTEKEVMAEQRMNGKPSKRRDASVEENLARFEEMKKGTPEGLRWCIRAKMSVDNPNKAMRDPVIYRCNPAPHHRTGTKWKIYPTYDFACPIVDSIEGVTHALRTIEYRDRNPQYQWFLDTLKLRHVQIWDFARMNFIRTLLSKRKLTKLVNQGVVWGWDDPRFPTIRGIRRRGMTIPALREFILKQGPSKNITNLDWTLIWATNKKYIDPVAPRHTAILKKDMVKAIVKGGPATPYTEEKPKHGKNPAVGMKKVVFGNTVIFDQKDAKSFKQDEEITLMSWGNAIVRKIETDPTSGIVKELELELHLEGDFKKTEKKVTWLSTEGQDLIPVELVDFDYLLNKDTLQEDDVLEDVLNKNTEFREDAVADCNVAELKEGDIIQFERKGYYRVDRAYVPGKPAVLFNIPTGKTGK

SEQ ID NO: 19 - DNA, 2639 bp, Aspergillus fumigatus
atgtcgccatcaatatcctacatttcaggccagcttaggcagctaatatactatcatctcgataacaatttgtgccgtaatgcgctgttcctcgccggtcgtttacatgcttacgagccccgaacggcggaagcgtcgtatttactcgctctctgccatcttcagaacgggcaagtcaaagccgcatacgattacagcaggaattttggatcgagaggcacccacctcggctgctcctatgtcttcgcgcaagcgtgcttggacctaggaaagtatctggaaggtatcacagcgttagagcggagtaaaggcctttgggcttcgaagaaccactggagtaagtattccatgcgctgatacttgcagtcgacctgcatttatttcgttgctttctgttactgacacatttccaccttgtgctctagataagcacagtgagacgcgaagacaacatctgccggatgcagccgcagtattctgcctgttaggtaaattatggcatgcgcataaggacatcaacaaagctgtggaatgctatgttgaatctctgaagctgaatcccttcatgtgggatgcgttccaagggttgtgcgacaccggtaagctttggagaataaacctaatacatctagccatggctaattgtgttctaccgccaaggagtcaatgtccgcgtgtcaaacatctacaagttgaattctgaattgctggccgtattgtcttcatcgccacaggcggatgctgagccaatatccgataagtctgcacacacgaatgggccactgcaagcgcaggcgaatgttaatccaagttccgatccttttgcctcgactacttctcgcagtgattcaggtaccagccatgggagctctgccttgtgggaaaaactaaatggaagcacagtaagcgtggcgtcatcgggagtgccagcatcaatcgtgcatgaaggagccgaaacgccgagtggtcaaagcagcggatctgatgagttccggttagctaacggaatgaacggcgcggatgcttcttgggaccctcctttagctcctgcaaggaaaaacagaacgatccaggcaataagcggcgagtatccaatggaccctcctcccaagatgaaacccactgggatccgaccaaggacaaggaccaggactgagccggaggaccaaatttcagcccagatagaccgggaggcaacaaatgcgcccagggtcggggaccgcaaacgaactgtttctggtcaggtagcgcatccaccgacgtcacaacccacagaaccaggagcaccccagcggcggagtgtgcgactcttcaaccagattaaacccacgaccagcaaattgtcggcgtccgcgctgggagtcaaggatgctagagaagtcaagaaagcgaaagccacaggtacgaaggggcgtacgacaaccaccaccatgggacgagtagtgagtggcagccgaaaacatgccagcgaacatcatgatgcagatggtaaagacggacggtcggtaccgtccgcccacactcacgccatctccaaaggcgctgctcaagaaagatcgaaagaaatcgaggcgttgacctggctgctggagctattctcgaaacttgcttctggattctttgccttgtgtcgctaccgatgcccagagtcaatccagatcttcaattcgctctctcaaggccaacgggaaacaccgtgggttctcgctcagattggacgagcgtactatgagcaggctatgtattccgaggcagaaaagtacttctaccgtgtgaagaccatggcaccctcgcgcttggaagacatggagatctactcgactgtcctttggcatctgaagaacgatgttgagttagcctatttggcgcatgagttgatggaaacagaccgcctgtcgccacaggcgtggtgcgccatcggtaattcgttttcccaccagcgagatcatgaccaggccttgaagtgctttaagcgggcaacccagctggatcctcagtttgcctacgggtttactcttcaagggcacgagtatgttgccaacgaagaatacgacaaggcgcttgatgcataccgtcacggtatcagcgcggatagtcggcattacaatgcttggtacggactgggcacggtttatgacaaaatgggcaaactggactttgccgaacaacacttccggaatgcggcaagcattaacccgaccaacgcagttttgatctgctgcattggattggtactggaaaaaatgaacaaccctaaagcggctctcgtgcaatatggtcgcgcttgttccttggcacctcattccgtactcgcgcgattccgcaaggcccgcgcattgatgaagctccaggagctcaaattagctctgtctgagttgaagattctcaaagacatggctccagacgaagctaacgtgcattatctgttgggtaagctctacaaaatgcttcacgacaaagccaatgccattaagcacttcacaactgctttgaacttggatccaaaggtatgcctatcacttccattccgtcaaattaccagacaatactaacggattcggttacaggcagcacaatacatcaaggatgccatggaatctcttgacgatgacgaggaggatgatgaggacatgagctga

SEQ ID NO: 20 - DNA, 2427 bp, Aspergillus fumigatus
atgtcgccatcaatatcctacatttcaggccagcttaggcagctaatatactatcatctcgataacaatttgtgccgtaatgcgctgttcctcgccggtcgtttacatgcttacgagccccgaacggcggaagcgtcgtatttactcgctctctgccatcttcagaacgggcaagtcaaagccgcatacgattacagcaggaattttggatcgagaggcacccacctcggctgctcctatgtcttcgcgcaagcgtgcttggacctaggaaagtatctggaaggtatcacagcgttagagcggagtaaaggcctttgggcttcgaagaaccactggaataagcacagtgagacgcgaagacaacatctgccggatgcagccgcagtattctgcctgttaggtaaattatggcatgcgcataaggacatcaacaaagctgtggaatgctatgttgaatctctgaagctgaatcccttcatgtgggatgcgttccaagggttgtgcgacaccggagtcaatgtccgcgtgtcaaacatctacaagttgaattctgaattgctggccgtattgtcttcatcgccacaggcggatgctgagccaatatccgataagtctgcacacacgaatgggccactgcaagcgcaggcgaatgttaatccaagttccgatccttttgcctcgactacttctcgcagtgattcaggtaccagccatgggagctctgccttgtgggaaaaactaaatggaagcacagtaagcgtggcgtcatcgggagtgccagcatcaatcgtgcatgaaggagccgaaacgccgagtggtcaaagcagcggatctgatgagttccggttagctaacggaatgaacggcgcggatgcttcttgggaccctcctttagctcctgcaaggaaaaacagaacgatccaggcaataagcggcgagtatccaatggaccctcctcccaagatgaaacccactgggatccgaccaaggacaaggaccaggactgagccggaggaccaaatttcagcccagatagaccgggaggcaacaaatgcgcccagggtcggggaccgcaaacgaactgtttctggtcaggtagcgcatccaccgacgtcacaacccacagaaccaggagcaccccagcggcggagtgtgcgactcttcaaccagattaaacccacgaccagcaaattgtcggcgtccgcgctgggagtcaaggatgctagagaagtcaagaaagcgaaagccacaggtacgaaggggcgtacgacaaccaccaccatgggacgagtagtgagtggcagccgaaaacatgccagcgaacatcatgatgcagatggtaaagacggacggtcggtaccgtccgcccacactcacgccatctccaaaggcgctgctcaagaaagatcgaaagaaatcgaggcgttgacctggctgctggagctattctcgaaacttgcttctggattctttgccttgtgtcgctaccgatgcccagagtcaatccagatcttcaattcgctctctcaaggccaacgggaaacaccgtgggttctcgctcagattggacgagcgtactatgagcaggctatgtattccgaggcagaaaagtacttctaccgtgtgaagaccatggcaccctcgcgcttggaagacatggagatctactcgactgtcctttggcatctgaagaacgatgttgagttagcctatttggcgcatgagttgatggaaacagaccgcctgtcgccacaggcgtggtgcgccatcggtaattcgttttcccaccagcgagatcatgaccaggccttgaagtgctttaagcgggcaacccagctggatcctcagtttgcctacgggtttactcttcaagggcacgagtatgttgccaacgaagaatacgacaaggcgcttgatgcataccgtcacggtatcagcgcggatagtcggcattacaatgcttggtacggactgggcacggtttatgacaaaatgggcaaactggactttgccgaacaacacttccggaatgcggcaagcattaacccgaccaacgcagttttgatctgctgcattggattggtactggaaaaaatgaacaaccctaaagcggctctcgtgcaatatggtcgcgcttgttccttggcacctcattccgtactcgcgcgattccgcaaggcccgcgcattgatgaagctccaggagctcaaattagctctgtctgagttgaagattctcaaagacatggctccagacgaagctaacgtgcattatctgttgggtaagctctacaaaatgcttcacgacaaagccaatgccattaagcacttcacaactgctttgaacttggatccaaaggcagcacaatacatcaaggatgccatggaatctcttgacgatgacgaggaggatgatgaggacatgagc

SEQ ID NO: 21 - protein, 809 aa, Aspergillus fumigatus
MSPSISYISGQLRQLIYYHLDNNLCRNALFLAGRLHAYEPRTAEASYLLALCHLQNGQVKAAYDYSRNFGSRGTHLGCSYVFAQACLDLGKYLEGITALERSKGLWASKNHWNKHSETRRQHLPDAAAVFCLLGKLWHAHKDINKAVECYVESLKLNPFMWDAFQGLCDTGVNVRVSNIYKLNSELLAVLSSSPQADAEPISDKSAHTNGPLQAQANVNPSSDPFASTTSRSDSGTSHGSSALWEKLNGSTVSVASSGVPASIVHEGAETPSGQSSGSDEFRLANGMNGADASWDPPLAPARKNRTIQAISGEYPMDPPPKMKPTGIRPRTRTRTEPEDQISAQIDREATNAPRVGDRKRTVSGQVAHPPTSQPTEPGAPQRRSVRLFNQIKPTTSKLSASALGVKDAREVKKAKATGTKGRTTTTTMGRVVSGSRKHASEHHDADGKDGRSVPSAHTHAISKGAAQERSKEIEALTWLLELFSKLASGFFALCRYRCPESIQIFNSLSQGQRETPWVLAQIGRAYYEQAMYSEAEKYFYRVKTMAPSRLEDMEIYSTVLWHLKNDVELAYLAHELMETDRLSPQAWCAIGNSFSHQRDHDQALKCFKRATQLDPQFAYGFTLQGHEYVANEEYDKALDAYRHGISADSRHYNAWYGLGTVYDKMGKLDFAEQHFRNAASINPTNAVLICCIGLVLEKMNNPKAALVQYGRACSLAPHSVLARFRKARALMKLQELKLALSELKILKDMAPDEANVHYLLGKLYKMLHDKANAIKHFTTALNLDPKAAQYIKDAMESLDDDEEDDEDMS

SEQ ID NO: 22 - DNA, 1836 bp, Aspergillus fumigatus
gcccactacggtttacaatcgggaattcccgatgaggtggactttgcattgtatcaccttgttcagatttcgaatcaacgatgggataaattcaagtttgagggtttccccttgcttgcggagaacctcatggcaaaggccctggatatctcccttgtcacaaccggggtgaagtgggagcttcagtatgatgttcttcaactcagtgatcgcgtcaatgagctgaactcgctacatggcacacgagatctgttggagaagatcaaacaaatgccagttacattgccggaagacaccctcgagacgtacgaattcaaccaccttctgcgcaacgttaaagaagcgaccctggtactacgcaatatggtccttctgaaagagaatgcctactatgtgtcacggtacgcgaaaggcctgctccgagacttcctcgtcattatgatcaacttgcccaatcagcctcgtctcaacgagatcaagaacgacgctttggacattgcagaggaggtcaccaagtttatgaagaccgatccggaagatccactttggatctcacttctcaattgtctcgggtcgtcagatcgtgctcacgtggtccgcgcactctgggctctcacccatttctccactgaattagacgagccagaggcgaaccgggcaatggaacggataccaaaagagactttgcagcagctctactttcacactcttctcgacttggacaaagatattctcagtggtgcattggacttctggtaccagtatacactttcgtccgagaacattgagactttgattgaggtcttcaacttgcctaccgtcttcgtcccccggatggtcgcactgttaacgcacgaaggccgaccgaacaagaaggaaactgtgttgcaagaagaaaaggtggcccccccaccgtcggatatccctcgtgtaccgcccgagctcatgaaagagctgatggagctttcggagcctgaacggagctcgcgttggctccggtgctgctttgtggaggacctcgagtgcgagatcacccaaattgccttgtggcaggcgtatcagagcagatttgcagaccctcgccttcctggtggcggtgttctccctgcggctgaatttatcaaaaatgtcagtacgactttcacgaacgcgcaagcacaggtgatcaatggccctggtgcagccacgaaattcatcatcaaaggcattcggcccctggagaccgcctataccttcgagggctttccctacatttactgcaagtgggcggacaactcgaagccaagcaagacgtgtcagcgtgctttcaagtcgccggcagaacttcgccatcacgtcttcacggaacacatgaacctcaagcctactgaaacgccgggacactataacctggagaaggcggagtcgcccgttcatacctgcctttgggacaactgcacgaaattccggtcgtctggtccgagtgccaatactgcaatggtcgctgggcacgtctccgcacatctgcccgaggaacgtgcgccagatgcggagccgccgacatccaaacgtgcggttctccaagagcgcatcgtccgcaaatggtactacctggacactccagtcaatgagcgaggcgagccgtttggagtggcctacaaggcggcgttagtattgcgtaaccttgcccgaaacctgcctacgggtattgcaccgcagtacaacgggctttcatggaagaaagccgtcttccttagtcatcgtccaaagatcatcgaagcatgggaccgcaaccgctcattgcgcaaggaacttaccgagttgatcatggtaatagaaaaagaggattattactga

SEQ ID NO: 23 - DNA, 1836 bp, Aspergillus fumigatus; identical to SEQ ID NO: 22

SEQ ID NO: 24 - protein, 611 aa, Aspergillus fumigatus
AHYGLQSGIPDEVDFALYHLVQISNQRWDKFKFEGFPLLAENLMAKALDISLVTTGVKWELQYDVLQLSDRVNELNSLHGTRDLLEKIKQMPVTLPEDTLETYEFNHLLRNVKEATLVLRNMVLLKENAYYVSRYAKGLLRDFLVIMINLPNQPRLNEIKNDALDIAEEVTKFMKTDPEDPLWISLLNCLGSSDRAHVVRALWALTHFSTELDEPEANRAMERIPKETLQQLYFHTLLDLDKDILSGALDFWYQYTLSSENIETLIEVFNLPTVFVPRMVALLTHEGRPNKKETVLQEEKVAPPPSDIPRVPPELMKELMELSEPERSSRWLRCCFVEDLECEITQIALWQAYQSRFADPRLPGGGVLPAAEFIKNVSTTFTNAQAQVINGPGAATKFIIKGIRPLETAYTFEGFPYIYCKWADNSKPSKTCQRAFKSPAELRHHVFTEHMNLKPTETPGHYNLEKAESPVHTCLWDNCTKFRSSGPSANTAMVAGHVSAHLPEERAPDAEPPTSKRAVLQERIVRKWYYLDTPVNERGEPFGVAYKAALVLRNLARNLPTGIAPQYNGLSWKKAVFLSHRPKIIEAWDRNRSLRKELTELIMVIEKEDYY

SEQ ID NO: 25 - DNA, 1542 bp, Aspergillus fumigatus
atggtctacatcggcatccccaagaactacacggcttcgccgtcttcctttgccggaactccgtccttgacgatcaattacgaggcaacgcaggatcttgattctaccaatgcttttgaaggtttgttgacgccggtgacacgtgtgagagcagcgctggacagaggctgacagtctacaggtccagagaaactcttggaggtgtggttcgcgccttccgctcaggaattaggtccagcgcagcccgccggtctgaaggctgttccggaggagatctggaaggacatgttggatctcgtcaattgccaggtcctctcgattgtttcgtcagaggatgtggacgcctacctgctctccgagtctagcatgttcgtttggcctcacaaactcatcttgaagacttgtggtaccaccactcttctgtctggtctcccacgcattctcgagattgccgctttgttcggtggcttccccaagtctaccgccccttctcgcggaatctccgtcgccgctgcgccctaccgcgtcttctacagccgcaagaacttcctgttccccgaccgccagcggggccctcaccgcagctggagagatgaagtgcggactatggataagctcttcctcaacggcagcgcctacatgattggcaagatgaatggcgagcactggtacttgtacctgactgaacctcataccatgctcaccccgccaacgagcccgggagccaagaccgagtttacggaaacggagaccaaggtcctcagtgtaccccagggcgctgctctgcagactgattcggaggatgagactttggaagtcttgatgaccgacttggatgaggagaacgccaagcagttctacctcgagaatgccactgccgttgcggagaaccgttatcgcaactcaaattcggagaagagtggccatgttgatgttttcagcaacacttcctccgatatcagcgattttgactccgacggaagccaggttctgcctccagagttgactaccgagggtcacgcgctcggaaccgtggtctctgaagcctgtggactttcctctgtgtatcctaaggagaagtatcccgattcgcgcatcgatgcctacctgtttacaccatgcggcttctccgccaacggcgtgattccgcctcctgagggaaaagctggaacccactacttcacagtacacgtcactccagagccgcactgttcatatgcgtcctttgagaccaacgtaccgcactcgcagaacggccagactaccgctggaatcatcaagcaagtggtcgacatcttcaagcctggtcgcttcagcgtgactctcttcgaggccaagccagcgctgagccaggtcgaagacgagtggaaggaagccaagtacctggccgctcgtcggaccgccaaaatggaacatgtggagggatatcgccgagtggaccggattgtccacgacctcgacggctatgagcttgtcttccgctattatgaacgcctggactggaaagggggggcccctcggctgggagaggagagatcttga

SEQ ID NO: 26 - DNA, 1479 bp, Aspergillus fumigatus
atggtctacatcggcatccccaagaactacacggcttcgccgtcttcctttgccggaactccgtccttgacgatcaattacgaggcaacgcaggatcttgattctaccaatgcttttgaaggtccagagaaactcttggaggtgtggttcgcgccttccgctcaggaattaggtccagcgcagcccgccggtctgaaggctgttccggaggagatctggaaggacatgttggatctcgtcaattgccaggtcctctcgattgtttcgtcagaggatgtggacgcctacctgctctccgagtctagcatgttcgtttggcctcacaaactcatcttgaagacttgtggtaccaccactcttctgtctggtctcccacgcattctcgagattgccgctttgttcggtggcttccccaagtctaccgccccttctcgcggaatctccgtcgccgctgcgccctaccgcgtcttctacagccgcaagaacttcctgttccccgaccgccagcggggccctcaccgcagctggagagatgaagtgcggactatggataagctcttcctcaacggcagcgcctacatgattggcaagatgaatggcgagcactggtacttgtacctgactgaacctcataccatgctcaccccgccaacgagcccgggagccaagaccgagtttacggaaacggagaccaaggtcctcagtgtaccccagggcgctgctctgcagactgattcggaggatgagactttggaagtcttgatgaccgacttggatgaggagaacgccaagcagttctacctcgagaatgccactgccgttgcggagaaccgttatcgcaactcaaattcggagaagagtggccatgttgatgttttcagcaacacttcctccgatatcagcgattttgactccgacggaagccaggttctgcctccagagttgactaccgagggtcacgcgctcggaaccgtggtctctgaagcctgtggactttcctctgtgtatcctaaggagaagtatcccgattcgcgcatcgatgcctacctgtttacaccatgcggcttctccgccaacggcgtgattccgcctcctgagggaaaagctggaacccactacttcacagtacacgtcactccagagccgcactgttcatatgcgtcctttgagaccaacgtaccgcactcgcagaacggccagactaccgctggaatcatcaagcaagtggtcgacatcttcaagcctggtcgcttcagcgtgactctcttcgaggccaagccagcgctgagccaggtcgaagacgagtggaaggaagccaagtacctggccgctcgtcggaccgccaaaatggaacatgtggagggatatcgccgagtggaccggattgtccacgacctcgacggctatgagcttgtcttccgctattatgaacgcctggactggaaagggggggcccctcggctgggagaggagagatct

SEQ ID NO: 27 - protein, 493 aa, Aspergillus fumigatus
MVYIGIPKNYTASPSSFAGTPSLTINYEATQDLDSTNAFEGPEKLLEVWFAPSAQELGPAQPAGLKAVPEEIWKDMLDLVNCQVLSIVSSEDVDAYLLSESSMFVWPHKLILKTCGTTTLLSGLPRILEIAALFGGFPKSTAPSRGISVAAAPYRVFYSRKNFLFPDRQRGPHRSWRDEVRTMDKLFLNGSAYMIGKMNGEHWYLYLTEPHTMLTPPTSPGAKTEFTETETKVLSVPQGAALQTDSEDETLEVLMTDLDEENAKQFYLENATAVAENRYRNSNSEKSGHVDVFSNTSSDISDFDSDGSQVLPPELTTEGHALGTVVSEACGLSSVYPKEKYPDSRIDAYLFTPCGFSANGVIPPPEGKAGTHYFTVHVTPEPHCSYASFETNVPHSQNGQTTAGIIKQVVDIFKPGRFSVTLFEAKPALSQVEDEWKEAKYLAARRTAKMEHVEGYRRVDRIVHDLDGYELVFRYYERLDWKGGAPRLGEERS

SEQ ID NO: 28 - DNA, 637 bp, Aspergillus fumigatus
atgggtcgcgttagaaccaaggtaagttacagatgaagcatcatgagttatcttcaaaaaagccccaaaagagtatcatttctgacgaaatgggtttttcttcaatagacagtcaagaggtccgccaaggtcatcatcgagcgctactaccccaagttgacgctcgactttgagaccaacaagcgtctttgcgatgagatcgctatcattgcctccaagcgccttcgcaacaaggtgggcaatccatcactgagccgtacaacagtcggaatttgacttgctgacgaaaactagattgctggttacaccacccaccttatgaagcgtatccagcgtggccctgtccgcggtatctctttcaagctgcaggaggaggagcgtgagcgcaaggatcagtacgttcctgaggtttccgctctggatgtttcccagaccgagtccggccagctcgatgtcgatgccgacaccaaggaccttctcaagtccatgggcgtaagttctgttctcaacgcggttgttcgtggttttaaagcagtccgttaacttatattgcccactacagttcgacaatctcaaggtcaacgttgtcaacgtctcccaacatcaggttcaggagcgcccccgccgcttccggtag

SEQ ID NO: 29 - DNA, 417 bp, Aspergillus fumigatus
atgggtcgcgttagaaccaagacagtcaagaggtccgccaaggtcatcatcgagcgctactaccccaagttgacgctcgactttgagaccaacaagcgtctttgcgatgagatcgctatcattgcctccaagcgccttcgcaacaagattgctggttacaccacccaccttatgaagcgtatccagcgtggccctgtccgcggtatctctttcaagctgcaggaggaggagcgtgagcgcaaggatcagtacgttcctgaggtttccgctctggatgtttcccagaccgagtccggccagctcgatgtcgatgccgacaccaaggaccttctcaagtccatgggcttcgacaatctcaaggtcaacgttgtcaacgtctcccaacatcaggttcaggagcgcccccgccgcttccgg

SEQ ID NO: 30 - protein, 139 aa, Aspergillus fumigatus
MGRVRTKTVKRSAKVIIERYYPKLTLDFETNKRLCDEIAIIASKRLRNKIAGYTTHLMKRIQRGPVRGISFKLQEEERERKDQYVPEVSALDVSQTESGQLDVDADTKDLLKSMGFDNLKVNVVNVSQHQVQERPRRFR

SEQ ID NO: 31 - DNA, 1035 bp, Aspergillus fumigatus
atggcggttggaaagtatgccaattcacttctattattgttctgaacgcttttagcatgtgtctggatacggtggtttacaggtactgatccgggaacaggaacaagcgcttgtcgaagggcaagaagggtgttaagaagaggaccgttgatcctttctccaggaaggacgaatactctgttaaggtatgtcgacgtggactgtgtaagtcgaccgcagctaatctatatcaggcgccttccactttccagatcagagagtatgttgcacgcatatgatgtcgaatgcaggataaaggcgattcacaatggtagtggagattatgctgactgaattatagtgtcgggaagactctggtcaaccgcaccagtggtctcaagaacgccaatgactccctgaagggtcgaattttcgaggtctcgctggctgacctgcagaatgatgaagaccatgctttccgcaaggttaagcttcgtgtggacgaggttcagggcaagaactgtttgaccaacttccacggtcttgatttcacaaccgacaaattgcgatccctcgtgcgcaagtggcagtcgctgatcgaagccatgtcactgtgaagacgaccgatgattatctccttcggctttttgctatcgccttcaccaagagacgcccgaaccagattaagaagaccacatatgctcgttcttctcaaatccgtgccatccgcaagaagatgattgaaatcatgcagagggaggcagccagctgctctctcgctcagctcactcacaagctcattcctgaggtcattggtcgtgagatcgagaaggctacccagggaatctatcctttgcagaatgtgtgtgaccctgttattcttactcgggatgaagactaactgcaatctaggtccatattcgcaaggtcaagcttcttaaggctcccaagttcgacctgggtgcactgctgaatctgcacggtgaatctacaaccgatgataagggccacaaggtcgagagagagttcaaggagcaggttctcgaaagcgtttaa

SEQ ID NO: 32 - DNA, 768 bp, Aspergillus fumigatus
atggcggttggaaagaacaagcgcttgtcgaagggcaagaagggtgttaagaagaggaccgttgatcctttctccaggaaggacgaatactctgttaaggcgccttccactttccagatcagagatgtcgggaagactctggtcaaccgcaccagtggtctcaagaacgccaatgactccctgaagggtcgaattttcgaggtctcgctggctgacctgcagaatgatgaagaccatgctttccgcaaggttaagcttcgtgtggacgaggttcagggcaagaactgtttgaccaacttccacggtcttgatttcacaaccgacaaattgcgatccctcgtgcgcaagtggcagtcgctgatcgaagccaatgtcactgtgaagacgaccgatgattatctccttcggctttttgctatcgccttcaccaagagacgcccgaaccagattaagaagaccacatatgctcgttcttctcaaatccgtgccatccgcaagaagatgattgaaatcatgcagagggaggcagccagctgctctctcgctcagctcactcacaagctcattcctgaggtcattggtcgtgagatcgagaaggctacccagggaatctatcctttgcagaatgtccatattcgcaaggtcaagcttcttaaggctcccaagttcgacctgggtgcactgctgaatctgcacggtgaatctacaaccgatgataagggccacaaggtcgagagagagttcaaggagcaggttctcgaaagcgtt

SEQ ID NO: 33 - protein, 256 aa, Aspergillus fumigatus
MAVGKNKRLSKGKKGVKKRTVDPFSRKDEYSVKAPSTFQIRDVGKTLVNRTSGLKNANDSLKGRIFEVSLADLQNDEDHAFRKVKLRVDEVQGKNCLTNFHGLDFTTDKLRSLVRKWQSLIEANVTVKTTDDYLLRLFAIAFTKRRPNQIKKTTYARSSQIRAIRKKMIEIMQREAASCSLAQLTHKLIPEVIGREIEKATQGIYPLQNVHIRKVKLLKAPKFDLGALLNLHGESTTDDKGHKVEREFKEQVLESV

SEQ ID NO: 34 - DNA, 614 bp, Aspergillus fumigatus
cctgtcggagatgatcaaaggcagcacctcgaattttcgaggaacactgcgaatagtttcaatcatgtatatggacccattttcccgtcaccagaagcaattatatgtaagtggtttttgcttctggcggaacggtcctgtgttgggaaattgacggctatcaatagcgcctgctaaacgggttatgtccctcaaagaaccgacgttgaaaatgtccaagtcccatgccgacagacgctcaaggatcattcttacggattcgcccgcagaaatctccaaaaagatcaatgctgcgctcacagactcggaattaaccattacatatgacccagtccgtcgacctggagtggcgaatttaatagagatcttgagtcacttcgatggacgaacttgcgatgagattgccatggaataccgttcagccagtcttcgcgctctaaaggaacatctggccagaacgttgtccaatcatcttgagccaataagagagaagtatctctcacttgtaggagatcagactgactaccttgattctatagcagaacagggttctgaagccgcgcgggccaacgctgaattgacaatggagcaagtcaaagtcgctatgggcttaa

SEQ ID NO: 35 - DNA, 552 bp, Aspergillus fumigatus
cctgtcggagatgatcaaaggcagcacctcgaattttcgaggaacactgcgaatagtttcaatcatgtatatggacccattttcccgtcaccagaagcaattatatcgcctgctaaacgggttatgtccctcaaagaaccgacgttgaaaatgtccaagtcccatgccgacagacgctcaaggatcattcttacggattcgcccgcagaaatctccaaaaagatcaatgctgcgctcacagactcggaattaaccattacatatgacccagtccgtcgacctggagtggcgaatttaatagagatcttgagtcacttcgatggacgaacttgcgatgagattgccatggaataccgttcagccagtcttcgcgctctaaaggaacatctggccagaacgttgtccaatcatcttgagccaataagagagaagtatctctcacttgtaggagatcagactgactaccttgattctatagcagaacagggttctgaagccgcgcgggccaacgctgaattgacaatggagcaagtcaaagtcgctatgggctta

SEQ ID NO: 36 - protein, 184 aa, Aspergillus fumigatus
PVGDDQRQHLEFSRNTANSFNHVYGPIFPSPEAIISPAKRVMSLKEPTLKMSKSHADRRSRIILTDSPAEISKKINAALTDSELTITYDPVRRPGVANLIEILSHFDGRTCDEIAMEYRSASLRALKEHLARTLSNHLEPIREKYLSLVGDQTDYLDSIAEQGSEAARANAELTMEQVKVAMGL

SEQ ID NO: 37 - DNA, 819 bp, Aspergillus fumigatus
atggcaacttcgactgggaccggatgggctcagctccggcagcaagcccgttcgcttgagactcaggtacggaactcgaaactacgctataatgaggctttactcgtgatttggatgttgacaataatgttcctagaccgagagtctgtttcacacctatgcgcagtatgcatcgatgacgaagctgcctccgaaaccctcagaagaagaacaacggattgaatcgcaactgaaggatcttcttgaaaaggtgtgcactttgaggccctctagtccagcccaacagacgatcatgctgacacgatccgatcatagcgtgaagccctcatctcccagctctcccgtctccttgactccgaagccactcttaccgcatctgccctgaaacagagcaatcttgcccgcaatcgcgaagtcctccaggatcatcgccgcgaattgcagcgcctgaacgccgcaatcgccgagtcccgcgaccgagccaatcttctgtctaacgtccgctccgacattgatgcctaccgcaattcaaaccccgccgcggctgaggcagactacatgctcgaggagcggggtcgtatagatgaaagccataacatgatagatggtgtcctaagccaggcgtatgcaatcaacgagagttttgggctacaacgtgaaaccctggccagcatcaatcgccgtatcgtcggtgctgccaataaggtaccaggaatgaatgcattgattggtaagattgggacgaagaggagacgtgacgcaatcatcttgggggctttcatcggcttttgtttcttgatggtgttcttcttccgatga

SEQ ID NO: 38 - DNA, 681 bp, Aspergillus fumigatus
atggcaacttcgactgggaccggatgggctcagctccggcagcaagcccgttcgcttgagactcagaccgagagtctgtttcacacctatgcgcagtatgcatcgatgacgaagctgcctccgaaaccctcagaagaagaacaacggattgaatcgcaactgaaggatcttcttgaaaagcgtgaagccctcatctcccagctctcccgtctccttgactccgaagccactcttaccgcatctgccctgaaacagagcaatcttgcccgcaatcgcgaagtcctccaggatcatcgccgcgaattgcagcgcctgaacgccgcaatcgccgagtcccgcgaccgagccaatcttctgtctaacgtccgctccgacattgatgcctaccgcaattcaaaccccgccgcggctgaggcagactacatgctcgaggagcggggtcgtatagatgaaagccataacatgatagatggtgtcctaagccaggcgtatgcaatcaacgagagttttgggctacaacgtgaaaccctggccagcatcaatcgccgtatcgtcggtgctgccaataaggtaccaggaatgaatgcattgattggtaagattgggacgaagaggagacgtgacgcaatcatcttgggggctttcatcggcttttgtttcttgatggtgttcttcttccga

SEQ ID NO: 39 - protein, 227 aa, Aspergillus fumigatus
MATSTGTGWAQLRQQARSLETQTESLFHTYAQYASMTKLPPKPSEEEQRIESQLKDLLEKREALISQLSRLLDSEATLTASALKQSNLARNREVLQDHRRELQRLNAAIAESRDRANLLSNVRSDIDAYRNSNPAAAEADYMLEERGRIDESHNMIDGVLSQAYAINESFGLQRETLASINRRIVGAANKVPGMNALIGKIGTKRRRDAIILGAFIGFCFLMVFFFR

SEQ ID NO: 40 - DNA, 1601 bp, Aspergillus fumigatus
atgtcacaaaatcgacctggggtgttctcgaatctgcgcatgggtggtaaggaacatccaaatgctgagtccaattgttcagaaaacattacccaggagcctgtggaactaactgctttgctttccgaccatacagaagtcgtccgcgagaaggtccaggatggactgacaggggaaactaaggagatttcgtactcacaatgtaaaatcgtcggcaatggatcgtttggtgtcgtctttcagacgaaaatgatgccaagcggcgaggatgctgccattaagagggtccttcaagacaagcgcttcaaagtatgtgtacattataagggcaattgccctcgctgcccaacccaaagatactgtcgctgacgagataccagaatcgagaactgcagattatgcggattgttcgccatcctaacatcgtagaattgaaagccttctattactcgaacggcgagagggtatgcgactctcctttgtctccccattcgttctagtttgccgtttgctgactaccctaccattgtctttcacagaaggatgaagtgtacctaaacctcgttctcgaatacgtaccagaaaccgtgtatcgggcgtcgcggtactttaataaactcaaaacgactatgccaatgttggaagtcaagctgtatatctatcaattgttccgttccctggcatacatccattcacaaggcatctgccaccgtgacatcaagccccagaatctcttacttgatccatccaccggcatcctcaaactctgcgactttggttcggccaagattctggtagagaatgagcccaacgtttcctatatctgttcccgctactatcgtgcgccggaattgatctttggcgccactaattacacaacaaagatcggtaagtcttgactgattcctccttcaagtttggtactgtcatgctgacgatcgtcaagacgtgtggtccacgggttgtgtgatggctgaactcatgcttggtcagccattgttccctggagagtcgggaattgaccaactggtggaaatcatcaaggttcttggaacccctactcgggagcagatccgcaccatgaacccaaactatatggagcacaaattccctcaaatcaagccacacccattcaacaaggtgaccacgctcttaaagaacttcttgcgaatatgcactgacttgatgaccccaggttttccggagagctcctcacgaggccattgatctgatctcagctttgctagaatacacgccgacacaacgtctctccgctatcgaggcgatgtgccacccgttcttcgacgaactcagagatcccaatacgcgactgcccgactctcggcaccctggtggcgctgctagagacctccccaatctctttgatttctccagacatggtttgttgtcacttgaggcccaaattcattcttccagatggcttattcgctgatcactcttttgtagaactttctattgcacctgcattgaacagccggctggttccccctcatgcacgcgccgctctcgaggcccgggggctagacattgacaacttcactcctctcacgaaggaggagatgatggcacgtctcgactga

SEQ ID NO: 41 - DNA, 1182 bp, Aspergillus fumigatus
atgtcacaaaatcgacctggggtgttctcgaatctgcgcatgggtgaagtcgtccgcgagaaggtccaggatggactgacaggggaaactaaggagatttcgtactcacaatgtaaaatcgtcggcaatggatcgtttggtgtcgtctttcagacgaaaatgatgccaagcggcgaggatgctgccattaagagggtccttcaagacaagcgcttcaaaaatcgagaactgcagattatgcggattgttcgccatcctaacatcgtagaattgaaagccttctattactcgaacggcgagaggaaggatgaagtgtacctaaacctcgttctcgaatacgtaccagaaaccgtgtatcgggcgtcgcggtactttaataaactcaaaacgactatgccaatgttggaagtcaagctgtatatctatcaattgttccgttccctggcatacatccattcacaaggcatctgccaccgtgacatcaagccccagaatctcttacttgatccatccaccggcatcctcaaactctgcgactttggttcggccaagattctggtagagaatgagcccaacgtttcctatatctgttcccgctactatcgtgcgccggaattgatctttggcgccactaattacacaacaaagatcgacgtgtggtccacgggttgtgtgatggctgaactcatgcttggtcagccattgttccctggagagtcgggaattgaccaactggtggaaatcatcaaggttcttggaacccctactcgggagcagatccgcaccatgaacccaaactatatggagcacaaattccctcaaatcaagccacacccattcaacaaggttttccggagagctcctcacgaggccattgatctgatctcagctttgctagaatacacgccgacacaacgtctctccgctatcgaggcgatgtgccacccgttcttcgacgaactcagagatcccaatacgcgactgcccgactctcggcaccctggtggcgctgctagagacctccccaatctctttgatttctccagacatgaactttctattgcacctgcattgaacagccggctggttccccctcatgcacgcgccgctctcgaggcccgggggctagacattgacaacttcactcctctcacgaaggaggagatgatggcacgtctcgac

SEQ ID NO: 42 - protein, 394 aa, Aspergillus fumigatus
MSQNRPGVFSNLRMGEVVREKVQDGLTGETKEISYSQCKIVGNGSFGVVFQTKMMPSGEDAAIKRVLQDKRFKNRELQIMRIVRHPNIVELKAFYYSNGERKDEVYLNLVLEYVPETVYRASRYFNKLKTTMPMLEVKLYIYQLFRSLAYIHSQGICHRDIKPQNLLLDPSTGILKLCDFGSAKILVENEPNVSYICSRYYRAPELIFGATNYTTKIDVWSTGCVMAELMLGQPLFPGESGIDQLVEIIKVLGTPTREQIRTMNPNYMEHKFPQIKPHPFNKVFRRAPHEAIDLISALLEYTPTQRLSAIEAMCHPFFDELRDPNTRLPDSRHPGGAARDLPNLFDFSRHELSIAPALNSRLVPPHARAALEARGLDIDNFTPLTKEEMMARLD

SEQ ID NO: 43 - DNA, 2209 bp, Aspergillus fumigatus
accgcgcgatcctgctgtgctgagtcagcattggattacttcattagacctcgacagtcaacagcatttctccacttaaccacctacaacctaagacagcacgggaactggttatatttgttcgagtcagttacttcagggtgttttattaaatcaatgtggaagtcttttaaagaaaagcatgccagcaagtttgggggaggctcggctgaagcggcagcctcagacggtggccaagatctgaccacgatattggatagatcccaacgcggggaattgacagtgcttgttgcgctaatcgcacagcggatgcgggatggaatagaacaaaacttctccaatgctcctccttcgtcaggccagtctgtcaactacgaagaaaaacggacaggctccctgccccaatctacagatgcacaagaagaccagtcatcctccggcagcgcagcgaacggttcaagaaccgaccctcaattcaaagacccggagactgcgacatgtgcactatctaaatatgatgactggagggactccgtccttcttcgaattggtgaagtcgtcaacagggatccagaacatggtgaagttcaggcgaatgaaaacccaccctcaggacagcaatcccagcagatccgctccgaggaggatgaccgttccattcgtaagctacgtgaggtctttccgccggtggagaccagcctctctcaattgccagaagcgaagaaactcttaattctccactcattgctactcctagttttgagtcttgaacactataacgcctggtctcgggtcctgatgctgttcgtgacgtccagcttagggctggacgtgaaattactaaacgaagatgaagtcaaagtggcgagggggttgcttgacactgctctggcactgtcgtctaacgccccaaggcaggatgagagtcgaagtcgcgattcatcccgaaaatggaaggttgggatcgcgtcagttgcgggtgctgccctgatcgggatcactggtggactggctgcgccccttgttgcagctgggcttggtactgtcatgggcggccttgggcttggcgccaccgccgcagcagggtatctcggagctctcgctggaagtggtgttgtcgtcggcggacttttcggtgcttatggtgggcggatgaccggtcgtatggttgacaagtacgcacgggaggtggatgattttgcctttctgccgattcgtggttctcggcatcgatccgaagacgaaagagaagctgcccatcaggatcaccggctgcgggttaccatcggcgtgaccggatggctgacagaggaggacaatttcgtgatcccgtggcgagtgatcggagcggaatcggaggtgtttggtctccgctgggaaaccgagcccctgatgaatctgggaaatgcgcttgaccttttggtaaccagcgccgcatggactgccggtgaacaagtcctgaagaagacattcctctcccaactcttgaccgctgtcgcgctgccgcttggccttctcaaggtcgcacgtgtggtggacaatccgtttagcgtagcgaaggctcgggcggacaaggctggggaggttctcgcggatgctcttatcagtaaagtgcagggcgagcgaccagtcaccctcattggctactccttagggtctcgggtgattttcgcttgccttcaaagcttggcgaaacggcgcgcgttcggcttggtggaatccgcaattctgatgggagctcccaccccgtcgaattcagaacaatggtgtcgcatccgcagtgttgtgagtggacgccttgtcaacgtgtactcggaaaacgactccgtgctggctctcttatatcgaacaagcagcctccagcttggggttgcaggcttgcagcctgttgaaggtgtctcaggcgttgagaatctggacgttagcgacctgatcagcggccatctccgttatcagtttctcgttggcaggatcttgagcgttgttggacttgagagcattgatgctcgcgaggtcgcactggaggaggccgcattagaagccaaagatcggaggcaggagcaggaaagggctcataacgaacgacaggctggatttatgggcgagggtcggtcaccaagccagcggctggaaagccaggaggatctgcagggcgaagaggacagattacagaaagagatgggaaaagcacgagtgcggcactcttaga

SEQ ID NO: 44 - DNA, 2209 bp, Aspergillus fumigatus; identical to SEQ ID NO: 43

SEQ ID NO: 45 - protein, 735 aa, Aspergillus fumigatus
TARSCCAESALDYFIRPRQSTAFLHLTTYNLRQHGNWLYLFESVTSGCFIKSMWKSFKEKHASKFGGGSAEAAASDGGQDLTTILDRSQRGELTVLVALIAQRMRDGIEQNFSNAPPSSGQSVNYEEKRTGSLPQSTDAQEDQSSSGSAANGSRTDPQFKDPETATCALSKYDDWRDSVLLRIGEVVNRDPEHGEVQANENPPSGQQSQQIRSEEDDRSIRKLREVFPPVETSLSQLPEAKKLLILHSLLLLVLSLEHYNAWSRVLMLFVTSSLGLDVKLLNEDEVKVARGLLDTALALSSNAPRQDESRSRDSSRKWKVGIASVAGAALIGITGGLAAPLVAAGLGTVMGGLGLGATAAAGYLGALAGSGVVVGGLFGAYGGRMTGRMVDKYAREVDDFAFLPIRGSRHRSEDEREAAHQDHRLRVTIGVTGWLTEEDNFVIPWRVIGAESEVFGLRWETEPLMNLGNALDLLVTSAAWTAGEQVLKKTFLSQLLTAVALPLGLLKVARVVDNPFSVAKARADKAGEVLADALISKVQGERPVTLIGYSLGSRVIFACLQSLAKRRAFGLVESAILMGAPTPSNSEQWCRIRSVVSGRLVNVYSENDSVLALLYRTSSLQLGVAGLQPVEGVSGVENLDVSDLISGHLRYQFLVGRILSVVGLESIDAREVALEEAALEAKDRRQEQERAHNERQAGFMGEGRSPSQRLESQEDLQGEEDRLQKEMGKARVRHS

SEQ ID NO: 46 - DNA, 510 bp, Aspergillus fumigatus
atggccgatatcgatgtcaaggttgctcaatggaagcttgttgaggttggccgtgttgtgctgatccgcagcggtccttacaccggcaagcttgctgccattgtcgagatcatcgaccacaagcgtgtacgtttttcaacggagaaattctgagcgcaggacggaaagatcatggtcggatgtgatattgacaaagaggcgcgatcataggtcctggttgacggtccttccaccgaggagaacaagatcgttccccgtcacgctcttcctctcgctcacgccactctcacccccttcgtcattcccaaactcccccgcgctgccggcactggccccgtcaagaagctctgggagaagaacgagatcgatggaaagtgggctaagagcaccattgctcagaagactgagcgcgctgagcggaggaagaaccttaccgacttcgagcgcttcaaggtcctcagactcaagaagcaggtacgttcagtttgcgaaactatgggagaattgtga

SEQ ID NO: 47 - DNA, 423 bp, Aspergillus fumigatus
atggccgatatcgatgtcaaggttgctcaatggaagcttgttgaggttggccgtgttgtgctgatccgcagcggtccttacaccggcaagcttgctgccattgtcgagatcatcgaccacaagcgtgtcctggttgacggtccttccaccgaggagaacaagatcgttccccgtcacgctcttcctctcgctcacgccactctcacccccttcgtcattcccaaactcccccgcgctgccggcactggccccgtcaagaagctctgggagaagaacgagatcgatggaaagtgggctaagagcaccattgctcagaagactgagcgcgctgagcggaggaagaaccttaccgacttcgagcgcttcaaggtcctcagactcaagaagcaggtacgttcagtttgcgaaactatgggagaattg

SEQ ID NO: 48 - protein, 141 aa, Aspergillus fumigatus
MADIDVKVAQWKLVEVGRVVLIRSGPYTGKLAAIVEIIDHKRVLVDGPSTEENKIVPRHALPLAHATLTPFVIPKLPRAAGTGPVKKLWEKNEIDGKWAKSTIAQKTERAERRKNLTDFERFKVLRLKKQVRSVCETMGEL

SEQ ID NO: 49 - DNA, 1413 bp, Aspergillus fumigatus
atggctctccgccggccattaacacttccgaggcacattctcaatggagcttgtttaggcttgcgaccagctgtgtctcgcgccgctctggcttatgggcaggagcagaggaaagggcttgcaacagcagttcccccggtcactcaaaatgcggctgggtccaaaggccccacggcaatggtcttcctcaacatgggtgggccatcgaagattgacgaagtggaagattttctgagcagattatttgtatgcattcctcaatatgcccgatgctaccaccatgtatgagtctgacaaactctctcttcctataccaggccgatggcgatctgattcctctcggacgacttcaatcatacctcggccctctcatcgctaagcgcagaaccccaaagatccaacggcaatactcggatattggtggagggtcaccgatcaggaaatggtccgagtatcagtgcgaggaaatgtgcagattgctagacaaaatcaatcccgaaacggctcctcacaagccttacgtcgcgttccggtacgccgaccctctgacggaagaaatgtacacaaagttgctggaagatggattcggcaacgggaaaggcgggcgcgctgtcgcgttcacacagtacccccaatattcgtgctccaccacgggtagctcgctgaacgagttgtggaaatggagaaccaggcttgagggtaagcgtgcaaatggcaacatggaccccgctggtgccatccagtggagtgtcattgatcgatggccaacgcaccctggcctcgtggaggctttcgcccggaacattgaggagcagctgaagacatacccagaggagaagcgaaacggtgtcgttctcttgttctcagcccacagtctgcccatgagtgttgtcaacagaggtgagactcatcttcttaccgaacaacaagatttgctcgctaacacatttcctaggcgacccatatcctgctgaagttgctgcaactgtgcatgctgtcatgcaaagattgaatttcagcaatccttaccgactgtgctggcagtcccaagtgggaccgtcagcttggcttggagcccaaactagcgatacggtcgaaaactatgtcaaacgtggacagaccgatattattctagttcccattgccttcaccagcgaccatattgagactctgtacgagttggatctggaagtgataaaggaagcaaactccccgggagtcaagagagccgagagtttgaatggtaaccccattttcattcaggcattagcagacattgcccaagagcacctccgtaagggagagaagtgctcactacagatgactctgcgctgtcaaggctgtaagagcgaacggtgcctggaacagaagaaattctttgctggcgaccgattttcttctcttgtagtttag

SEQ ID NO: 50 - DNA, 1284 bp, Aspergillus fumigatus
atggctctccgccggccattaacacttccgaggcacattctcaatggagcttgtttaggcttgcgaccagctgtgtctcgcgccgctctggcttatgggcaggagcagaggaaagggcttgcaacagcagttcccccggtcactcaaaatgcggctgggtccaaaggccccacggcaatggtcttcctcaacatgggtgggccatcgaagattgacgaagtggaagattttctgagcagattatttgccgatggcgatctgattcctctcggacgacttcaatcatacctcggccctctcatcgctaagcgcagaaccccaaagatccaacggcaatactcggatattggtggagggtcaccgatcaggaaatggtccgagtatcagtgcgaggaaatgtgcagattgctagacaaaatcaatcccgaaacggctcctcacaagccttacgtcgcgttccggtacgccgaccctctgacggaagaaatgtacacaaagttgctggaagatggattcggcaacgggaaaggcgggcgcgctgtcgcgttcacacagtacccccaatattcgtgctccaccacgggtagctcgctgaacgagttgtggaaatggagaaccaggcttgagggtaagcgtgcaaatggcaacatggaccccgctggtgccatccagtggagtgtcattgatcgatggccaacgcaccctggcctcgtggaggctttcgcccggaacattgaggagcagctgaagacatacccagaggagaagcgaaacggtgtcgttctcttgttctcagcccacagtctgcccatgagtgttgtcaacagaggcgacccatatcctgctgaagttgctgcaactgtgcatgctgtcatgcaaagattgaatttcagcaatccttaccgactgtgctggcagtcccaagtgggaccgtcagcttggcttggagcccaaactagcgatacggtcgaaaactatgtcaaacgtggacagaccgatattattctagttcccattgccttcaccagcgaccatattgagactctgtacgagttggatctggaagtgataaaggaagcaaactccccgggagtcaagagagccgagagtttgaatggtaaccccattttcattcaggcattagcagacattgcccaagagcacctccgtaagggagagaagtgctcactacagatgactctgcgctgtcaaggctgtaagagcgaacggtgcctggaacagaagaaattctttgctggcgaccgattttcttctcttgtagtt

SEQ ID NO: 51 - protein, 428 aa, Aspergillus fumigatus
MALRRPLTLPRHILNGACLGLRPAVSRAALAYGQEQRKGLATAVPPVTQNAAGSKGPTAMVFLNMGGPSKIDEVEDFLSRLFADGDLIPLGRLQSYLGPLIAKRRTPKIQRQYSDIGGGSPIRKWSEYQCEEMCRLLDKINPETAPHKPYVAFRYADPLTEEMYTKLLEDGFGNGKGGRAVAFTQYPQYSCSTTGSSLNELWKWRTRLEGKRANGNMDPAGAIQWSVIDRWPTHPGLVEAFARNIEEQLKTYPEEKRNGVVLLFSAHSLPMSVVNRGDPYPAEVAATVHAVMQRLNFSNPYRLCWQSQVGPSAWLGAQTSDTVENYVKRGQTDIILVPIAFTSDHIETLYELDLEVIKEANSPGVKRAESLNGNPIFIQALADIAQEHLRKGEKCSLQMTLRCQGCKSERCLEQKKFFAGDRFSSLVV

SEQ ID NO: 52 - DNA, 1536 bp, Aspergillus fumigatus
atgatttatctccggtcctcgttgctgaggtctggattggctcgagatcctgctcgcctgtgttcacaatgcttctcacgactctcaccatcacgacgacctgtcgcagttcgcagcttcttctcctcatctcggctgcgggctggcattgccgatcatgaatcaactccctcgactgtccaaaagacctatttttctgccaatcggaccgcagatggcttacttgcatccttatccgccgtcaatagctcccctcgaagtattgccgacaatgcgttatcacagggtgcagccagttcggagtcgattacttcacagtctacttcacaagagttacctcatcgccggaggaagcggttaaaggaagaggcggccaagaataatgctgcagaaaccgaactccctcctgatgcctcgtctcaattgtccaccctctcatcagccctccctgcgacttccctgcgccgcaagctggctgcgtttctcgccctcacaaagcctcgtctctcgttcctgatcgtgttgacgactacctccgcttatgggatgtacccgatctcctctcttctcacacttgacccttcaatgactcccctaccgaccctctcgacctcaaccttgacctttctctacctgaccacaggaaccttcttgtcttcatgcagcgccaataccttgaatatgctccttgaacctaaatacgatgccctcatgtcacggacacggaaccggccgttagtgcgggggctactctcacgccgtgctgcggtattgtttgcgattgcgactgctgctgcaggtctcggtttgttatacattggaacgaaccctacgactactgcgctctccgccagtaatatctgtctctatgcctttgtgtatacgccgctgaagcgtatatcagtgatcaacacctgggtaggcgccgtggtaggaggcattcctccgttgatgggttggaccgctgcagcaggccagacagcgaccactggccacgacagctggcgggacatgttgttcagcaaggatagcatcggtggttggctcctgggtggcattctctttgcatggcagtttcctcatttcaatgctttgtcctacatgatccgtgaagagtacaaggcagccgggtacaggatgctcgcatggactaatcccgccgcaaatgcccgtgtcgcactacgatattctcttctcatgtttcctttctccgtcggtctctggtgggtaggagttgtcggtaatggtttcctggttggaagcacggcggccaatggctggctagtcaaagaggcctacaaattctggcggcaccaaggcgccaacggcagtgctcgacgcctcttctgggccagtatttggcagctgccaatcctccttgtcggtggtctggtcacgaagaaaggtctctgggatggtgtctggaacaatgttttcggtcagcctgtggaagacgaggatgactatctctgggaggatgaggatgaagtggcagaggcggagcgcaagatgatacctgcgaagacgagtagctcg

SEQ ID NO: 53 - DNA, 1536 bp, Aspergillus fumigatus; identical to SEQ ID NO: 52

SEQ ID NO: 54 - protein, 512 aa, Aspergillus fumigatus
MIYLRSSLLRSGLARDPARLCSQCFSRLSPSRRPVAVRSFFSSSRLRAGIADHESTPSTVQKTYFSANRTADGLLASLSAVNSSPRSIADNALSQGAASSESITSQSTSQELPHRRRKRLKEEAAKNNAAETELPPDASSQLSTLSSALPATSLRRKLAAFLALTKPRLSFLIVLTTTSAYGMYPISSLLTLDPSMTPLPTLSTSTLTFLYLTTGTFLSSCSANTLNMLLEPKYDALMSRTRNRPLVRGLLSRRAAVLFAIATAAAGLGLLYIGTNPTTTALSASNICLYAFVYTPLKRISVINTWVGAVVGGIPPLMGWTAAAGQTATTGHDSWRDMLFSKDSIGGWLLGGILFAWQFPHFNALSYMIREEYKAAGYRMLAWTNPAANARVALRYSLLMFPFSVGLWWVGVVGNGFLVGSTAANGWLVKEAYKFWRHQGANGSARRLFWASIWQLPILLVGGLVTKKGLWDGVWNNVFGQPVEDEDDYLWEDEDEVAEAERKMIPAKTSSS

SEQ ID NO: 55 - DNA, 1626 bp, Aspergillus fumigatus
atgctcaacgccgcggttgctgccccgcgatgttttgtatatcccactgatcgcgcagcaatgcgcttgggctttgctcttcgtctctcctctcctgcacctctcttctcaacagcacctttccgtcgacagttgcatgcttccggcgtccgatcaattgaacctgttatctttcgaaatagccttgaaaagactcttgaggctcatcgatcctccaatcgagccagtctgatccgcaaggtgattaaccacgattgtcctgctgaaacgccccctccaattttaccacttgagaatcgtgctggtcatgatcaatcatctcaaaaggcctcctccgtgtcaaatgcagagtcagagtccccccggtcttctgcgcctgcgagacgagcgcagaggaaggcccgttcgcccagccaagtagccaccccgcagccccagacaacagaatatccacaactgcaatggcatgcagatgaaaccaagggccgaccggcacaaagtccttggctgaagtacttgactaccgattggaaaacgcccgatgccgtttcgcgtctcgacgcggagatccgcgctcttgagctctacatgacaccgaccccgtcggagcggactgagatagatcggctggttgcagatatgggtaggttgctagcgggaatcgtccccagcccgccccaggtaaccggttcatggcggacgcgatttgccttgagccactcgggtctcgattttgtcttacctgtcccggattcagaccgatccacccgtgacgttcgcaagccgagtgccacacggcccaaggtgctccagacttacaaaaagctcttacatgaagtgggacatgcgcttcagcagtccccctcgttcgcggagcgagtccgcatcataggcagccgtttccccgtcctctcagccatccatcgccccacgggccgcctgctgcagttccactgcggtgaagggctaccggcctctgtcgaatacatcatggattaccaggccgagtatccctcgatccggccgctctacgtgaccgctcgcctgatcctggaggcgcggggtaggtatggccgtactcagatgtctattgaatccgatgccctcgtaatgcttctcgtggccttcctcaaaatgaaccacgggcgttttcagcggcccgactgtctcggcgagcagctgatcgcgtttctgcgcgcctacggcagcgatattgacctgaccaccaccggtgtgtccgtcgatccccccagttggttcaatgctagtacggtcaaacgcgccagcgccctgtacgcgcccgatgatctacccgcgcatctgcgcggccagcgctccctcatcagcctcaagagaacagcagccgccagacgcaatctgcctgccgccagccggctgtgcgtgcaggaccccaccaattacatgaatgatctgggccgcagctgcgtgcgtacgttggaactccagcacacgttctcgcttgctcatgaccgtctcggcgcaagtctcaagcgctgggatgacagtgaaccggccgcgaacgttagtatcctgacacgggccctgcaagcaaacttttctgattttgaaaatctacgcgccaaatcgcttaagctcaacgcgacc

SEQ ID NO: 56 - DNA, 1626 bp, Aspergillus fumigatus; identical to SEQ ID NO: 55

SEQ ID NO: 57 - protein, 542 aa, Aspergillus fumigatus
MLNAAVAAPRCFVYPTDRAAMRLGFALRLSSPAPLFSTAPFRRQLHASGVRSIEPVIFRNSLEKTLEAHRSSNRASLIRKVINHDCPAETPPPILPLENRAGHDQSSQKASSVSNAESESPRSSAPARRAQRKARSPSQVATPQPQTTEYPQLQWHADETKGRPAQSPWLKYLTTDWKTPDAVSRLDAEIRALELYMTPTPSERTEIDRLVADMGRLLAGIVPSPPQVTGSWRTRFALSHSGLDFVLPVPDSDRSTRDVRKPSATRPKVLQTYKKLLHEVGHALQQSPSFAERVRIIGSRFPVLSAIHRPTGRLLQFHCGEGLPASVEYIMDYQAEYPSIRPLYVTARLILEARGRYGRTQMSIESDALVMLLVAFLKMNHGRFQRPDCLGEQLIAFLRAYGSDIDLTTTGVSVDPPSWFNASTVKRASALYAPDDLPAHLRGQRSLISLKRTAAARRNLPAASRLCVQDPTNYMNDLGRSCVRTLELQHTFSLAHDRLGASLKRWDDSEPAANVSILTRALQANFSDFENLRAKSLKLNAT

SEQ ID NO: 58 - DNA, 2356 bp, Aspergillus fumigatus
atggggttggaggatccgcggcgagcagtccaggcagagtcgtatctggagactacccggtcgcagctcgtcgatgcgcttatatcacggccgaatcttgagagtggcttgggtcgaaggaattttgttcctcctaggcaggtcccggttgtcgacgccatggagcggacgaaaaatgctatacaatcaaataattccagctcgcgggcgcaactttcggatgcattacccgagagcgagaaatcacagagtgcagggcaggttattgtcccgacacggatgcaggaacttctcgaccgcggtcgtcccattgaggcggcccaattttttctggagacacatgccgcttcattgaaaggcatatcctcagacaggaaggaaatggctacgaaggtattcttcgtcaactgcaaagaggacaatgtgtttattgccagaagtgtgtttgagcggttggaagaggtggatagaatcacgcctgagatgtggaagacgttgatgctggctttggcgaagaaagggtgcattgaatcagttgcgagtgtttatacgcgatacatgcgcaagttcccttgccctccggagatggtcgacgttgtgctgcggagtttgcttgaatcccaccgactcaccactgcgaaatggttcctcttgcgcaatctacaacatgaccgtgattgcggtttgtgcggagcctacctctccggcctgtggcggaagacaagaagcatcgagttattgaacgggcagttgaaaaagatattgaccattcttccaaagttcgaaaaacaacctagcgacaaactatttaacccggtgatcaaggcatatgttgaatttgggcgggttgccgatgccgaagctctggtgcatgatatgaccactttatatgggatccctcttcgttgccgaactcagggcctattggtatacgccaaggctctgaactgtgactgggagggagttgacgcaggactgcaagagatgcacaaactcaagctgacgagacgccggcgagacttccttcctatttttgatcgaatatttctggagtactgggtctcacattcgggaattgagattcgcaattttgtgttccggtaccttgataaattcgacattgtccccgatcgcgtgctctacaagcacatcttggaggctttcgtggagaaaggagacaaggaaatgattgctgaatttacaagtatggcgaagcagcgatcgtggaatatccccataaacgagcagcaattcttggagatactacggtctcgtcgcctcgcattggaaggagcaccagtggggttctggcaaatgctgcaggcagcgcgtgtcaagtacgggcacagctccacgtctcagcgtatcatgggctacgaccaacagtcattccctttgccggaagtcaacagcatgccatatacacagaatccgctatcgtggtaccagagaacgatgcaagagaccacgccgtcgaagcctgtcgaccaatatcagaaactccataagcagatgacccattttctgcacgctggaaagctgaaggaagcattgaagtgctttcaaaatgctaaaaatgcccggttccagatgaggcagcttcacgttgaattggcggtcatagcgactttgcttgaggacggccttagtgcagcgcgcagtctcatcgagtctgaatggcggactatccgtcaccttgtccgcttctctcctatcttctttcgtcaagtcatggcggtcgacgaggatgcgggtggccacattgtccagatggcagtcctacgcttctaccagctttgttggtctacgaaacacatgaaggtcaagcatcaccttactgttgcgacaagccgtcgcttgatctcccaacataagccggaaatggctctggagcttctgacggccgtgtacaagtcccgttataggtttgcagcgacctttgacggggtttgcatgaagatgtttgcgcgcgccttcgccgcgacagacaacattctaggcttacgatggtgtattcttactgctttatcacgcgatagtgcactcaatcatgattttgtggtggaagttcgccgaatcttgggcactctaagtccaccttccgcggtggatgccaccgccggccccgtcactcatgagcagctggaatacctttattacatcgccgatctgctcgaggagaagaatgaggggtgcgccccgatatgggagctcaagcatgacgccacactgaagcagtcttcgaggcgacagttgaagcaacctcttgacgcaagccgtcttttcaaccagtccgatgtccgcgagaccgtcaagcgatgggacgaagaatacgagctggaggcagtgctgggta

SEQ ID NO: 59 - DNA, 2356 bp, Aspergillus fumigatus; identical to SEQ ID NO: 58

SEQ ID NO: 60 - protein, 785 aa, Aspergillus fumigatus
MGLEDPRRAVQAESYLETTRSQLVDALISRPNLESGLGRRNFVPPRQVPVVDAMERTKNAIQSNNSSSRAQLSDALPESEKSQSAGQVIVPTRMQELLDRGRPIEAAQFFLETHAASLKGISSDRKEMATKVFFVNCKEDNVFIARSVFERLEEVDRITPEMWKTLMLALAKKGCIESVASVYTRYMRKFPCPPEMVDVVLRSLLESHRLTTAKWFLLRNLQHDRDCGLCGAYLSGLWRKTRSIELLNGQLKKILTILPKFEKQPSDKLFNPVIKAYVEFGRVADAEALVHDMTTLYGIPLRCRTQGLLVYAKALNCDWEGVDAGLQEMHKLKLTRRRRDFLPIFDRIFLEYWVSHSGIEIRNFVFRYLDKFDIVPDRVLYKHILEAFVEKGDKEMIAEFTSMAKQRSWNIPINEQQFLEILRSRRLALEGAPVGFWQMLQAARVKYGHSSTSQRIMGYDQQSFPLPEVNSMPYTQNPLSWYQRTMQETTPSKPVDQYQKLHKQMTHFLHAGKLKEALKCFQNAKNARFQMRQLHVELAVIATLLEDGLSAARSLIESEWRTIRHLVRFSPIFFRQVMAVDEDAGGHIVQMAVLRFYQLCWSTKHMKVKHHLTVATSRRLISQHKPEMALELLTAVYKSRYRFAATFDGVCMKMFARAFAATDNILGLRWCILTALSRDSALNHDFVVEVRRILGTLSPPSAVDATAGPVTHEQLEYLYYIADLLEEKNEGCAPIWELKHDATLKQSSRRQLKQPLDASRLFNQSDVRETVKRWDEEYELEAVLG

SEQ ID NO: 61 - DNA, 21 bp, Artificial Sequence
  Description of Artificial Sequence Synthetic primer
atgaaggcgtaagttccttgc

SEQ ID NO: 62 - DNA, 19 bp, Artificial Sequence
  Description of Artificial Sequence Synthetic primer
gtgtggaggaagaaagagc

SEQ ID NO: 63 - DNA, 20 bp, Artificial Sequence
  Description of Artificial Sequence Synthetic primer
ggaagacgggcagttagtcc

SEQ ID NO: 64 - DNA, 21 bp, Artificial Sequence
  Description of Artificial Sequence Synthetic primer
cccaggctttacactttatgc

SEQ ID NO: 65 - DNA, 35 bp, Artificial Sequence
  Description of Artificial Sequence Synthetic primer
ggccacgcgtcgactagtagnnnnnnnnnngatat

SEQ ID NO: 66 - DNA, 35 bp, Artificial Sequence
  Description of Artificial Sequence Synthetic primer
ggccacgcgtcgactagtacnnnnnnnnnnacgtc

SEQ ID NO: 67 - DNA, 35 bp, Artificial Sequence
  Description of Artificial Sequence Synthetic primer
ggccacgcgtcgactagtacnnnnnnnnnntggac

SEQ ID NO: 68 - DNA, 35 bp, Artificial Sequence
  Description of Artificial Sequence Synthetic primer
ggccacgcgtcgactagtacnnnnnnnnnnacgtg

SEQ ID NO: 69 - DNA, 21 bp, Artificial Sequence
  Description of Artificial Sequence Synthetic primer
cgaagttgacgttcagtatgc

SEQ ID NO: 70 - DNA, 20 bp, Artificial Sequence
  Description of Artificial Sequence Synthetic primer
tgaccatgattacgccaagc

SEQ ID NO: 71 - DNA, 20 bp, Artificial Sequence
  Description of Artificial Sequence Synthetic primer
ggccacgcgtcgactagtac

SEQ ID NO: 72 - DNA, 19 bp, Artificial Sequence
  Description of Artificial Sequence Synthetic primer
gttggatctttgggttctg

SEQ ID NO: 73 - DNA, 20 bp, Artificial Sequence
  Description of Artificial Sequence Synthetic primer
cgcgaatctgatgacatagc

SEQ ID NO: 74 - DNA, 20 bp, Artificial Sequence
  Description of Artificial Sequence Synthetic primer
ctcttcgcttcatcgtaccc

SEQ ID NO: 75 - DNA, 20 bp, Artificial Sequence
  Description of Artificial Sequence Synthetic primer
attagtccatgcgagcatcc

SEQ ID NO: 76 - DNA, 20 bp, Artificial Sequence
  Description of Artificial Sequence Synthetic primer
gcctgagcctagtccatcac

SEQ ID NO: 77 - DNA, 20 bp, Artificial Sequence
  Description of Artificial Sequence Synthetic primer
ctcgcaggtcgatttcactc

SEQ ID NO: 78 - DNA, 20 bp, Artificial Sequence
  Description of Artificial Sequence Synthetic primer
ggaggaaaccttgtcaccac

SEQ ID NO: 79 - DNA, 20 bp, Artificial Sequence
  Description of Artificial Sequence Synthetic primer
taccgagaaggaggtcatgg

SEQ ID NO: 80 - DNA, 20 bp, Artificial Sequence
  Description of Artificial Sequence Synthetic primer
tccagtcaaggttggtgatg

SEQ ID NO: 81 - DNA, 19 bp, Artificial Sequence
  Description of Artificial Sequence Synthetic primer
cgagaccatcctacctcag

SEQ ID NO: 82 - DNA, 20 bp, Artificial Sequence
  Description of Artificial Sequence Synthetic primer
acactcaccgccttaaccac

SEQ ID NO: 83 - DNA, 20 bp, Artificial Sequence
  Description of Artificial Sequence Synthetic primer
agtgcccttcattcagttcc

SEQ ID NO: 84 - DNA, 21 bp, Artificial Sequence
  Description of Artificial Sequence Synthetic primer
gcgactttgagggaactatcc

SEQ ID NO: 85 - DNA, 20 bp, Artificial Sequence
  Description of Artificial Sequence Synthetic primer
caccacccaccttatgaagc

SEQ ID NO: 86 - DNA, 20 bp, Artificial Sequence
  Description of Artificial Sequence Synthetic primer
accaggagaatcagcgacac

SEQ ID NO: 87 - DNA, 20 bp, Artificial Sequence
  Description of Artificial Sequence Synthetic primer
gggacgaagaatacgagctg

SEQ ID NO: 88 - DNA, 19 bp, Artificial Sequence
  Description of Artificial Sequence Synthetic primer
ctgaatatcgacggtttcc

SEQ ID NO: 89 - DNA, 19 bp, Artificial Sequence
  Description of Artificial Sequence Synthetic primer
gcacattggctgaatatcg

SEQ ID NO: 90 - DNA, 24 bp, Artificial Sequence
  Description of Artificial Sequence Synthetic primer
ttgacaccagaccaactggtaatg

SEQ ID NO: 91 - DNA, 3052 bp, Aspergillus fumigatus
cgagccgctccaagctattcgggatgagactccggacgcgatccaggagaacacgattggattagaacaattgaaggaggccatgtccaaagaacgagtcgtaggacggagcagaaggatacatcgcgctgagagcgccagaaatgaccgcccggacggagatgcttgggatgggaatgcgcttggctatgcgtccaggactaaggggaagttctttgtcgttgagacaggcaatgaacctggcaattgattcggcatgcctctttcccaaaagccatttcaatattactgcacaatagcctgcttcatcttgcgtgcgacgggctgcacccctgctatgagagaccgcgcgatcctgctgtgctgagtcagcattggattacttcattagacctcgacagtcaacagcatttctccacttaaccacctacaacctaagacagcacgggaactggttatatttgttcgagtcagttacttcagggtgttttattaaatcaatgtggaagtcttttaaagaaaagcatgccagcaagtttgggggaggctcggctgaagcggcagcctcagacggtggccaagatctgaccacgatattggatagatcccaacgcggggaattgacagtgcttgttgcgctaatcgcacagcggatgcgggatggaatagaacaaaacttctccaatgctcctccttcgtcaggccagtctgtcaactacgaagaaaaacggacaggctccctgccccaatctacagatgcacaagaagaccagtcatcctccggcagcgcagcgaacggttcaagaaccgaccctcaattcaaagacccggagactgcgacatgtgcactatctaaatatgatgactggagggactccgtccttcttcgaattggtgaagtcgtcaacagggatccagaacatggtgaagttcaggcgaatgaaaacccaccctcaggacagcaatcccagcagatccgctccgaggaggatgaccgttccattcgtaagctacgtgaggtctttccgccggtggagaccagcctctctcaattgccagaagcgaagaaactcttaattctccactcattgctactcctagttttgagtcttgaacactataacgcctggtctcgggtcctgatgctgttcgtgacgtccagcttagggctggacgtgaaattactaaacgaagatgaagtcaaagtggcgagggggttgcttgacactgctctggcactgtcgtctaacgccccaaggcaggatgagagtcgaagtcgcgattcatcccgaaaatggaaggttgggatcgcgtcagttgcgggtgctgccctgatcgggatcactggtggactggctgcgccccttgttgcagctgggcttggtactgtcatgggcggccttgggcttggcgccaccgccgcagcagggtatctcggagctctcgctggaagtggtgttgtcgtcggcggacttttcggtgcttatggtgggcggatgaccggtcgtatggttgacaagtacgcacgggaggtggatgattttgcctttctgccgattcgtggttctcggcatcgatccgaagacgaaagagaagctgcccatcaggatcaccggctgcgggttaccatcggcgtgaccggatggctgacagaggaggacaatttcgtgatcccgtggcgagtgatcggagcggaatcggaggtgtttggtctccgctgggaaaccgagcccctgatgaatctgggaaatgcgcttgaccttttggtaaccagcgccgcatggactgccggtgaacaagtcctgaagaagacattcctctcccaactcttgaccgctgtcgcgctgccgcttggccttctcaaggtcgcacgtgtggtggacaatccgtttagcgtagcgaaggctcgggcggacaaggctggggaggttctcgcggatgctcttatcagtaaagtgcagggcgagcgaccagtcaccctcattggctactccttagggtctcgggtgattttcgcttgccttcaaagcttggcgaaacggcgcgcgttcggcttggtggaatccgcaattctgatgggagctcccaccccgtcgaattcagaacaatggtgtcgcatccgcagtgttgtgagtggacgccttgtcaacgtgtactcggaaaacgactccgtgctggctctcttatatcgaacaagcagcctccagcttggggttgcaggcttgcagcctgttgaaggtgtctcaggcgttgagaatctggacgttagcgacctgatcagcggccatctccgttatcagtttctcgttggcaggatcttgagcgttgttggacttgagagcattgatgctcgcgaggtcgcactggaggaggccgcattagaagccaaagatcggaggcaggagcaggaaagggctcataacgaacgacaggctggatttatgggcgagggtcggtcaccaagccagcggctggaaagccaggaggatctgcagggcgaagaggacagattacagaaagagatgggaaaagcacgagtgcggcactcttagatagtacatttcaaagcgaatggaacaatagctatgatgatttttccttttctttctggttatattagagatggacgtctaaaatcattatcccacaatgatcgaagcagtggaaatgatacatgagggacacgcaatcaggtcagtcagcagtcgcagtctacccaaggtaaactcacggacagcagctaatatccgacagatccgggacaattcatctgaatttgaggctaaaatatgaccaaccaagcctccaaccaaccccgccagtcactggggctgcaaagcttggtctagtgaaagtagccgatcactagtccaggcaaccgtgcatcaaatggcctgtatgcgaacatggtccctggttaacggaagccagtctgtttccaagcgaggtcaatttcaacggtgtcctctctagtggttgtcttcctgcaaccaggcatggtatgatttgtcggcctttagtgccatttcctgagtcaagcaggacaggtcaa

SEQ ID NO: 92 - DNA, 2052 bp, Aspergillus fumigatus
atgtggaagtcttttaaagaaaagcatgccagcaagtttgggggaggctcggctgaagcggcagcctcagacggtggccaagatctgaccacgatattggatagatcccaacgcggggaattgacagtgcttgttgcgctaatcgcacagcggatgcgggatggaatagaacaaaacttctccaatgctcctccttcgtcaggccagtctgtcaactacgaagaaaaacggacaggctccctgccccaatctacagatgcacaagaagaccagtcatcctccggcagcgcagcgaacggttcaagaaccgaccctcaattcaaagacccggagactgcgacatgtgcactatctaaatatgatgactggagggactccgtccttcttcgaattggtgaagtcgtcaacagggatccagaacatggtgaagttcaggcgaatgaaaacccaccctcaggacagcaatcccagcagatccgctccgaggaggatgaccgttccattcgtaagctacgtgaggtctttccgccggtggagaccagcctctctcaattgccagaagcgaagaaactcttaattctccactcattgctactcctagttttgagtcttgaacactataacgcctggtctcgggtcctgatgctgttcgtgacgtccagcttagggctggacgtgaaattactaaacgaagatgaagtcaaagtggcgagggggttgcttgacactgctctggcactgtcgtctaacgccccaaggcaggatgagagtcgaagtcgcgattcatcccgaaaatggaaggttgggatcgcgtcagttgcgggtgctgccctgatcgggatcactggtggactggctgcgccccttgttgcagctgggcttggtactgtcatgggcggccttgggcttggcgccaccgccgcagcagggtatctcggagctctcgctggaagtggtgttgtcgtcggcggacttttcggtgcttatggtgggcggatgaccggtcgtatggttgacaagtacgcacgggaggtggatgattttgcctttctgccgattcgtggttctcggcatcgatccgaagacgaaagagaagctgcccatcaggatcaccggctgcgggttaccatcggcgtgaccggatggctgacagaggaggacaatttcgtgatcccgtggcgagtgatcggagcggaatcggaggtgtttggtctccgctgggaaaccgagcccctgatgaatctgggaaatgcgcttgaccttttggtaaccagcgccgcatggactgccggtgaacaagtcctgaagaagacattcctctcccaactcttgaccgctgtcgcgctgccgcttggccttctcaaggtcgcacgtgtggtggacaatccgtttagcgtagcgaaggctcgggcggacaaggctggggaggttctcgcggatgctcttatcagtaaagtgcagggcgagcgaccagtcaccctcattggctactccttagggtctcgggtgattttcgcttgccttcaaagcttggcgaaacggcgcgcgttcggcttggtggaatccgcaattctgatgggagctcccaccccgtcgaattcagaacaatggtgtcgcatccgcagtgttgtgagtggacgccttgtcaacgtgtactcggaaaacgactccgtgctggctctcttatatcgaacaagcagcctccagcttggggttgcaggcttgcagcctgttgaaggtgtctcaggcgttgagaatctggacgttagcgacctgatcagcggccatctccgttatcagtttctcgttggcaggatcttgagcgttgttggacttgagagcattgatgctcgcgaggtcgcactggaggaggccgcattagaagccaaagatcggaggcaggagcaggaaagggctcataacgaacgacaggctggatttatgggcgagggtcggtcaccaagccagcggctggaaagccaggaggatctgcagggcgaagaggacagattacagaaagagatgggaaaagcacgagtgcggcactcttag

SEQ ID NO: 93 - DNA, 2052 bp, Aspergillus fumigatus; identical to SEQ ID NO: 92

SEQ ID NO: 94 - protein, 683 aa, Aspergillus fumigatus
MWKSFKEKHASKFGGGSAEAAASDGGQDLTTILDRSQRGELTVLVALIAQRMRDGIEQNFSNAPPSSGQSVNYEEKRTGSLPQSTDAQEDQSSSGSAANGSRTDPQFKDPETATCALSKYDDWRDSVLLRIGEVVNRDPEHGEVQANENPPSGQQSQQIRSEEDDRSIRKLREVFPPVETSLSQLPEAKKLLILHSLLLLVLSLEHYNAWSRVLMLFVTSSLGLDVKLLNEDEVKVARGLLDTALALSSNAPRQDESRSRDSSRKWKVGIASVAGAALIGITGGLAAPLVAAGLGTVMGGLGLGATAAAGYLGALAGSGVVVGGLFGAYGGRMTGRMVDKYAREVDDFAFLPIRGSRHRSEDEREAAHQDHRLRVTIGVTGWLTEEDNFVIPWRVIGAESEVFGLRWETEPLMNLGNALDLLVTSAAWTAGEQVLKKTFLSQLLTAVALPLGLLKVARVVDNPFSVAKARADKAGEVLADALISKVQGERPVTLIGYSLGSRVIFACLQSLAKRRAFGLVESAILMGAPTPSNSEQWCRIRSVVSGRLVNVYSENDSVLALLYRTSSLQLGVAGLQPVEGVSGVENLDVSDLISGHLRYQFLVGRILSVVGLESIDAREVALEEAALEAKDRRQEQERAHNERQAGFMGEGRSPSQRLESQEDLQGEEDRLQKEMGKARVRHS

SEQ ID NO: 95 - DNA, 3814 bp, Aspergillus fumigatus
ctttagtagcagttgtttctgaatattgcagtcatttcaaagagtccacgatgaagctcagaggagctgcttacgattatatcgctaaaagtacattaagggacgctctttgaaactttgtgttggacgtcacttatacagatcgaagggtttgcaggaaaataggagactactccatgtaggtaggtagcattttactatcgacgctgatagttgtcgtatgatctgagctggatccggagagcgatcggaggactacggcacgtgacaaagcctgcgagtgcaccgccatgcaaactttccgctggagctcagcaaaaagtccaccgaaattgccctgttgaatattccaacccaattgtgcccattgtcgtcccccaacttttctttcgggcattgtccccattagttgagctttatctcagtttactgtttatatcaatgcttcgcaggtgacgcccatagaacccgccgcctgcggtgcaagaggttctaccaccatgttctcgaaaccaaccaacagccccccggctgtcccctctcgaaatgctctccgagttctgcgtagattagcgcttgccggctcaactgtgggcagtttctgcacggtcgcggccatcacctacgatgtccatcgccgagtccgagtcgccgagcgcattgtcgagaacaaacgggccttacagacctccgcacccaactacgatgccacgtctgccgcgaaacgactcgcccgtatgatggaagcagcggaagcaggcgaatttatgggattggcgtcgttgaaggaggcggacagaaagatccgagaagggcaagctacgcaggatgacgatgtggtagctttgcaccaggaggggggctttcgacgtccgggcatggggttggaggatccgcggcgagcagtccaggcagagtcgtatctggagactacccggtcgcagctcgtcgatgcgcttatatcacggccgaatcttgagagtggcttgggtcgaaggaattttgttcctcctaggcaggtcccggttgtcgacgccatggagcggacgaaaaatgctatacaatcaaataattccagctcgcgggcgcaactttcggatgcattacccgagagcgagaaatcacagagtgcagggcaggttattgtcccgacacggatgcaggaacttctcgaccgcggtcgtcccattgaggcggcccaattttttctggagacacatgccgcttcattgaaaggcatatcctcagacaggaaggaaatggctacgaaggtattcttcgtcaactgcaaagaggacaatgtgtttattgccagaagtgtgtttgagcggttggaagaggtggatagaatcacgcctgagatgtggaagacgttgatgctggctttggcgaagaaagggtgcattgaatcagttgcgagtgtttatacgcgatacatgcgcaagttcccttgccctccggagatggtcgacgttgtgctgcggagtttgcttgaatcccaccgactcaccactgcgaaatggttcctcttgcgcaatctacaacatgaccgtgattgcggtttgtgcggagcctacctctccggcctgtggcggaagacaagaagcatcgagttattgaacgggcagttgaaaaagatattgaccattcttccaaagttcgaaaaacaacctagcgacaaactatttaacccggtgatcaaggcatatgttgaatttgggcgggttgccgatgccgaagctctggtgcatgatatgaccactttatatgggatccctcttcgttgccgaactcagggcctattggtatacgccaaggctctgaactgtgactgggagggagttgacgcaggactgcaagagatgcacaaactcaagctgacgagacgccggcgagacttccttcctatttttgatcgaatatttctggagtactgggtctcacattcgggaattgagattcgcaattttgtgttccggtaccttgataaattcgacattgtccccgatcgcgtgctctacaagcacatcttggaggctttcgtggagaaaggagacaaggaaatgattgctgaatttacaagtatggcgaagcagcgatcgtggaatatccccataaacgagcagcaattcttggagatactacggtctcgtcgcctcgcattggaaggagcaccagtggggttctggcaaatgctgcaggcagcgcgtgtcaagtacgggcacagctccacgtctcagcgtatcatgggctacgaccaacagtcattccctttgccggaagtcaacagcatgccatatacacagaatccgctatcgtggtaccagagaacgatgcaagagaccacgccgtcgaagcctgtcgaccaatatcagaaactccataagcagatgacccattttctgcacgctggaaagctgaaggaagcattgaagtgctttcaaaatgctaaaaatgcccggttccagatgaggcagcttcacgttgaattggcggtcatagcgactttgcttgaggacggccttagtgcagcgcgcagtctcatcgagtctgaatggcggactatccgtcaccttgtccgcttctctcctatcttctttcgtcaagtcatggcggtcgacgaggatgcgggtggccacattgtccagatggcagtcctacgcttctaccagctttgttggtctacgaaacacatgaaggtcaagcatcaccttactgttgcgacaagccgtcgcttgatctcccaacataagccggaaatggctctggagcttctgacggccgtgtacaagtcccgttataggtttgcagcgacctttgacggggtttgcatgaagatgtttgcgcgcgccttcgccgcgacagacaacattctaggcttacgatggtgtattcttactgctttatcacgcgatagtgcactcaatcatgattttgtggtggaagttcgccgaatcttgggcactctaagtccaccttccgcggtggatgccaccgccggccccgtcactcatgagcagctggaatacctttattacatcgccgatctgctcgaggagaagaatgaggggtgcgccccgatatgggagctcaagcatgacgccacactgaagcagtcttcgaggcgacagttgaagcaacctcttgacgcaagccgtcttttcaaccagtccgatgtccgcgagaccgtcaagcgatgggacgaagaatacgagctggaggcagtgctgggtaggattgacaatgacccgaactcgattactgctcgatggaacgagagtcgtcgttcacatcagaaagaggcgggtttatgacgatcggtctgataccatctaggaagtggttgcaactgcaacgcctggccgagcctttattgaatcatgtgataggctccttagccactgtatatatactattgacactctgtactttactctcctgtgcggtttgtgactgccattatttctcatcggaaatagaatagagatggtgagattggatgcaaatatcacggatatggccgaaaccccttggctctccttttgcaggcaacaacgccgaacggtatccaataataaacactgtaattgatcggatgggatcagaccagaagatgctcttttccggtcctttcagtgatcatacggtgttcctgaattttctctcaccaataagaatctagcactgcggaaggaggaaagccagtctttcacatgtcacgccactgtccactcttggcccctttccagtaaaaacacttaaagttctccgatctttttctatctccgccatcccatccttttcatcaattgct

SEQ ID NO: 96 - DNA, 2814 bp, Aspergillus fumigatus
atgttctcgaaaccaaccaacagccccccggctgtcccctctcgaaatgctctccgagttctgcgtagattagcgcttgccggctcaactgtgggcagtttctgcacggtcgcggccatcacctacgatgtccatcgccgagtccgagtcgccgagcgcattgtcgagaacaaacgggccttacagacctccgcacccaactacgatgccacgtctgccgcgaaacgactcgcccgtatgatggaagcagcggaagcaggcgaatttatgggattggcgtcgttgaaggaggcggacagaaagatccgagaagggcaagctacgcaggatgacgatgtggtagctttgcaccaggaggggggctttcgacgtccgggcatggggttggaggatccgcggcgagcagtccaggcagagtcgtatctggagactacccggtcgcagctcgtcgatgcgcttatatcacggccgaatcttgagagtggcttgggtcgaaggaattttgttcctcctaggcaggtcccggttgtcgacgccatggagcggacgaaaaatgctatacaatcaaataattccagctcgcgggcgcaactttcggatgcattacccgagagcgagaaatcacagagtgcagggcaggttattgtcccgacacggatgcaggaacttctcgaccgcggtcgtcccattgaggcggcccaattttttctggagacacatgccgcttcattgaaaggcatatcctcagacaggaaggaaatggctacgaaggtattcttcgtcaactgcaaagaggacaatgtgtttattgccagaagtgtgtttgagcggttggaagaggtggatagaatcacgcctgagatgtggaagacgttgatgctggctttggcgaagaaagggtgcattgaatcagttgcgagtgtttatacgcgatacatgcgcaagttcccttgccctccggagatggtcgacgttgtgctgcggagtttgcttgaatcccaccgactcaccactgcgaaatggttcctcttgcgcaatctacaacatgaccgtgattgcggtttgtgcggagcctacctctccggcctgtggcggaagacaagaagcatcgagttattgaacgggcagttgaaaaagatattgaccattcttccaaagttcgaaaaacaacctagcgacaaactatttaacccggtgatcaaggcatatgttgaatttgggcgggttgccgatgccgaagctctggtgcatgatatgaccactttatatgggatccctcttcgttgccgaactcagggcctattggtatacgccaaggctctgaactgtgactgggagggagttgacgcaggactgcaagagatgcacaaactcaagctgacgagacgccggcgagacttccttcctatttttgatcgaatatttctggagtactgggtctcacattcgggaattgagattcgcaattttgtgttccggtaccttgataaattcgacattgtccccgatcgcgtgctctacaagcacatcttggaggctttcgtggagaaaggagacaaggaaatgattgctgaatttacaagtatggcgaagcagcgatcgtggaatatccccataaacgagcagcaattcttggagatactacggtctcgtcgcctcgcattggaaggagcaccagtggggttctggcaaatgctgcaggcagcgcgtgtcaagtacgggcacagctccacgtctcagcgtatcatgggctacgaccaacagtcattccctttgccggaagtcaacagcatgccatatacacagaatccgctatcgtggtaccagagaacgatgcaagagaccacgccgtcgaagcctgtcgaccaatatcagaaactccataagcagatgacccattttctgcacgctggaaagctgaaggaagcattgaagtgctttcaaaatgctaaaaatgcccggttccagatgaggcagcttcacgttgaattggcggtcatagcgactttgcttgaggacggccttagtgcagcgcgcagtctcatcgagtctgaatggcggactatccgtcaccttgtccgcttctctcctatcttctttcgtcaagtcatggcggtcgacgaggatgcgggtggccacattgtccagatggcagtcctacgcttctaccagctttgttggtctacgaaacacatgaaggtcaagcatcaccttactgttgcgacaagccgtcgcttgatctcccaacataagccggaaatggctctggagcttctgacggccgtgtacaagtcccgttataggtttgcagcgacctttgacggggtttgcatgaagatgtttgcgcgcgccttcgccgcgacagacaacattctaggcttacgatggtgtattcttactgctttatcacgcgatagtgcactcaatcatgattttgtggtggaagttcgccgaatcttgggcactctaagtccaccttccgcggtggatgccaccgccggccccgtcactcatgagcagctggaatacctttattacatcgccgatctgctcgaggagaagaatgaggggtgcgccccgatatgggagctcaagcatgacgccacactgaagcagtcttcgaggcgacagttgaagcaacctcttgacgcaagccgtcttttcaaccagtccgatgtccgcgagaccgtcaagcgatgggacgaagaatacgagctggaggcagtgctgggtaggattgacaatgacccgaactcgattactgctcgatggaacgagagtcgtcgttcacatcagaaagaggcgggtttatga

SEQ ID NO: 97 - DNA, 2814 bp, Aspergillus fumigatus; identical to SEQ ID NO: 96

SEQ ID NO: 98 - protein, 937 aa, Aspergillus fumigatus
MFSKPTNSPPAVPSRNALRVLRRLALAGSTVGSFCTVAAITYDVHRRVRVAERIVENKRALQTSAPNYDATSAAKRLARMMEAAEAGEFMGLASLKEADRKIREGQATQDDDVVALHQEGGFRRPGMGLEDPRRAVQAESYLETTRSQLVDALISRPNLESGLGRRNFVPPRQVPVVDAMERTKNAIQSNNSSSRAQLSDALPESEKSQSAGQVIVPTRMQELLDRGRPIEAAQFFLETHAASLKGISSDRKEMATKVFFVNCKEDNVFIARSVFERLEEVDRITPEMWKTLMLALAKKGCIESVASVYTRYMRKFPCPPEMVDVVLRSLLESHRLTTAKWFLLRNLQHDRDCGLCGAYLSGLWRKTRSIELLNGQLKKILTILPKFEKQPSDKLFNPVIKAYVEFGRVADAEALVHDMTTLYGIPLRCRTQGLLVYAKALNCDWEGVDAGLQEMHKLKLTRRRRDFLPIFDRIFLEYWVSHSGIEIRNFVFRYLDKFDIVPDRVLYKHILEAFVEKGDKEMIAEFTSMAKQRSWNIPINEQQFLEILRSRRLALEGAPVGFWQMLQAARVKYGHSSTSQRIMGYDQQSFPLPEVNSMPYTQNPLSWYQRTMQETTPSKPVDQYQKLHKQMTHFLHAGKLKEALKCFQNAKNARFQMRQLHVELAVIATLLEDGLSAARSLIESEWRTIRHLVRFSPIFFRQVMAVDEDAGGHIVQMAVLRFYQLCWSTKHMKVKHHLTVATSRRLISQHKPEMALELLTAVYKSRYRFAATFDGVCMKMFARAFAATDNILGLRWCILTALSRDSALNHDFVVEVRRILGTLSPPSAVDATAGPVTHEQLEYLYYIADLLEEKNEGCAPIWELKHDATLKQSSRRQLKQPLDASRLFNQSDVRETVKRWDEEYELEAVLGRIDNDPNSITARWNESRRSHQKEAGL

SEQ ID NO: 99 - DNA, 2401 bp, Aspergillus fumigatus
acatggatgaatcactgcccgatatcgagaagtcgcttgaaccgtacctctcacattttggttatcggagtgacggatcggatcagagcgcagtatttgatcttttagaaagtgagcgtttcactcataacaagagccccttgacagacattcgcaagggttaacctcatggttgtggtttggtttggtttgctctgcttataattctctcggcatgtcggtaatcaatccacttttctctggggcccttctacacatgcagcaatttcattggtcatctgagatatacagatttgtaaggcagtatcagtatcatcaggatacgcttctatgtagtatgagctcaaagaacagatagttgagaagtagtactgatatcacgtggtttaagtattttgtcggagtgctaagcattacaagtggcatatcctttcagaatagaaaaccatcaggcaaacgcgattatcaccagaatttccatctcagtctacagattggatatgcagagatccaattgccacagcagtcaagtcgtcagaattctgcgtggagcgaggagacctcgcattcgtgcaggaaaacctgtgacgtcgagagaagttttccgcagatggagtagctcagagacggcgaaatcatcttcagctgcaaatcagaccatattctctgggatccagccaaccggcgtaccgcaccttggaaattatctgggagctctacgtgaatgggtacggctacaaaatgctgcgaaggagggaacaagattgtttttttctatcgtcgacttgcatgcgttgacagtgccacaagacgcctcccagctacgaaattggagaaaagagacatttgcaacactcattgccgttggtttagacccgaatcgctcaacgattttctaccagtccgccgtatgcagtatgaatatgggttgtaaagtttactgaccatgtcattaggtccatgcacacgccgaactattttggattttgtgcacaatagcctctatgggatatctctcccgaatgacacagtggaaggtaatacccagatcaagtgccaacctggtccaatggtcagaaggaacagctagctgacgtcggccagagcaaactccagttgcctgataacgcaaatctggaagactcgacggcgaggtcaaggttgcgattggggttgttctcataccctgttctgcaggcggcggatattctagttcataggtatgctgcccaatcgttggtacattttcacattactgatgtagatcagagctactcatgttcctgtcggagatgatcaaaggcagcacctcgaattttcgaggaacactgcgaatagtttcaatcatgtatatggacccattttcccgtcaccagaagcaattatatgtaagtggtttttgcttctggcggaacggtcctgtgttgggaaattgacggctatcaatagcgcctgctaaacgggttatgtccctcaaagaaccgacgttgaaaatgtccaagtcccatgccgacagacgctcaaggatcattcttacggattcgcccgcagaaatctccaaaaagatcaatgctgcgctcacagactcggaattaaccattacatatgacccagtccgtcgacctggagtggcgaatttaatagagatcttgagtcacttcgatggacgaacttgcgatgagattgccatggaataccgttcagccagtcttcgcgctctaaaggaacatctggccagaacgttgtccaatcatcttgagccaataagagagaagtatctctcacttgtaggagatcagactgactaccttgattctatagcagaacagggttctgaagccgcgcgggccaacgctgaattgacaatggagcaagtcaaagtcgctatgggcttaatttagcccttcatatacgttgtcatcatgctccgccgggtacacatgtaggctcagttggtcatactctgatgatcagttcatgatggtggccactattgacaattacccaccactattgcctgttcaggtactactcaaacgctgattatacaatataatgcatgcatatatatctacgtgtgtgtgtgtgtgtatacatatgtatgaatatgtgcatagctaatcaaatctcgcttgggttggtgaaagaaatgcgacatatgctttagagaacattctcgcctgggattgaattcatttcacaaccgtatagaggtacatcatctagttattggcagggatacgtcatccatactggataatggctgctgcctacagacccagactggtaggtatgtccactgcgtggagagcactaaagaggcccagcttggtgtgcggcaagtgggagcattactaggagcatgcagtgtccttcgtacatgtaccttttacatatgctc

SEQ ID NO: 100 - DNA, 1401 bp, Aspergillus fumigatus
atgcagagatccaattgccacagcagtcaagtcgtcagaattctgcgtggagcgaggagacctcgcattcgtgcaggaaaacctgtgacgtcgagagaagttttccgcagatggagtagctcagagacggcgaaatcatcttcagctgcaaatcagaccatattctctgggatccagccaaccggcgtaccgcaccttggaaattatctgggagctctacgtgaatgggtacggctacaaaatgctgcgaaggagggaacaagattgtttttttctatcgtcgacttgcatgcgttgacagtgccacaagacgcctcccagctacgaaattggagaaaagagacatttgcaacactcattgccgttggtttagacccgaatcgctcaacgattttctaccagtccgccgtatgcagtatgaatatgggttgtaaagtttactgaccatgtcattaggtccatgcacacgccgaactattttggattttgtgcacaatagcctctatgggatatctctcccgaatgacacagtggaaggtaatacccagatcaagtgccaacctggtccaatggtcagaaggaacagctagctgacgtcggccagagcaaactccagttgcctgataacgcaaatctggaagactcgacggcgaggtcaaggttgcgattggggttgttctcataccctgttctgcaggcggcggatattctagttcataggtatgctgcccaatcgttggtacattttcacattactgatgtagatcagagctactcatgttcctgtcggagatgatcaaaggcagcacctcgaattttcgaggaacactgcgaatagtttcaatcatgtatatggacccattttcccgtcaccagaagcaattatatgtaagtggtttttgcttctggcggaacggtcctgtgttgggaaattgacggctatcaatagcgcctgctaaacgggttatgtccctcaaagaaccgacgttgaaaatgtccaagtcccatgccgacagacgctcaaggatcattcttacggattcgcccgcagaaatctccaaaaagatcaatgctgcgctcacagactcggaattaaccattacatatgacccagtccgtcgacctggagtggcgaatttaatagagatcttgagtcacttcgatggacgaacttgcgatgagattgccatggaataccgttcagccagtcttcgcgctctaaaggaacatctggccagaacgttgtccaatcatcttgagccaataagagagaagtatctctcacttgtaggagatcagactgactaccttgattctatagcagaacagggttctgaagccgcgcgggccaacgctgaattgacaatggagcaagtcaaagtcgctatgggcttaatttag

SEQ ID NO: 101 - DNA, 1200 bp, Aspergillus fumigatus
atgcagagatccaattgccacagcagtcaagtcgtcagaattctgcgtggagcgaggagacctcgcattcgtgcaggaaaacctgtgacgtcgagagaagttttccgcagatggagtagctcagagacggcgaaatcatcttcagctgcaaatcagaccatattctctgggatccagccaaccggcgtaccgcaccttggaaattatctgggagctctacgtgaatgggtacggctacaaaatgctgcgaaggagggaacaagattgtttttttctatcgtcgacttgcatgcgttgacagtgccacaagacgcctcccagctacgaaattggagaaaagagacatttgcaacactcattgccgttggtttagacccgaatcgctcaacgattttctaccagtccgccgtccatgcacacgccgaactattttggattttgtgcacaatagcctctatgggatatctctcccgaatgacacagtggaagaaggaacagctagctgacgtcggccagagcaaactccagttgcctgataacgcaaatctggaagactcgacggcgaggtcaaggttgcgattggggttgttctcataccctgttctgcaggcggcggatattctaatcagagctactcatgttcctgtcggagatgatcaaaggcagcacctcgaattttcgaggaacactgcgaatagtttcaatcatgtatatggacccattttcccgtcaccagaagcaattatatcgcctgctaaacgggttatgtccctcaaagaaccgacgttgaaaatgtccaagtcccatgccgacagacgctcaaggatcattcttacggattcgcccgcagaaatctccaaaaagatcaatgctgcgctcacagactcggaattaaccattacatatgacccagtccgtcgacctggagtggcgaatttaatagagatcttgagtcacttcgatggacgaacttgcgatgagattgccatggaataccgttcagccagtcttcgcgctctaaaggaacatctggccagaacgttgtccaatcatcttgagccaataagagagaagtatctctcacttgtaggagatcagactgactaccttgattctatagcagaacagggttctgaagccgcgcgggccaacgctgaattgacaatggagcaagtcaaagtcgctatgggcttaatttag

SEQ ID NO: 102 - protein, 399 aa, Aspergillus fumigatus
MQRSNCHSSQVVRILRGARRPRIRAGKPVTSREVFRRWSSSETAKSSSAANQTIFSGIQPTGVPHLGNYLGALREWVRLQNAAKEGTRLFFSIVDLHALTVPQDASQLRNWRKETFATLIAVGLDPNRSTIFYQSAVHAHAELFWILCTIASMGYLSRMTQWKKEQLADVGQSKLQLPDNANLEDSTARSRLRLGLFSYPVLQAADILIRATHVPVGDDQRQHLEFSRNTANSFNHVYGPIFPSPEAIISPAKRVMSLKEPTLKMSKSHADRRSRIILTDSPAEISKKINAALTDSELTITYDPVRRPGVANLIEILSHFDGRTCDEIAMEYRSASLRALKEHLARTLSNHLEPIREKYLSLVGDQTDYLDSIAEQGSEAARANAELTMEQVKVAMGLI

SEQ ID NO: 103 - DNA, 3805 bp, Aspergillus fumigatus
cacccattttgtagtaattctcgagaaatccgctctgaatacccaccaggggaaagatgtcaattatcactatgagaaacaaaaggctgctattttacctcttcgggacgtgttggacgcgtcgcggggcatgctatgtttgtcctgtttgcaccaaacaggaattgccgtaaaatgggaacagagtttgttagctcaccgcctttggattttcactgctaaaacaaaaaaaaagtgagatttaccatcacgacagggctggatctgttctggtcggaaggagacggcaggcgatttggatttctatttcgcccttgcaaagtaattggatgcaaagggttctgtgttctattttgcgtcttgcaacttgatggaattcgaagcctgagctgtcctgagcttcaaaatatcttctctcgccctcgggcgacctcaaaccacttttctttcccatattttgtttggagcggcctgcgccagctgcgcaacagagttccaagatgcctcaccgcgcggcatccccagcggtgtcggaaaatgagttcgacatcacaggcgctctcttccagaacgacagcgactccgacaacgaacagccctcggccaagtccaaacgacaacctccgaagaaggttccctcgcaggcgctcgacttcctcggcgatgtgaatgaagacgacaatgacgacgaggcttttattgccgagcagcaaacttctgccaaccgcaaggcctcgaatctcaaaggtcgcactgttaagaagggtggtggtttccaagccatgggcttgagtgccaatctgttaaaggcaatcgctcggaaaggcttctcggtaccgactcccattcagcgcaagaccattcccgttattatggacgaccaggatgtagttggtatggcacggactggttcaggaaagacggccgctttcgttatcccaatgatcgagaaattgaagagccatagcaccaaggttggagcccgcggtctggtcttgtccccatcgagagagctggcactgcagacattgaaagtcgtcaaggaactgggtagaggcactgacctgaagtcggttcttcttgttggtggagacagcctggaggagcaattcgcgatgatcgccggcaatccagatattattattgcaacacctggtcgattcctgcatttgaaggtggaaatgaacctggacttgtccagtatccgctatgtcgttttcgacgaggctgatcgactgtttgagatgggtttcgccgcgcaactaacagagattttgcacggtcttcctgcgaatcggcagactctcctgttctctgccactcttccgaagtcccttgtcgagtttgcccgcgccggcttgcaggaacctacactagtccgtctggataccgagagcaagatctcgcccgatcttcagaatgctttcttctctgtgaaatcctcagagaaggaaggggccttgctctacatccttcatgaggtcatcaagatgccgactgggccaaccgaggtttcgcaacaaaggaaagaagaagatgcaagcgccaagaacttgaagaacaagaagaggaagagagcggaaatggagaaggctgtcaatacgagagagtctccaaccaagcattcgacaatcgtttttgccgcgacgaagcaccatgtcgattatctttactcactactctgcgaggcgggattcgccgtctcctacgtttacggctctttagatcaaaccgcccgaaagatccaggttcaaaactttagaacgggcatgaccaatatcctcgttgtcaccgacgttgcggctagaggtattgatattcctatcctggcaaatgtcattaattacgatttcccctcccagccaaagatttttgttcaccgtgtcggccgaactgctcgtgccgggcgcaagggctggagttacagtctggtccgcgatgcagacgctccttatttacttgatttgcaactattcctaggaagaaggctggttgttggccgcgaattcggggatcaagtgaacttcgccgaagacgttgtaacgggcagtctacctagggatgggctctctcaaagctgtgagtgggtaaccaaggtcctggacgataatgcagaccttgcagcccaacgcacggtcgctgctaagggtgagaagctctatatgcgaacccggaacgcggcatctcttgagagtgcgaagcgatcgaaacaggtggtttcttccgacaattggacaagcgtacacccgctcttccaagatgaaactagtaatctggaggccgagcgggagaagatgctcgcccgtattggtggttaccggccgccagagacaatctttgaggtcaacaaccggcggatgggcaaacatgagaacgtcgacgctctcgatacgattaagagagttcgtagcactctagagtccaagaagaagcgtgcacaagcgaatgaaaagtccgagttcctcgaagacggtcccgacgacggaaaggcagtcaacgaagccaaagaaaccgagagcgagggagcattttctgatgaggacgacgacgttcccaccggcgtggcagataacatgtcgatggcatccgattcagagctggaagtcaccttctcgtcatattcaaaatcaaaggacaacaaggcgaagaaagcgagcgcggcatctttccagaaccctgaatacttcatgtcctataccccgaataacacctccctggcggaggaccgagcttatggtgtgcattccggtaccaactccaacttcgcccaggcctcacgcagcgcgaccatggatctggcaggcgacgatggaggccgcgggttcggcgaggctcgtacgctgatgcgctgggacaagcgacacaagaagtacgtggctcgacagaatgacgaggacggctccaagggcacacgcctcgtccgcggtgagagcggggcgaagatcgcagccagcttccggagcggacggttcgacgcttggaagagagaaaatcgtctcggccgcttgcctcgggtgggtgaggccgaagctgctaatctcgctgctggtctcaacgcagccatctcaggcaagcggttcaggcatcgcaaggagcaggcgcccaagaaggctgatcctctccgtggtgattacgagaagatgaagaagaaggctgaactcgccaaggagcgagcaatgtctaaggctggcggcgctgcaccacgtggcaagagcgagctgaagagtacggacgatatccggattgcgcgcaaattgaagcagaagagacgagagaagaatgctcgtccctcgaggaagaagtaaccgggaggttaattttcagagtcatgtatataccaggaattgcctatgatacctgctactgtgtaatttttttttatactaaagcccaatccaaaacactcttgggaacgaccgaggccaaacattcctggtgacagagattacctttcagcacttatgccaaaccacaaagctctctcagtaaaaccgccaatacgttaacacccctccaatactccgtacagcagctctcatcgcatcactgagaaaatgtcgcttgatattctacccaacagtgaagaagacccacagacccaagccgaaaatttcaaggtgaccggaattttctcggccgcaaagggagaacaagatagaatgcaaatggacgatctcccaatctccatcagctggactattgattccgacggtgaatagtggagcatctgtgctgcgctattgggttctccgtgacgccgctcctttggtgactcgttttctttgctttttccttcagcacttgg

SEQ ID NO: 104 - DNA, 2805 bp, Aspergillus fumigatus
atgcctcaccgcgcggcatccccagcggtgtcggaaaatgagttcgacatcacaggcgctctcttccagaacgacagcgactccgacaacgaacagccctcggccaagtccaaacgacaacctccgaagaaggttccctcgcaggcgctcgacttcctcggcgatgtgaatgaagacgacaatgacgacgaggcttttattgccgagcagcaaacttctgccaaccgcaaggcctcgaatctcaaaggtcgcactgttaagaagggtggtggtttccaagccatgggcttgagtgccaatctgttaaaggcaatcgctcggaaaggcttctcggtaccgactcccattcagcgcaagaccattcccgttattatggacgaccaggatgtagttggtatggcacggactggttcaggaaagacggccgctttcgttatcccaatgatcgagaaattgaagagccatagcaccaaggttggagcccgcggtctggtcttgtccccatcgagagagctggcactgcagacattgaaagtcgtcaaggaactgggtagaggcactgacctgaagtcggttcttcttgttggtggagacagcctggaggagcaattcgcgatgatcgccggcaatccagatattattattgcaacacctggtcgattcctgcatttgaaggtggaaatgaacctggacttgtccagtatccgctatgtcgttttcgacgaggctgatcgactgtttgagatgggtttcgccgcgcaactaacagagattttgcacggtcttcctgcgaatcggcagactctcctgttctctgccactcttccgaagtcccttgtcgagtttgcccgcgccggcttgcaggaacctacactagtccgtctggataccgagagcaagatctcgcccgatcttcagaatgctttcttctctgtgaaatcctcagagaaggaaggggccttgctctacatccttcatgaggtcatcaagatgccgactgggccaaccgaggtttcgcaacaaaggaaagaagaagatgcaagcgccaagaacttgaagaacaagaagaggaagagagcggaaatggagaaggctgtcaatacgagagagtctccaaccaagcattcgacaatcgtttttgccgcgacgaagcaccatgtcgattatctttactcactactctgcgaggcgggattcgccgtctcctacgtttacggctctttagatcaaaccgcccgaaagatccaggttcaaaactttagaacgggcatgaccaatatcctcgttgtcaccgacgttgcggctagaggtattgatattcctatcctggcaaatgtcattaattacgatttcccctcccagccaaagatttttgttcaccgtgtcggccgaactgctcgtgccgggcgcaagggctggagttacagtctggtccgcgatgcagacgctccttatttacttgatttgcaactattcctaggaagaaggctggttgttggccgcgaattcggggatcaagtgaacttcgccgaagacgttgtaacgggcagtctacctagggatgggctctctcaaagctgtgagtgggtaaccaaggtcctggacgataatgcagaccttgcagcccaacgcacggtcgctgctaagggtgagaagctctatatgcgaacccggaacgcggcatctcttgagagtgcgaagcgatcgaaacaggtggtttcttccgacaattggacaagcgtacacccgctcttccaagatgaaactagtaatctggaggccgagcgggagaagatgctcgcccgtattggtggttaccggccgccagagacaatctttgaggtcaacaaccggcggatgggcaaacatgagaacgtcgacgctctcgatacgattaagagagttcgtagcactctagagtccaagaagaagcgtgcacaagcgaatgaaaagtccgagttcctcgaagacggtcccgacgacggaaaggcagtcaacgaagccaaagaaaccgagagcgagggagcattttctgatgaggacgacgacgttcccaccggcgtggcagataacatgtcgatggcatccgattcagagctggaagtcaccttctcgtcatattcaaaatcaaaggacaacaaggcgaagaaagcgagcgcggcatctttccagaaccctgaatacttcatgtcctataccccgaataacacctccctggcggaggaccgagcttatggtgtgcattccggtaccaactccaacttcgcccaggcctcacgcagcgcgaccatggatctggcaggcgacgatggaggccgcgggttcggcgaggctcgtacgctgatgcgctgggacaagcgacacaagaagtacgtggctcgacagaatgacgaggacggctccaagggcacacgcctcgtccgcggtgagagcggggcgaagatcgcagccagcttccggagcggacggttcgacgcttggaagagagaaaatcgtctcggccgcttgcctcgggtgggtgaggccgaagctgctaatctcgctgctggtctcaacgcagccatctcaggcaagcggttcaggcatcgcaaggagcaggcgcccaagaaggctgatcctctccgtggtgattacgagaagatgaagaagaaggctgaactcgccaaggagcgagcaatgtctaaggctggcggcgctgcaccacgtggcaagagcgagctgaagagtacggacgatatccggattgcgcgcaaattgaagcagaagagacgagagaagaatgctcgtccctcgaggaagaagtaa

SEQ ID NO: 105 - DNA, 2805 bp, Aspergillus fumigatus; identical to SEQ ID NO: 104

SEQ ID NO: 106 - protein, 934 aa, Aspergillus fumigatus; identical to SEQ ID NO: 3

SEQ ID NO: 107 - DNA, 2413 bp, Aspergillus fumigatus
cctcctcttcttccactgcctcatcatcacggtatggattgtcatcagccatagtgagaatagtgaatgggtttagaagttacagcctagaaatgagagggaaagagaggtacattgaaagcaagaagatatggtcggtatttacatcacctccttctctgccttgagtgcctaccgctctgctgcctgcgtgcccaccgacttcaccagtgacgcgatctcgtcatcggagcaacgcgggggaaacacgaacatcacgtgaatgcgccaagacttgatgaccccaaattattactgattggtcaaacttccagcactgttccgtcatcaaccacctaagggcctagatatgttctccagttacagatccttcgtgccacgattctttcattgagtggtcaaatactactcgacgtatttttgtgggcttcagtttgtggctaatgttagaccgatagacgacggccaacctttttaatacactatcatcgcacctccccatggctctccgccggccattaacacttccgaggcacattctcaatggagcttgtttaggcttgcgaccagctgtgtctcgcgccgctctggcttatgggcaggagcagaggaaagggcttgcaacagcagttcccccggtcactcaaaatgcggctgggtccaaaggccccacggcaatggtcttcctcaacatgggtgggccatcgaagattgacgaagtggaagattttctgagcagattatttgtatgcattcctcaatatgcccgatgctaccaccatgtatgagtctgacaaactctctcttcctataccaggccgatggcgatctgattcctctcggacgacttcaatcatacctcggccctctcatcgctaagcgcagaaccccaaagatccaacggcaatactcggatattggtggagggtcaccgatcaggaaatggtccgagtatcagtgcgaggaaatgtgcagattgctagacaaaatcaatcccgaaacggctcctcacaagccttacgtcgcgttccggtacgccgaccctctgacggaagaaatgtacacaaagttgctggaagatggattcggcaacgggaaaggcgggcgcgctgtcgcgttcacacagtacccccaatattcgtgctccaccacgggtagctcgctgaacgagttgtggaaatggagaaccaggcttgagggtaagcgtgcaaatggcaacatggaccccgctggtgccatccagtggagtgtcattgatcgatggccaacgcaccctggcctcgtggaggctttcgcccggaacattgaggagcagctgaagacatacccagaggagaagcgaaacggtgtcgttctcttgttctcagcccacagtctgcccatgagtgttgtcaacagaggtgagactcatcttcttaccgaacaacaagatttgctcgctaacacatttcctaggcgacccatatcctgctgaagttgctgcaactgtgcatgctgtcatgcaaagattgaatttcagcaatccttaccgactgtgctggcagtcccaagtgggaccgtcagcttggcttggagcccaaactagcgatacggtcgaaaactatgtcaaacgtggacagaccgatattattctagttcccattgccttcaccagcgaccatattgagactctgtacgagttggatctggaagtgataaaggaagcaaactccccgggagtcaagagagccgagagtttgaatggtaaccccattttcattcaggcattagcagacattgcccaagagcacctccgtaagggagagaagtgctcactacagatgactctgcgctgtcaaggctgtaagagcgaacggtgcctggaacagaagaaattctttgctggcgaccgattttcttctcttgtagtttagtgtatttctatatggctgttctctgccgttgtatacaatactaggggccattttctttcttcagggcgttaagttgtgttgtcaacgaggtgacccattcacggagcacagcagagcaatgtatatattgttgaaaaagtccaagcatataatctcatttcgttgttggtcttcaatggttgagaatatataaaatcattaggaatgaaggatttccgggtcaacctttgacaattattttgccaccatttaacgttgcacatatgcaacgataaagcacgacccagtcgtagtgttggtgtcccttatcgagaaattgcatgagattactccaaccgctcaaaatctcaaaccctgataagaaaaattgattattgaacaaactctcatagcgtcgtttcgtaattactggttggtcggaatcgggctgacgcaaagcccaacggccacgaagctgctccagctccgttcccagttctcgtgaagaattggtaatcatg

SEQ ID NO: 108 - DNA, 1413 bp, Aspergillus fumigatus; identical to SEQ ID NO: 49

SEQ ID NO: 109 - DNA, 1287 bp, Aspergillus fumigatus
atggctctccgccggccattaacacttccgaggcacattctcaatggagcttgtttaggcttgcgaccagctgtgtctcgcgccgctctggcttatgggcaggagcagaggaaagggcttgcaacagcagttcccccggtcactcaaaatgcggctgggtccaaaggccccacggcaatggtcttcctcaacatgggtgggccatcgaagattgacgaagtggaagattttctgagcagattatttgccgatggcgatctgattcctctcggacgacttcaatcatacctcggccctctcatcgctaagcgcagaaccccaaagatccaacggcaatactcggatattggtggagggtcaccgatcaggaaatggtccgagtatcagtgcgaggaaatgtgcagattgctagacaaaatcaatcccgaaacggctcctcacaagccttacgtcgcgttccggtacgccgaccctctgacggaagaaatgtacacaaagttgctggaagatggattcggcaacgggaaaggcgggcgcgctgtcgcgttcacacagtacccccaatattcgtgctccaccacgggtagctcgctgaacgagttgtggaaatggagaaccaggcttgagggtaagcgtgcaaatggcaacatggaccccgctggtgccatccagtggagtgtcattgatcgatggccaacgcaccctggcctcgtggaggctttcgcccggaacattgaggagcagctgaagacatacccagaggagaagcgaaacggtgtcgttctcttgttctcagcccacagtctgcccatgagtgttgtcaacagaggcgacccatatcctgctgaagttgctgcaactgtgcatgctgtcatgcaaagattgaatttcagcaatccttaccgactgtgctggcagtcccaagtgggaccgtcagcttggcttggagcccaaactagcgatacggtcgaaaactatgtcaaacgtggacagaccgatattattctagttcccattgccttcaccagcgaccatattgagactctgtacgagttggatctggaagtgataaaggaagcaaactccccgggagtcaagagagccgagagtttgaatggtaaccccattttcattcaggcattagcagacattgcccaagagcacctccgtaagggagagaagtgctcactacagatgactctgcgctgtcaaggctgtaagagcgaacggtgcctggaacagaagaaattctttgctggcgaccgattttcttctcttgtagtttag

SEQ ID NO: 110 - protein, 428 aa, Aspergillus fumigatus; identical to SEQ ID NO: 51

SEQ ID NO: 111 - DNA, 2865 bp, Aspergillus fumigatus
ccttgatcgaatcccaagcctgtagcctggcagaacagagcaaagcaaactggcatctcctgctgaagcctaaggtcactagacaaaggaacggcagaaccgcatgaacgaggagcataaacaaggcgggatcgtctttcaacacgcgggcggtgagcttatcagcgccgattgcggggtggtgcagcaaatcaagtcagtcgccactttcggcgaccatgacaaagcgcaagattggctttactaaaccgcctactttttttttatcaaagagacttgggtttgtcagcttttctttatcttctgaaagagcgcttctctggtcaagctgttcaccaaatccccatcactactgttccctttgtcgttattttcgtcgcattgcatctacaacaaagaaaacgggctcgacgaaccctgcgagatccatacttcctggggtggcggtcttcttagtccttatcgcatagcggggtgctcgaccagaagtccctgccacgatgagtgcaatcctttctgcagacgatttgaacgatttcatttctcccggggttgcttgcatcaagcccgttgagagtctaccacaaaaagaatcccagtcggaggtatctttcctgtcttaccagtcatctgttgatatcagccaataggctaacgctcatttccaattcaatagaatccctatgaggtgacaaaggaagacaaagttcaaccggaaaaccttcccccggctcagatttcattgactgattgccttgcatgctccggatgtgtcacgtctgcggaagcagtgttgatatccttgcaatcacatacggaggttctcaatactcttgattcgtaccccgaattgccgcttggttctacaagctaccaaagaggcacacaaaaagttggatcagcagacagcgatggtcgcatctttgttgctagcgtcagccctcaagtcagggcgagcttggcagccacatacggaatcaccgagcgggaggcgaaatatatgattgaccaatttcttatgggccctcacggtctcagagctggtggaaaacatggcaatgggtttacatgggttgtggacacgaacgttatgcgtgaagcagtgttggctctgacagcggacgaggtcacgagctctttattatcaactggatcgggcagccttcccaagagtccaattctttcgtccgcttgccccggctggatatgttatgctgaaaaaacacacccttttatccttccgcatttatctcgcctcaagtctcctcaggcgttgagcggcacatttctgaagtcagtgctaagcaaggcacttggggtcccgccttctcagatatggcatttagctatcatgccatgcttcgacaagaagctggaagctagccgggaagagctgacagacattgcatgggcttcaaccttcacccagtcacagacaacacccgtccgcgacgttgactgtgtcataaccacccgtgagctactaactttagccactgctagggggctttctctacccaatttgccgctcaaaccattgcccgcgtcatgtttaactccatttccagatcaagccctagaatcatttttgttctctaagagctcgtcgggccaaacagtcgaatcagggacatctggaggctatcttcatcacgtcctccaaatcttccaagccagaaaccccggcagcaagattgtcacccagcgtgggcgcaacgccgatgttgtggaatatgtgctcatgtcgtctggggatgagcctctttttagggcggctcggtattatggcttcaggaatatacaaaatctcgtcagaaaacttaaacccgcacgcgtgtcaagactgccaggcgccaagccgcaagcggtctcttcaagtgcaaatcgacgacagcccatgtcaaggaacgcagctccggctggaacaggcgctgattatgcatatgttgaagtcatggcttgtcctggcggctgtaccaatggtggtgggcaaataaggattgaagatgcccgggaggctgttccgaacgcactaaaagagacatcgactgaaactcctgtggctgcaccgaaacccacgccgcatgagcagcgtgcctggctagcccgggtagatgaagcgtactactctgcggactcggatagcgagggatctgtcacgacggagccggtttctgtcctgtcaagggataaccagattcatgagtttttgaactattggtcagagaaggttgatatacccctttcccggctcgcgtacacgtcctatcgcgaagtggagagcgacgtgggtaagacgaagaatgcgcccaacgaaactgctcgtgttgtggaattggcaggaaagatcggaggtggttggtgatatcgcactcgctgtctggaaaacattgcatatatccattatacccttcaatttgttgtggatgagaacgctctttgcatcactggttggttgcatgctagtacgcatttggagagatacccctttattatagatttctcatttgttgggcatttgagaggatttataagtcatgagattgctacaggaagccccatagacgtatagagatcagggtagagcatgccaccttagcttttacactactccgtacggtcatgatcaaactcttgaatttaatatttcgattgcggacaatcaaagcaatgtatggagtaaacatgtggtagagcaggtacctgaggcataacgtgctagtccagaataggctagctccatcaaaccccgaatcttccatcgggatgacaaagcagcctagagcatttggcagaaaaagtgctctaccccaagtctgaattctctccaacaactcgccttcaatcacccacgccccagtttat

SEQ ID NO: 112 - DNA, 1865 bp, Aspergillus fumigatus
atgagtgcaatcctttctgcagacgatttgaacgatttcatttctcccggggttgcttgcatcaagcccgttgagagtctaccacaaaaagaatcccagtcggaggtatctttcctgtcttaccagtcatctgttgatatcagccaataggctaacgctcatttccaattcaatagaatccctatgaggtgacaaaggaagacaaagttcaaccggaaaaccttcccccggctcagatttcattgactgattgccttgcatgctccggatgtgtcacgtctgcggaagcagtgttgatatccttgcaatcacatacggaggttctcaatactcttgattcgtaccccgaattgccgcttggttctacaagctaccaaagaggcacacaaaaagttggatcagcagacagcgatggtcgcatctttgttgctagcgtcagccctcaagtcagggcgagcttggcagccacatacggaatcaccgagcgggaggcgaaatatatgattgaccaatttcttatgggccctcacggtctcagagctggtggaaaacatggcaatgggtttacatgggttgtggacacgaacgttatgcgtgaagcagtgttggctctgacagcggacgaggtcacgagctctttattatcaactggatcgggcagccttcccaagagtccaattctttcgtccgcttgccccggctggatatgttatgctgaaaaaacacacccttttatccttccgcatttatctcgcctcaagtctcctcaggcgttgagcggcacatttctgaagtcagtgctaagcaaggcacttggggtcccgccttctcagatatggcatttagctatcatgccatgcttcgacaagaagctggaagctagccgggaagagctgacagacattgcatgggcttcaaccttcacccagtcacagacaacacccgtccgcgacgttgactgtgtcataaccacccgtgagctactaactttagccactgctagggggctttctctacccaatttgccgctcaaaccattgcccgcgtcatgtttaactccatttccagatcaagccctagaatcatttttgttctctaagagctcgtcgggccaaacagtcgaatcagggacatctggaggctatcttcatcacgtcctccaaatcttccaagccagaaaccccggcagcaagattgtcacccagcgtgggcgcaacgccgatgttgtggaatatgtgctcatgtcgtctggggatgagcctctttttagggcggctcggtattatggcttcaggaatatacaaaatctcgtcagaaaacttaaacccgcacgcgtgtcaagactgccaggcgccaagccgcaagcggtctcttcaagtgcaaatcgacgacagcccatgtcaaggaacgcagctccggctggaacaggcgctgattatgcatatgttgaagtcatggcttgtcctggcggctgtaccaatggtggtgggcaaataaggattgaagatgcccgggaggctgttccgaacgcactaaaagagacatcgactgaaactcctgtggctgcaccgaaacccacgccgcatgagcagcgtgcctggctagcccgggtagatgaagcgtactactctgcggactcggatagcgagggatctgtcacgacggagccggtttctgtcctgtcaagggataaccagattcatgagtttttgaactattggtcagagaaggttgatatacccctttcccggctcgcgtacacgtcctatcgcgaagtggagagcgacgtgggtaagacgaagaatgcgcccaacgaaactgctcgtgttgtggaattggcaggaaagatcggaggtggttggtga

SEQ ID NO: 113 - DNA, 1725 bp, Aspergillus fumigatus
atgagtgcaatcctttctgcagacgatttgaacgatttcatttctcccggggttgcttgcatcaagcccgttgagagtctaccacaaaaagaatcccagtcggagaatccctatgaggtgacaaaggaagacaaagttcaaccggaaaaccttcccccggctcagatttcattgactgattgccttgcatgctccggatgtgtcacgtctgcggaagcagtgttgatatccttgcaatcacatacggaggttctcaatactcttgattccgatggtcgcatctttgttgctagcgtcagccctcaagtcagggcgagcttggcagccacatacggaatcaccgagcgggaggcgaaatatatgattgaccaatttcttatgggccctcacggtctcagagctggtggaaaacatggcaatgggtttacatgggttgtggacacgaacgttatgcgtgaagcagtgttggctctgacagcggacgaggtcacgagctctttattatcaactggatcgggcagccttcccaagagtccaattctttcgtccgcttgccccggctggatatgttatgctgaaaaaacacacccttttatccttccgcatttatctcgcctcaagtctcctcaggcgttgagcggcacatttctgaagtcagtgctaagcaaggcacttggggtcccgccttctcagatatggcatttagctatcatgccatgcttcgacaagaagctggaagctagccgggaagagctgacagacattgcatgggcttcaaccttcacccagtcacagacaacacccgtccgcgacgttgactgtgtcataaccacccgtgagctactaactttagccactgctagggggctttctctacccaatttgccgctcaaaccattgcccgcgtcatgtttaactccatttccagatcaagccctagaatcatttttgttctctaagagctcgtcgggccaaacagtcgaatcagggacatctggaggctatcttcatcacgtcctccaaatcttccaagccagaaaccccggcagcaagattgtcacccagcgtgggcgcaacgccgatgttgtggaatatgtgctcatgtcgtctggggatgagcctctttttagggcggctcggtattatggcttcaggaatatacaaaatctcgtcagaaaacttaaacccgcacgcgtgtcaagactgccaggcgccaagccgcaagcggtctcttcaagtgcaaatcgacgacagcccatgtcaaggaacgcagctccggctggaacaggcgctgattatgcatatgttgaagtcatggcttgtcctggcggctgtaccaatggtggtgggcaaataaggattgaagatgcccgggaggctgttccgaacgcactaaaagagacatcgactgaaactcctgtggctgcaccgaaacccacgccgcatgagcagcgtgcctggctagcccgggtagatgaagcgtactactctgcggactcggatagcgagggatctgtcacgacggagccggtttctgtcctgtcaagggataaccagattcatgagtttttgaactattggtcagagaaggttgatatacccctttcccggctcgcgtacacgtcctatcgcgaagtggagagcgacgtgggtaagacgaagaatgcgcccaacgaaactgctcgtgttgtggaattggcaggaaagatcggaggtggttggtga

SEQ ID NO: 114 - protein, 574 aa, Aspergillus fumigatus
MSAILSADDLNDFISPGVACIKPVESLPQKESQSENPYEVTKEDKVQPENLPPAQISLTDCLACSGCVTSAEAVLISLQSHTEVLNTLDSDGRIFVASVSPQVRASLAATYGITEREAKYMIDQFLMGPHGLRAGGKHGNGFTWVVDTNVMREAVLALTADEVTSSLLSTGSGSLPKSPILSSACPGWICYAEKTHPFILPHLSRLKSPQALSGTFLKSVLSKALGVPPSQIWHLAIMPCFDKKLEASREELTDIAWASTFTQSQTTPVRDVDCVITTRELLTLATARGLSLPNLPLKPLPASCLTPFPDQALESFLFSKSSSGQTVESGTSGGYLHHVLQIFQARNPGSKIVTQRGRNADVVEYVLMSSGDEPLFRAARYYGFRNIQNLVRKLKPARVSRLPGAKPQAVSSSANRRQPMSRNAAPAGTGADYAYVEVMACPGGCTNGGGQIRIEDAREAVPNALKETSTETPVAAPKPTPHEQRAWLARVDEAYYSADSDSEGSVTTEPVSVLSRDNQIHEFLNYWSEKVDIPLSRLAYTSYREVESDVGKTKNAPNETARVVELAGKIGGGW

SEQ ID NO: 115 - DNA, 1510 bp, Aspergillus fumigatus
taagagttgcaggtatgagcctggtaaatcaagtcagtcacggatagcacaacagaacctcattttgtcctgaaagaatgaaccaaaaggccaactcagactctttgcaaatgcaaggaagaggtaatgagaatgttttgggagaagcttaaatgtagctttgccggaacggagaattgagtaaagccggtcatgaggcgccaagaccccagcgaaaaagcagccctaggccgcacgcaaccccgttcggcgagttgctactggctgttaagcgagactcttgtgggcgaagaccgcaacacccgaaattcgcgatccagtagcccagagcgacttgtgtgcgtttcggacgactttgacaatcccgactcttcgacaacaaattcccatcaccgccctcccggagtctgtcgaccgtgagtttgaaacctacgccctatcgaatttctggactgtcactgaagaatccgtttttgtcgtttttttaggaagccttcgccatggccgatatcgatgtcaaggttgctcaatggaagcttgttgaggttggccgtgttgtgctgatccgcagcggtccttacaccggcaagcttgctgccattgtcgagatcatcgaccacaagcgtgtacgtttttcaacggagaaattctgagcgcaggacggaaagatcatggtcggatgtgatattgacaaagaggcgcgatcataggtcctggttgacggtccttccaccgaggagaacaagatcgttccccgtcacgctcttcctctcgctcacgccactctcacccccttcgtcattcccaaactcccccgcgctgccggcactggccccgtcaagaagctctgggagaagaacgagatcgatggaaagtgggctaagagcaccattgctcagaagactgagcgcgctgagcggaggaagaaccttaccgacttcgagcgcttcaaggtcctcagactcaagaagcaggtacgttcagtttgcgaaactatgggagaattgtgatggcacattggagggcattcttggcaactctgcactcgcttttcgcgagagggaagaggagcaattacttgtattatgatttgcgactggttactgacatctggtgatttaacaggctcgctacgaggtccagaaggctcacgccaaggtcagggctgctgctcctaagtcatagatgttttcatgaggctcggtgcatagtatgaaggggtaccttgggacggttttacatggctgagggttttattctatttcagcaaaaattaagctgtatccactacaatgacagccaaaaaatgattcaaaaccttgatatcctgacacgggtcatcctgctatgtcatcagattcgcgcacccgattagtacttggctctggtttatagccgtctccttagacattaattgggaattaaacattttagactcaagatcacggaatatgtaagaaagtatcgttatgtacattactgagttggattggctcgttatgactcgtat

SEQ ID NO: 116 - DNA, 685 bp, Aspergillus fumigatus
atggccgatatcgatgtcaaggttgctcaatggaagcttgttgaggttggccgtgttgtgctgatccgcagcggtccttacaccggcaagcttgctgccattgtcgagatcatcgaccacaagcgtgtacgtttttcaacggagaaattctgagcgcaggacggaaagatcatggtcggatgtgatattgacaaagaggcgcgatcataggtcctggttgacggtccttccaccgaggagaacaagatcgttccccgtcacgctcttcctctcgctcacgccactctcacccccttcgtcattcccaaactcccccgcgctgccggcactggccccgtcaagaagctctgggagaagaacgagatcgatggaaagtgggctaagagcaccattgctcagaagactgagcgcgctgagcggaggaagaaccttaccgacttcgagcgcttcaaggtcctcagactcaagaagcaggtacgttcagtttgcgaaactatgggagaattgtgatggcacattggagggcattcttggcaactctgcactcgcttttcgcgagagggaagaggagcaattacttgtattatgatttgcgactggttactgacatctggtgatttaacaggctcgctacgaggtccagaaggctcacgccaaggtcagggctgctgctcctaagtcatag

SEQ ID NO: 117 - DNA, 465 bp, Aspergillus fumigatus
atggccgatatcgatgtcaaggttgctcaatggaagcttgttgaggttggccgtgttgtgctgatccgcagcggtccttacaccggcaagcttgctgccattgtcgagatcatcgaccacaagcgtgtcctggttgacggtccttccaccgaggagaacaagatcgttccccgtcacgctcttcctctcgctcacgccactctcacccccttcgtcattcccaaactcccccgcgctgccggcactggccccgtcaagaagctctgggagaagaacgagatcgatggaaagtgggctaagagcaccattgctcagaagactgagcgcgctgagcggaggaagaaccttaccgacttcgagcgcttcaaggtcctcagactcaagaagcaggctcgctacgaggtccagaaggctcacgccaaggtcagggctgctgctcctaagtcatagatgttttcatgaggc

SEQ ID NO: 118 - protein, 149 aa, Aspergillus fumigatus
MADIDVKVAQWKLVEVGRVVLIRSGPYTGKLAAIVEIIDHKRVLVDGPSTEENKIVPRHALPLAHATLTPFVIPKLPRAAGTGPVKKLWEKNEIDGKWAKSTIAQKTERAERRKNLTDFERFKVLRLKKQARYEVQKAHAKVRAAAPKS

SEQ ID NO: 119 - DNA, 1942 bp, Aspergillus fumigatus
tacacccagcacacctaccctagccacagatttcttggaccacgggaacccgaacaaagagtctctggccgaactctggtctaatttcctagagcaggagtcacaggccagtgggaatccagaatcgccgaaaccatgaagagtttcaaaaggtggctctgttctgagccgtatagaaaactagcatcttctctaagatacggtggttccacatttatatatgcttgccgactggctttggtctcctcttcttttccattctagacttgcttcgtagtaataacactgatatcacccgcgtgtttgcgacttttgcatcaaaccagcttccccaccgctttctttctgccaccatagcgggggacctcgttattgagcggacaagtcgtcgttggctttttctgcacgtttggcctatgcttcgtttattcagctctggtacagctgggaagttgactgatacactctcctctctgatttcttggtactccagattgacaatgactaccggggctggtacgatctctcattccaacacctatcatcgtattcctcgccgttaactgaccaatccaccagtgcaaaggttccgtccagtggtggtatcgggtccctctgggactgggaagtcgaccttgctcaagagactcttcgctgaataccccgatactttcgatttatccgtgtctcgtacgtctaaccccttgccaaccctcattgactatgcctgcgaattgtttcttttggtggaattgcgctgaacggtgtttgttatatttagataccactcgagctccccgtcccggggaagaaaatggacgtgagtattacttcacaactaaagaagatttcctggatcttgtgagcaagaatgcctttatcgagcatgcgcagtttggtggcaattactacggtactactgtgcaggcagtgaaggatgttgcgcagaagggcaagatctgcgttctcgacattgagatgaggtaataatagtcctgcaacgtgaactgatatgaccggagaagcagaggaaatccatcatcaaatggattgtagttcaacccaaacaacagctgacgactgaattgcaatagggcgtgaaacaagtcaagcgcaccgatcttgatgctcgattcttatttttagcacccccgtcccttgaagaactagagaaaagactgcgtgggagagcaaccgagactgaggagagcttgacggtatggctgtcctccacattccttcacttccccaactcgccagactgtcccgctggaattctaactttgcgtcagaaacgccttgcccaagctaaaaatgaattggaatatgcggcgcagcctggctctcatgataagattgtcgtgaacgatgacctggagaaggcttataaggaactgcgggattggattgtcgacggtggtaactttggagcgcgtcaatgatttattgggcatgtctcggcgtgttttatttatcagcgctgctgtatactttagcgcccgtagatactgtcggttgcgatactgaaaacaatgcatcatctgccttggtaacttcggtccacagaaacccataatcaaggaggtcctttcgtcgtcgacgaacataagagagattaattacatgaacatcaagactatgctaacaattcgaatgttggtctcttttctgtctggagacgacaaatctaggaaaggtggtagactcagtcactctcttgaacggagaggagaaaattaagcaaaactaaaaaagagaacaaagtctgatgagcaatatgagggctgaaaaggatatctgtaaagaggctgctagaataaaatggaagatgccgattgagaaggcaatggaggaagagaaggggtcatttatcgcagtttgggcgtggaccagaaatgactgcagtatgtttatggaccatgccagccggagctattggact

SEQ ID NO: 120 - DNA, 943 bp, Aspergillus fumigatus
atgactaccggggctggtacgatctctcattccaacacctatcatcgtattcctcgccgttaactgaccaatccaccagtgcaaaggttccgtccagtggtggtatcgggtccctctgggactgggaagtcgaccttgctcaagagactcttcgctgaataccccgatactttcgatttatccgtgtctcgtacgtctaaccccttgccaaccctcattgactatgcctgcgaattgtttcttttggtggaattgcgctgaacggtgtttgttatatttagataccactcgagctccccgtcccggggaagaaaatggacgtgagtattacttcacaactaaagaagatttcctggatcttgtgagcaagaatgcctttatcgagcatgcgcagtttggtggcaattactacggtactactgtgcaggcagtgaaggatgttgcgcagaagggcaagatctgcgttctcgacattgagatggaggtaataatagtcctgcaacgtgaactgatatgaccggagaagcagaggaaatccatcatcaaatggattgtagttcaacccaaacaacagctgacgactgaattgcaatagggcgtgaaacaagtcaagcgcaccgatcttgatgctcgattcttatttttagcacccccgtcccttgaagaactagagaaaagactgcgtgggagagcaaccgagactgaggagagcttgacggtatggctgtcctccacattccttcacttccccaactcgccagactgtcccgctggaattctaactttgcgtcagaaacgccttgcccaagctaaaaatgaattggaatatgcggcgcagcctggctctcatgataagattgtcgtgaacgatgacctggagaaggcttataaggaactgcgggattggattgtcgacggtggtaactttggagcgcgtcaatga

SEQ ID NO: 121 - DNA, 603 bp, Aspergillus fumigatus
atgactaccggggctgtgcaaaggttccgtccagtggtggtatcgggtccctctgggactgggaagtcgaccttgctcaagagactcttcgctgaataccccgatactttcgatttatccgtgtctcataccactcgagctccccgtcccggggaagaaaatggacgtgagtattacttcacaactaaagaagatttcctggatcttgtgagcaagaatgcctttatcgagcatgcgcagtttggtggcaattactacggtactactgtgcaggcagtgaaggatgttgcgcagaagggcaagatctgcgttctcgacattgagatggagggcgtgaaacaagtcaagcgcaccgatcttgatgctcgattcttatttttagcacccccgtcccttgaagaactagagaaaagactgcgtgggagagcaaccgagactgaggagagcttgacgaaacgccttgcccaagctaaaaatgaattggaatatgcggcgcagcctggctctcatgataagattgtcgtgaacgatgacctggagaaggcttataaggaactgcgggattggattgtcgacggtggtaactttggagcgcgtcaatga

SEQ ID NO: 122 - protein, 200 aa, Aspergillus fumigatus
MTTGAVQRFRPVVVSGPSGTGKSTLLKRLFAEYPDTFDLSVSHTTRAPRPGEENGREYYFTTKEDFLDLVSKNAFIEHAQFGGNYYGTTVQAVKDVAQKGKICVLDIEMEGVKQVKRTDLDARFLFLAPPSLEELEKRLRGRATETEESLTKRLAQAKNELEYAAQPGSHDKIVVNDDLEKAYKELRDWIVDGGNFGARQ

SEQ ID NO: 123 - DNA, 3108 bp, Aspergillus fumigatus
aaaccaggcgcatatgttccgctgccgccctgcgcaggagagggctgataaggattgccatatccagacgacgggggctgctgggatgctgggggaggaggcgagcgcatcatgggaacggtagatacatgctgaggcacaggatggtggagtggaggtgactgcgcaggcggtggtgcgaaagactggcggtacatgttgatttttttttcccttgttacaataagtgagaagctagtgatgaacaaaagacttgcgactattctgtctcgtcttcttgtcttctaccaaccgaagaggggggatgttggaaatcggacagtttgagtatgagtgatgttgaagtgtgtttatacgtggctggactcggtctgatcgccggagagctctcaccttttccgcccacggttccccaccatagtggccactacacacacttgtccgttctccaaaacccacgctgctcgcactgaataatatacacaagaagtgcttacaacatgttagaagccttcgaagtcttgacaacatctggggtggtgctgtggtcgaagtcgtatgcgccggtcggagcgcatgttgtcaacagcctaatcaacgatgtcttcattgaggagaaggttcgagcgcagaatcaggcagcgagcagtgcagctcctatctacaagaaggaaaagtatactctgaaatggaagcaagtaaaggatttcaatctgatatttgtggtatgttcacgccgctcgttgattcaatggcgccactgaccgattccataggctgtatatcaatctctgctacatcttggttggatcgacaaactcttggataatgtttcgaccatattcatcgacttatataaggatgagctaaggagcacacgggctaggattattgagtacccattcgataagtacttcgaccagcaggtgcgagagcttgaggacaatgctggggctcctacatcagaatctctcgtagtagagatcaacgagagaaaggaccctcttgtctcatcagataacggcgggccacctccgccacccgtgcctggtctgctgaaaggtatctgacgtcgataatttttctctgctagtgatcatattgctaactacctccgaagcgcaacgtccagttgcgcagggcgtggcgacctcggacgagggttcgccaccccaaaccccagatctttctcgatcgtcaacgcccatttcaggtcatctattgaccgcgaaaggagggcctgctggccgcgcctctcgtcgcgcacgcaaagcggccaacgcgagcgctaccgcttcttctggagatgaaagcattcggaaggggaaaacattgaaaagtggaaaaaagatgcgcaagtgggatgctgatggctttgcggatgaggacgacggcaaggtcctcgattactccgcccccgcagatggtgaggacgcaccggctcctgtagtcgaggctgttgcgcaggaatcctggggacgccgaacaggcaagggccaatttgtgctgaaagatctaggggatgaagtccattccattcttgagaatgctgatcatgaaaagacaaagtcttcctcgtccacgggctttgttgggtctggagtcaacgcacttggtggattcttccgtaatattgtcggcggcaaggtccttactgaggctgacttggagaaacccttgaaagccatggaagaccatttgctgaagaagaacgttgcgcgcgaagcggccgtccgtctatgtcaaggcgtccagcgcgaattagttggcaagaagacaggcaactttcaaagtgttgatgcagcactgcgctccgcaatggagtcctcgttgcgcaaaatattgacgccaacgtcatctctcgatctactgcgtgagatcgatgctgttagatctccgacgagcaaaggacaggctcctcgcccatatgtcatttccatcgtgggcgtgaacggtgttgggaagtcgacaaatctgggcaaaatttgttacttccttctccagaataactatcgtgttctgattgcagcctgtgacaccttccgctctggagccgtggagcagttacgagtccatgctcgcaatttgaaggaacttagtacccgggagaatgctggagaggttgaactctacgagaagggatatggaaaggatgcagcgaatgtagcgaaggatgcagtggagtacggtgcggcgaatcatttcgacgttgtgttgattgatactgccggtcgccgtcataacgaccaacgccttatgtcttcgctcgagaagttcgccaagttcgccaaaccagataagatcttcatggtcggtgaagctctggtcggtacggacagcgtgatgcaggctcgcaacttcaaccaagctttcggcactgggagaaacctcgatgggttcatcatcagtaaatgtgataccgttggtgacatggtaggtacgcttgtcagcatggtgcatgctacaggcattcctattgtttttctgggtgtaggccagcactatggtgatttgaggggcctaagtgttccttgggctgtcaatctgctgatgaagtgagcgcgcagcttttgccttgtaatagagaattatattatctcgcttctgatcacgctgggtcttggatggccttcttcaaaaataagttactgcgattgtacatctcgcgcttcgctaaagatagagaaacgaaagatagacaaagaacgggggaaaaaattgacaaaaagaatgacctacgcaggattcgaacctgcaatctcttgatccgtagtcaagcgccttaccattgggccagcaggccttcttgaaggttttcacccagaaatatgctttatcaggaatctgggaacatccgcataaactccaataataagcttgtgcctgtcattgttactacgctaaagtgactctctagtgtggctcttatgcaggagaaaccatacaggatattgtaacggtgaatgcattttttctgccttgaggtatgacaccattttgttttggaggtggactcgatcacgagctcactatcccggcctcatgggaggattatgacacccttggttcctgaatacttggtcttggctggaattacttcaatgacac

SEQ ID NO: 124 - DNA, 2059 bp, Aspergillus fumigatus; identical to SEQ ID NO: 10

SEQ ID NO: 125 - DNA, 1884 bp, Aspergillus fumigatus
atgttagaagccttcgaagtcttgacaacatctggggtggtgctgtggtcgaagtcgtatgcgccggtcggagcgcatgttgtcaacagcctaatcaacgatgtcttcattgaggagaaggttcgagcgcagaatcaggcagcgagcagtgcagctcctatctacaagaaggaaaagtatactctgaaatggaagcaagtaaaggatttcaatctgatatttgtgcttggttggatcgacaaactcttggataatgtttcgaccatattcatcgacttatataaggatgagctaaggagcacacgggctaggattattgagtacccattcgataagtacttcgaccagcaggtgcgagagcttgaggacaatgctggggctcctacatcagaatctctcgtagtagagatcaacgagagaaaggaccctcttgtctcatcagataacggcgggccacctccgccacccgtgcctgcctcggacgagggttcgccaccccaaaccccagatctttctcgatcgtcaacgcccatttcaggtcatctattgaccgcgaaaggagggcctgctggccgcgcctctcgtcgcgcacgcaaagcggccaacgcgagcgctaccgcttcttctggagatgaaagcattcggaaggggaaaacattgaaaagtggaaaaaagatgcgcaagtgggatgctgatggctttgcggatgaggacgacggcaaggtcctcgattactccgcccccgcagatggtgaggacgcaccggctcctgtagtcgaggctgttgcgcaggaatcctggggacgccgaacaggcaagggccaatttgtgctgaaagatctaggggatgaagtccattccattcttgagaatgctgatcatgaaaagacaaagtcttcctcgtccacgggctttgttgggtctggagtcaacgcacttggtggattcttccgtaatattgtcggcggcaaggtccttactgaggctgacttggagaaacccttgaaagccatggaagaccatttgctgaagaagaacgttgcgcgcgaagcggccgtccgtctatgtcaaggcgtccagcgcgaattagttggcaagaagacaggcaactttcaaagtgttgatgcagcactgcgctccgcaatggagtcctcgttgcgcaaaatattgacgccaacgtcatctctcgatctactgcgtgagatcgatgctgttagatctccgacgagcaaaggacaggctcctcgcccatatgtcatttccatcgtgggcgtgaacggtgttgggaagtcgacaaatctgggcaaaatttgttacttccttctccagaataactatcgtgttctgattgcagcctgtgacaccttccgctctggagccgtggagcagttacgagtccatgctcgcaatttgaaggaacttagtacccgggagaatgctggagaggttgaactctacgagaagggatatggaaaggatgcagcgaatgtagcgaaggatgcagtggagtacggtgcggcgaatcatttcgacgttgtgttgattgatactgccggtcgccgtcataacgaccaacgccttatgtcttcgctcgagaagttcgccaagttcgccaaaccagataagatcttcatggtcggtgaagctctggtcggtacggacagcgtgatgcaggctcgcaacttcaaccaagctttcggcactgggagaaacctcgatgggttcatcatcagtaaatgtgataccgttggtgacatggtaggtacgcttgtcagcatggtgcatgctacaggcattcctattgtttttctgggtgtaggccagcactatggtgatttgaggggcctaagtgttccttgggctgtcaatctgctgatgaagtga

SEQ ID NO: 126 - protein, 641 aa, Aspergillus fumigatus; identical to SEQ ID NO: 12

SEQ ID NO: 127 - DNA, 2564 bp, Aspergillus fumigatus
cagcgaaggtatcggagaggagttgaccagaactggggagtagtcatggttatgagacgaaaggtcgacggttcagaaggatttttgcgggacttgattgagagttgtgagcgcgacagatggattggatcaaggattcaattgatcagagctcggctcagatcatgtatccagtcttccgatgattccgattattgtgaaactgggttaaatcgcgccagaggggcaggtatcgtgacggagagggggtatatcgtcgaatggaggttgtgagtgacgacggccgacgattgcgcagttcaaagcggcgaagaggtcttggcactctcggtccaaacacgttgcccgttctctccaactaaacggaactgacgaagctccttcagaggacactctcctctaccgattcatttccttaatctattccttctctttctccggactgcagtgacttccctttcagccaattgcccgctccactgtgcggcattcgatataccatgcggtggtgcctcactcttctggcattctgcttcttggcagttgtacgtgcattaagtagctccggcagtcgtctgttggttgttttggaagatgccacagaaaaggaattatactcgaaattatgggctgacctagaaggtgctctaacctactgaacttctacgttaatatgctaatattaattggtagctcgaggatataacctcgacttcgaatcccccaagaatgacaagctcagcctgttcgaactcggagaccgagtctacgaccacatgcttctcctgcctcccaagtcaaagggttagcgttacccttagacatgtccatatgctctgctttgtacatctcaattgacctcttggccaggctatggaccctcccttacccccaagaatatcattgatttcatgaacaaggacggtaacgtcctcctcgccttgtcgggcaagtccacaaccgccagcgctatcagctcgctgctattggagctcgatctccatctccctgtcgatcgttcctctgtcaccgtcgatcacttcaactacgatacactttctgcctccgataagcatgatgttctgctactccaccgaccaggcaagttgaggtccgataccaaggctttctttgatggcgagggcgttgtagcatttcccagagccgtcccccacaccctgggcgatgcaaaccctctcattgcgcctattctgcgagcgcccgccactgcgtatagttacaaccccaaggaggacgcgtcgtcagttgaggatgttgcagctacgggttcgcagttggctctggtctcggccatgcaggctagaaactccgctcggttcactctactgggatccgtggagagtctgcaggatcagtggttttctgcgactgtcaaggctcctggtgatgggaagcagatgaagacggtcaaccaggaattcgccaagcagcttactgcgtggacattcaaggaaaccggagtcctcaaggtcggaaagatcgagcatcatctggctgaagatggtgaaatcactcccgagaagctgaaccctaagatctatcgaataaagaatgaaactgtaagtgacagccatctgaggttccattgcctatttgcatgctcacccttctcaacaggtctttagcattgaactttccgaatacaactatgatcgttacgcgcccttcgaggttccaactggcgatgccgtccagctcgagtttaccatgctgtctcccttccatcgcctgaacttggaacccgtccgtcgaacagataacagtacagtttacagcacacgattcaccacccccgatcagcatggaatcttctccttccgagtgaactacaagcgcccgttcctcacgaacatcgaagaaaaacttgaggtgaccgttcgtcatttcgctcataacgagtacccccgaagctggaaaatcagcggtggatgggtctggattgcgggtctgtggtccgtcatcgctggcttcttagtattcgttgttgcatggctttactcagcgccttctgccgccgcactgaacacaaagaagacacaataatcctctatagaatctcaacgaatgatacttcggaatgaagcgtgcacttacgccggaagcccataattcaaaagtatgtacagtcatatgccataagctagtagtgttacatgaaatccggaatctcaagacattccgccaactgggaactccaaaaccatgccaggagatgagataaaaagccgcacaataactcagaacagcggatctaggcatctggccagcctagtgagccgcgagcttgaaaatttatagtcctctagtcttctcagggtccatctgaacttcattcccgctgatctccttaacggtttcgaaggcgggcttgccggcgtatagttgctggtgggattgggatttgcctccttcgagctccctggaaaccgagataggctgatttccacggaatcgctttcgcatgtcgatcaacccacgcctagattgtgcccgttgtcagcactatccaatgtaattaaagcacgcatatccgttccacccac

SEQ ID NO: 128 - DNA, 1564 bp, Aspergillus fumigatus; identical to SEQ ID NO: 13

SEQ ID NO: 129 - DNA, 1383 bp, Aspergillus fumigatus
atgcggtggtgcctcactcttctggcattctgcttcttggcagttgtacgtgcattaagtagctccggcagtcgtctgttggttgttttggaagatgccacagaaaaggaattatactcgaaattatgggctgacctagaaggatataacctcgacttcgaatcccccaagaatgacaagctcagcctgttcgaactcggagaccgagtctacgaccacatgcttctcctgcctcccaagtcaaagggctatggaccctcccttacccccaagaatatcattgatttcatgaacaaggacggtaacgtcctcctcgccttgtcgggcaagtccacaaccgccagcgctatcagctcgctgctattggagctcgatctccatctccctgtcgatcgttcctctgtcaccgtcgatcacttcaactacgatacactttctgcctccgataagcatgatgttctgctactccaccgaccaggcaagttgaggtccgataccaaggctttctttgatggcgagggcgttgtagcatttcccagagccgtcccccacaccctgggcgatgcaaaccctctcattgcgcctattctgcgagcgcccgccactgcgtatagttacaaccccaaggaggacgcgtcgtcagttgaggatgttgcagctacgggttcgcagttggctctggtctcggccatgcaggctagaaactccgctcggttcactctactgggatccgtggagagtctgcaggatcagtggttttctgcgactgtcaaggctcctggtgatgggaagcagatgaagacggtcaaccaggaattcgccaagcagcttactgcgtggacattcaaggaaaccggagtcctcaaggtcggaaagatcgagcatcatctggctgaagatggtgaaatcactcccgagaagctgaaccctaagatctatcgaataaagaatgaaactgtctttagcattgaactttccgaatacaactatgatcgttacgcgcccttcgaggttccaactggcgatgccgtccagctcgagtttaccatgctgtctcccttccatcgcctgaacttggaacccgtccgtcgaacagataacagtacagtttacagcacacgattcaccacccccgatcagcatggaatcttctccttccgagtgaactacaagcgcccgttcctcacgaacatcgaagaaaaacttgaggtgaccgttcgtcatttcgctcataacgagtacccccgaagctggaaaatcagcggtggatgggtctggattgcgggtctgtggtccgtcatcgctggcttcttagtattcgttgttgcatggctttactcagcgccttctgccgccgcactgaacacaaagaagacacaataa

SEQ ID NO: 130 - protein, 460 aa, Aspergillus fumigatus; identical to SEQ ID NO: 15

SEQ ID NO: 131 - DNA, 3376 bp, Aspergillus fumigatus
gcccaggcaacatctaccttatggcatcgcagagcgagaattgctcaacatctttgaggtctttctgcttgaattcccggggacccttcttctccttcttgctcttcgccttggcctttttatttcgggcctgagcattgctcttggctcctcggacctgttggtgaaagctcgaaagcaaaggcgccattgttctttgtcgaagaggcaattgacatgttgagagaatgctccttgccgccgacggcaggatcgagccgaaggaggacatggtttgaagagtggattgatgcgagctttcagactaactcctgggagacggagatgttttcttctccgaaagctctctctcagtgatgcgggcaggagaaacgtaacgccggcggagtcccttttgagtcagatgcccctctgtactattcaattttcggggaattcaacagcccacttgttacgctctcgcaggtcgatttcactcgaggcggattttgagggccgcaatgtctcagtatcagcttactgtggccaccagggccaatcagccctatgtacttcctgtcctactggtcgcaacttccatcaacgaggcacgaccaagcccagtgatatcgatcacctatgaggatactgcggttcttcgtgaaggagacaaggccgtcgtgcaatacactggagctagcggtaatcctatctttggccttatcaatgctgttcaggaactccgcaaagacttccccttccttaacagcaaggatgagaagctggtaagaggcgccatggagccttactgctgatgagcactgataagtgatactaaccctccttttataggagaatgaatggctgtctcagttggaagcatttgctcctctagatttcaaggcccttgaccctgaattgcagcgcctcgatacccacctcctgctgagatctttcgtcgtcggttacgctctctcgacggccgacattgccctttggggtgccatccgaggcaaccgtgtcgcagttgccgcgatcaagaagggctcacttgtcaatgtgactcgttggttctatttcttggaggatctgtgcccgtgggccacatctacactggaggtcttgaaccaggctgtgcgagagaagaaggccgccaaggcgaaggagggagctagctacgacatcgctcttctcaacactgaaaaaggcgtggtgacaaggtttcctcccgagccttcaggttatcttcacatcggtcacgcaaaagctgcgctgctcaacgactactttgcccacgagaagtataatggcacccttcttgtccgctttgacgacacaaatccttcgaacgagaagctcgagttccaggacgcgatcattgaagatcttgctctcatgggcatcaagcccgacaagatgagctacaccagtgactactttgacgagctttaccagtacgcccttcaaatcatcaaggacggtaacgcctacgccgacgataccgagaaggaggtcatggctgagcagagaatgaatggaaaacccagcaagcgtcgtgatgcatccgtcgaggagaaccttgcccgcttcgaggagatgaagaagggtacccctgagggtctccgttggtgtatccgagccaagatgtctgtcgataaccccaacaaggccatgcgtgatcctgtcatttaccgctgcaaccctgcccctcaccaccgcactgggacgaagtggaagatctatcctacctatgacttcgcctgccctatcgtcgattcaattgagggtgtgactcatgccctcagaaccattgaataccgcgatcgcaaccctcagtaccagtggttcttggacacgctcaagcttcgccatgtccaaatctgggattttgctcgcatgaacttcattcgcaccttgctgtctaagagaaaacttaccaagctcgttaaccaaggtgtcgtctggggatgggatgagtaagtttacctttgcttgcaaacggattcttgtcttactaacgatgtcagtcctcgtttccccaccatccgagtaagtaacatgcctagtcatgcctgcgaatccccttattcatccggcattttttaccatctcacctacttcccatgtactgttaacttcccacgctaatcctttcatagggcatccgacgaaggggaatgactatccctgctctgagagaattcattcttaagcagggacccagcaagaacatcaccaaccttgactggaccctgatctgggcgaccaacaagaagtacattgatcctgtcgcacctcgtcacactgccattctcaagaaggatatggtcaaggcgatcgtcaagggaggcccggctacaccttacacggaagagaaacctaagcacggcaagaaccctgcagttggtatgaagaaggtggtttttggtaacacggtcattttcgaccagaaagatgccaagagcttcaagcaagatgaagagatcaccttgatgagctggggtaatgccattgtccgtaagatcgagaccgatcctacctcaggcatcgtcaaggagctggagctggagctccacctggaaggtgacttcaaaaagaccgagaagaaggtcacgtggctctctactgagggacaggacctaattcccgttgaattggtcgatttcgactatctcctcaacaaggacaccctgcaggaggacgacgtccttgaggatgtcctgaacaagaacaccgagttcagagaggacgctgttgctgactgcaacgtcgctgaactgaaagaaggtgacatcatccagtttgagcgcaagggctattaccgtgttgaccgggcctatgtaccgggcaagccggctgttttgttcaacattcccacgggcaagacgggcaaatagatgaatagttcgatatttgggacttaacgaagccacgcatgtgtctacaacgctgtagatattagaagcactgaaagagaattcaatttacattgttaggatgctaaatcgctgatttacgacaggtgtaagtcttgttaattgcaagaataccagtacttatagttatttcccttcttctttgcagcaacgataccttgatgctccctacaaggaagcttacacaatatggtctaaggtgtgctctatctatacgccatactagctgccgcggtctgcgctacccgcgccatcgcgccatcttctactccgtacaatctccacaatttctcccagctgcattgagatgtgattccatgagaagcgaaagatgatcttcatgacgtcgaagactaaactaccttacctcttggccggacacgagccatccggcgagaaccgacagcgttgttccatggcttactagttggcctgtggtcatcgtctacttgatcatcttt

SEQ ID NO: 132 - DNA, 2376 bp, Aspergillus fumigatus; identical to SEQ ID NO: 16

SEQ ID NO: 133 - DNA, 2148 bp, Aspergillus fumigatus
atgtctcagtatcagcttactgtggccaccagggccaatcagccctatgtacttcctgtcctactggtcgcaacttccatcaacgaggcacgaccaagcccagtgatatcgatcacctatgaggatactgcggttcttcgtgaaggagacaaggccgtcgtgcaatacactggagctagcggtaatcctatctttggccttatcaatgctgttcaggaactccgcaaagacttccccttccttaacagcaaggatgagaagctggagaatgaatggctgtctcagttggaagcatttgctcctctagatttcaaggcccttgaccctgaattgcagcgcctcgatacccacctcctgctgagatctttcgtcgtcggttacgctctctcgacggccgacattgccctttggggtgccatccgaggcaaccgtgtcgcagttgccgcgatcaagaagggctcacttgtcaatgtgactcgttggttctatttcttggaggatctgtgcccgtgggccacatctacactggaggtcttgaaccaggctgtgcgagagaagaaggccgccaaggcgaaggagggagctagctacgacatcgctcttctcaacactgaaaaaggcgtggtgacaaggtttcctcccgagccttcaggttatcttcacatcggtcacgcaaaagctgcgctgctcaacgactactttgcccacgagaagtataatggcacccttcttgtccgctttgacgacacaaatccttcgaacgagaagctcgagttccaggacgcgatcattgaagatcttgctctcatgggcatcaagcccgacaagatgagctacaccagtgactactttgacgagctttaccagtacgcccttcaaatcatcaaggacggtaacgcctacgccgacgataccgagaaggaggtcatggctgagcagagaatgaatggaaaacccagcaagcgtcgtgatgcatccgtcgaggagaaccttgcccgcttcgaggagatgaagaagggtacccctgagggtctccgttggtgtatccgagccaagatgtctgtcgataaccccaacaaggccatgcgtgatcctgtcatttaccgctgcaaccctgcccctcaccaccgcactgggacgaagtggaagatctatcctacctatgacttcgcctgccctatcgtcgattcaattgagggtgtgactcatgccctcagaaccattgaataccgcgatcgcaaccctcagtaccagtggttcttggacacgctcaagcttcgccatgtccaaatctgggattttgctcgcatgaacttcattcgcaccttgctgtctaagagaaaacttaccaagctcgttaaccaaggtgtcgtctggggatgggatgatcctcgtttccccaccatccgaggcatccgacgaaggggaatgactatccctgctctgagagaattcattcttaagcagggacccagcaagaacatcaccaaccttgactggaccctgatctgggcgaccaacaagaagtacattgatcctgtcgcacctcgtcacactgccattctcaagaaggatatggtcaaggcgatcgtcaagggaggcccggctacaccttacacggaagagaaacctaagcacggcaagaaccctgcagttggtatgaagaaggtggtttttggtaacacggtcattttcgaccagaaagatgccaagagcttcaagcaagatgaagagatcaccttgatgagctggggtaatgccattgtccgtaagatcgagaccgatcctacctcaggcatcgtcaaggagctggagctggagctccacctggaaggtgacttcaaaaagaccgagaagaaggtcacgtggctctctactgagggacaggacctaattcccgttgaattggtcgatttcgactatctcctcaacaaggacaccctgcaggaggacgacgtccttgaggatgtcctgaacaagaacaccgagttcagagaggacgctgttgctgactgcaacgtcgctgaactgaaagaaggtgacatcatccagtttgagcgcaagggctattaccgtgttgaccgggcctatgtaccgggcaagccggctgttttgttcaacattcccacgggcaagacgggcaaatag

SEQ ID NO: 134 - protein, 715 aa, Aspergillus fumigatus; identical to SEQ ID NO: 18

SEQ ID NO: 135 - DNA, 3639 bp, Aspergillus fumigatus
acgcttttcgcttgtcatactcaactctagaccggttttcatctcccgatatgcctttttgatttcccttggaggctcttgctttgcctggagggcgtcacgagtcctggacgtcatccattcactttggcttcgacacggcctcacctcggagtcgaaatctggaattcacaacgtgagggtggccagaccgatgaaaggatattgtaacgctgcaattgcttactccaaaccgtagctctttgtgccgtatcctttgttgtgctggacgtggactactagtctccgcattaaaagggaaacttttgcgtcgtcgtttcctcaccttgtttgtcgacacgtccgagacttaatcatccgaaaagaacggcctgcgatcacggccgacctcatgctcgcctatcgctcgagactatcccctcaccgactctgacccgcgtaccctggcctcacaacaccaacacttctcctctagtttcgcgtacctagaacacgcaaacatgtcgccatcaatatcctacatttcaggccagcttaggcagctaatatactatcatctcgataacaatttgtgccgtaatgcgctgttcctcgccggtcgtttacatgcttacgagccccgaacggcggaagcgtcgtatttactcgctctctgccatcttcagaacgggcaagtcaaagccgcatacgattacagcaggaattttggatcgagaggcacccacctcggctgctcctatgtcttcgcgcaagcgtgcttggacctaggaaagtatctggaaggtatcacagcgttagagcggagtaaaggcctttgggcttcgaagaaccactggagtaagtattccatgcgctgatacttgcagtcgacctgcatttatttcgttgctttctgttactgacacatttccaccttgtgctctagataagcacagtgagacgcgaagacaacatctgccggatgcagccgcagtattctgcctgttaggtaaattatggcatgcgcataaggacatcaacaaagctgtggaatgctatgttgaatctctgaagctgaatcccttcatgtgggatgcgttccaagggttgtgcgacaccggtaagctttggagaataaacctaatacatctagccatggctaattgtgttctaccgccaaggagtcaatgtccgcgtgtcaaacatctacaagttgaattctgaattgctggccgtattgtcttcatcgccacaggcggatgctgagccaatatccgataagtctgcacacacgaatgggccactgcaagcgcaggcgaatgttaatccaagttccgatccttttgcctcgactacttctcgcagtgattcaggtaccagccatgggagctctgccttgtgggaaaaactaaatggaagcacagtaagcgtggcgtcatcgggagtgccagcatcaatcgtgcatgaaggagccgaaacgccgagtggtcaaagcagcggatctgatgagttccggttagctaacggaatgaacggcgcggatgcttcttgggaccctcctttagctcctgcaaggaaaaacagaacgatccaggcaataagcggcgagtatccaatggaccctcctcccaagatgaaacccactgggatccgaccaaggacaaggaccaggactgagccggaggaccaaatttcagcccagatagaccgggaggcaacaaatgcgcccagggtcggggaccgcaaacgaactgtttctggtcaggtagcgcatccaccgacgtcacaacccacagaaccaggagcaccccagcggcggagtgtgcgactcttcaaccagattaaacccacgaccagcaaattgtcggcgtccgcgctgggagtcaaggatgctagagaagtcaagaaagcgaaagccacaggtacgaaggggcgtacgacaaccaccaccatgggacgagtagtgagtggcagccgaaaacatgccagcgaacatcatgatgcagatggtaaagacggacggtcggtaccgtccgcccacactcacgccatctccaaaggcgctgctcaagaaagatcgaaagaaatcgaggcgttgacctggctgctggagctattctcgaaacttgcttctggattctttgccttgtgtcgctaccgatgcccagagtcaatccagatcttcaattcgctctctcaaggccaacgggaaacaccgtgggttctcgctcagattggacgagcgtactatgagcaggctatgtattccgaggcagaaaagtacttctaccgtgtgaagaccatggcaccctcgcgcttggaagacatggagatctactcgactgtcctttggcatctgaagaacgatgttgagttagcctatttggcgcatgagttgatggaaacagaccgcctgtcgccacaggcgtggtgcgccatcggtaattcgttttcccaccagcgagatcatgaccaggccttgaagtgctttaagcgggcaacccagctggatcctcagtttgcctacgggtttactcttcaagggcacgagtatgttgccaacgaagaatacgacaaggcgcttgatgcataccgtcacggtatcagcgcggatagtcggcattacaatgcttggtacggactgggcacggtttatgacaaaatgggcaaactggactttgccgaacaacacttccggaatgcggcaagcattaacccgaccaacgcagttttgatctgctgcattggattggtactggaaaaaatgaacaaccctaaagcggctctcgtgcaatatggtcgcgcttgttccttggcacctcattccgtactcgcgcgattccgcaaggcccgcgcattgatgaagctccaggagctcaaattagctctgtctgagttgaagattctcaaagacatggctccagacgaagctaacgtgcattatctgttgggtaagctctacaaaatgcttcacgacaaagccaatgccattaagcacttcacaactgctttgaacttggatccaaaggtatgcctatcacttccattccgtcaaattaccagacaatactaacggattcggttacaggcagcacaatacatcaaggatgccatggaatctcttgacgatgacgaggaggatgatgaggacatgagctgatttgcagtccacttcccacacatcgatccaattctggatcttacttgctacccccttcttcaccaggcgcctgctattcgggcttgcatttatgagagtcggcgttaaatttgatcttggattttcgcagcattagcattagcattataagggcgttttacaggccgggttggttagaggtgcatttaccctgcatcaggttttccaaaacggagctgaggctcgtggatcttgtttcttgtatattagcgtgggttggctttgtcggggctggtcattggctgcatatgagagacgaagacgcctgaacgacagatccatggacatagatgtctgtcgaataaggcacttgcataatctgcgatgtcattcttatgcctattgttatccttccggaaatatattccatcgtatttgagcctgaaccacggatttactccagaataacttataccactctactgcgcacgactgtagaagtaattccagaagctgcaggc

SEQ ID NO: 136 - DNA, 2639 bp, Aspergillus fumigatus; identical to SEQ ID NO: 19

SEQ ID NO: 137 - DNA, 2430 bp, Aspergillus fumigatus
atgtcgccatcaatatcctacatttcaggccagcttaggcagctaatatactatcatctcgataacaatttgtgccgtaatgcgctgttcctcgccggtcgtttacatgcttacgagccccgaacggcggaagcgtcgtatttactcgctctctgccatcttcagaacgggcaagtcaaagccgcatacgattacagcaggaattttggatcgagaggcacccacctcggctgctcctatgtcttcgcgcaagcgtgcttggacctaggaaagtatctggaaggtatcacagcgttagagcggagtaaaggcctttgggcttcgaagaaccactggaataagcacagtgagacgcgaagacaacatctgccggatgcagccgcagtattctgcctgttaggtaaattatggcatgcgcataaggacatcaacaaagctgtggaatgctatgttgaatctctgaagctgaatcccttcatgtgggatgcgttccaagggttgtgcgacaccggagtcaatgtccgcgtgtcaaacatctacaagttgaattctgaattgctggccgtattgtcttcatcgccacaggcggatgctgagccaatatccgataagtctgcacacacgaatgggccactgcaagcgcaggcgaatgttaatccaagttccgatccttttgcctcgactacttctcgcagtgattcaggtaccagccatgggagctctgccttgtgggaaaaactaaatggaagcacagtaagcgtggcgtcatcgggagtgccagcatcaatcgtgcatgaaggagccgaaacgccgagtggtcaaagcagcggatctgatgagttccggttagctaacggaatgaacggcgcggatgcttcttgggaccctcctttagctcctgcaaggaaaaacagaacgatccaggcaataagcggcgagtatccaatggaccctcctcccaagatgaaacccactgggatccgaccaaggacaaggaccaggactgagccggaggaccaaatttcagcccagatagaccgggaggcaacaaatgcgcccagggtcggggaccgcaaacgaactgtttctggtcaggtagcgcatccaccgacgtcacaacccacagaaccaggagcaccccagcggcggagtgtgcgactcttcaaccagattaaacccacgaccagcaaattgtcggcgtccgcgctgggagtcaaggatgctagagaagtcaagaaagcgaaagccacaggtacgaaggggcgtacgacaaccaccaccatgggacgagtagtgagtggcagccgaaaacatgccagcgaacatcatgatgcagatggtaaagacggacggtcggtaccgtccgcccacactcacgccatctccaaaggcgctgctcaagaaagatcgaaagaaatcgaggcgttgacctggctgctggagctattctcgaaacttgcttctggattctttgccttgtgtcgctaccgatgcccagagtcaatccagatcttcaattcgctctctcaaggccaacgggaaacaccgtgggttctcgctcagattggacgagcgtactatgagcaggctatgtattccgaggcagaaaagtacttctaccgtgtgaagaccatggcaccctcgcgcttggaagacatggagatctactcgactgtcctttggcatctgaagaacgatgttgagttagcctatttggcgcatgagttgatggaaacagaccgcctgtcgccacaggcgtggtgcgccatcggtaattcgttttcccaccagcgagatcatgaccaggccttgaagtgctttaagcgggcaacccagctggatcctcagtttgcctacgggtttactcttcaagggcacgagtatgttgccaacgaagaatacgacaaggcgcttgatgcataccgtcacggtatcagcgcggatagtcggcattacaatgcttggtacggactgggcacggtttatgacaaaatgggcaaactggactttgccgaacaacacttccggaatgcggcaagcattaacccgaccaacgcagttttgatctgctgcattggattggtactggaaaaaatgaacaaccctaaagcggctctcgtgcaatatggtcgcgcttgttccttggcacctcattccgtactcgcgcgattccgcaaggcccgcgcattgatgaagctccaggagctcaaattagctctgtctgagttgaagattctcaaagacatggctccagacgaagctaacgtgcattatctgttgggtaagctctacaaaatgcttcacgacaaagccaatgccattaagcacttcacaactgctttgaacttggatccaaaggcagcacaatacatcaaggatgccatggaatctcttgacgatgacgaggaggatgatgaggacatgagctga

SEQ ID NO: 138 - protein, 809 aa, Aspergillus fumigatus; identical to SEQ ID NO: 21

SEQ ID NO: 139 - DNA, 2684 bp, Aspergillus fumigatus
acctgttcgacgctcacactcaaatgcgcaccagtcgccgcagccaaccggagcacatacacattccaccggctcaatggggtcgaatggtccacaaggaatgtccgaaaattgggaaggaagagatcaaagacaagttccaatgtccacgcgtgaggtagcaacgcccgggaacagacctggcctttttgcgaccctgcacgatcgagggaaggtggctaagcaacccgatttctatgatccttactttccgctcaaatatctggaggtaccgaaatgtacgtgagccctgccgtcaaattactaccgcctgcttacttgtcatggcctataactgaccctgccctgcagccgatcatatttataaaagagcccactacggtttacaatcgggaattcccgatgaggtggactttgcattgtatcaccttgttcagatttcgaatcaacgatgggataaattcaagtttgagggtttccccttgcttgcggagaacctcatggcaaaggccctggatatctcccttgtcacaaccggggtgaagtgggagcttcagtatgatgttcttcaactcagtgatcgcgtcaatgagctgaactcgctacatggcacacgagatctgttggagaagatcaaacaaatgccagttacattgccggaagacaccctcgagacgtacgaattcaaccaccttctgcgcaacgttaaagaagcgaccctggtactacgcaatatggtccttctgaaagagaatgcctactatgtgtcacggtacgcgaaaggcctgctccgagacttcctcgtcattatgatcaacttgcccaatcagcctcgtctcaacgagatcaagaacgacgctttggacattgcagaggaggtcaccaagtttatgaagaccgatccggaagatccactttggatctcacttctcaattgtctcgggtcgtcagatcgtgctcacgtggtccgcgcactctgggctctcacccatttctccactgaattagacgagccagaggcgaaccgggcaatggaacggataccaaaagagactttgcagcagctctactttcacactcttctcgacttggacaaagatattctcagtggtgcattggacttctggtaccagtatacactttcgtccgagaacattgagactttgattgaggtcttcaacttgcctaccgtcttcgtcccccggatggtcgcactgttaacgcacgaaggccgaccgaacaagaaggaaactgtgttgcaagaagaaaaggtggcccccccaccgtcggatatccctcgtgtaccgcccgagctcatgaaagagctgatggagctttcggagcctgaacggagctcgcgttggctccggtgctgctttgtggaggacctcgagtgcgagatcacccaaattgccttgtggcaggcgtatcagagcagatttgcagaccctcgccttcctggtggcggtgttctccctgcggctgaatttatcaaaaatgtcagtacgactttcacgaacgcgcaagcacaggtgatcaatggccctggtgcagccacgaaattcatcatcaaaggcattcggcccctggagaccgcctataccttcgagggctttccctacatttactgcaagtgggcggacaactcgaagccaagcaagacgtgtcagcgtgctttcaagtcgccggcagaacttcgccatcacgtcttcacggaacacatgaacctcaagcctactgaaacgccgggacactataacctggagaaggcggagtcgcccgttcatacctgcctttgggacaactgcacgaaattccggtcgtctggtccgagtgccaatactgcaatggtcgctgggcacgtctccgcacatctgcccgaggaacgtgcgccagatgcggagccgccgacatccaaacgtgcggttctccaagagcgcatcgtccgcaaatggtactacctggacactccagtcaatgagcgaggcgagccgtttggagtggcctacaaggcggcgttagtattgcgtaaccttgcccgaaacctgcctacgggtattgcaccgcagtacaacgggctttcatggaagaaagccgtcttccttagtcatcgtccaaagatcatcgaagcatgggaccgcaaccgctcattgcgcaaggaacttaccgagttgatcatggtaatagaaaaagaggattattactgaacttggggcgcgagagagacgagatgaacagtaaatgatcctcttcaatgacggctgttcgttcttctttcaaactcacggtcctcgtccttattcttctgcatggccattgatgtacaataataccctttttcttccacatgatatctttcgataatcttttcttgctttttcttctttcatttctcatgaggaactcctcacctgaatgaaaaggtggttgttttgctcctaccgttcgcccttcaagctaggtgagggttcactcgcaatctaaaacacacggcgtctgggtttgggggggtaggaatctgctgggtgatgcacggtttcctttaacgatgtactataggtagatcgcagccaggagaaaagaaaagatactaatacgctaggtgctcaatgattccaggtcacttggaaaattgatcggagcgtgagagctcctatcaattttatcccttaagccgcttcaaa

SEQ ID NO: 140 - DNA, 1707 bp, Aspergillus fumigatus
atggcaaaggccctggatatctcccttgtcacaaccggggtgaagtgggagcttcagtatgatgttcttcaactcagtgatcgcgtcaatgagctgaactcgctacatggcacacgagatctgttggagaagatcaaacaaatgccagttacattgccggaagacaccctcgagacgtacgaattcaaccaccttctgcgcaacgttaaagaagcgaccctggtactacgcaatatggtccttctgaaagagaatgcctactatgtgtcacggtacgcgaaaggcctgctccgagacttcctcgtcattatgatcaacttgcccaatcagcctcgtctcaacgagatcaagaacgacgctttggacattgcagaggaggtcaccaagtttatgaagaccgatccggaagatccactttggatctcacttctcaattgtctcgggtcgtcagatcgtgctcacgtggtccgcgcactctgggctctcacccatttctccactgaattagacgagccagaggcgaaccgggcaatggaacggataccaaaagagactttgcagcagctctactttcacactcttctcgacttggacaaagatattctcagtggtgcattggacttctggtaccagtatacactttcgtccgagaacattgagactttgattgaggtcttcaacttgcctaccgtcttcgtcccccggatggtcgcactgttaacgcacgaaggccgaccgaacaagaaggaaactgtgttgcaagaagaaaaggtggcccccccaccgtcggatatccctcgtgtaccgcccgagctcatgaaagagctgatggagctttcggagcctgaacggagctcgcgttggctccggtgctgctttgtggaggacctcgagtgcgagatcacccaaattgccttgtggcaggcgtatcagagcagatttgcagaccctcgccttcctggtggcggtgttctccctgcggctgaatttatcaaaaatgtcagtacgactttcacgaacgcgcaagcacaggtgatcaatggccctggtgcagccacgaaattcatcatcaaaggcattcggcccctggagaccgcctataccttcgagggctttccctacatttactgcaagtgggcggacaactcgaagccaagcaagacgtgtcagcgtgctttcaagtcgccggcagaacttcgccatcacgtcttcacggaacacatgaacctcaagcctactgaaacgccgggacactataacctggagaaggcggagtcgcccgttcatacctgcctttgggacaactgcacgaaattccggtcgtctggtccgagtgccaatactgcaatggtcgctgggcacgtctccgcacatctgcccgaggaacgtgcgccagatgcggagccgccgacatccaaacgtgcggttctccaagagcgcatcgtccgcaaatggtactacctggacactccagtcaatgagcgaggcgagccgtttggagtggcctacaaggcggcgttagtattgcgtaaccttgcccgaaacctgcctacgggtattgcaccgcagtacaacgggctttcatggaagaaagccgtcttccttagtcatcgtccaaagatcatcgaagcatgggaccgcaaccgctcattgcgcaaggaacttaccgagttgatcatggtaatagaaaaagaggattattactga

SEQ ID NO: 141 - DNA, 1707 bp, Aspergillus fumigatus; identical to SEQ ID NO: 140

SEQ ID NO: 142 - protein, 568 aa, Aspergillus fumigatus
MAKALDISLVTTGVKWELQYDVLQLSDRVNELNSLHGTRDLLEKIKQMPVTLPEDTLETYEFNHLLRNVKEATLVLRNMVLLKENAYYVSRYAKGLLRDFLVIMINLPNQPRLNEIKNDALDIAEEVTKFMKTDPEDPLWISLLNCLGSSDRAHVVRALWALTHFSTELDEPEANRAMERIPKETLQQLYFHTLLDLDKDILSGALDFWYQYTLSSENIETLIEVFNLPTVFVPRMVALLTHEGRPNKKETVLQEEKVAPPPSDIPRVPPELMKELMELSEPERSSRWLRCCFVEDLECEITQIALWQAYQSRFADPRLPGGGVLPAAEFIKNVSTTFTNAQAQVINGPGAATKFIIKGIRPLETAYTFEGFPYIYCKWADNSKPSKTCQRAFKSPAELRHHVFTEHMNLKPTETPGHYNLEKAESPVHTCLWDNCTKFRSSGPSANTAMVAGHVSAHLPEERAPDAEPPTSKRAVLQERIVRKWYYLDTPVNERGEPFGVAYKAALVLRNLARNLPTGIAPQYNGLSWKKAVFLSHRPKIIEAWDRNRSLRKELTELIMVIEKEDYY

SEQ ID NO: 143 - DNA, 2542 bp, Aspergillus fumigatus
aggcacacagtgcactgtactgactcagttgttcctccaggcctcgataacacgacttacgtctagtgtcatctccttccacgtttccaattatcttctcttttccttccaaaaaaattaaaaagcagatactctcgtctgctagacccgatactttgaccggtatcggtatcttccagtacgagggtgctcggcatatagtcacaattcctttttcaagaggctttttcctcctctccctatcctttcgccctccctagcatccctttcgagcttgccttaatttcgtccatctggcctgtgtgccattctcttcattgcgatcaagggccttctctctcaggcggacacagcccccctgttcgtctgggcagcagatagtgcttcctaggcttctgtgtctacggtcaactacatatacaccactgcgttgctcctcatctcaataatcggtcttgcaccagcatacgtcacacaattcattacgtcaatcagcggaaatggtctacatcggcatccccaagaactacacggcttcgccgtcttcctttgccggaactccgtccttgacgatcaattacgaggcaacgcaggatcttgattctaccaatgcttttgaaggtttgttgacgccggtgacacgtgtgagagcagcgctggacagaggctgacagtctacaggtccagagaaactcttggaggtgtggttcgcgccttccgctcaggaattaggtccagcgcagcccgccggtctgaaggctgttccggaggagatctggaaggacatgttggatctcgtcaattgccaggtcctctcgattgtttcgtcagaggatgtggacgcctacctgctctccgagtctagcatgttcgtttggcctcacaaactcatcttgaagacttgtggtaccaccactcttctgtctggtctcccacgcattctcgagattgccgctttgttcggtggcttccccaagtctaccgccccttctcgcggaatctccgtcgccgctgcgccctaccgcgtcttctacagccgcaagaacttcctgttccccgaccgccagcggggccctcaccgcagctggagagatgaagtgcggactatggataagctcttcctcaacggcagcgcctacatgattggcaagatgaatggcgagcactggtacttgtacctgactgaacctcataccatgctcaccccgccaacgagcccgggagccaagaccgagtttacggaaacggagaccaaggtcctcagtgtaccccagggcgctgctctgcagactgattcggaggatgagactttggaagtcttgatgaccgacttggatgaggagaacgccaagcagttctacctcgagaatgccactgccgttgcggagaaccgttatcgcaactcaaattcggagaagagtggccatgttgatgttttcagcaacacttcctccgatatcagcgattttgactccgacggaagccaggttctgcctccagagttgactaccgagggtcacgcgctcggaaccgtggtctctgaagcctgtggactttcctctgtgtatcctaaggagaagtatcccgattcgcgcatcgatgcctacctgtttacaccatgcggcttctccgccaacggcgtgattccgcctcctgagggaaaagctggaacccactacttcacagtacacgtcactccagagccgcactgttcatatgcgtcctttgagaccaacgtaccgcactcgcagaacggccagactaccgctggaatcatcaagcaagtggtcgacatcttcaagcctggtcgcttcagcgtgactctcttcgaggccaagccagcgctgagccaggtcgaagacgagtggaaggaagccaagtacctggccgctcgtcggaccgccaaaatggaacatgtggagggatatcgccgagtggaccggattgtccacgacctcgacggctatgagcttgtcttccgctattatgaacgcctggactggaaagggggggcccctcggctgggagaggagagatcttgaagaacaggccatagcacgttgagataatcttttgcttgcggaatttggttggacattcttttgagatggaaatggtttcattctgcattttttctacgtctgcccaatattcgctgaacagccctgtgctcacttcatttgagctcgcagagtatccttcgacaacatgagcgttcgtttcgtgtacaaagctcattgactccctgtactgtcgttactgttctgattttgcattgagctacccgagcgtttgcagtgatcatgtgattatatgattcgtattcatacgttctttcagtcgtatgcgaatattttttataacatatatctaggatatgtatccaagttcaagaaggtagactcgtagtagaatgtgttgatccagttgatggccgacgccaactggtatcgattacggattggcagacgtgccagatcagtccggagtttcttttttgttgggatcgcatcacagctccaacacgacattcaactttcaa

SEQ ID NO: 144 - DNA, 1542 bp, Aspergillus fumigatus; identical to SEQ ID NO: 25

SEQ ID NO: 145 - DNA, 1482 bp, Aspergillus fumigatus
atggtctacatcggcatccccaagaactacacggcttcgccgtcttcctttgccggaactccgtccttgacgatcaattacgaggcaacgcaggatcttgattctaccaatgcttttgaaggtccagagaaactcttggaggtgtggttcgcgccttccgctcaggaattaggtccagcgcagcccgccggtctgaaggctgttccggaggagatctggaaggacatgttggatctcgtcaattgccaggtcctctcgattgtttcgtcagaggatgtggacgcctacctgctctccgagtctagcatgttcgtttggcctcacaaactcatcttgaagacttgtggtaccaccactcttctgtctggtctcccacgcattctcgagattgccgctttgttcggtggcttccccaagtctaccgccccttctcgcggaatctccgtcgccgctgcgccctaccgcgtcttctacagccgcaagaacttcctgttccccgaccgccagcggggccctcaccgcagctggagagatgaagtgcggactatggataagctcttcctcaacggcagcgcctacatgattggcaagatgaatggcgagcactggtacttgtacctgactgaacctcataccatgctcaccccgccaacgagcccgggagccaagaccgagtttacggaaacggagaccaaggtcctcagtgtaccccagggcgctgctctgcagactgattcggaggatgagactttggaagtcttgatgaccgacttggatgaggagaacgccaagcagttctacctcgagaatgccactgccgttgcggagaaccgttatcgcaactcaaattcggagaagagtggccatgttgatgttttcagcaacacttcctccgatatcagcgattttgactccgacggaagccaggttctgcctccagagttgactaccgagggtcacgcgctcggaaccgtggtctctgaagcctgtggactttcctctgtgtatcctaaggagaagtatcccgattcgcgcatcgatgcctacctgtttacaccatgcggcttctccgccaacggcgtgattccgcctcctgagggaaaagctggaacccactacttcacagtacacgtcactccagagccgcactgttcatatgcgtcctttgagaccaacgtaccgcactcgcagaacggccagactaccgctggaatcatcaagcaagtggtcgacatcttcaagcctggtcgcttcagcgtgactctcttcgaggccaagccagcgctgagccaggtcgaagacgagtggaaggaagccaagtacctggccgctcgtcggaccgccaaaatggaacatgtggagggatatcgccgagtggaccggattgtccacgacctcgacggctatgagcttgtcttccgctattatgaacgcctggactggaaagggggggcccctcggctgggagaggagagatcttga

SEQ ID NO: 146 - protein, 493 aa, Aspergillus fumigatus; identical to SEQ ID NO: 27

SEQ ID NO: 147 - DNA, 1637 bp, Aspergillus fumigatus
aaagatagagaagacgttgcgcggaaccttttgaggaccctcagctctatttgagtgtctgtcacgaggtccatatctggcgatacggagggctggctgtacaattaggttactcttatttcctactgatctagtgatataaagtttggatgcattatgtaaattaaatctcgggggcaaatgaacatttcgaatatcgttataaaactcacagaaagtgctgtcaatggcacaaatttagcaatcaatactcatctgagtatgttgttgataagtccgaaaacaacctaaatatttacctcattaggaaaggtgcactccgtagttacctcgactcgcggttagtctggtgactaagttcttggcgttgtgatgagggcaagtcctatcatgtgatcatagttagggtttccacacaccaggctctccaatatagcaagaaaatagaaggattaggtctcgtctccgaacatccatcccgccagcacacaaccgccaaaatgggtcgcgttagaaccaaggtaagttacagatgaagcatcatgagttatcttcaaaaaagccccaaaagagtatcatttctgacgaaatgggtttttcttcaatagacagtcaagaggtccgccaaggtcatcatcgagcgctactaccccaagttgacgctcgactttgagaccaacaagcgtctttgcgatgagatcgctatcattgcctccaagcgccttcgcaacaaggtgggcaatccatcactgagccgtacaacagtcggaatttgacttgctgacgaaaactagattgctggttacaccacccaccttatgaagcgtatccagcgtggccctgtccgcggtatctctttcaagctgcaggaggaggagcgtgagcgcaaggatcagtacgttcctgaggtttccgctctggatgtttcccagaccgagtccggccagctcgatgtcgatgccgacaccaaggaccttctcaagtccatgggcgtaagttctgttctcaacgcggttgttcgtggttttaaagcagtccgttaacttatattgcccactacagttcgacaatctcaaggtcaacgttgtcaacgtctcccaacatcaggttcaggagcgcccccgccgcttccggtagatgcgcgcacccctcgagcctcgaaaaaaaagttaccgattgtcttcggtcgatctatggcgtgctcaatcacacttgctctggctgacttcgcagctatgatgtagcctagagacacaggaatgaacataattctctctgagaaaggtgtcgctgattctcctggtggagatgacgcttgattgccaaaatttctccttttgcttactgtccgtttcagctcgggcgctgcgtagaaggttctctctgcatgatgcgcaggatgtcatcagagagtcgaaacctttggtgcgaactgcaccatcaactgcaccgcattggatcagatccatattaatcagtctatctacagaagtaaattgggtatcgtcataagcacaaagacgccgtagaaccacaaatcgaaccaccccatcgaattctgtcgtgaccaggctcacgccaaacccgctgagattgaagcatatcatgatcagatttcctttggcacgtagccttca

SEQ ID NO: 148 - DNA, 637 bp, Aspergillus fumigatus; identical to SEQ ID NO: 28

SEQ ID NO: 149 - DNA, 420 bp, Aspergillus fumigatus
atgggtcgcgttagaaccaagacagtcaagaggtccgccaaggtcatcatcgagcgctactaccccaagttgacgctcgactttgagaccaacaagcgtctttgcgatgagatcgctatcattgcctccaagcgccttcgcaacaagattgctggttacaccacccaccttatgaagcgtatccagcgtggccctgtccgcggtatctctttcaagctgcaggaggaggagcgtgagcgcaaggatcagtacgttcctgaggtttccgctctggatgtttcccagaccgagtccggccagctcgatgtcgatgccgacaccaaggaccttctcaagtccatgggcttcgacaatctcaaggtcaacgttgtcaacgtctcccaacatcaggttcaggagcgcccccgccgcttccggtag

SEQ ID NO: 150 - protein, 139 aa, Aspergillus fumigatus; identical to SEQ ID NO: 30

SEQ ID NO: 151 - DNA, 2037 bp, Aspergillus fumigatus
aaggtagtaggtgcagatattgttgatagacatttcaaaatgtattagttacatgattacttacttagatgtaatctttcgataatactttctagtcttgttgagttcagaaggccagtgtgtgctgaaaatgacagcgacctatgcggtgcccgtgtagcgaagagcactggctggaaataagaagtttattagaggagcctcatgatgcataatcattgtaagcgcacgatgcacaataatatatccgaatttctccagatgacactaagataataacgaaaatatcacatgacgttgtgggcaggtatgtattatgtaatctgatcggtagggccgatgtctcgcttagcggacttttctgtgggattgcaatttcaacttattattccgccgaccagcaacaaagcggttactcgactcgactccctccaccagagcccgtggtgtgatatacctgtctgtctttgatcctcgcaagatagacttgagtcgcagttatggcggttggaaagtatgccaattcacttctattattgttctgaacgcttttagcatgtgtctggatacggtggtttacaggtactgatccgggaacaggaacaagcgcttgtcgaagggcaagaagggtgttaagaagaggaccgttgatcctttctccaggaaggacgaatactctgttaaggtatgtcgacgtggactgtgtaagtcgaccgcagctaatctatatcaggcgccttccactttccagatcagagagtatgttgcacgcatatgatgtcgaattgcaggataaaggcgattcacaatggtagtggagattatgctgactgaattatagtgtcgggaagactctggtcaaccgcaccagtggtctcaagaacgccaatgactccctgaagggtcgaattttcgaggtctcgctggctgacctgcagaatgatgaagaccatgctttccgcaaggttaagcttcgtgtggacgaggttcagggcaagaactgtttgaccaacttccacggtcttgatttcacaaccgacaaattgcgatccctcgtgcgcaagtggcagtcgctgatcgaagccaatgtcactgtgaagacgaccgatgattatctccttcggctttttgctatcgccttcaccaagagacgcccgaaccagattaagaagaccacatatgctcgttcttctcaaatccgtgccatccgcaagaagatgattgaaatcatgcagagggaggcagccagctgctctctcgctcagctcactcacaagctcattcctgaggtcattggtcgtgagatcgagaaggctacccagggaatctatcctttgcagaatgtgtgtgaccctgttattcttactcgggatgaagactaactgcaatctaggtccatattcgcaaggtcaagcttcttaaggctcccaagttcgacctgggtgcactgctgaatctgcacggtgaatctacaaccgatgataagggccacaaggtcgagagagagttcaaggagcaggttctcgaaagcgtttaagtggactgaattaccagtatgctggttattcgggacattgatttgtacctacctgtatgcttggattctttttttatgagttaaaatgggaaaagaacttttgtcgcggcatcatgtctttattgactggtggtgtcgttaacttctatgtcctttgagaatggagcttgcaaagaaaactttgcccttattcaaatatttaattggacaattccgatcaaagtttagcagtagaatacctgctataccagtgatgtgctgatgcaacgggcacctgcagtttactttcagttgattcaaattctatattaacagagcccttttaccacaccactgacctggtattagtatagtgtctcgccctaggagactaaagaattgctagaagtatggttatacataatgttgaatagttagtatgatttattaatattattttcagtgcactgatatatatcataatgctactaaatatagctaccctaagatttatatagaga

SEQ ID NO: 152 - DNA, 1037 bp, Aspergillus fumigatus
atggcggttggaaagtatgccaattcacttctattattgttctgaacgcttttagcatgtgtctggatacggtggtttacaggtactgatccgggaacaggaacaagcgcttgtcgaagggcaagaagggtgttaagaagaggaccgttgatcctttctccaggaaggacgaatactctgttaaggtatgtcgacgtggactgtgtaagtcgaccgcagctaatctatatcaggcgccttccactttccagatcagagagtatgttgcacgcatatgatgtcgaattgcaggataaaggcgattcacaatggtagtggagattatgctgactgaattatagtgtcgggaagactctggtcaaccgcaccagtggtctcaagaacgccaatgactccctgaagggtcgaattttcgaggtctcgctggctgacctgcagaatgatgaagaccatgctttccgcaaggttaagcttcgtgtggacgaggttcagggcaagaactgtttgaccaacttccacggtcttgatttcacaaccgacaaattgcgatccctcgtgcgcaagtggcagtcgctgatcgaagccaatgtcactgtgaagacgaccgatgattatctccttcggctttttgctatcgccttcaccaagagacgcccgaaccagattaagaagaccacatatgctcgttcttctcaaatccgtgccatccgcaagaagatgattgaaatcatgcagagggaggcagccagctgctctctcgctcagctcactcacaagctcattcctgaggtcattggtcgtgagatcgagaaggctacccagggaatctatcctttgcagaatgtgtgtgaccctgttattcttactcgggatgaagactaactgcaatctaggtccatattcgcaaggtcaagcttcttaaggctcccaagttcgacctgggtgcactgctgaatctgcacggtgaatctacaaccgatgataagggccacaaggtcgagagagagttcaaggagcaggttctcgaaagcgtttaa

SEQ ID NO: 153 - DNA, 771 bp, Aspergillus fumigatus
atggcggttggaaagaacaagcgcttgtcgaagggcaagaagggtgttaagaagaggaccgttgatcctttctccaggaaggacgaatactctgttaaggcgccttccactttccagatcagagatgtcgggaagactctggtcaaccgcaccagtggtctcaagaacgccaatgactccctgaagggtcgaattttcgaggtctcgctggctgacctgcagaatgatgaagaccatgctttccgcaaggttaagcttcgtgtggacgaggttcagggcaagaactgtttgaccaacttccacggtcttgatttcacaaccgacaaattgcgatccctcgtgcgcaagtggcagtcgctgatcgaagccaatgtcactgtgaagacgaccgatgattatctccttcggctttttgctatcgccttcaccaagagacgcccgaaccagattaagaagaccacatatgctcgttcttctcaaatccgtgccatccgcaagaagatgattgaaatcatgcagagggaggcagccagctgctctctcgctcagctcactcacaagctcattcctgaggtcattggtcgtgagatcgagaaggctacccagggaatctatcctttgcagaatgtccatattcgcaaggtcaagcttcttaaggctcccaagttcgacctgggtgcactgctgaatctgcacggtgaatctacaaccgatgataagggccacaaggtcgagagagagttcaaggagcaggttctcgaaagcgtttaa

SEQ ID NO: 154 - protein, 256 aa, Aspergillus fumigatus; identical to SEQ ID NO: 33

SEQ ID NO: 155 - DNA, 1819 bp, Aspergillus fumigatus
aattcatcagcataacgaacccccaacgacttcgaaaaaaaagcccgattcgaaagaattgcgcattcaacataccatggtgggcggcttcgtgtcgtgtcgtacgggtattgtcgacaatgaggattgaagatgggccaggtcaatttgggatgttcgttgtgggactagggtttttttctgtgttggtgcggtcacgctgcggctgggctaagcgggcacgtgactgtggctgactgcctggtgacgcccccccccggaggaacccccaaccggcagccagataggctcgggaggatcatcgtcgaatgatggcattgttcttggtctcagtggatgggttattaatgactgcctggacggctggatgactccgtcgctgatttagcattgtgatccacgatttatgtttcatttctggggcgcgggtttactaccatcacttttgtcactaccatcacttttatactgagtttctgaccccgaccccgaaccagactatggcaacttcgactgggaccggatgggctcagctccggcagcaagcccgttcgcttgagactcaggtacggaactcgaaactacgctataatgaggctttactcgtgatttggatgttgacaataatgttcctagaccgagagtctgtttcacacctatgcgcagtatgcatcgatgacgaagctgcctccgaaaccctcagaagaagaacaacggattgaatcgcaactgaaggatcttcttgaaaaggtgtgcactttgaggccctctagtccagcccaacagacgatcatgctgacacgatccgatcatagcgtgaagccctcatctcccagctctcccgtctccttgactccgaagccactcttaccgcatctgccctgaaacagagcaatcttgcccgcaatcgcgaagtcctccaggatcatcgccgcgaattgcagcgcctgaacgccgcaatcgccgagtcccgcgaccgagccaatcttctgtctaacgtccgctccgacattgatgcctaccgcaattcaaaccccgccgcggctgaggcagactacatgctcgaggagcggggtcgtatagatgaaagccataacatgatagatggtgtcctaagccaggcgtatgcaatcaacgagagttttgggctacaacgtgaaaccctggccagcatcaatcgccgtatcgtcggtgctgccaataaggtaccaggaatgaatgcattgattggtaagattgggacgaagaggagacgtgacgcaatcatcttgggggctttcatcggcttttgtttcttgatggtgttcttcttccgatgagatgctggtgctccgtataccgccgatcttcctgtgttataattccttgctcaacgttatctacatcggagaccgcacggcgttcgggtgttttcatgtactccttttctgcatgcaagcactaatcacaatggtcatggcgtttcaggttgtctatttcacatttatgtacatacagggtcagactgctgtagccctagggctcaccgcatgatcactcttggtttcggacttgcggattcaccttggtttcttcccgcccattcctcagccggtagcttcgactcgagactgattcttctctcctggattaatttgcgaaccccgttgttcaatccgtctagctcgccttcctctgccggcccgctacccgcccatcggatgcgacagttctcgtccagcagatagacataaccaactttactgttcatcattccgattgcttctttcaacccatccgtaagacctttgcgcacaaggaaataccgttcgtgctgctc

SEQ ID NO: 156 - DNA, 819 bp, Aspergillus fumigatus; identical to SEQ ID NO: 37

SEQ ID NO: 157 - DNA, 684 bp, Aspergillus fumigatus
atggcaacttcgactgggaccggatgggctcagctccggcagcaagcccgttcgcttgagactcagaccgagagtctgtttcacacctatgcgcagtatgcatcgatgacgaagctgcctccgaaaccctcagaagaagaacaacggattgaatcgcaactgaaggatcttcttgaaaagcgtgaagccctcatctcccagctctcccgtctccttgactccgaagccactcttaccgcatctgccctgaaacagagcaatcttgcccgcaatcgcgaagtcctccaggatcatcgccgcgaattgcagcgcctgaacgccgcaatcgccgagtcccgcgaccgagccaatcttctgtctaacgtccgctccgacattgatgcctaccgcaattcaaaccccgccgcggctgaggcagactacatgctcgaggagcggggtcgtatagatgaaagccataacatgatagatggtgtcctaagccaggcgtatgcaatcaacgagagttttgggctacaacgtgaaaccctggccagcatcaatcgccgtatcgtcggtgctgccaataaggtaccaggaatgaatgcattgattggtaagattgggacgaagaggagacgtgacgcaatcatcttgggggctttcatcggcttttgtttcttgatggtgttcttcttccgatga

SEQ ID NO: 158 - protein, 227 aa, Aspergillus fumigatus; identical to SEQ ID NO: 39

SEQ ID NO: 159 - DNA, 2601 bp, Aspergillus fumigatus
tataatttctgaactgtagactccaaattgcaaatcctctcgcctttcaagtgattccactcctgcctccttttcgtcttctttttttcccctttttatcttttcattttatcatctttgttactgttttcccaaacgaatccattattatttctttcccttggaggttccctactgtggtcttgtgtctgcactcttgccaagcccattggtccttgctctgagcctatcacttgcgattcgcccgcccataagtccgcctctctcaacctttccatctcacgcgcacctccactcaacatccaccattcggatattccgcccatccaaagcgaacacccctccttctgctccaccatcgattgcagtctgcccaaaacggacttcagaactcccttctacgctattttccgccattcactgttgaagtgcagccctccatactctcgatagcaactgcccaacccccctcttactgccaaccccacaagttgcccgtgatgtcacaaaatcgacctggggtgttctcgaatctgcgcatgggtggtaaggaacatccaaatgctgagtccaattgttcagaaaacattacccaggagcctgtggaactaactgctttgctttccgaccatacagaagtcgtccgcgagaaggtccaggatggactgacaggggaaactaaggagatttcgtactcacaatgtaaaatcgtcggcaatggatcgtttggtgtcgtctttcagacgaaaatgatgccaagcggcgaggatgctgccattaagagggtccttcaagacaagcgcttcaaagtatgtgtacattataagggcaattgccctcgctgcccaacccaaagatactgtcgctgacgagataccagaatcgagaactgcagattatgcggattgttcgccatcctaacatcgtagaattgaaagccttctattactcgaacggcgagagggtatgcgactctcctttgtctccccattcgttctagtttgccgtttgctgactaccctaccattgtctttcacagaaggatgaagtgtacctaaacctcgttctcgaatacgtaccagaaaccgtgtatcgggcgtcgcggtactttaataaactcaaaacgactatgccaatgttggaagtcaagctgtatatctatcaattgttccgttccctggcatacatccattcacaaggcatctgccaccgtgacatcaagccccagaatctcttacttgatccatccaccggcatcctcaaactctgcgactttggttcggccaagattctggtagagaatgagcccaacgtttcctatatctgttcccgctactatcgtgcgccggaattgatctttggcgccactaattacacaacaaagatcggtaagtcttgactgattcctccttcaagtttggtactgtcatgctgacgatcgtcaagacgtgtggtccacgggttgtgtgatggctgaactcatgcttggtcagccattgttccctggagagtcgggaattgaccaactggtggaaatcatcaaggttcttggaacccctactcgggagcagatccgcaccatgaacccaaactatatggagcacaaattccctcaaatcaagccacacccattcaacaaggtgaccacgctcttaaagaacttcttgcgaatatgcactgacttgatgaccccaggttttccggagagctcctcacgaggccattgatctgatctcagctttgctagaatacacgccgacacaacgtctctccgctatcgaggcgatgtgccacccgttcttcgacgaactcagagatcccaatacgcgactgcccgactctcggcaccctggtggcgctgctagagacctccccaatctctttgatttctccagacatggtttgttgtcacttgaggcccaaattcattcttccagatggcttattcgctgatcactcttttgtagaactttctattgcacctgcattgaacagccggctggttccccctcatgcacgcgccgctctcgaggcccgggggctagacattgacaacttcactcctctcacgaaggaggagatgatggcacgtctcgactgagtcgatacgtttctcgttacgaatcaaacgccccctcgatagtgcttatatccgcacaactgcgcgaggatatgatgtcatgcgatgcattttgctttgtgaactgggatgtccttggaccagaagtggcttttatggaacttgtcgaccagagtcgctgactagacacccgggagttggcatcttttccgaacgtatgaggatacaaggcaaatccccatatttaaaagaaaaaggagaaaacaggagaaaaaaaaggaatgggaaatacagaaacatcgcattctggtcacaacataagcggttttcgtgaggcggtggtgcatcatgacaatatcatgcaacggggttgaggaattgtcatagtctggagttttcgaggtgtctgagacagctcttgggaaaggaaaaaaagtgataccctttagtgtgcgagtctgccttcggggatttaagtttgcatagcttcattctcgtttgaaggaggacatgaccattcc

SEQ ID NO: 160 - DNA, 1601 bp, Aspergillus fumigatus; identical to SEQ ID NO: 40

SEQ ID NO: 161 - DNA, 1185 bp, Aspergillus fumigatus
atgtcacaaaatcgacctggggtgttctcgaatctgcgcatgggtgaagtcgtccgcgagaaggtccaggatggactgacaggggaaactaaggagatttcgtactcacaatgtaaaatcgtcggcaatggatcgtttggtgtcgtctttcagacgaaaatgatgccaagcggcgaggatgctgccattaagagggtccttcaagacaagcgcttcaaaaatcgagaactgcagattatgcggattgttcgccatcctaacatcgtagaattgaaagccttctattactcgaacggcgagaggaaggatgaagtgtacctaaacctcgttctcgaatacgtaccagaaaccgtgtatcgggcgtcgcggtactttaataaactcaaaacgactatgccaatgttggaagtcaagctgtatatctatcaattgttccgttccctggcatacatccattcacaaggcatctgccaccgtgacatcaagccccagaatctcttacttgatccatccaccggcatcctcaaactctgcgactttggttcggccaagattctggtagagaatgagcccaacgtttcctatatctgttcccgctactatcgtgcgccggaattgatctttggcgccactaattacacaacaaagatcgacgtgtggtccacgggttgtgtgatggctgaactcatgcttggtcagccattgttccctggagagtcgggaattgaccaactggtggaaatcatcaaggttcttggaacccctactcgggagcagatccgcaccatgaacccaaactatatggagcacaaattccctcaaatcaagccacacccattcaacaaggttttccggagagctcctcacgaggccattgatctgatctcagctttgctagaatacacgccgacacaacgtctctccgctatcgaggcgatgtgccacccgttcttcgacgaactcagagatcccaatacgcgactgcccgactctcggcaccctggtggcgctgctagagacctccccaatctctttgatttctccagacatgaactttctattgcacctgcattgaacagccggctggttccccctcatgcacgcgccgctctcgaggcccgggggctagacattgacaacttcactcctctcacgaaggaggagatgatggcacgtctcgactga

SEQ ID NO: 162 - protein, 394 aa, Aspergillus fumigatus; identical to SEQ ID NO: 42

SEQ ID NO: 163 - DNA, 2539 bp, Aspergillus fumigatus
ttgaggatttgcggaaccttcacgatggcacttgcacccacaatagcaatacccagagccttggagatagcaagagacgtgcaggcagcatccttggtgacatccagatcgaggaggagggcgctgtggcatggcgatccgatcagggagatgatgacatcgtggacaggttgtgggagagcggacgagataggacggagaacgggttgaagagggtcaataacgaccctttgaagcgtatccatggcgagagtctacagggcaaaagacggctcagggccttggttgaagaggagtttgccaacggagcacggtggttccaaaagatcagcaacaaccatcgggacggaatggtgcattcattcctgcggttgtggtctctgtgcctcgccggggaatttctgccctgtcaatcgcgactcttccgagactcactatctcatgatctagatatcgtcctatcgtgatttcaatatccctccgtccatgttccttccgccatgatttatctccggtcctcgttgctgaggtctggattggctcgagatcctgctcgcctgtgttcacaatgcttctcacgactctcaccatcacgacgacctgtcgcagttcgcagcttcttctcctcatctcggctgcgggctggcattgccgatcatgaatcaactccctcgactgtccaaaagacctatttttctgccaatcggaccgcagatggcttacttgcatccttatccgccgtcaatagctcccctcgaagtattgccgacaatgcgttatcacagggtgcagccagttcggagtcgattacttcacagtctacttcacaagagttacctcatcgccggaggaagcggttaaaggaagaggcggccaagaataatgctgcagaaaccgaactccctcctgatgcctcgtctcaattgtccaccctctcatcagccctccctgcgacttccctgcgccgcaagctggctgcgtttctcgccctcacaaagcctcgtctctcgttcctgatcgtgttgacgactacctccgcttatgggatgtacccgatctcctctcttctcacacttgacccttcaatgactcccctaccgaccctctcgacctcaaccttgacctttctctacctgaccacaggaaccttcttgtcttcatgcagcgccaataccttgaatatgctccttgaacctaaatacgatgccctcatgtcacggacacggaaccggccgttagtgcgggggctactctcacgccgtgctgcggtattgtttgcgattgcgactgctgctgcaggtctcggtttgttatacattggaacgaaccctacgactactgcgctctccgccagtaatatctgtctctatgcctttgtgtatacgccgctgaagcgtatatcagtgatcaacacctgggtaggcgccgtggtaggaggcattcctccgttgatgggttggaccgctgcagcaggccagacagcgaccactggccacgacagctggcgggacatgttgttcagcaaggatagcatcggtggttggctcctgggtggcattctctttgcatggcagtttcctcatttcaatgctttgtcctacatgatccgtgaagagtacaaggcagccgggtacaggatgctcgcatggactaatcccgccgcaaatgcccgtgtcgcactacgatattctcttctcatgtttcctttctccgtcggtctctggtgggtaggagttgtcggtaatggtttcctggttggaagcacggcggccaatggctggctagtcaaagaggcctacaaattctggcggcaccaaggcgccaacggcagtgctcgacgcctcttctgggccagtatttggcagctgccaatcctccttgtcggtggtctggtcacgaagaaaggtctctgggatggtgtctggaacaatgttttcggtcagcctgtggaagacgaggatgactatctctgggaggatgaggatgaagtggcagaggcggagcgcaagatgatacctgcgaagacgagtagctcgtgattcttacttttgtttatcagcgcaattcgacactgatattgttttgtttacagcactttatcatactagactgcctttttggcatgggaggctggcgtcgttgtataacaaattctttttaaacttcactcatgatgatccgcaacacgtagatatagcttcaacatgaattgattcaggtagttttctgtatagcaccttaaacggggggttcttgctatatcgaacctctaccccaacttataagattgagttctgtatcacaatctacttgcacttaaaccaagactatatcggtgtttctatgagacaactatttacagatcgcccccgtgagaagagattgggtatcatgtctatcctaatgtacaattcagctgagcggcttgcggcaactgcatcttcatacactcattcatcctttaaacgaagatggtaggtttctaatgcttctttcgtcatcatgctaggagccttgaccatggcatcatcgccaccct

SEQ ID NO: 164 - DNA, 1539 bp, Aspergillus fumigatus
atgatttatctccggtcctcgttgctgaggtctggattggctcgagatcctgctcgcctgtgttcacaatgcttctcacgactctcaccatcacgacgacctgtcgcagttcgcagcttcttctcctcatctcggctgcgggctggcattgccgatcatgaatcaactccctcgactgtccaaaagacctatttttctgccaatcggaccgcagatggcttacttgcatccttatccgccgtcaatagctcccctcgaagtattgccgacaatgcgttatcacagggtgcagccagttcggagtcgattacttcacagtctacttcacaagagttacctcatcgccggaggaagcggttaaaggaagaggcggccaagaataatgctgcagaaaccgaactccctcctgatgcctcgtctcaattgtccaccctctcatcagccctccctgcgacttccctgcgccgcaagctggctgcgtttctcgccctcacaaagcctcgtctctcgttcctgatcgtgttgacgactacctccgcttatgggatgtacccgatctcctctcttctcacacttgacccttcaatgactcccctaccgaccctctcgacctcaaccttgacctttctctacctgaccacaggaaccttcttgtcttcatgcagcgccaataccttgaatatgctccttgaacctaaatacgatgccctcatgtcacggacacggaaccggccgttagtgcgggggctactctcacgccgtgctgcggtattgtttgcgattgcgactgctgctgcaggtctcggtttgttatacattggaacgaaccctacgactactgcgctctccgccagtaatatctgtctctatgcctttgtgtatacgccgctgaagcgtatatcagtgatcaacacctgggtaggcgccgtggtaggaggcattcctccgttgatgggttggaccgctgcagcaggccagacagcgaccactggccacgacagctggcgggacatgttgttcagcaaggatagcatcggtggttggctcctgggtggcattctctttgcatggcagtttcctcatttcaatgctttgtcctacatgatccgtgaagagtacaaggcagccgggtacaggatgctcgcatggactaatcccgccgcaaatgcccgtgtcgcactacgatattctcttctcatgtttcctttctccgtcggtctctggtgggtaggagttgtcggtaatggtttcctggttggaagcacggcggccaatggctggctagtcaaagaggcctacaaattctggcggcaccaaggcgccaacggcagtgctcgacgcctcttctgggccagtatttggcagctgccaatcctccttgtcggtggtctggtcacgaagaaaggtctctgggatggtgtctggaacaatgttttcggtcagcctgtggaagacgaggatgactatctctgggaggatgaggatgaagtggcagaggcggagcgcaagatgatacctgcgaagacgagtagctcgtga

SEQ ID NO: 165 - DNA, 1539 bp, Aspergillus fumigatus; identical to SEQ ID NO: 164

SEQ ID NO: 166 - protein, 512 aa, Aspergillus fumigatus; identical to SEQ ID NO: 54

SEQ ID NO: 167 - DNA, 2679 bp, Aspergillus fumigatus
tcacattcactgcaggtcgttgtactccggagttatgcaatgatcgtcccgtttaattatcttccgaactttggactcgtatattttagctgcgtgtcacggtgatcatgatcttcattcatctctttatctgattcgatcaataccgggagtactgcaaggaggacaagtagacaggcatctcgagaattcggtgagaaagcaaggtacagaagagaactactccgtactctgtactctgtagagaaaggcaggaggttcaaacatgattggcccgtggagaataagaaaatatcatgccttaggtccaaaggctagtgctcacatgaccttatcagttgagtcaggtgatcttatcgttgtcccagagagatgtgaagaattattgcaccggggagcacgcaaggaaaccattctatcctatctcgtccctttagattaccacaggacatctacatcttgaaccttaccattccaaattacagactgcctctgagtacatgctcaacgccgcggttgctgccccgcgatgttttgtatatcccactgatcgcgcagcaatgcgcttgggctttgctcttcgtctctcctctcctgcacctctcttctcaacagcacctttccgtcgacagttgcatgcttccggcgtccgatcaattgaacctgttatctttcgaaatagccttgaaaagactcttgaggctcatcgatcctccaatcgagccagtctgatccgcaaggtgattaaccacgattgtcctgctgaaacgccccctccaattttaccacttgagaatcgtgctggtcatgatcaatcatctcaaaaggcctcctccgtgtcaaatgcagagtcagagtccccccggtcttctgcgcctgcgagacgagcgcagaggaaggcccgttcgcccagccaagtagccaccccgcagccccagacaacagaatatccacaactgcaatggcatgcagatgaaaccaagggccgaccggcacaaagtccttggctgaagtacttgactaccgattggaaaacgcccgatgccgtttcgcgtctcgacgcggagatccgcgctcttgagctctacatgacaccgaccccgtcggagcggactgagatagatcggctggttgcagatatgggtaggttgctagcgggaatcgtccccagcccgccccaggtaaccggttcatggcggacgcgatttgccttgagccactcgggtctcgattttgtcttacctgtcccggattcagaccgatccacccgtgacgttcgcaagccgagtgccacacggcccaaggtgctccagacttacaaaaagctcttacatgaagtgggacatgcgcttcagcagtccccctcgttcgcggagcgagtccgcatcataggcagccgtttccccgtcctctcagccatccatcgccccacgggccgcctgctgcagttccactgcggtgaagggctaccggcctctgtcgaatacatcatggattaccaggccgagtatccctcgatccggccgctctacgtgaccgctcgcctgatcctggaggcgcggggtaggtatggccgtactcagatgtctattgaatccgatgccctcgtaatgcttctcgtggccttcctcaaaatgaaccacgggcgttttcagcggcccgactgtctcggcgagcagctgatcgcgtttctgcgcgcctacggcagcgatattgacctgaccaccaccggtgtgtccgtcgatccccccagttggttcaatgctagtacggtcaaacgcgccagcgccctgtacgcgcccgatgatctacccgcgcatctgcgcggccagcgctccctcatcagcctcaagagaacagcagccgccagacgcaatctgcctgccgccagccggctgtgcgtgcaggaccccaccaattacatgaatgatctgggccgcagctgcgtgcgtacgttggaactccagcacacgttctcgcttgctcatgaccgtctcggcgcaagtctcaagcgctgggatgacagtgaaccggccgcgaacgttagtatcctgacacgggccctgcaagcaaacttttctgattttgaaaatctacgcgccaaatcgcttaagctcaacgcgacctagcaatgaactgggccagagccttggagcttgggacattgcagcctatcttatttctcacttccattactgatagtaaattatatatgagataatgtggagtccggcaacgtgtttcgtcacctgggctaatccctgtcactggccactatgtagatgcagttgactcaatagagggtcgctataatcaaacatcagacaatgcacgagtagaacagatggaatatgcttgtaagagaacgcccgcttcctagtcaagcagctctgaagagcaaaacaatatatccaaacccgctctattcgaacgaaaatgcggaaataaccatgacagacaaagaagagacaagccaggccagggcaaaagtagtctatgacatgaattcaacgatcaagatcacgccggtgtatgagaactccagggttagcaataccaacaatcgacatgtatggcaccgcaaggaaaagcaccgcggaaccatacgaggatatacgatcgatcaattgctgcccctctgtcagcagctatttccattgctgacgggatgctgatggg

SEQ ID NO: 168 - DNA, 1629 bp, Aspergillus fumigatus
atgctcaacgccgcggttgctgccccgcgatgttttgtatatcccactgatcgcgcagcaatgcgcttgggctttgctcttcgtctctcctctcctgcacctctcttctcaacagcacctttccgtcgacagttgcatgcttccggcgtccgatcaattgaacctgttatctttcgaaatagccttgaaaagactcttgaggctcatcgatcctccaatcgagccagtctgatccgcaaggtgattaaccacgattgtcctgctgaaacgccccctccaattttaccacttgagaatcgtgctggtcatgatcaatcatctcaaaaggcctcctccgtgtcaaatgcagagtcagagtccccccggtcttctgcgcctgcgagacgagcgcagaggaaggcccgttcgcccagccaagtagccaccccgcagccccagacaacagaatatccacaactgcaatggcatgcagatgaaaccaagggccgaccggcacaaagtccttggctgaagtacttgactaccgattggaaaacgcccgatgccgtttcgcgtctcgacgcggagatccgcgctcttgagctctacatgacaccgaccccgtcggagcggactgagatagatcggctggttgcagatatgggtaggttgctagcgggaatcgtccccagcccgccccaggtaaccggttcatggcggacgcgatttgccttgagccactcgggtctcgattttgtcttacctgtcccggattcagaccgatccacccgtgacgttcgcaagccgagtgccacacggcccaaggtgctccagacttacaaaaagctcttacatgaagtgggacatgcgcttcagcagtccccctcgttcgcggagcgagtccgcatcataggcagccgtttccccgtcctctcagccatccatcgccccacgggccgcctgctgcagttccactgcggtgaagggctaccggcctctgtcgaatacatcatggattaccaggccgagtatccctcgatccggccgctctacgtgaccgctcgcctgatcctggaggcgcggggtaggtatggccgtactcagatgtctattgaatccgatgccctcgtaatgcttctcgtggccttcctcaaaatgaaccacgggcgttttcagcggcccgactgtctcggcgagcagctgatcgcgtttctgcgcgcctacggcagcgatattgacctgaccaccaccggtgtgtccgtcgatccccccagttggttcaatgctagtacggtcaaacgcgccagcgccctgtacgcgcccgatgatctacccgcgcatctgcgcggccagcgctccctcatcagcctcaagagaacagcagccgccagacgcaatctgcctgccgccagccggctgtgcgtgcaggaccccaccaattacatgaatgatctgggccgcagctgcgtgcgtacgttggaactccagcacacgttctcgcttgctcatgaccgtctcggcgcaagtctcaagcgctgggatgacagtgaaccggccgcgaacgttagtatcctgacacgggccctgcaagcaaacttttctgattttgaaaatctacgcgccaaatcgcttaagctcaacgcgacctag

SEQ ID NO: 169 - DNA, 1629 bp, Aspergillus fumigatus; identical to SEQ ID NO: 168

SEQ ID NO: 170 - protein, 542 aa, Aspergillus fumigatus; identical to SEQ ID NO: 57

SEQ ID NO: 171 - DNA, 1573 bp, Aspergillus fumigatus
  modified_base (683): a, c, g, t, unknown or other
tggtgcttagggacctgtgagtttgtgacacaacccaccccggaaccagggattctccaccgcgcccccgaccgaccattttggacttggttagtaagctccagtgaccagaaagcttaccagctagcttcacttgagatatcacaagatgctttggtcggcctgccacgaggtcttgctccatgtcaaccacgaggattcagtctctgcctcatctcagtccaggcgaggtttctttgctggatcttgcggctgatgaccctcgcgatgtggtgtccctgtccgacaaggaagcgttgattttgcagctctacaatcaaatccaggaactggaactggaaaaggcacttcttgaacaaggtacgcgtcaaatttattttttttgtaattttcttttttttgtttctctgatgtagcttcttgggccctccaaatctgtcagcccaggttactgattcccactagagctggaaccggcttctggggacaatctggatgagcaacttgcaatcgcagaacgtgagcttctcgaggcaagggccacgtacacggtcaggagaaaggccaccagtactgtcctgatgactgatccaacattaaaagctgttcacttgaaagctatatcacctgttgaaaggttttacctcctttcagtttatgcgagcatttcaattctgcattcncctatactaacgcttcatagagctcttctacccctggtcaaccggcgtgatgtgttgtctttggcacatgagaacctaatgaatgcgcacaacgcgactttgagggaactatccaatttagaagtacaaaatctagagctacaccagaggaatcaagagctagcgcggcagcttcttgagtccgcgaaggatgatgattcatggagagaagcactggatgatgacgacctcaaggcacaacttgagcagctagaggccgatcgcaaaaagagcaaatcaagatgggaagtcatgaaaagcgttgcaagtgctattgttgtgggaagtggagtgaactgggctgaagacgatgagcttacagctctagtcattgatgaatctgatgattaaataatcgcctcgaaattaatgatttcgaacaatttggtagtattgacttctccgaccggcgtactacaggatatgacttccatttatgactagtagagtaaacctcattcattatttccaactagggcggtatatacacagtatcgtcttggtcgaatcagcagaacggctgaggaagctcggttaggtaccgtaagtgtcgcccagactgccgataacctgaagacgcgccagcgcccactagaacaatcttcagtggccgtgagacccacgtgacatcatcgtccatccacactaacaagacttgactgtcagaccatatgatttctgctgggtgtcccaaataataatcttcatacatacattggccctgaagccctggaatcatggagaaaattcgaatacagatggaaggcaattagaacccgataaggtgggcgctaattgatggatgcgtctaagaatgttatgcattaatcagcgatttacgggtaaccatg

SEQ ID NO: 172 - DNA, 573 bp, Aspergillus fumigatus
  modified_base (183): a, c, g, t, unknown or other
caacttgcaatcgcagaacgtgagcttctcgaggcaagggccacgtacacggtcaggagaaaggccaccagtactgtcctgatgactgatccaacattaaaagctgttcacttgaaagctatatcacctgttgaaaggttttacctcctttcagtttatgcgagcatttcaattctgcattcncctatactaacgcttcatagagctcttctacccctggtcaaccggcgtgatgtgttgtctttggcacatgagaacctaatgaatgcgcacaacgcgactttgagggaactatccaatttagaagtacaaaatctagagctacaccagaggaatcaagagctagcgcggcagcttcttgagtccgcgaaggatgatgattcatggagagaagcactggatgatgacgacctcaaggcacaacttgagcagctagaggccgatcgcaaaaagagcaaatcaagatgggaagtcatgaaaagcgttgcaagtgctattgttgtgggaagtggagtgaactgggctgaagacgatgagcttacagctctagtcattgatgaatctgatgattaa

SEQ ID NO: 173 - DNA, 573 bp, Aspergillus fumigatus; identical to SEQ ID NO: 172
  modified_base (183): a, c, g, t, unknown or other

SEQ ID NO: 174 - protein, 190 aa, Aspergillus fumigatus
QLAIAERELLEARATYTVRRKATSTVLMTDPTLKAVHLKAISPVERFYLLSVYASISILHSPILTLHRALLPLVNRRDVLSLAHENLMNAHNATLRELSNLEVQNLELHQRNQELARQLLESAKDDDSWREALDDDDLKAQLEQLEADRKKSKSRWEVMKSVASAIVVGSGVNWAEDDELTALVIDESDD

SEQ ID NO: 175 - DNA, 2593 bp, Aspergillus fumigatus
tgtcaaagaaagtggatatatatgctgcttgttctcacctggtaatccatcttgaatcacgtccactaagaatcagaaaaactgagcgagtggacaaaaacgggtgatacgcatcgataagcttcgactggaaattcggagatcgccatatatttgagttatgtaagcaccgccgcaggcgtctcattgggctggggaagctatcaacaaccacccagagcttcttgaacttaactccgggggtgcatgactaatagtttcaataatggacgtcggatgctttgtaaatcaacggcggtcctacaatggggatctatgcacagttcgttacataggtaaagttgagggcaccaccggcgagtggctcggagtggaatgggatgaccccacgcgggggaagcattctggagaacacaacggagtgagatattttacatgtatgaaagtattttcaagactggatagagcggattgactgacttgaacggaaggtagaaggaaacaccccacggctggttcgttcgtgcgcccttcgcgacggaccgacagacctcgaggcttccttgaggcagtgcgtcacaagtatgcttctgagttccaagaagaactcgcaagacagcagtcaggcgaagtctctgctgcgcgggaaatcatcaaatttagtagcaaagtagtggaagaggtcggcttcgacaagatccggaagaaacttgcagagctccaggaattgaaaatcgtgctcctggatcgcctatgcatcgcaggagttctccctcatagagcgagtctacatgagcttgcagaggcttgcaaggagatagaacagacatgtcctaagatcgttgacctcgatctgagttacaacttactggaaagctgggttgacattgcaaacatatgtcaacagctgaagcgcttgaagacattgaagctgatgttggtcattcagtacatctgtgagaagcatgctgacagttggcagcggaaatcgtctaggtcctcgacaggagggtctgatattcgacggtatcacaacactacacttggacgagactctactcgaatgggacgaggtatgctgcacaaagctactgcttggttaccagtgagctgactgactcttggtcccctttagatttcagctttgacatatcaattcccgtcactctctgctctgtctgcctccgcaaatcagattacccagatcttgacacctatcacggataccatcacgaccttgacactggaaaacaatgacatctcttcgctatcctcattagcatgtctgacctctttgagcaagctcgagcacctctcgctgagagagaatcgtatcgggaaagtctatgcgtctggcatggaaggaaactctcttcagttttccgaaaatctcagatcggtggacctatccagaaacaatatcgattcttggctgtttgtgaatgaacttcaacgcgtatttcctgggctgcaatctttgcgcatatcaggaaatcccctgtacgacaagcctgttgccccctcgaacgtcacaaatttaccggagaagccaatgacggtggacgaggcctatatgctaacactctctcgactcgcttccatccaaacgctcaactacagcaagataacttcccaagaccgaagtaatggcgaactctattacctttccctcattggcaaggagttatccgcgtatccggaaagcgcagaacgcgagattcttgctacacatccccgctatcaggagctttgtgagaaatacggagcgcccacaatcaggagagccgagctggcaggcgctgccgtgaatccgcgctctgttgccgcccgagtagtgaagttggctttttgcttgcactcatcagttagttccggtgcaaaccaagaacaatttcgagttcagaagatcccgagatcctttaatacatatcaagtcaaggcaatcgcctcccgcctgttcaatttgccgccttaccagtgccgactagtctgggagaccaacgagttagaccctattcatcaggagaaaaaggacgatggagacgattgggatagtgatgaggatgaagccacagctattggattgggggagagtaacaagctcacaccggcgacggaggatggaaagttcatcagaagagaggttgagctcctggattcaacgcgagacataggcttttggttccaacccgatactgttgaggcaaggatcagagtagaagttgcaacatcgaattgacaactatttcgacataggagttgtgaaggagttccttaacctacattgtcgcgggtggggtaatatatatagttgagcacgactcgtgcgttaggatacaacgaggtcaacgacagaaaagtagaacctcttacgcaagcgcctgtggcctctgttacccgaatggatttagaggccttcttggttattcttaaacaatatgatatgtgtagtgtatattcaagctggagggcaacactgactgctgttttaggactaggtcggttttgggtggcagattatcaccgcaactgttttgtgataacagtgatatcatttccctttcatataacaatttaatactcagacatcgtc

SEQ ID NO: 176 - DNA, 1974 bp, Aspergillus fumigatus
atggacgtcggatgctttgtaaatcaacggcggtcctacaatggggatctatgcacagttcgttacataggtaaagttgagggcaccaccggcgagtggctcggagtggaatgggatgaccccacgcgggggaagcattctggagaacacaacggagtgagatattttacatgtatgaaagtattttcaagactggatagagcggattgactgacttgaacggaaggtagaaggaaacaccccacggctggttcgttcgtgcgcccttcgcgacggaccgacagacctcgaggcttccttgaggcagtgcgtcacaagtatgcttctgagttccaagaagaactcgcaagacagcagtcaggcgaagtctctgctgcgcgggaaatcatcaaatttagtagcaaagtagtggaagaggtcggcttcgacaagatccggaagaaacttgcagagctccaggaattgaaaatcgtgctcctggatcgcctatgcatcgcaggagttctccctcatagagcgagtctacatgagcttgcagaggcttgcaaggagatagaacagacatgtcctaagatcgttgacctcgatctgagttacaacttactggaaagctgggttgacattgcaaacatatgtcaacagctgaagcgcttgaagacattgaagctgatgttggtcattcagtacatctgtgagaagcatgctgacagttggcagcggaaatcgtctaggtcctcgacaggagggtctgatattcgacggtatcacaacactacacttggacgagactctactcgaatgggacgaggtatgctgcacaaagctactgcttggttaccagtgagctgactgactcttggtcccctttagatttcagctttgacatatcaattcccgtcactctctgctctgtctgcctccgcaaatcagattacccagatcttgacacctatcacggataccatcacgaccttgacactggaaaacaatgacatctcttcgctatcctcattagcatgtctgacctctttgagcaagctcgagcacctctcgctgagagagaatcgtatcgggaaagtctatgcgtctggcatggaaggaaactctcttcagttttccgaaaatctcagatcggtggacctatccagaaacaatatcgattcttggctgtttgtgaatgaacttcaacgcgtatttcctgggctgcaatctttgcgcatatcaggaaatcccctgtacgacaagcctgttgccccctcgaacgtcacaaatttaccggagaagccaatgacggtggacgaggcctatatgctaacactctctcgactcgcttccatccaaacgctcaactacagcaagataacttcccaagaccgaagtaatggcgaactctattacctttccctcattggcaaggagttatccgcgtatccggaaagcgcagaacgcgagattcttgctacacatccccgctatcaggagctttgtgagaaatacggagcgcccacaatcaggagagccgagctggcaggcgctgccgtgaatccgcgctctgttgccgcccgagtagtgaagttggctttttgcttgcactcatcagttagttccggtgcaaaccaagaacaatttcgagttcagaagatcccgagatcctttaatacatatcaagtcaaggcaatcgcctcccgcctgttcaatttgccgccttaccagtgccgactagtctgggagaccaacgagttagaccctattcatcaggagaaaaaggacgatggagacgattgggatagtgatgaggatgaagccacagctattggattgggggagagtaacaagctcacaccggcgacggaggatggaaagttcatcagaagagaggttgagctcctggattcaacgcgagacataggcttttggttccaacccgatactgttgaggcaaggatcagagtagaagttgcaacatcgaattga

SEQ ID NO: 177 - DNA, 1830 bp, Aspergillus fumigatus
atggacgtcggatgctttgtaaatcaacggcggtcctacaatggggatctatgcacagttcgttacataggtaaagttgagggcaccaccggcgagtggctcggagtggaatgggatgaccccacgcgggggaagcattctggagaacacaacggagtgagatattttacatgtagaaggaaacaccccacggctggttcgttcgtgcgcccttcgcgacggaccgacagacctcgaggcttccttgaggcagtgcgtcacaagtatgcttctgagttccaagaagaactcgcaagacagcagtcaggcgaagtctctgctgcgcgggaaatcatcaaatttagtagcaaagtagtggaagaggtcggcttcgacaagatccggaagaaacttgcagagctccaggaattgaaaatcgtgctcctggatcgcctatgcatcgcaggagttctccctcatagagcgagtctacatgagcttgcagaggcttgcaaggagatagaacagacatgtcctaagatcgttgacctcgatctgagttacaacttactggaaagctgggttgacattgcaaacatatgtcaacagctgaagcgcttgaagacattgaagctgatgttggtcattcagtacatctgtgagaagcatgctgacagttggcagcggaaatcgtctaggtcctcgacaggagggtctgatattcgacggtatcacaacactacacttggacgagactctactcgaatgggacgaggtatgctgcacaaagctactgcttggttaccaattacccagatcttgacacctatcacggataccatcacgaccttgacactggaaaacaatgacatctcttcgctatcctcattagcatgtctgacctctttgagcaagctcgagcacctctcgctgagagagaatcgtatcgggaaagtctatgcgtctggcatggaaggaaactctcttcagttttccgaaaatctcagatcggtggacctatccagaaacaatatcgattcttggctgtttgtgaatgaacttcaacgcgtatttcctgggctgcaatctttgcgcatatcaggaaatcccctgtacgacaagcctgttgccccctcgaacgtcacaaatttaccggagaagccaatgacggtggacgaggcctatatgctaacactctctcgactcgcttccatccaaacgctcaactacagcaagataacttcccaagaccgaagtaatggcgaactctattacctttccctcattggcaaggagttatccgcgtatccggaaagcgcagaacgcgagattcttgctacacatccccgctatcaggagctttgtgagaaatacggagcgcccacaatcaggagagccgagctggcaggcgctgccgtgaatccgcgctctgttgccgcccgagtagtgaagttggctttttgcttgcactcatcagttagttccggtgcaaaccaagaacaatttcgagttcagaagatcccgagatcctttaatacatatcaagtcaaggcaatcgcctcccgcctgttcaatttgccgccttaccagtgccgactagtctgggagaccaacgagttagaccctattcatcaggagaaaaaggacgatggagacgattgggatagtgatgaggatgaagccacagctattggattgggggagagtaacaagctcacaccggcgacggaggatggaaagttcatcagaagagaggttgagctcctggattcaacgcgagacataggcttttggttccaacccgatactgttgaggcaaggatcagagtagaagttgcaacatcgaattga

SEQ ID NO: 178 - protein, 609 aa, Aspergillus fumigatus
MDVGCFVNQRRSYNGDLCTVRYIGKVEGTTGEWLGVEWDDPTRGKHSGEHNGVRYFTCRRKHPTAGSFVRPSRRTDRPRGFLEAVRHKYASEFQEELARQQSGEVSAAREIIKFSSKVVEEVGFDKIRKKLAELQELKIVLLDRLCIAGVLPHRASLHELAEACKEIEQTCPKIVDLDLSYNLLESWVDIANICQQLKRLKTLKLMLVIQYICEKHADSWQRKSSRSSTGGSDIRRYHNTTLGRDSTRMGRGMLHKATAWLPITQILTPITDTITTLTLENNDISSLSSLACLTSLSKLEHLSLRENRIGKVYASGMEGNSLQFSENLRSVDLSRNNIDSWLFVNELQRVFPGLQSLRISGNPLYDKPVAPSNVTNLPEKPMTVDEAYMLTLSRLASIQTLNYSKITSQDRSNGELYYLSLIGKELSAYPESAEREILATHPRYQELCEKYGAPTIRRAELAGAAVNPRSVAARVVKLAFCLHSSVSSGANQEQFRVQKIPRSFNTYQVKAIASRLFNLPPYQCRLVWETNELDPIHQEKKDDGDDWDSDEDEATAIGLGESNKLTPATEDGKFIRREVELLDSTRDIGFWFQPDTVEARIRVEVATSN

SEQ ID NO: 179 - DNA, 1867 bp, Aspergillus fumigatus
caactatttcgacataggagttgtgaaggagttccttaacctacattgtcgcgggtggggtaatatatatagttgagcacgactcgtgcgttaggatacaacgaggtcaacgacagaaaagtagaacctcttacgcaagcgcctgtggcctctgttacccgaatggatttagaggccttcttggttattcttaaacaatatgatatgtgtagtgtatattcaagctggagggcaacactgactgctgttttaggactaggtcggttttgggtggcagattatcaccgcaactgttttgtgataacagtgatatcatttccctttcatataacaatttaatactcagacatcgtcatggcagaatactggaaatcagctgtaagtgcccttcattcagttccgcagacttcttcgtgataatctttacgtggggaagtccggcatcaactgacagcaatattctagccccggttctggtgcaaacaatgcaagatattcattcgggatacacccttcgagaaaacccagcatgaagcgagtgccaaacaccagggaaaccttaagcgtttcctacgagatatccaccgggaaaatgaacggaagcaaagagaaactcagaaggcgaaggatgaagtcgagcgattaaggcaaactgtcgcaggaaaaccaggtgcaaaagacagcggcgcaacagcttggaaacacgcctcggctgcccctccaccggcagaacgacctgtgtccctggaagagagaaagaagcagatagcgcagctggcagagatgggaattgctatcccggacgaataccgtggtgaactcgcgctcgctggcgaatggcagacggtatccgaacgagttattcgaccagatgacgatacagaggaaggaaagcctggtagctctatcggcgttcggaaacgcaagatggaaggcgatgaggaggagcaggaggcgcgacaggaggccgagagattcgtgagtcagggttggggctcgaggactcggcagtatcctggggagcagagcgatgcagacctggatgcacttctaaattctaccaaggatgtaaagaaggtcaagttgtcggcgccggatgaagggtcgaaagagaaggctagcaaagagggtgctacaccaagcaacgatacggaccaggctgcggctcaggagtcagaactaccatcagtcaagtctgagggtaaagaagcggcgcagcttgctacaacagataccccagcggtgaagcaggaagaggaggcggcacctacaggagttgtttttaagaagcgcaagccgaaggtcctgaggaaatagtcgaatttgcagctgctggatatctattatctaccatgcgcataaatgtacagatgatgcgttatggttgcgcacggtccaatatgcctcgcctgccggtgctcacatgaagcgatcatgggttcttgtctcactgcgctccagtgttcgaaaaccgggatgatgcctctggcctccagtcgtccgtcgccgaagcaacccagctcatagaagatctggattctcgattcgcttgcgcggaaagcctagtctggtcttgcgttagcctatatccagattgagtgtatcgattcgagcttatgcgggttgtctgataatattctgccttacattggcagagagacgttggagcacatagggtggatggagaatgatcagttcttacgtatgtaagcatgatgtctagctcagaaaagagtcccatataccatgcgactcgttggcagcatccacctcttcctttggagtgcaatctacaatagcatgcatacgaaacaaatttcgttgacaaggagacccagggcgagaagagtaatatagcaagccagct

SEQ ID NO: 180 - DNA, 963 bp, Aspergillus fumigatus
atggcagaatactggaaatcagctgtaagtgcccttcattcagttccgcagacttcttcgtgataatctttacgtggggaagtccggcatcaactgacagcaatattctagccccggttctggtgcaaacaatgcaagatattcattcgggatacacccttcgagaaaacccagcatgaagcgagtgccaaacaccagggaaaccttaagcgtttcctacgagatatccaccgggaaaatgaacggaagcaaagagaaactcagaaggcgaaggatgaagtcgagcgattaaggcaaactgtcgcaggaaaaccaggtgcaaaagacagcggcgcaacagcttggaaacacgcctcggctgcccctccaccggcagaacgacctgtgtccctggaagagagaaagaagcagatagcgcagctggcagagatgggaattgctatcccggacgaataccgtggtgaactcgcgctcgctggcgaatggcagacggtatccgaacgagttattcgaccagatgacgatacagaggaaggaaagcctggtagctctatcggcgttcggaaacgcaagatggaaggcgatgaggaggagcaggaggcgcgacaggaggccgagagattcgtgagtcagggttggggctcgaggactcggcagtatcctggggagcagagcgatgcagacctggatgcacttctaaattctaccaaggatgtaaagaaggtcaagttgtcggcgccggatgaagggtcgaaagagaaggctagcaaagagggtgctacaccaagcaacgatacggaccaggctgcggctcaggagtcagaactaccatcagtcaagtctgagggtaaagaagcggcgcagcttgctacaacagataccccagcggtgaagcaggaagaggaggcggcacctacaggagttgtttttaagaagcgcaagccgaaggtcctgaggaaatag

SEQ ID NO: 181 - DNA, 876 bp, Aspergillus fumigatus
atggcagaatactggaaatcagctccccggttctggtgcaaacaatgcaagatattcattcgggatacacccttcgagaaaacccagcatgaagcgagtgccaaacaccagggaaaccttaagcgtttcctacgagatatccaccgggaaaatgaacggaagcaaagagaaactcagaaggcgaaggatgaagtcgagcgattaaggcaaactgtcgcaggaaaaccaggtgcaaaagacagcggcgcaacagcttggaaacacgcctcggctgcccctccaccggcagaacgacctgtgtccctggaagagagaaagaagcagatagcgcagctggcagagatgggaattgctatcccggacgaataccgtggtgaactcgcgctcgctggcgaatggcagacggtatccgaacgagttattcgaccagatgacgatacagaggaaggaaagcctggtagctctatcggcgttcggaaacgcaagatggaaggcgatgaggaggagcaggaggcgcgacaggaggccgagagattcgtgagtcagggttggggctcgaggactcggcagtatcctggggagcagagcgatgcagacctggatgcacttctaaattctaccaaggatgtaaagaaggtcaagttgtcggcgccggatgaagggtcgaaagagaaggctagcaaagagggtgctacaccaagcaacgatacggaccaggctgcggctcaggagtcagaactaccatcagtcaagtctgagggtaaagaagcggcgcagcttgctacaacagataccccagcggtgaagcaggaagaggaggcggcacctacaggagttgtttttaagaagcgcaagccgaaggtcctgaggaaatag

SEQ ID NO: 182 - protein, 291 aa, Aspergillus fumigatus
MAEYWKSAPRFWCKQCKIFIRDTPFEKTQHEASAKHQGNLKRFLRDIHRENERKQRETQKAKDEVERLRQTVAGKPGAKDSGATAWKHASAAPPPAERPVSLEERKKQIAQLAEMGIAIPDEYRGELALAGEWQTVSERVIRPDDDTEEGKPGSSIGVRKRKMEGDEEEQEARQEAERFVSQGWGSRTRQYPGEQSDADLDALLNSTKDVKKVKLSAPDEGSKEKASKEGATPSNDTDQAAAQESELPSVKSEGKEAAQLATTDTPAVKQEEEAAPTGVVFKKRKPKVLRK

SEQ ID NO: 183 - DNA, 2193 bp, Aspergillus fumigatus
gagcaactgccagaaatccagacgtgcaactcctccgcaaaaaaaagcgttgtctcccaaaacggaggctgttaagttatcgccacggggaaatagcccaaaaggaactcgtcacagctggaatcaacaccaagtaccgaagaaacaagcgagcagcggctgtttggcttctgcagctgcacaaaaaatgggaacgaagtgaatgaggttagatagagatgaggatggatcaagaagcgccctccagatgtagcaatgaagagatgatgttgcaagaagaggtgaaacaagctggcggcacgggatcaggctaggctagatagggttagcaacgagggtgacatcacgtgagaacgggcatcgtgatatggatgacaattaacatcataaacactcttcgttcagttgctgtgactcctgacgcgtaaggggatctggggtgaagtcaagcaatagactctctgacagatttgactttagagaaagtaaataacaccactatggacatctcgcaagaaaccgttgataaaatacgacgtttcgcgcaaaagcgccaaaaagcggaggagttctacgaggaacactcggtaaatccagctaattttgacgcttacaatcgcaagttggatgagacgttggcagagctgcaggctcaagtcaaacgtcatgaggatgagctccgcaaggtacgtcaacaagttgcctagaatataagccgactgtcacaagagatttcatgcatgaattaggaatactgacaagaggaacagctacgcatgaccaccacgatcgagttcgctcaaattggggcagatccttgggcccgcatctcagaagtgcgcagagccaagaaagcgtatgattctcttctgcaatcggaaacgcgactgccgagtccaggctcgcccttgccttcattacttgcggttgacgaggcgtctcgtctcgtcaaggagagcaagacctcaatctcactgacggcggagaaactgtctgcggatcgtcagcgcttgaaagcggaagaagccaatttgcgcgatgcgcaactgatcaaagacgggttggagaaaaggattgagcggctgaacgcagaaaaatcgagtcaagtccagaaaactcctgcgcagcttgcgtatgatctcgtcaaggagcagcaggaaaagatcgagagacttgatactaccacagaagagctaaagtcctctctctataaatttgtcgaagacacacttgccccaatgcttgctgcagaaaatctgggcggtcccactgtcggagatgcgttggaaatttcggacactaccttaaaagcgggctacactagccatgggaagcctaagaaaccaaaaactccggccgtggggacttctgacagtggccaacagcggattgacgagcttgttcgtcgccaaactgcgcaggagggcaacgagcaggcaacccttttgaacaaaagagaggcggccgccgctgaaatgcgagctcttcttactgctctgttagatgcggattactcctatgtcgaccttccgcacgagtcagcggcctcgcgctttctagtaagagcgaaggtagctcaattccatccgcgcgatgccaggaagcttcggttaattgattttgggcgctcattagtcgattgaggtggctacatgtaccgtactacatctcccagcttacaaatggtatcacatttcaccaaacatctgggaaaagacaaacagacgccatccccacggatatatagcgactcaaccgaaagccagtaagatatctagagccggcgaaaaccacgtgtttcaacgaagaagcggccccgaaagccactggtagcataacgccttgagaatgcgagagatacatcaaaagcttatcagaaagttcaatgctcgaggtcaaaaatataccgttaatgccatacaagaaacatggaagaagaaagaccgtagccgggtatcagatcggcatcattccgatgctggtagaagtactcttggccgtattctttgctttggagccagttcgggaacccgccgagcgcttgttgacttgatcgctgggctcccttctaggtcgcggcgtttttatttttgaactcgaccctgtagcgttcttgcggtggaagcgcttcttggacgaagtctttcttttcttggaacggctagtctcggtgtcttcatactcttcggatga

SEQ ID NO: 184 - DNA, 1448 bp, Aspergillus fumigatus
atgtctgcttctccatccgcactgcaatcgaccaagcggcccttggaggacccttcttcgccgtccggaccaaatgatcagccagaagctaaacgtcctgccttggacaaagtagtaaagggaaacgagtcggagacctatacggatgccaaggctgagccttccgctgcgccaagtgctactgctgatggccagggcgacactgttgttcctgatgctccaaatggtaagggtgcatccacggagacgcagccaattcagtcgaccgcgtctcatggcgagcgcgctacttctcagcccgaacagcagcgcccacaagatgaatccagctggattcacattcgcgctgtaatttctagccaggaagctgccacagtcattggcaagggtggagaaaacgtatctcagattcgtcgtttgtctggagcaaagtgcactgtcagcgactactcccgtggtgcagtcgaacgtattttgaccgtgagcggcccacaggatgccgttgccaaggttggttttttgatctatccttcgctgtttgaaagattgctaattcagagtaggcgtttggtttgatcatccgtacattgaacaatgaacctcttgatgccccctctaccgcccaatccaagacataccctctgcgtttgctgatcccccatctccttattggctccatcattggcaaaggtggttcacgcattcgcgaaattcaggaagcttctggtgcccgactgaatgcatccgattcgtgccttcccttgtcctctgagcggtcacttgtaattctcggcgttgccgattctgtccacatcgctacctactacgtcgccgtaaccctcgttgagcagctcactgagcgctttggaggtcctgcagcctcagcttatgccactcgcagcggtggccctgctggagcagtgcctggcggtatgcaggttgtcccgtatgttccacagcccgctggtggtcaatatggccatccagaacatttcaagagacaccatcaccaccccaatcgcgctgctgcaggcgcctatggggtcccttaccttcacggtcagcctgctcccgcaccagtggcccagccggctttgcattatggagctgctccccatgccccttacgcaggagctggcccccatcagcctgctccatacggcgcaccgcagcccgctcaggcacgcggcgctcctacccctgccacacccgttggaggtgtcatgcctggtcagccattgactcagcagatctacatccccaacgacatggttggtgccatcatcggaaagggcggtgcgaagatcaatgagattcgacacctcagtggcagtgtgatcaagattaatgagcctcaagagaacagcaatgagcgtttggtgactattactggaacccaggaatgcaaccaaatggctctgtacatgctttactcgcgacttggttag

SEQ ID NO: 185 - DNA, 1395 bp, Aspergillus fumigatus
atgtctgcttctccatccgcactgcaatcgaccaagcggcccttggaggacccttcttcgccgtccggaccaaatgatcagccagaagctaaacgtcctgccttggacaaagtagtaaagggaaacgagtcggagacctatacggatgccaaggctgagccttccgctgcgccaagtgctactgctgatggccagggcgacactgttgttcctgatgctccaaatggtaagggtgcatccacggagacgcagccaattcagtcgaccgcgtctcatggcgagcgcgctacttctcagcccgaacagcagcgcccacaagatgaatccagctggattcacattcgcgctgtaatttctagccaggaagctgccacagtcattggcaagggtggagaaaacgtatctcagattcgtcgtttgtctggagcaaagtgcactgtcagcgactactcccgtggtgcagtcgaacgtattttgaccgtgagcggcccacaggatgccgttgccaaggcgtttggtttgatcatccgtacattgaacaatgaacctcttgatgccccctctaccgcccaatccaagacataccctctgcgtttgctgatcccccatctccttattggctccatcattggcaaaggtggttcacgcattcgcgaaattcaggaagcttctggtgcccgactgaatgcatccgattcgtgccttcccttgtcctctgagcggtcacttgtaattctcggcgttgccgattctgtccacatcgctacctactacgtcgccgtaaccctcgttgagcagctcactgagcgctttggaggtcctgcagcctcagcttatgccactcgcagcggtggccctgctggagcagtgcctggcggtatgcaggttgtcccgtatgttccacagcccgctggtggtcaatatggccatccagaacatttcaagagacaccatcaccaccccaatcgcgctgctgcaggcgcctatggggtcccttaccttcacggtcagcctgctcccgcaccagtggcccagccggctttgcattatggagctgctccccatgccccttacgcaggagctggcccccatcagcctgctccatacggcgcaccgcagcccgctcaggcacgcggcgctcctacccctgccacacccgttggaggtgtcatgcctggtcagccattgactcagcagatctacatccccaacgacatggttggtgccatcatcggaaagggcggtgcgaagatcaatgagattcgacacctcagtggcagtgtgatcaagattaatgagcctcaagagaacagcaatgagcgtttggtgactattactggaacccaggaatgcaaccaaatggctctgtacatgctttactcgcgacttggttag

SEQ ID NO: 186 - protein, 464 aa, Aspergillus fumigatus
MSASPSALQSTKRPLEDPSSPSGPNDQPEAKRPALDKVVKGNESETYTDAKAEPSAAPSATADGQGDTVVPDAPNGKGASTETQPIQSTASHGERATSQPEQQRPQDESSWIHIRAVISSQEAATVIGKGGENVSQIRRLSGAKCTVSDYSRGAVERILTVSGPQDAVAKAFGLIIRTLNNEPLDAPSTAQSKTYPLRLLIPHLLIGSIIGKGGSRIREIQEASGARLNASDSCLPLSSERSLVILGVADSVHIATYYVAVTLVEQLTERFGGPAASAYATRSGGPAGAVPGGMQVVPYVPQPAGGQYGHPEHFKRHHHHPNRAAAGAYGVPYLHGQPAPAPVAQPALHYGAAPHAPYAGAGPHQPAPYGAPQPAQARGAPTPATPVGGVMPGQPLTQQIYIPNDMVGAIIGKGGAKINEIRHLSGSVIKINEPQENSNERLVTITGTQECNQMALYMLYSRLG

SEQ ID NO: 187 - DNA, 2121 bp, Aspergillus fumigatus
taagttatcgccacggggaaatagcccaaaaggaactcgtcacagctggaatcaacaccaagtaccgaagaaacaagcgagcagcggctgtttggcttctgcagctgcacaaaaaatgggaacgaagtgaatgaggttagatagagatgaggatggatcaagaagcgccctccagatgtagcaatgaagagatgatgttgcaagaagaggtgaaacaagctggcggcacgggatcaggctaggctagatagggttagcaacgagggtgacatcacgtgagaacgggcatcgtgatatggatgacaattaacatcataaacactcttcgttcagttgctgtgactcctgacgcgtaaggggatctggggtgaagtcaagcaatagactctctgacagatttgactttagagaaagtaaataacaccactatggacatctcgcaagaaaccgttgataaaatacgacgtttcgcgcaaaagcgccaaaaagcggaggagttctacgaggaacactcggtaaatccagctaattttgacgcttacaatcgcaagttggatgagacgttggcagagctgcaggctcaagtcaaacgtcatgaggatgagctccgcaaggtacgtcaacaagttgcctagaatataagccgactgtcacaagagatttcatgcatgaattaggaatactgacaagaggaacagctacgcatgaccaccacgatcgagttcgctcaaattggggcagatccttgggcccgcatctcagaagtgcgcagagccaagaaagcgtatgattctcttctgcaatcggaaacgcgactgccgagtccaggctcgcccttgccttcattacttgcggttgacgaggcgtctcgtctcgtcaaggagagcaagacctcaatctcactgacggcggagaaactgtctgcggatcgtcagcgcttgaaagcggaagaagccaatttgcgcgatgcgcaactgatcaaagacgggttggagaaaaggattgagcggctgaacgcagaaaaatcgagtcaagtccagaaaactcctgcgcagcttgcgtatgatctcgtcaaggagcagcaggaaaagatcgagagacttgatactaccacagaagagctaaagtcctctctctataaatttgtcgaagacacacttgccccaatgcttgctgcagaaaatctgggcggtcccactgtcggagatgcgttggaaatttcggacactaccttaaaagcgggctacactagccatgggaagcctaagaaaccaaaaactccggccgtggggacttctgacagtggccaacagcggattgacgagcttgttcgtcgccaaactgcgcaggagggcaacgagcaggcaacccttttgaacaaaagagaggcggccgccgctgaaatgcgagctcttcttactgctctgttagatgcggattactcctatgtcgaccttccgcacgagtcagcggcctcgcgctttctagtaagagcgaaggtagctcaattccatccgcgcgatgccaggaagcttcggttaattgattttgggcgctcattagtcgattgaggtggctacatgtaccgtactacatctcccagcttacaaatggtatcacatttcaccaaacatctgggaaaagacaaacagacgccatccccacggatatatagcgactcaaccgaaagccagtaagatatctagagccggcgaaaaccacgtgtttcaacgaagaagcggccccgaaagccactggtagcataacgccttgagaatgcgagagatacatcaaaagcttatcagaaagttcaatgctcgaggtcaaaaatataccgttaatgccatacaagaaacatggaagaagaaagaccgtagccgggtatcagatcggcatcattccgatgctggtagaagtactcttggccgtattctttgctttggagccagttcgggaacccgccgagcgcttgttgacttgatcgctgggctcccttctaggtcgcggcgtttttatttttgaactcgaccctgtagcgttcttgcggtggaagcgcttcttggacgaagtctttcttttcttggaacggctagtctcggtgtcttcatactcttcggatga

SEQ ID NO: 188 - DNA, 1143 bp, Aspergillus fumigatus
atggacatctcgcaagaaaccgttgataaaatacgacgtttcgcgcaaaagcgccaaaaagcggaggagttctacgaggaacactcggtaaatccagctaattttgacgcttacaatcgcaagttggatgagacgttggcagagctgcaggctcaagtcaaacgtcatgaggatgagctccgcaaggtacgtcaacaagttgcctagaatataagccgactgtcacaagagatttcatgcatgaattaggaatactgacaagaggaacagctacgcatgaccaccacgatcgagttcgctcaaattggggcagatccttgggcccgcatctcagaagtgcgcagagccaagaaagcgtatgattctcttctgcaatcggaaacgcgactgccgagtccaggctcgcccttgccttcattacttgcggttgacgaggcgtctcgtctcgtcaaggagagcaagacctcaatctcactgacggcggagaaactgtctgcggatcgtcagcgcttgaaagcggaagaagccaatttgcgcgatgcgcaactgatcaaagacgggttggagaaaaggattgagcggctgaacgcagaaaaatcgagtcaagtccagaaaactcctgcgcagcttgcgtatgatctcgtcaaggagcagcaggaaaagatcgagagacttgatactaccacagaagagctaaagtcctctctctataaatttgtcgaagacacacttgccccaatgcttgctgcagaaaatctgggcggtcccactgtcggagatgcgttggaaatttcggacactaccttaaaagcgggctacactagccatgggaagcctaagaaaccaaaaactccggccgtggggacttctgacagtggccaacagcggattgacgagcttgttcgtcgccaaactgcgcaggagggcaacgagcaggcaacccttttgaacaaaagagaggcggccgccgctgaaatgcgagctcttcttactgctctgttagatgcggattactcctatgtcgaccttccgcacgagtcagcggcctcgcgctttctagtaagagcgaaggtagctcaattccatccgcgcgatgccaggaagcttcggttaattgattttgggcgctcattagtcgattga

SEQ ID NO: 189 - DNA, 1035 bp, Aspergillus fumigatus
atggacatctcgcaagaaaccgttgataaaatacgacgtttcgcgcaaaagcgccaaaaagcggaggagttctacgaggaacactcggtaaatccagctaattttgacgcttacaatcgcaagttggatgagacgttggcagagctgcaggctcaagtcaaacgtcatgaggatgagctccgcaagttcgctcaaattggggcagatccttgggcccgcatctcagaagtgcgcagagccaagaaagcgtatgattctcttctgcaatcggaaacgcgactgccgagtccaggctcgcccttgccttcattacttgcggttgacgaggcgtctcgtctcgtcaaggagagcaagacctcaatctcactgacggcggagaaactgtctgcggatcgtcagcgcttgaaagcggaagaagccaatttgcgcgatgcgcaactgatcaaagacgggttggagaaaaggattgagcggctgaacgcagaaaaatcgagtcaagtccagaaaactcctgcgcagcttgcgtatgatctcgtcaaggagcagcaggaaaagatcgagagacttgatactaccacagaagagctaaagtcctctctctataaatttgtcgaagacacacttgccccaatgcttgctgcagaaaatctgggcggtcccactgtcggagatgcgttggaaatttcggacactaccttaaaagcgggctacactagccatgggaagcctaagaaaccaaaaactccggccgtggggacttctgacagtggccaacagcggattgacgagcttgttcgtcgccaaactgcgcaggagggcaacgagcaggcaacccttttgaacaaaagagaggcggccgccgctgaaatgcgagctcttcttactgctctgttagatgcggattactcctatgtcgaccttccgcacgagtcagcggcctcgcgctttctagtaagagcgaaggtagctcaattccatccgcgcgatgccaggaagcttcggttaattgattttgggcgctcattagtcgattga

SEQ ID NO: 190 - protein, 344 aa, Aspergillus fumigatus
MDISQETVDKIRRFAQKRQKAEEFYEEHSVNPANFDAYNRKLDETLAELQAQVKRHEDELRKFAQIGADPWARISEVRRAKKAYDSLLQSETRLPSPGSPLPSLLAVDEASRLVKESKTSISLTAEKLSADRQRLKAEEANLRDAQLIKDGLEKRIERLNAEKSSQVQKTPAQLAYDLVKEQQEKIERLDTTTEELKSSLYKFVEDTLAPMLAAENLGGPTVGDALEISDTTLKAGYTSHGKPKKPKTPAVGTSDSGQQRIDELVRRQTAQEGNEQATLLNKREAAAAEMRALLTALLDADYSYVDLPHESAASRFLVRAKVAQFHPRDARKLRLIDFGRSLVD

SEQ ID NO: 191 - DNA, 2000 bp, Aspergillus fumigatus
tgaacctatgtaccatccactcctgagtcctgctcaggggaaatgcatgtgagcaggtgccttctcacctctgctcctttaatacttcattgcggggatattattccgtgctggacagcttctgcaagatcagtaattatgtaggaacacgaaaacaatgttctacacattagttttctcctgatgaccatcttctctttgatcaatgcagcagacaacaaatcctgtccaatcatcccaacatactgtttcctaccttgattggcgcaaatccggcgacaataatcaagggtagagtgatggactaggctcaggcggtttgactttgcaaatccaattgaattctgatcagtgctaagccgatgatggcgttgagcgaccaaggacaatttccgactgagctcagcatttccctcgctcgcgactcgcgaggcgcggtccaggacgggaggtcttggaaaggctgaacggtcaatcaatcatcaaatgggtaggatggtgcgtaaatagcccacatgcgcataaaagtggatgagtgccgcatcggtatcttcgttcttgtttccttttccgtctcaccccaagatcatgggtcatgaattgataatagtatcgtcaactggcttagcgtgatgactacgtatctttttgagcgtcaagttcggtcgtttcaatcggagtggttttctccgccaacgcctagtccattgctcccaagagaggggctgataaatgacggcgctcaggggcccaggggcttcatgtaggcacaatggaagtaaaacgtatgtacgaagaatggatcatctctcaaagtactatactccgtactcttgcttgtattctgatatttcagttcaataatcgaaaaatagtgacgcgcggtatggtgcagatcttatgcagataggacagtcagcccgccagtattacggagaacctaaggtactcatagtaaagtatgatgatacggccgcatacgacctctaattcatcgaatactccagaagcggctagccactttcctgaaatcgaggtctatccatggactcagcatcttgtacggagtacaaactcctgccagcttattccaactgatgaccctggcccttatatgagcaagcaggatatgattgggagacttgagttgtgtttggaaccagctggtcagccacggaatgatattaggcttactaaggtagctattgtctcacacaggtatgcactgcggaacatggattaattgatacttatgaaccctaactgctttggaggggaggtgatactctacctgagggaaaggcgaacaattacagagtaatgataatgcataaataatatctcatgatgttaccctgcctcgtcaaacatggagtctcgctttcgattgtgaatccacttctccatttttacgtcagactaattccatttaatcaatacctctcgtatggggtaaatcaatgcaccggaaatttacatatgatatcaaaagtagtagttctccgactagggaggatggagcaaggtaatctatatttattggctgctcgtttgcaggccaaaccaaaaccaacatgctctgtatcttagcctccgtatctgatctcatatctttgaggacattgattggattgtcgatcccaatgacggcgtgctgagactgttctaaagagagctcaaaagaaatagagcaaaagtaagatcatctacggactatctacgtcctataggagtttcactctgtgataatggctcacgcccacataaactgtccatgacgaaatcgatacagcctcaagaaagccaacaattttagtttggaagtcgaatcacaccacataggttaaccagcaaaagcgcagacctccaataaaggttcgctcatcgtccccttctgcgtctactttggagctaaagctgcgcgtctgtacatcttgacctccttcggcctctcctttgcgctgtgctcagttttcccttcttttgtctcttggactgga

SEQ ID NO: 192 - DNA, 4336 bp, Aspergillus fumigatus
acgattggccaatcgtttgctgtaatgcttggcggcttcttggcattgcccagcccaaggtgctcacgtgggtgggatgatgggcatgtcgactgtcgtgtgcgtcatcgggctgggaggcggcaaatgctcgcgcaccgacccacggaattcggccaagattctggatcgattgccccttatgtatgcgaaggtttggggatgtgattggtgtgcagcgtaaacgagtacggattgataagagtgccaagattggaaccactgttcacgtcatggaggggggtttccggcacgccggttaggagtgagacagagtgacttaaatatgtcgcgatacccccgcaacgacgggagaagaaaatctggttctacaacctaggctttaatttccaagttgatttcaagtcgttgtgtatggactctcactcgcttgaagatacgatatacatgaagcctccttaggataactgcactggaatagaacattttcgttatataacatgggtgcgcagaagaagcaatcctctgataactctcaggctttgtctcagcctgctcatgcgcttcgctatgaggatgtccttcgtgagctggccgtcgatccagaccagggtctgaccgtgggggaggcgaagcgaagactccaacaatatggtccgaatgaactggaagggggagagggtgtttccattgtcaagattgtcattaggcagatcgcaaatgccatgatgctggttagtctctcccttttgttagacagacaacgacagacaactgacgacagcaggtcctgatcatcgccatggcggtcagtttcggcattcaatcctggattgagggtggtgtcattggggctgtcattggtctaaacattgtggtaggtgtctatcaagactatgccgcagagaaaacgatggattcccttcgatcgttgagttctcccaccggaactgtcacgcgtgatggcaaaaccagtaccattcctgccaacgagattgtccccggggacatgattgaactcaaagttggagacacggttcctgcggatcttcggtgcgtaaagagttaccagaaaatatcgttcccgttcactaaccgccgataggctcgttgatgcaatgaatttcgaaaccgacgaggccctcctgactggcgagtcgctgccggtgcagaaagaggtcgataccacttttgacccagacacgggacctggtgatcggttgaatattgcgtatagttcctccacggtgacgagaggccgcgctcggggcgttgttatcagcacggggatgcaaaccgagattggagccatcgccgctgctctccgcgccagcgactccaagaggcggcccgtcaagcgcggtcccgagggagaaaccaagaagcgatggtacgtgcaggcgtggaccctgacttgcacggatgccgtgggacggttcctgggaatcaacgttggcacaccgctgcagcgcaaactctcgaaactggctttggcccttttcgccattgccatcatcttcgccatcattgtcatgggcgtcaatgggttccgcaatgacaaggaggtcatcatctacgcagtcgcaacaggtcttgcaatgatcccggcctgcctggtggtggtcctgaccattaccatggcggttggaaccaagcaaatggttgaaagacatgtcattgtccggaagcttgattcccttgaagccctgggcgctgtgactaacatttgctcagacaaaacgggaaccctcacccaggggcgcatggtcgccaaacgggcgtggattccctctgttggcaccttctcggtggggtcttctaataaccccttgaacccggaagagggcgaccttagcctgctgcctgacccgccggtcaaagttggccctgacgctcatggagagccttcccggccggaggatctgctcaaggacaatcctcttttggagcaatatctgaacgtggccgccatggcaaaccttgcccatgtgcacaggtccgagcacaatgaatggcaggctcgtggtgaaccgaccgacattgcgattcaggtgtttgcctcgcgctttaactggggacgcgatcgctggaccaaaggcgagaagcctgtttggcgccaaaaagctgaatatccatttgattctactgtgaaaaagatgtctgtcatctttaagaacaccaatgatgatcgggagatgatcttcaccaagggcgcggtggaacgtgtcatcgaggcttgcaccaccgtcacctggacagctggttccgacccgatcgccttggacgagaacataaaggaggaaatcttgcagaacatggaagctctcgcaaaggagggcctgcgagttctctgcctcgcctgtcgggagaatcacaaccccgtcaaaggggaagttgtccctgcgcgtgaggaagtggagaaagatctcactttctgtggactcatcggtctgtatgaccctccccgacctgagacggccggcgctattgacgagtgctaccgcgctgggatttcggttcacatggtcacgggcgaccacccgggtactgcgcgagcgattgcagcgcaggttggcatcattccagccaacatggacagtcttgccaaagatgttgcggacgccatggtgatgactgccagtcagtttgataagttgacagacgaggaaatcgatgcgttgcctactctgcctgctgtgattgctcggtgcgctcccaacacaaaggttcggatgattgatgcccttcatcgtcggggtcggtttgcggcaatgactggtgatggagtcaatgactcgccatcgttgaagcgggcagatgttggaattgctatgggacaatcggggtccgatgtggccaaagacgcttcggaactggtgttgactgatgacaactttgcgtcgattatcaacggaattgaggagggtcgtcgtatttttgataatattcagaaattcgtcctgcatcttctggcggaaaacgtcggtctcgccctcacgcttctgataggtctgtgtttcaaagatgacaacgggcaatctgtgtttcctattgctcctgtcgaaattctatggatcatcatgatcacctccgggctcccggacatgggactgggtatggagatcgcggctccggatatcatggaccgtcctcctcaaagcgtaagtacatcagtcactctttctgggatcatatactaacgacaacagaaacaaggtattttcacctgggaagtcatagtcgacaccatggtctacggcgtctggatggcggctctctgtctcgcctctttctcgctcgtcctctttggttggggagatggcaacctggcctcgggctgcaacagcgactacagccccgaatgcgacggtgtgttccgcgcccgagctacgaccttcgtgtgcatgacatggttcgctctcttcctcgcctgggagatgattgatatgcgccgcagcttcttccgcatgcagcccaattccaagcgctatttcacccagtggatgtttgacgtctggcgcaacaagtttctgttcagcggcatcatgattggattcgtcacgaccttccccatcctgtacatccctgtgatcaatgacgttgtcttcaagcatgtcggtatctcctgggagtggggtgtggtgtttgtcgaagcgattcttttcttcgctggctgcgaggcctggaagtggtgcaagcgcatttatttcaggcacaccagccaaaaggaaactggcagagaaagggtacttcgggactttagccgttataccaccatgagcaggtccgagacccaagcaacgggtgacctcaacgtggaaaagtcgatggtttagatctggcaataggccaggatagcagcagatggagggcagggaagcgactgcgtgtatttgcatgcatctaattggtggccactggttttcattaagcagaatgtacatagatatgaatagaagtagcgatttatcttttttttccgcctttcggtttcccccccttctgcaggttcacgagtaattgactttatcctcactctagagtctagttgatatggtatgtctttgtaaatatgcaatcagtctcagtttatcatattcattctgattcactataactggattatataattctgtatgcatacttatcgccttatctaaaaaggcagtaatttggcacatccacacggttccttgatatatactctcgtccatgggtacatcataggcttcatacccttgatatatatcgggaacccttcagcacaacagacaagtcaacaacaaactgatgaaaatagattagatagtattacagtactacctatgtagttcta

SEQ ID NO: 193 - DNA, 3336 bp, Aspergillus fumigatus
atgggtgcgcagaagaagcaatcctctgataactctcaggctttgtctcagcctgctcatgcgcttcgctatgaggatgtccttcgtgagctggccgtcgatccagaccagggtctgaccgtgggggaggcgaagcgaagactccaacaatatggtccgaatgaactggaagggggagagggtgtttccattgtcaagattgtcattaggcagatcgcaaatgccatgatgctggttagtctctcccttttgttagacagacaacgacagacaactgacgacagcaggtcctgatcatcgccatggcggtcagtttcggcattcaatcctggattgagggtggtgtcattggggctgtcattggtctaaacattgtggtaggtgtctatcaagactatgccgcagagaaaacgatggattcccttcgatcgttgagttctcccaccggaactgtcacgcgtgatggcaaaaccagtaccattcctgccaacgagattgtccccggggacatgattgaactcaaagttggagacacggttcctgcggatcttcggtgcgtaaagagttaccagaaaatatcgttcccgttcactaaccgccgataggctcgttgatgcaatgaatttcgaaaccgacgaggccctcctgactggcgagtcgctgccggtgcagaaagaggtcgataccacttttgacccagacacgggacctggtgatcggttgaatattgcgtatagttcctccacggtgacgagaggccgcgctcggggcgttgttatcagcacggggatgcaaaccgagattggagccatcgccgctgctctccgcgccagcgactccaagaggcggcccgtcaagcgcggtcccgagggagaaaccaagaagcgatggtacgtgcaggcgtggaccctgacttgcacggatgccgtgggacggttcctgggaatcaacgttggcacaccgctgcagcgcaaactctcgaaactggctttggcccttttcgccattgccatcatcttcgccatcattgtcatgggcgtcaatgggttccgcaatgacaaggaggtcatcatctacgcagtcgcaacaggtcttgcaatgatcccggcctgcctggtggtggtcctgaccattaccatggcggttggaaccaagcaaatggttgaaagacatgtcattgtccggaagcttgattcccttgaagccctgggcgctgtgactaacatttgctcagacaaaacgggaaccctcacccaggggcgcatggtcgccaaacgggcgtggattccctctgttggcaccttctcggtggggtcttctaataaccccttgaacccggaagagggcgaccttagcctgctgcctgacccgccggtcaaagttggccctgacgctcatggagagccttcccggccggaggatctgctcaaggacaatcctcttttggagcaatatctgaacgtggccgccatggcaaaccttgcccatgtgcacaggtccgagcacaatgaatggcaggctcgtggtgaaccgaccgacattgcgattcaggtgtttgcctcgcgctttaactggggacgcgatcgctggaccaaaggcgagaagcctgtttggcgccaaaaagctgaatatccatttgattctactgtgaaaaagatgtctgtcatctttaagaacaccaatgatgatcgggagatgatcttcaccaagggcgcggtggaacgtgtcatcgaggcttgcaccaccgtcacctggacagctggttccgacccgatcgccttggacgagaacataaaggaggaaatcttgcagaacatggaagctctcgcaaaggagggcctgcgagttctctgcctcgcctgtcgggagaatcacaaccccgtcaaaggggaagttgtccctgcgcgtgaggaagtggagaaagatctcactttctgtggactcatcggtctgtatgaccctccccgacctgagacggccggcgctattgacgagtgctaccgcgctgggatttcggttcacatggtcacgggcgaccacccgggtactgcgcgagcgattgcagcgcaggttggcatcattccagccaacatggacagtcttgccaaagatgttgcggacgccatggtgatgactgccagtcagtttgataagttgacagacgaggaaatcgatgcgttgcctactctgcctgctgtgattgctcggtgcgctcccaacacaaaggttcggatgattgatgcccttcatcgtcggggtcggtttgcggcaatgactggtgatggagtcaatgactcgccatcgttgaagcgggcagatgttggaattgctatgggacaatcggggtccgatgtggccaaagacgcttcggaactggtgttgactgatgacaactttgcgtcgattatcaacggaattgaggagggtcgtcgtatttttgataatattcagaaattcgtcctgcatcttctggcggaaaacgtcggtctcgccctcacgcttctgataggtctgtgtttcaaagatgacaacgggcaatctgtgtttcctattgctcctgtcgaaattctatggatcatcatgatcacctccgggctcccggacatgggactgggtatggagatcgcggctccggatatcatggaccgtcctcctcaaagcgtaagtacatcagtcactctttctgggatcatatactaacgacaacagaaacaaggtattttcacctgggaagtcatagtcgacaccatggtctacggcgtctggatggcggctctctgtctcgcctctttctcgctcgtcctctttggttggggagatggcaacctggcctcgggctgcaacagcgactacagccccgaatgcgacggtgtgttccgcgcccgagctacgaccttcgtgtgcatgacatggttcgctctcttcctcgcctgggagatgattgatatgcgccgcagcttcttccgcatgcagcccaattccaagcgctatttcacccagtggatgtttgacgtctggcgcaacaagtttctgttcagcggcatcatgattggattcgtcacgaccttccccatcctgtacatccctgtgatcaatgacgttgtcttcaagcatgtcggtatctcctgggagtggggtgtggtgtttgtcgaagcgattcttttcttcgctggctgcgaggcctggaagtggtgcaagcgcatttatttcaggcacaccagccaaaaggaaactggcagagaaagggtacttcgggactttagccgttataccaccatgagcaggtccgagacccaagcaacgggtgacctcaacgtggaaaagtcgatggtttag

SEQ ID NO: 194 - DNA, 3180 bp, Aspergillus fumigatus
atgggtgcgcagaagaagcaatcctctgataactctcaggctttgtctcagcctgctcatgcgcttcgctatgaggatgtccttcgtgagctggccgtcgatccagaccagggtctgaccgtgggggaggcgaagcgaagactccaacaatatggtccgaatgaactggaagggggagagggtgtttccattgtcaagattgtcattaggcagatcgcaaatgccatgatgctggtcctgatcatcgccatggcggtcagtttcggcattcaatcctggattgagggtggtgtcattggggctgtcattggtctaaacattgtggtaggtgtctatcaagactatgccgcagagaaaacgatggattcccttcgatcgttgagttctcccaccggaactgtcacgcgtgatggcaaaaccagtaccattcctgccaacgagattgtccccggggacatgattgaactcaaagttggagacacggttcctgcggatcttcggctcgttgatgcaatgaatttcgaaaccgacgaggccctcctgactggcgagtcgctgccggtgcagaaagaggtcgataccacttttgacccagacacgggacctggtgatcggttgaatattgcgtatagttcctccacggtgacgagaggccgcgctcggggcgttgttatcagcacggggatgcaaaccgagattggagccatcgccgctgctctccgcgccagcgactccaagaggcggcccgtcaagcgcggtcccgagggagaaaccaagaagcgatggtacgtgcaggcgtggaccctgacttgcacggatgccgtgggacggttcctgggaatcaacgttggcacaccgctgcagcgcaaactctcgaaactggctttggcccttttcgccattgccatcatcttcgccatcattgtcatgggcgtcaatgggttccgcaatgacaaggaggtcatcatctacgcagtcgcaacaggtcttgcaatgatcccggcctgcctggtggtggtcctgaccattaccatggcggttggaaccaagcaaatggttgaaagacatgtcattgtccggaagcttgattcccttgaagccctgggcgctgtgactaacatttgctcagacaaaacgggaaccctcacccaggggcgcatggtcgccaaacgggcgtggattccctctgttggcaccttctcggtggggtcttctaataaccccttgaacccggaagagggcgaccttagcctgctgcctgacccgccggtcaaagttggccctgacgctcatggagagccttcccggccggaggatctgctcaaggacaatcctcttttggagcaatatctgaacgtggccgccatggcaaaccttgcccatgtgcacaggtccgagcacaatgaatggcaggctcgtggtgaaccgaccgacattgcgattcaggtgtttgcctcgcgctttaactggggacgcgatcgctggaccaaaggcgagaagcctgtttggcgccaaaaagctgaatatccatttgattctactgtgaaaaagatgtctgtcatctttaagaacaccaatgatgatcgggagatgatcttcaccaagggcgcggtggaacgtgtcatcgaggcttgcaccaccgtcacctggacagctggttccgacccgatcgccttggacgagaacataaaggaggaaatcttgcagaacatggaagctctcgcaaaggagggcctgcgagttctctgcctcgcctgtcgggagaatcacaaccccgtcaaaggggaagttgtccctgcgcgtgaggaagtggagaaagatctcactttctgtggactcatcggtctgtatgaccctccccgacctgagacggccggcgctattgacgagtgctaccgcgctgggatttcggttcacatggtcacgggcgaccacccgggtactgcgcgagcgattgcagcgcaggttggcatcattccagccaacatggacagtcttgccaaagatgttgcggacgccatggtgatgactgccagtcagtttgataagttgacagacgaggaaatcgatgcgttgcctactctgcctgctgtgattgctcggtgcgctcccaacacaaaggttcggatgattgatgcccttcatcgtcggggtcggtttgcggcaatgactggtgatggagtcaatgactcgccatcgttgaagcgggcagatgttggaattgctatgggacaatcggggtccgatgtggccaaagacgcttcggaactggtgttgactgatgacaactttgcgtcgattatcaacggaattgaggagggtcgtcgtatttttgataatattcagaaattcgtcctgcatcttctggcggaaaacgtcggtctcgccctcacgcttctgataggtctgtgtttcaaagatgacaacgggcaatctgtgtttcctattgctcctgtcgaaattctatggatcatcatgatcacctccgggctcccggacatgggactgggtatggagatcgcggctccggatatcatggaccgtcctcctcaaagcgtaagtattttcacctgggaagtcatagtcgacaccatggtctacggcgtctggatggcggctctctgtctcgcctctttctcgctcgtcctctttggttggggagatggcaacctggcctcgggctgcaacagcgactacagccccgaatgcgacggtgtgttccgcgcccgagctacgaccttcgtgtgcatgacatggttcgctctcttcctcgcctgggagatgattgatatgcgccgcagcttcttccgcatgcagcccaattccaagcgctatttcacccagtggatgtttgacgtctggcgcaacaagtttctgttcagcggcatcatgattggattcgtcacgaccttccccatcctgtacatccctgtgatcaatgacgttgtcttcaagcatgtcggtatctcctgggagtggggtgtggtgtttgtcgaagcgattcttttcttcgctggctgcgaggcctggaagtggtgcaagcgcatttatttcaggcacaccagccaaaaggaaactggcagagaaagggtacttcgggactttagccgttataccaccatgagcaggtccgagacccaagcaacgggtgacctcaacgtggaaaagtcgatggtttag

SEQ ID NO: 195 - protein, 1059 aa, Aspergillus fumigatus
MGAQKKQSSDNSQALSQPAHALRYEDVLRELAVDPDQGLTVGEAKRRLQQYGPNELEGGEGVSIVKIVIRQIANAMMLVLIIAMAVSFGIQSWIEGGVIGAVIGLNIVVGVYQDYAAEKTMDSLRSLSSPTGTVTRDGKTSTIPANEIVPGDMIELKVGDTVPADLRLVDAMNFETDEALLTGESLPVQKEVDTTFDPDTGPGDRLNIAYSSSTVTRGRARGVVISTGMQTEIGAIAAALRASDSKRRPVKRGPEGETKKRWYVQAWTLTCTDAVGRFLGINVGTPLQRKLSKLALALFAIAIIFAIIVMGVNGFRNDKEVIIYAVATGLAMIPACLVVVLTITMAVGTKQMVERHVIVRKLDSLEALGAVTNICSDKTGTLTQGRMVAKRAWIPSVGTFSVGSSNNPLNPEEGDLSLLPDPPVKVGPDAHGEPSRPEDLLKDNPLLEQYLNVAAMANLAHVHRSEHNEWQARGEPTDIAIQVFASRFNWGRDRWTKGEKPVWRQKAEYPFDSTVKKMSVIFKNTNDDREMIFTKGAVERVIEACTTVTWTAGSDPIALDENIKEEILQNMEALAKEGLRVLCLACRENHNPVKGEVVPAREEVEKDLTFCGLIGLYDPPRPETAGAIDECYRAGISVHMVTGDHPGTARAIAAQVGIIPANMDSLAKDVADAMVMTASQFDKLTDEEIDALPTLPAVIARCAPNTKVRMIDALHRRGRFAAMTGDGVNDSPSLKRADVGIAMGQSGSDVAKDASELVLTDDNFASIINGIEEGRRIFDNIQKFVLHLLAENVGLALTLLIGLCFKDDNGQSVFPIAPVEILWIIMITSGLPDMGLGMEIAAPDIMDRPPQSVSIFTWEVIVDTMVYGVWMAALCLASFSLVLFGWGDGNLASGCNSDYSPECDGVFRARATTFVCMTWFALFLAWEMIDMRRSFFRMQPNSKRYFTQWMFDVWRNKFLFSGIMIGFVTTFPILYIPVINDVVFKHVGISWEWGVVFVEAILFFAGCEAWKWCKRIYFRHTSQKETGRERVLRDFSRYTTMSRSETQATGDLNVEKSMV

SEQ ID NO: 196 - DNA, 2059 bp, Aspergillus fumigatus
cgccgaactatggggcatatcggtagccaacaccactttactatactgtcacgcgagtcataaatcgcagatggcccagttccagccctgacaagagtgtcacacttgttctattacggaatgggccgcttcccgatgtaaccgcacaagccgctatcttccctggaatgttggcacaaggctacctattcaggcgctcaagaatgccgatgcctgaattcgcagttgactggcaagtttcatgagcgatgtggcttcacttttggccagaaacattggctgttccatctcagcgagcagggtttcaaacttggatgccttggtgcctttccacatgaggttaagcaccattggctttggatattttgagacgcatactgtgctgtggcacagtcagtgcatttgtagccagtggctgtgttgtcatggtcgtgaaggtggattagagtctgttggttgctgaagatgtataggagaaaatagcatataaggggactgagatgtctgtggttgttcttgtttccaagccaacagcacatttctcagaattggcatctcatcacagagtccgtgtaattatcacattagcaaaagccacaatgtcccaccccgatctatcgaccattctcgaggtctaccccgaatgcgaagtcacctgctacgggtacgctcccagccaacggcgccgctgcagaatgcgaaccaggaaagacaaccgagacagggcttcgtaccttctcgaagagggcaccagatatcttcagcgcggcctccccgtcgacggtctgcttattgagctagccccgctagttctctgcacacgcttccaccaataccaggcagacgacttggtccgggactggcgggccaagctgcgggagttccagcagcagactcttctgaatgctatgctgaaatcgctccaagagcttgtggacagtcgggcgcgttcgcgtgctgctaggtcggcgggtcggcgtctcccagaaagagtttcgagtccgacacggctggagaggtcggctgctattgtaacagaggaagaaccggctgctcctgaaagagaggaagaggaggaagaaagaggagatagggaggacgagcctgaaccagaacccgaacctgaacctgaacttacccccagccggtcatctacggagacttcctcgccagccgttgaggctcatgtcgcggagccaaccgttcctcagactgagtccagaagagtcactcgcaaaccaatcgaaggagactgtactatctgcctgtgtcctctacgagaacaagacagtgatgaaaacggcgagggatcagaagatcgcgatgatgagaatgaggatgatgcggcgggcacagggtccggcacagcgtccgacgagcacgacgcccccgaagaacatgacgatgacgaccttgtatattgcaaaaaccagtgcggtacgaactatcacaaagcctgtattgacgtgtggcatgctactcagcgtacatttgaaactccacgtggggatcctatcggcctgtcctgcccgtactgtcgagcagcatggtcctcctgatttcttcattctgcggggagtctcggcgtttgttacttattttgtgatcccaattccttctcacgttcttgttgcaatacgtcagatatttccttgtacagttatgcctgggggtgattatgagtacataacggccttgttcagaattatattaatagcagtttcctggttctgcaaatatagtcgtgttggccttcaagcttgtagctgagataaatggatacatagatggctctcttctatcgtgatgatcattcccacacgttgttcctctcttcgcctgtgtctcgatctcttgaatctcgcctcgacctcggctccagctagaagcatcgaatacaatagctcctgacaatggactgttctctgaaggatgagtggtctcaatgaatgtgaccgcttgccgcgattggcctcgaagacttaaaggagcaggcttcttttctgggcagagcggagtgaagcttctgaaccgcctcaggcaccgcgaacctggagct

SEQ ID NO: 197 - DNA, 1059 bp, Aspergillus fumigatus
atgtctgtggttgttcttgtttccaagccaacagcacatttctcagaattggcatctcatcacagagtccgtgtaattatcacattagcaaaagccacaatgtcccaccccgatctatcgaccattctcgaggtctaccccgaatgcgaagtcacctgctacgggtacgctcccagccaacggcgccgctgcagaatgcgaaccaggaaagacaaccgagacagggcttcgtaccttctcgaagagggcaccagatatcttcagcgcggcctccccgtcgacggtctgcttattgagctagccccgctagttctctgcacacgcttccaccaataccaggcagacgacttggtccgggactggcgggccaagctgcgggagttccagcagcagactcttctgaatgctatgctgaaatcgctccaagagcttgtggacagtcgggcgcgttcgcgtgctgctaggtcggcgggtcggcgtctcccagaaagagtttcgagtccgacacggctggagaggtcggctgctattgtaacagaggaagaaccggctgctcctgaaagagaggaagaggaggaagaaagaggagatagggaggacgagcctgaaccagaacccgaacctgaacctgaacttacccccagccggtcatctacggagacttcctcgccagccgttgaggctcatgtcgcggagccaaccgttcctcagactgagtccagaagagtcactcgcaaaccaatcgaaggagactgtactatctgcctgtgtcctctacgagaacaagacagtgatgaaaacggcgagggatcagaagatcgcgatgatgagaatgaggatgatgcggcgggcacagggtccggcacagcgtccgacgagcacgacgcccccgaagaacatgacgatgacgaccttgtatattgcaaaaaccagtgcggtacgaactatcacaaagcctgtattgacgtgtggcatgctactcagcgtacatttgaaactccacgtggggatcctatcggcctgtcctgcccgtactgtcgagcagcatggtcctcctga

SEQ ID NO: 198 - DNA, 1059 bp, Aspergillus fumigatus; identical to SEQ ID NO: 197

SEQ ID NO: 199 - protein, 352 aa, Aspergillus fumigatus
MSVVVLVSKPTAHFSELASHHRVRVIITLAKATMSHPDLSTILEVYPECEVTCYGYAPSQRRRCRMRTRKDNRDRASYLLEEGTRYLQRGLPVDGLLIELAPLVLCTRFHQYQADDLVRDWRAKLREFQQQTLLNAMLKSLQELVDSRARSRAARSAGRRLPERVSSPTRLERSAAIVTEEEPAAPEREEEEEERGDREDEPEPEPEPEPELTPSRSSTETSSPAVEAHVAEPTVPQTESRRVTRKPIEGDCTICLCPLREQDSDENGEGSEDRDDENEDDAAGTGSGTASDEHDAPEEHDDDDLVYCKNQCGTNYHKACIDVWHATQRTFETPRGDPIGLSCPYCRAAWSS

(19) United States
(12) Patent Application Publication
(10) Pub. No.: US 2010/0120029 A1
(43) Pub. Date: May 13, 2010

(54) Mutagenesis of aspergillus fungi and genes essential for growth

(75) Inventors: Marie-Claire Grosjean-Cournoyer, Curis Au Mont D'Or (FR); Christophe Didier D'Enfert, Paris (FR); Arnaud Firon, Orly (FR); Francois Villalba, Ecully (FR); Marc-Henri Lebrun, Lyon (FR); Roland Beffa, Saint Genis Laval (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Bayer Cropscience SA and Institut Pasteur

(21) Appl. No.: 12/379,877

(22) Filed: Mar. 3, 2009

Related U.S. Application Data
(62) Division of application No. 10/507,416, filed on May 27, 2005, now abandoned.

(30) Foreign Application Priority Data
Mar. 13, 2003 (IB) PCT/IB03/01374

(51) Int. Cl. C12Q 1/68; C07H 21/04; C12N 15/63; C12N 1/15; C07K 14/37; G01N 33/53

(52) U.S. Cl. 435/6; 536/23.1; 435/320.1; 435/254.11; 530/350; 435/7.1

(57) ABSTRACT

The present invention is directed to polynucleotides encoding proteins Essential For the Growth (EFG) of filamentous fungi. The invention also deals with namely polypeptides encoded by said polynucleotides, screening assays for identifying compounds capable of inhibiting said EFG proteins activities, pharmaceutical or phytosanitary compositions comprising such compounds.

This is a division of U.S. patent application Ser. NO. 10/507,416, filed on May 27, 2005, incorporated herein by reference.

The present invention is directed to polynucleotides encoding proteins Essential For the Growth (EFG) of filamentous fungi. The invention also deals with namely polypeptides encoded by said polynucleotides, screening assays for identifying compounds capable of inhibiting said EFG protein activities, pharmaceutical or phytosanitary compositions comprising such compounds.

### BACKGROUND

The opportunistic pathogen Aspergillus fumigatus is the cause of the most frequent deadly airborne fungal infection in developed countries. In order to identify novel antifungal drug targets, the inventors investigated the genome of A. fumigatus for genes that are necessary for efficient fungal growth.

Aspergillus fumigatus is a saprophytic filamentous fungus that disseminates through the release of asexual spores (conidia) into air^{1,2}. They are daily inhaled without major consequences for human health. However, in immuno-compromised hosts, A. fumigatus can cause a usually fatal infection, termed invasive pulmonary aspergillosis^{1,3} (IPA). With the increasing number of immuno-deficient patients and the development of severe immuno-suppressive therapies, A. fumigatus has become the most prevalent airborne fungal pathogen⁴. Because of a difficult diagnosis during lifetime and the lack of non-toxic efficient antifungal treatments, IPA is associated with a mortality rate as high as 85%.

Currently available drugs belong to two families: polyenes (e.g. amphotericin B) and azoles (e.g. itraconazole), both of them targeting fungal membranes^{1,5}. Relative toxicity and side effects, in addition to an often-late diagnostic, limit their use^{1,5}. Recently, new antifungal compounds of the candin family (caspofungin) which target the enzyme responsible for cell wall β(1,3)-glucan biosynthesis came on the market⁶.

Besides, A. fumigatus is closely related to phytopathogenic fungis. There is a high need of new fungicidal compounds against such fungi. Thus, EFG fungal genes identified by the inventors have a strong utility in the phytopathology field : the identified EFG genes are useful for identifying new fungicidal compositions in screening assays. Knowing the EFG described further, homologous genes of A. fumigatus can be isolated from other fungi, namely: Botrytis cinerea, Mycosphaerella graminicola, Stagnospora nodorum, Blumeria graminis, Colleotrichum lindemuthianum, Puccinia graminis, Leptosphaeria maculans, Fusarium oxysporum, Fusarium graminearum, Venturia inaequalis, most preferably Magnaporthe fungi, even more preferably Magnaporthe grisea.

A rational approach to increase the antifungal arsenal relies on the identification of novel targets involved in various aspects of the fungal biology^{7,8}. Although genes necessary for virulence are seen as potential candidates⁹, no genuine virulence factor has been identified in A. fumigatus yet^{2,10}. On the other hand, studies which have addressed the role of proposed virulence factors, e.g. adhesins, toxic secondary metabolites or secreted proteases, by direct mutagenesis have only identified genes involved in melanin biosynthesis necessary for conidia pigmentation as important for virulence^{2,37,38,39}. Therefore, it appears that the search for A. fumigatus virulence factors has only identified genes that protect conidia from the host response (pigment biosynthesis prevents complement binding and phagocytosis) and metabolic pathway genes (reduced level of an essential nutrient at the site of infection).

Alternative attractive antifungal targets lie among gene products that are essential for fungal growth ex vivo^{11,12}. Compendia of essential genes have been obtained for Saccharomyces cerevisiae through various approaches including systematic gene inactivation or insertional mutagenesis in a diploid background followed by the analysis of meiotic progenies^{13,14}. More recently, a set of genes critical for growth of the dimorphic yeast Candida albicans has also been defined using inducible expression of antisense RNA molecules¹⁵. Among the 86 C. albicans genes identified, 38% have no known homologues in available databases¹⁵. Differences in essential biological processes between the yeasts S. cerevisiae and C. albicans highlight the need to study the larger and more complex filamentous fungal genomes to reveal species-specific and filamentous-specific targets.

A. fumigatus is haploid and devoid of a sexual cycle¹⁶, preventing the application of strategies that use classical genetics to define essential genes. The inventors have now demonstrated that the parasexual genetic cycle can be used to demonstrate the essential function of A. fumigatus genes. The inventors have used techniques of chemical haploidization of artificial diploid strains^{17,18}. In this setting, a heterozygous A. fumigatus diploid is generated by targeted gene replacement or by random insertional mutagenesis and subjected to haploidization with or without the selective pressure corresponding to the introduced mutation. The absence of haploid progenies under selective condition only is indicative of the inactivation of a gene essential for A. fumigatus growth (FIG. 1). Using this approach, the inventors have demonstrated that the FKS1 gene, encoding the 1,3-β-D-glucan synthase catalytic subunit, and the smcA gene, encoding a member of the SMC (structural maintenance of chromosome) protein family, are essential for A. fumigatus growth. However, their results have shown that the currently used insertional mutagenesis schemes for A. fumigatus which rely on integration of a heterologous DNA molecule by DNA-mediated transformation^{19,20} lead to frequent genomic rearrangements that hamper a high-throughput analysis (data not shown). So there was a need for new techniques allowing a reliable identification of EFG genes.

Transposon mutagenesis has been used widely in bacteria^{21,22} and yeasts^{23,24} to elucidate various biological questions but it was only very recently that it has been applied to the filamentous fungus kingdom^{25,26}. In particular, the impala160 transposable element from Fusarium oxysporum, a Class II transposable element of the Tcl-mariner family²⁷, has been shown to transpose efficiently in Fusarium species²⁸, A. nidulans²⁹ and Magnaporthe grisea³⁰. However, transposon mutagenesis has not been used yet for a reliable identification of genes essential for growth of Aspergillus fungi, especially A. fumigatus. Such identification needs appropriated protocol settings and is absolutely not obvious, practically speaking. Furthermore, as it will be shown, the EFG genes of A. fumigatus identified by the inventors were until now not known, and some of them are surprisingly totally specific for Aspergilli or for filamentous ascomycetes, being not found in ascomycetous yeasts. The new EFG genes from A. fumigatus described in this application are as such neither described nor suggested in the prior art.

### SUMMARY OF THE INVENTION

The inventors have succeeded in the identification of EFG genes, by using an in vivo transposon mutagenesis system for A. fumigatus. The inventors have shown that impala160 (see FR 2 791 361) and its derivatives also transpose in A. fumigatus and can be used to generate a collection of random heterozygous diploids. Screening by parasexual genetics of such a collection has resulted in the complete characterization without prior sequence information of 210 new A. fumigatus genes which are necessary for efficient fungal growth.

The present invention thus pertains, according to a first aspect, to nucleic acid molecules, including in particular the complete cDNA sequence, encoding the EFG protein, as well as the corresponding translation product. Oligonucleotide probes or primers hybridizing specifically with a EFG genomic DNA or cDNA sequence are also part of the present invention, as well as DNA amplification and detection methods using said primers and probes.

A further aspect of the invention consists of recombinant vectors comprising any of the nucleic acid sequences described above, and in particular of recombinant vectors comprising a EFG regulatory sequence or a sequence encoding a EFG protein, as well as of cell hosts comprising said nucleic acid sequences or recombinant vectors.

The invention is also directed to methods for the screening of substances or molecules that inhibit the expression of the EFG genes, as well as with methods for the screening of substances or molecules that interact with and/or inhibit the activity of a EFG polypeptide.

Another object of the invention is to develop new compositions, either pharmaceutical or phytosanitarical, capable of inhibiting or preferably completely suppressing the toxic effect of filamentous fungi.

More precisely, the invention relates, according to a first aspect, to a nucleic acid encoding an Essential For Growth (EFG) polypeptide selected from the group consisting of :

(i) a nucleic acid molecule encoding a polypeptide comprising the amino acid sequence depicted in one of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170;

(ii) a nucleic acid molecule comprising the nucleic acid sequence as depicted in one of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168;

(iii) a nucleic sequence having at least 80, 85, 90, 95, 98, 99% identity with a sequence of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168;

(iv) a nucleic acid molecule which hybridizes under stringent conditions to
(a) a nucleic acid as defined in (i), (ii) and (iii), or
(b) a complementary strand of (a);

(v) a nucleic acid the sequence of which is degenerated as a result of the genetic code to the sequence of a nucleic acid as defined in (i), (ii), (iii) and (iv).

The invention also relates to an isolated nucleic acid, said nucleic acid comprising a nucleotide sequence encoding:
i) a EFG polypeptide comprising an amino acid sequence having at least 80% identity to a sequence of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170; or
ii) a biologically active fragment of said polypeptide.

The invention also relates to an isolated nucleic acid, said nucleic acid comprising a nucleotide sequence encoding:
i) a EFG polypeptide comprising an amino acid sequence which is orthologous to a sequence of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170; or
ii) a biologically active fragment of said polypeptide.

The invention also relates to an isolated nucleic acid sequence mentioned above encoding a polypeptide of A. fumigatus exhibiting a biological function associated to fungal growth, said nucleic acid comprising a sequence of SEQ ID NO 2, 5, 8 , 14, 17, 20, 23, 26, 29, 32, 35, 38, 41, 44, 47, 50, 53, 56, 59, 93, 97, 101, 105, 109, 113, 117, 121, 125, 129, 133, 137, 141, 145, 149, 153, 157, 161, 165, 169. The sequences of SEQ ID NO 2, 5, 8, 14, 17, 20, 23, 26, 29, 32, 35, 38, 41, 44, 47, 50, 53, 56, 59, 93, 97, 101, 105, 109, 113, 117, 121, 125, 129, 133, 137, 141, 145, 149, 153, 157, 161, 165, 169 are issued respectively from the sequences of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168, and are also defined as ORF NO 2, 5, 8, 14, 17, 20, 23, 26, 29, 32, 35, 38, 41, 44, 47, 50, 53, 56, 59, 93, 97, 101, 105, 109, 113, 117, 121, 125, 129, 133, 137, 141, 145, 149, 153, 157, 161, 165, 169. For instance SEQ ID No 2 is ORF NO 2, is issued from SEQ ID No 1, and encodes for the protein of SEQ ID No 3; SEQ ID No 5 is ORF NO 5, is issued from SEQ ID No 4, and encodes for the protein of SEQ ID No 6.

ORF (open reading frame) are representative fragments of the EFG genes of the invention, between a start codon and a stop codon or between two stop codons encoding the EFG polypeptides of the invention.

The biological function associated to fungal growth is preferably chosen in the group consisting of protein synthesis, protein maturation, protein transport, nuclear architecture, RNA processing, nucleotide metabolism, chromatine structure, cell cycle control.

The invention also relates to said nucleic acid operably linked to a promoter, to an expression cassette comprising said nucleic acid, to a host cell comprising said expression cassette.

According to another aspect the invention relates to a biologically active polypeptide encoded by a nucleic acid described above.

The invention also relates to a polypeptide comprising an amino acid sequence of at least 80% amino acid sequence identity to a sequence of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170.

According to a further aspect, the invention relates to a method of identifying a candidate inhibitor of EFG polypeptide, said method comprising:

i) contacting a EFG polypeptide according to claim 5 or 6 with a test compound ;
ii) determining whether said compound selectively binds to said polypeptide, said binding indicating that said compound is a candidate inhibitor.

The invention also relates to a method of identifying a candidate inhibitor of EFG polypeptide, said method comprising:

a) contacting said polypeptide with a test compound;
b) determining whether said compound selectively inhibits the activity of said polypeptide, said inhibition indicating that said compound is a candidate inhibitor.

According to a further aspect, the invention relates to a method for locating at least one gene essential for the growth of a haploid fungus, said method comprising the following successive steps:

generation of diploid strain from haploid fungal strain ;

mutagenesis of said diploid strain;

haploidisation of the diploid transformant strain, in selection conditions such that the absence of haploid progeny is indicative of mutagenesis occurring in said essential gene;

said mutagenesis being an in vivo transposon mutagenesis.

Preferably, the fungus belongs to the Aspergillus genus or the Penicillium genus. Preferably, the fungus is Aspergillus fumigatus.

Preferably, the transposon is the impala160 transposon or its derivatives, the selection medium is a benomyl-containing medium, the transposon impala160 is carried by a plasmid pNIpyr, the diploid strain is chosen between CEA225, CEA 226, CEA 227, and CEA 280:

strain DH5α (pNIpyr): this strain (CNCM I-2815) is derivated from strain DH5α of E. coli K-12 transformed by a derivative of pBR322 carrying the impala160 transposon of Fusarium oxysporum, in which has been inserted the pyrG gene of Aspergillus nidulans; transformed in a niaD- pyrG- of Aspergillus fumigatus, this plasmid confers prototrophy to uridin and uracil, and allows to select transposition of impala160: pyr by growth in a medium that contains nitrate as the sole nitrogen source.

CEA 225 (CNCM I-2816), CEA 226 (CNCM I-2817), CEA 227 (CNCM I-2818), and CEA 280 are diploid strains of Aspergillus fumigatus derivated from the strain CBS144-89 by gene transformation, spontaneous mutagenesis, cross and transformation by plasmid pNIpyr. Genotype: pyrG1/pyrG1 w1/F2/+ +/r7F1 cnx1/+niaD1/niaD2 X/X::pNipyr.

The invention also provides a method for locating at least one gene essential for the growth of a fungus of the Penicillium genus which exhibits a parasexual cycle and the diploids of which are stable.

Further, the heterozygous diploid strains are useful tools for direct screening of active molecules against A. fumigatus. Accordingly, the invention provides a method of screening of compounds that are active against A. fumigatus comprising:
preparing an A. fumigatus strain that is heterozygous for an EFG gene (heterozygous EFGn/efgn);
preparing an A. fumigatus strain that is homozygous for the EFG gene (homozygous EFGn/EFGn);
comparing the effect of a candidate compound on the heterozygous EFGn/efgn and on the homozygous EFGn/EFGn,
   the higher inhibiting effect on the heterozygous EFGn/efgn than on the homozygous EFGn/EFGn indicating that the compound is an inhibitor.

This method involves typically the comparison for one EFG gene (for instance for EFG1 gene (n=1), comparison between EFG1/efg1 strain and EFG1/EFG1 strain).

Thus, a population of heterozygous diploid A. fumigatus G/g::impala can be screened in order to identify one or more strains that are more sensitive to a fungicidal compound which action mechanism is unknown : the characterization of the gene G will allow to identify the action mechanism of said fungicidal compound.

According to a further aspect, the invention relates to an isolated nucleic acid sequence described above, obtainable by a method of locating described above.

According to a further aspect, the invention relates to a composition capable of inhibiting haploid fungal growth, said composition comprising at least one compound capable of inhibiting the expression of at least one EFG gene the nucleic acid sequence of which is described above.

The composition is typically either pharmaceutical or fungicidal.

### BRIEF DESCRIPTION OF THE SEQUENCE LISTING

SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168 are cDNA sequences encoding Aspergillus fumigatus EFG protein. In the whole application, the expression “SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168” means the group consisting of SEQ ID No 1, SEQ ID No 4, SEQ ID No 7, SEQ ID No 10, SEQ ID NO 13, SEQ ID No 16, SEQ ID No 19, SEQ ID No 22, SEQ ID No 25, SEQ ID No 28, SEQ ID No 31, SEQ ID No 34, SEQ ID No 37, SEQ ID No 40, SEQ ID No 43, SEQ ID NO 46, SEQ ID No 49, SEQ ID No 52, of SEQ ID No 55, SEQ ID No 58, SEQ ID No 92, SEQ ID No 96, SEQ ID No 100, SEQ ID No 104, SEQ ID No 108, SEQ ID No 112, SEQ ID No 116, SEQ ID No 120, SEQ ID No 124, SEQ ID No 128, SEQ ID No 132, SEQ ID No 136, SEQ ID No 140, SEQ ID No 144, SEQ ID No 148, SEQ ID No 152, SEQ ID No 156, SEQ ID No 160, of SEQ ID No 164, SEQ ID No 168.

SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170 are the amino acid sequences of Aspergillus fumigatus EFG polypeptides. In the whole application, the expression “SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170” means the group consisting of SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID N′27, SEQ ID NO 30, SEQ ID NO 33, SEQ ID NO 36, SEQ ID NO 39, SEQ ID NO 42, SEQ ID NO 45, SEQ ID NO 48, SEQ ID NO 51, SEQ ID NO 54, of SEQ ID NO 57, SEQ ID NO 60, SEQ ID NO 94, SEQ ID NO 98, SEQ ID NO 102, SEQ ID NO 106, SEQ ID NO 110, SEQ ID NO 114, SEQ ID NO 118, SEQ ID NO 122, SEQ ID NO 126, SEQ ID NO 130, SEQ ID NO 134, SEQ ID NO 138, SEQ ID NO 142, SEQ ID NO 146, SEQ ID NO 150, SEQ ID NO 154, SEQ ID NO 158, SEQ ID NO 162, of SEQ ID NO 166, SEQ ID NO 170.

An analogous construction is to be applied when having the expression “SEQ ID NO 2, 5, 8, 14, 17, 20, 23, 26, 29, 32, 35, 38, 41, 44, 47, 50, 53, 56, 59, 93, 97, 101, 105, 109, 113, 117, 121, 125, 129, 133, 137, 141, 145, 149, 153, 157, 161, 165, 169”.

TABLE 1
A. fumigatus essential genes^{a}

| | | A. fumigatus genome sequence version of November 2001 | | | | | | A. fumigatus genome sequence version of February 2003 | | | | | | | |
|---|---|---|---|---|---|---|---|---|---|---|---|---|---|---|---|
| | | Nucleic | | Amino | Contig | Contig | | | Nucleic | | Amino | Contig | | Coordinates of | |
| Clone | | acid | ORF | acid | TIGR | length | impala160::pyrG | gene | acid | ORF | acid | TIGR | Contig length | gene SEQ_ID | impala160::pyrG |
| Id | Strain Id | SEQ_ID | SEQ_ID | SEQ_ID | n^{o} | (kb) | location | SEQ_ID | SEQ_ID | SEQ_ID | SEQ_ID | n^{o} | (kb) | on contig | location |
| 10-80 | CEA231 | 1 | 2 | 3 | 131 | 27.2 | 15399 | 103 | 104 | 105 | 106 | 4940 | 205712 | 54154-50350 | 53934 |
| 10-291 | CEA233 | 4 | 5 | 6 | 164 | 45.3 | 40601 | 111 | 112 | 113 | 114 | 4865 | 598154 | 3495-6359 | 4705 |
| 7-1-19 | CEA254 | 7 | 8 | 9 | 43 | 93.1 | 72592 | 119 | 120 | 121 | 122 | 4911 | 85632 | 43163-41221 | 42151 |
| 10-3-7 | CEA255 | 10 | 11 | 12 | 408 | 71.1 | 53190 | 123 | 124 | 125 | 126 | 4899 | 1041326 | 441274-438167 | 440657 |
| 2-6-4 | CEA256 | 13 | 14 | 15 | 110 | 207.8 | 105657 | 127 | 128 | 129 | 130 | 4938 | 1796676 | 582107-579544 | 581561 |
| 2-1-1 | CEA257 | 16 | 17 | 18 | 493 | 42.2 | 14886 | 131 | 132 | 133 | 134 | 4951 | 825238 | 8362-11737 | 11010 |
| 2-10- | CEA258 | 19 | 20 | 21 | 190 | 22.1 | 7131 | 135 | 136 | 137 | 138 | 4912 | 206341 | 46084-42446 | 43492 |
| 16 | | | | | | | | | | | | | | | |
| 5-4-21 | CEA259 | 22 | 23 | 24 | 1327 | 4.7 | 2928 | 139 | 140 | 141 | 142 | 4963 | 622595 | 373462-376145 | 375531 |
| 2-10- | CEA260 | 25 | 26 | 27 | 493 | 42.8 | 36145 | 143 | 144 | 145 | 146 | 4849 | 22910 | 12560-15101 | 13405 |
| 21 | | | | | | | | | | | | | | | |
| 7-5-9 | CEA261 | 28 | 29 | 30 | 93 | 53.7 | 11506 | 147 | 148 | 149 | 150 | 4857 | 593293 | 164191-165827 | 164994 |
| 10-2- | CEA262 | 31 | 32 | 33 | 1366 | 3.6 | 1870 | 151 | 152 | 153 | 154 | 4903 | 416417 | 3571-1535 | 3157 |
| 18 | | | | | | | | | | | | | | | |
| 9-11 | CEA230 | 34 | 35 | 36 | 1754 | 6.3 | 603 | 99 | 100 | 101 | 102 | 4899 | 1041326 | 9642-7242 | 7285 |
| 4-3-3 | CEA263 | 37 | 38 | 39 | 838 | 9.8 | 8261 | 155 | 156 | 157 | 158 | 4944 | 310661 | 159432-161250 | 159931 |
| 11-6- | CEA264 | 40 | 41 | 42 | 846 | 16.7 | 3278 | 159 | 160 | 161 | 162 | 4899 | 1041326 | 65039-62439 | 64261 |
| 11 | | | | | | | | | | | | | | | |
| 8-47 | CEA228 | 43 | 44 | 45 | 960 | 5.1 | 223 | 91 | 92 | 93 | 94 | 4842 | 368858 | 234347-231296 | 234245 |
| 10-304 | CEA234 | 46 | 47 | 48 | 408 | 71.1 | 55903 | 115 | 116 | 117 | 118 | 4899 | 1041326 | 443110-444619 | 443430 |
| 10-175 | CEA232 | 49 | 50 | 51 | 221 | 76.1 | 67069 | 107 | 108 | 109 | 110 | 4938 | 1796676 | 211008-213420 | 211352 |
| 11-4-9 | CEA265 | 52 | 53 | 54 | 573 | 36.5 | 13244 | 163 | 164 | 165 | 166 | 4826 | 811005 | 355652-358190 | 356111 |
| 2-10- | CEA266 | 55 | 56 | 57 | 585 | 39.5 | 24940 | 167 | 168 | 169 | 170 | 4898 | 422562 | 329309-331987 | 330474 |
| 18 | | | | | | | | | | | | | | | |
| 8-62 | CEA229 | 58 | 59 | 60 | 221 | 76.1 | 60969 | 95 | 96 | 97 | 98 | 4938 | 1796676 | 215653-219466 | 217453 |
| 6-8-13 | CEA280 | | | | 6 | 212.7 | 208370 | 171 | 172 | 173 | 174 | 4925 | 1085193 | 997952-996381 | 997591 |
| 5-3-11 | CEA281.1 | | | | 792 | 13.7 | 1353 | 175 | 176 | 177 | 178 | 4839 | 130518 | 10030-12622 | 12332 |
| 5-3-11 | CEA281.2 | | | | 792 | 13.7 | 1353 | 179 | 180 | 181 | 182 | 4839 | 130518 | 12269-14135 | 12332 |
| 10-4- | CEA282.1 | | | | 443 | 89.1 | 67662 | 183 | 184 | 185 | 186 | 4929 | 586561 | 328110-325663 | 328147 |
| 20 | | | | | | | | | | | | | | | |
| 10-4- | CEA282.2 | | | | 443 | 89.1 | 67662 | 187 | 188 | 189 | 190 | 4929 | 586561 | 328075-330267 | 328147 |
| 20 | | | | | | | | | | | | | | | |
| 11-6- | CEA283 | | | | 716 | 14.3 | 9480 | 191 | | | | 4910 | 185565 | 9638-11637 | 10637 |
| 20 | | | | | | | | | | | | | | | |
| 4-3-4 | CEA284.1 | | | | 652 | 29.4 | 18411 | 192 | 193 | 194 | 195 | 4899 | 1041326 | 472441-476776 | 476988 |
| 4-3-4 | CEA284.2 | | | | 652 | 29.4 | 18411 | 196 | 197 | 198 | 199 | 4899 | 1041326 | 477626-479684 | 476988 |

bis

| | | March | March | | | Location of | | | | |
|---|---|---|---|---|---|---|---|---|---|---|
| | | 2002 | 2003 | Size | | imp160::pyrG | | | | |
| | | Amino | Amino | introns + | Size | relative to | | | | |
| Clone | Protein | acid | acid | exons | protein | A in start | S. cerevisiae closest | Essential in | | |
| Id | Id | SEQ_ID | SEQ_ID | (nt) | (aa) | codon (nt) | homologue | S. cerevisiae | Function | Functional category |
| 10-80 | CEA231_prot | 3 | 106 | 2805 | 934 | −280 | DBP10/YDL031w | yes | ATP-dependent RNA helicase | RNA processing |
| 10-291 | CEA233_prot | 6 | 114 | 1865 | 574 | 733 | NAR1/YNL240c | yes | Nuclear architecture related protein | Nuclear architecture |
| 7-1-19 | CEA254_prot | 9 | 122 | 943 | 200 | 511 | GUK1/YDR454c | yes | Guanylate kinase | Nucleotide |
| | | | | | | | | | | metabolism |
| 10-3-7 | CEA255_prot | 12 | 126 | 2108 | 657 | 57 | SRP101/YDR292c | yes | Signal recognition particle receptor - alpha | Protein transport |
| | | | | | | | | | subunit | |
| 2-6-4 | CEA256_prot | 15 | 130 | 1564 | 460 | 46 | WBP1/YEL002c | yes | Oligosaccharyl transferase beta subunit | Protein modification |
| 2-1-1 | CEA257_prot | 18 | 134 | 2376 | 715 | 2148 | YGL245w | yes | Glutamate-tRNA synthetase | Protein synthesis |
| 2-10-16 | CEA258_prot | 21 | 138 | 2639 | 809 | 2191 | CDC27/YBL084c | yes | Cell division control protein | Cell cycle control |
| 5-4-21 | CEA259_prot | 24 | 142 | 1707 | 568 | 1569 | RSC9/YML127w | yes | Component of the chromatin remodeling | Chromatin structure |
| | | | | | | | | | complex | |
| 2-10-21 | CEA260_prot | 27 | 146 | 1542 | 493 | 345 | SPE2/YOL052c | yes | S-adenosylmethionine decarboxylase | Metabolism |
| 7-5-9 | CEA261_prot | 30 | 150 | 637 | 139 | 303 | RPL17A/YKL180w | no | Ribosomal protein of the large subunit of the | Protein synthesis |
| | | | | | | | RPL17B/YJL177w | | ribosome (L17) | |
| 10-2-18 | CEA262_prot | 33 | 154 | 1037 | 256 | −131 | RPL1A/YGL135w | no | Ribosomal protein of the large subunit of the | Protein synthesis |
| | | | | | | | RPL1B/YPL220w | | ribosome (L1) | |
| 9-11 | CEA230_prot | 36 | 102 | 1401 | 399 | 1316 | MSW1/YDR268w | no | Mitochondrial tryptophanyl-tRNA synthetase | Protein synthesis |
| 4-3-3 | CEA263_prot | 39 | 158 | 819 | 227 | −1 | GOS1/YHL031c | no | SNARE protein | Protein transport |
| 11-6-11 | CEA264_prot | 42 | 162 | 1601 | 394 | 278 | RIM11/YMR139w | no | Serine/threonine-protein kinase | Cell cycle control |
| 8-47 | CEA228_prot | 45 | 94 | 2052 | 683 | −398 | YFL034w | no | Probable membrane protein; Yfl034wp | Unknown |
| 10-304 | CEA234_prot | 48 | 118 | 461 | 141 | −179 | RPL14A/YHL001w | no | Ribosomal protein of the large subunit of the | Protein synthesis |
| | | | | | | | RPL14B/YKL006w | | ribosome (L14) | |
| 10-175 | CEA232_prot | 51 | 110 | 1413 | 428 | −105 | HEM15/YOR176w | yes | Ferrochelatase | Heme biosynthesis |
| 11-4-9 | CEA265_prot | 54 | 166 | 1539 | 512 | −40 | COX10/YPL172c | no | Protoheme IX farnesyltransferase | Heme biosynthesis |
| 2-10-18 | CEA266_prot | 57 | 170 | 1629 | 542 | 195 | TRF4/YOL115w | no | Topoisomerase | DNA replication |
| 8-62 | CEA229_prot | 60 | 98 | 2814 | 937 | 1300 | no hit found | | unknown function | Unknown |
| 6-8-13 | CEA280_prot | | 174 | 573 | 190 | 138 | no hit found | | weak homology to S. pombe GTPase activator | unknown |
| | | | | | | | | | protein and to myocilin | |
| 5-3-11 | CEA281.1_prot | | 178 | 1974 | 609 | 2037 | PAC2/YER007w | no | tubulin folding cofactor E | Cytoskeleton |
| 5-3-11 | CEA281.2_prot | | 182 | 963 | 291 | −290 | no hit found | | unknown function | unknown |
| 10-4-20 | CEA282.1_prot | | 186 | 1448 | 464 | −537 | PBP2/YBR233w | no | hnRNP complex protein/PAB1-binding protein | RNAprocessin |
| 10-4-20 | CEA282.2_prot | | 190 | 1143 | 344 | −427 | SEC3/YER008c | yes | unknown function | Protein secretion |
| 4-3-4 | CEA284.1_prot | | 195 | 3336 | 1059 | 4049 | ENA5/YDR038c | no | P-type ATPase | Ion transport |
| 4-3-4 | CEA284.2_prot | | 199 | 1059 | 352 | −1137 | no hit found | | secondary metabolite biosynthesis protein | unknown |

^{a}For each identified integration of impala160::pyrG, the corresponding TIGR contig number is indicated (www.tigr.org) with its length (kb) and the position of the TA where transposon integration occur (bp).

### BRIEF DESCRIPTION OF THE DRAWINGS

FIG. 1: Strategy for the identification of essential genes in A. fumigatus. A stable diploid strain heterozygous for spore color markers (w1, r7) is randomly mutagenized with the transposable element impala160::pyrG (imp::pyr). During haploidization on benomyl containing media, random loss of chromosomes gives rise to two sub-populations of colored haploid conidia (w1, or r7): one bearing the transposon-inactivated allele (population A) and one bearing wild type allele (population B). The ability to form haploid progenies on non selective haploidization medium and the inability on selective haploidization medium (without uridine and uracile) leads to the identification of mutant strains with an insertion in an essential gene.

FIG. 2: In vivo random transposon mutagenesis in A. fumigatus. (A) Schematic representation of impala160::pyrG transposition in a A. fumigatus strain transformed by pNIpyr. Expression of the nitrate reductase gene (niaD) is prevented by the presence of the transposable element impala160::pyG (imp::pyr) into the promoter region. Positive selection of transposition events is obtained by selection of nitrate-utilizing revertants which appear as a result of the excision of imp::pyr and the restoration of a functional niaD promoter. Selection of imp::pyr reintegration events is ensured by the presence of pyrG in the transposable element when transposition events are induced in a A. fumigatus pyrG strain and in the absence of uridine and uracile. (B) Southern blot analysis of parental diploid transformants (lane 1: CEA225; lane 4: CEA226; lane 7: CEA227) and diploid revertants (lane 2: Rev 225-1; lane 3: Rev 225-2; lane 5: Rev 226-1; lane 6: Rev 226-2; lane 8: Rev 227-1; lane 9: Rev 227-2). Hybridization with a probe for impala160::pyrG revealed integration of the transposable element into the promoter of the niaD gene in the three parental transformants (arrowheads) and integration at apparent random sites in the genome of the diploid revertants.

FIG. 3: Parasexual screening. Haploidization of 10 diploid revertants on non-selective (A) and selective (B) media.

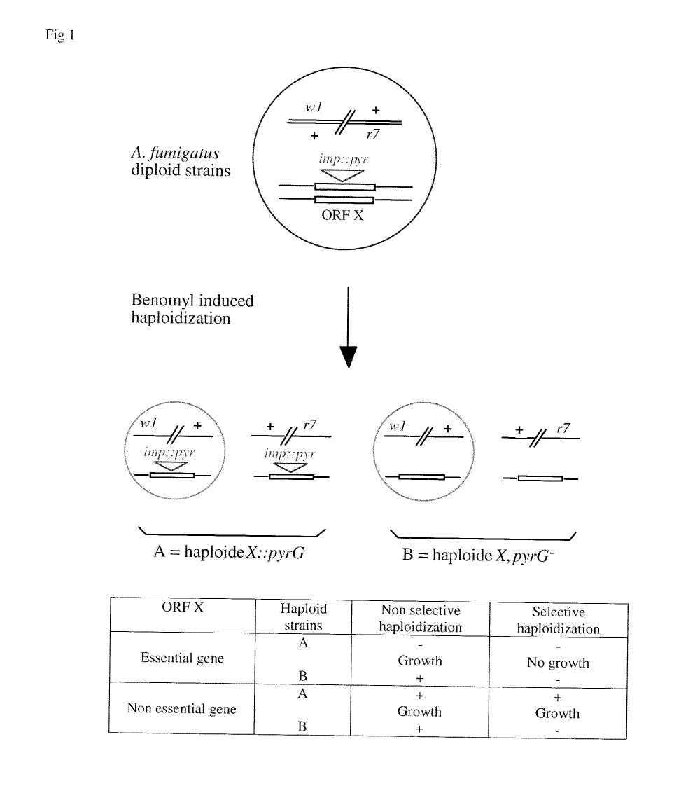

Random segregation of chromosomes is visualized by the production of differently colored haploid conidia (see FIG. 1: allele w1 and r7). On selective haploidization medium, in the case of plasmid integration in an essential gene, a residual growth phenotype is observed (arrowheads). For these revertants, haploid spores obtained on non selective haploidization medium were tested for the absence of the transposable element in order to confirm the essential phenotype.

### DETAILED DESCRIPTION OF THE INVENTION

The present invention is based on the discovery of novel molecules, referred to herein as EFG protein and nucleic acid molecules, encoding proteins Essential For Growth expressed in Aspergillus fumigatus.

An artificial A. fumigatus diploid strain with one copy of the impala160 transposon from Fusarium oxysporum integrated into its genome was used to generate a library of diploid strains with random transposon integration. Among ca. 2,300 heterozygous diploid strains screened by parasexual genetics, 1.2% have a copy of the transposable element integrated into a gene essential for fungal growth. Homologues of genes essential for Saccharomyces cerevisiae growth have been identified, as well as genes that do not share homologues in other fungal species.

The term “EFG genes” refers to genes that are necessary for efficient fungal growth. An efficient growth refers to the normal growth of this fungus in absence of inhibitor of at least one of these genes. Inhibitors may be used to inhibit normal expression of at least one of the EFG genes identified herein.

Isolated EFG proteins of the present invention, have an amino acid sequences sufficiently homologous to the amino acid sequence of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170, or are encoded by a nucleotide sequence sufficiently homologous to one of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least about 30-40% identity, preferably 40-50% identity, more preferably 50-60%, and even more preferably 60-70%, 70-80%, 80, 90%, 95%, 97%, 98%, 99% or 99.8% identity across the amino acid sequences of the domains are defined herein as sufficiently homologous. Furthermore, amino acid or nucleotide sequences which share at least 30%, preferably 40%, more preferably 60%, 70%, 80%, 90%, 95%, 97%, 98%, 99% or 99.8% identity and share a common functional activity are defined herein as sufficiently homologous.

Homologues are thus defined as those genes or gene products that show a significant level of identity or similarity at the nucleotide or amino acid level, respectively, as indicated above.

Orthologous genes are defined herein as those genes or gene products from two different species which, upon individual comparison to the gene set of the other species, appear reciprocally as the closest homologues.

As used interchangeably herein, a “EFG activity”, “biological activity of EFG” or “functional activity of EFG”, refers to an activity exerted by a EFG protein, polypeptide or nucleic acid molecule as determined in vivo, or in vitro, according to appropriate techniques.

The level of inhibition of EFG activity may depend on the number of the EFG genes that are inhibited and on the EFG genes inhibited. The inhibition of at least one EFG gene results in an inhibition of at least 5, 10, 20, 40, 60, 80, 90, 95% of the efficient growth. Preferably, the inhibition is of 100%, meaning the total suppression of growth and of the toxic effect of the fungus.

In one embodiment, a EFG activity is a direct activity, such as an association with a EFG-target molecule or most preferably EFG activity. As used herein, a “target molecule” is a molecule with which a EFG protein binds or interacts in nature, such that EFG-mediated function is achieved. Alternatively, a EFG activity is an indirect activity, such as an activity mediated by interaction of the EFG protein with a EFG target molecule such that the target molecule modulates a downstream cellular activity (e.g., interaction of an EFG molecule with a EFG target molecule can modulate the activity of that target molecule on an intracellular signaling pathway).

### I. EFG Nucleic Acids

The inventors have identified and completely characterized 21 EFG cDNA indicated in Table 1 and Table ibis. For instance EFG2 refers to a 1735 nucleotide (nt) sequence in length (SEQ ID NO 4) which comprises the nucleic acid of SEQ ID NO 5 of 1592 nt in length, which encodes the protein of SEQ ID NO 6, which is 530 amino acid residues in length.

One aspect of the invention pertains to purified or isolated nucleic acid molecules that encode EFG proteins or biologically active portions thereof, as well as nucleic acid fragments thereof. Fragments may be used for example as hybridization probes to identify EFG-encoding nucleic acids (e.g., EFG mRNA) and fragments for use as probes (e.g. for detection of EFG nucleic acid molecules) or primers (e.g. for sequencing, genotyping, amplification or mutation of EFG nucleic acid molecules). As used herein, the term “nucleic acids” and “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. Throughout the present specification, the expression “nucleotide sequence” may be employed to designate indifferently a polynucleotide or a nucleic acid. More precisely, the expression “nucleotide sequence” encompasses the nucleic material itself and is thus not restricted to the sequence information (i.e., the succession of letters chosen among the four base letters) that biochemically characterizes a specific DNA or RNA molecule. Also, used interchangeably herein are terms “nucleic acids”, “oligonucleotides”, and “polynucleotides”.

An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having a nucleotide sequence of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168, or a portion thereof, can be isolated using standard molecular biology techniques and the sequence information provided herein. Using all or portion of the nucleic acid sequence of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168, as a hybridization probe, EFG nucleic acid molecules can be isolated using standard hybridization and cloning techniques (e.g., as described in Sambrook, J., Fritsh, E. F., and Maniatis, T. Molecular Cloning. A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of a sequence of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168, can be isolated by the polymerase chain reaction (PCR) using synthetic oligonucleotide primers designed based upon a sequence of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to EFG nucleotide sequences can be prepared by standard synthetic techniques, e.g., using an automated DNA synthesizer.

In a preferred embodiment, an isolated nucleic acid molecule of the invention comprises, consists essentially of, or consists of the nucleotide sequence of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168, or fragments thereof. The sequences of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168, correspond to A. fumigatus EFG cDNA.

Also encompassed by the EFG nucleic acids of the invention are nucleic acid molecules which are complementary to EFG nucleic acids described herein. Preferably, a complementary nucleic acid is sufficiently complementary to a nucleotide sequence of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168, such that it can hybridize to a nucleotide sequence of SEQ NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168.

Another object of the invention is a purified, isolated, or recombinant nucleic acid encoding a EFG polypeptide comprising an amino acid sequence of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170, or fragments thereof. Preferred polynucleotides of the invention also include purified, isolated, or recombinant EFG cDNAs consisting of, consisting essentially of, or comprising a sequence of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168. Particularly preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, 1000 or 2000 nucleotides (upper lengths of the fragments to be adapted to the length of the nucleotide sequence) of a sequence of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168, or the complements thereof.

Moreover, the nucleic acid molecule of the invention can comprise only a portion of a nucleic acid sequence of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168, for example a fragment which can be used as a probe or primer or a fragment encoding a biologically active portion of a EFG protein. The nucleotide sequence determined from the cloning of the EFG genes allows for the generation of probes and primers designed for use in identifying and/or cloning other EFG family members, as well as EFG homologues from other species. The probe/primer typically comprises substantially purified oligonucleotide. The oligonucleotide typically comprises a region of nucleotide sequence that hybridizes under stringent conditions to at least about 12, preferably about 25, more preferably about 40, or 75 consecutive nucleotides of a sequence of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168, or a sequence complementary thereto.

A nucleic acid fragment encoding a “biologically active portion of a EFG protein” can be prepared by isolating a portion of a nucleotide sequence of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168, which encodes a polypeptide having a EFG biological activity (the biological activities of the EFG proteins described herein), expressing the encoded portion of the EFG protein (e.g., by recombinant expression in vitro) and assessing the activity of the encoded portion of the EFG protein.

The invention further encompasses nucleic acid molecules that differ from a nucleotide sequence of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168, due to degeneracy of the genetic code and thus encode the same EFG proteins as those encoded by a nucleotide sequence of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168. In another embodiment, an isolated nucleic acid molecule of the invention comprises a nucleotide sequence encoding a protein having an amino acid sequence of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170.

In addition to the EFG nucleotide sequences of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168, it will be appreciated by those skilled in the art that DNA sequence polymorphisms that lead to changes in the amino acid sequences of the EFG proteins may exist within a population (e.g., the fungal population). Such genetic polymorphism in the EFG genes may exist among individuals within a population due to natural allelic variation. As used herein, the terms “gene” and “recombinant gene” refer to nucleic acid molecules comprising an open reading frame encoding an EFG protein, preferably a fungal EFG protein. Such natural allelic variations can typically result in 1-5% variance in the nucleotide sequence of a EFG gene. Any and all such nucleotide variations and resulting amino acid polymorphisms in EFG genes that are the result of natural allelic variation and, most preferably, that do not alter the functional activity of a EFG protein are intended to be within the scope of the invention.

Nucleic acid molecules corresponding to natural allelic variants and homologues of the EFG cDNAs of the invention can be isolated based on their homology to the EFG nucleic acids disclosed herein using the cDNAs disclosed herein, or a portion thereof, as a hybridization probe according to standard hybridization techniques under stringent hybridization conditions.

As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 70%, more preferably at least about 80%, even more preferably at least about 85%, 90%, 95% or 98% homologous to each other typically remain hybridized to each other. Stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6 * sodium chloride/sodium citrate (SSC) at about 45° C., followed by one or more washes in 0.2 *SSC, 0.1% SDS at 50-65° C. Preferably, an isolated nucleic acid molecule of the invention that hybridizes under stringent conditions to the sequence of SEQ ID NO:1 corresponds to a naturally-occurring nucleic acid molecule. As used herein, a “naturally-occurring” nucleic acid molecule refers to a RNA or DNA molecule having a nucleotide sequence that occurs in nature (e.g., encodes a natural protein).

In addition to naturally-occurring allelic variants of the EFG sequences that may exist in the population, the skilled artisan will further appreciate that changes can be introduced by mutation into a nucleotide sequence of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168, thereby leading to changes in the amino acid sequence of the encoded EFG proteins, without altering the functional ability of the EFG proteins. For example, nucleotide substitutions leading to amino acid substitutions at “non-essential” amino acid residues can be made in a sequence of NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170. A “non-essential” amino acid residue is a residue that can be altered from the wild-type sequence of EFG (e.g., the sequence of SEQ ID NO:1) without altering the biological activity, whereas an “essential” amino acid residue is required for biological activity. For example, amino acid residues that are conserved among the EFG proteins of the present invention, are predicted to be less unamenable to alteration. Furthermore, additional conserved amino acid residues may be amino acids that are conserved between the EFG proteins of the present invention and other members of the Aspergillus family and/or of other fungi.

Thus, the invention further encompasses nucleic acid molecules that are homologous to the nucleic acids of A. fumigatus described above and that are isolated from target phytopathogenic fungi, namely Botrytis cinerea, Mycosphaerella graminicola, Stagnospora nodorum, Blumeria graminis, Colleotrichum lindemuthianum, Puccinia graminis, Leptosphaeria maculans, Fusarium oxysporum, Fusarium graminearum, Venturia inaequalis, most preferably fungi of the genus Magnaporthe, even most preferably Magnaporthe grisea.

Accordingly, another aspect of the invention pertains to nucleic acid molecules encoding EFG proteins and biologically active fragments thereof that contain changes in amino acid residues that are not essential for activity. Such EFG proteins differ in amino acid sequence of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170, yet retain biological activity. In one embodiment, the isolated nucleic acid molecule comprises a nucleotide sequence encoding a protein, wherein the protein comprises an amino acid sequence at least about 60% homologous to an amino acid sequence of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170. Preferably, the protein encoded by the nucleic acid molecule is at least about 65-70% homologous to a sequence of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170, more preferably sharing at least about 75-80% identity with SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170, even more preferably sharing at least about 85%, 90%, 92%, 95%, 97%, 98%, 99% or 99.8% identity with a sequence of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170.

An isolated nucleic acid molecule encoding a EFG protein homologous to a protein of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170, can be created by introducing one or more nucleotide substitutions, additions or deletions into a nucleotide sequence of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168, such that one or more amino acid substitutions, additions or deletions are introduced into the encoded protein. Mutations can be introduced into a sequence of NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168, by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a EFG protein is preferably replaced with another amino acid residue from the same side chain family. Alternatively, in another embodiment, mutations can be introduced randomly along all or part of a EFG coding sequence, such as by saturation mutagenesis. Following mutagenesis the encoded protein can be expressed recombinantly and the activity of the protein can be determined.

The biological EFG activity of the protein fragments and mutants described above can be assayed according to the tests known from the one skilled in the art.

Primers and probes of the invention can be prepared by any suitable method, including, for example, cloning and restriction of appropriate sequences and direct chemical synthesis by a method such as the phosphodiester method of Narang S A, Hsiung H M, Brousseau R, Methods Enzymol 1979; 68:90-98, the phosphodiester method of Brown E L, Belagaje R, Ryan M J, Khorana H G, Methods Enzymol 1979; 68:109-151, the diethylphosphoramidite method of Beaucage et al., Tetrahedron Lett 1981, 22: 1859-1862 and the solid support method described in EP 0 707 592.

Any of the polynucleotides of the present invention can be labeled, if desired, by incorporating any label known in the art to be detectable by spectroscopic, photochemical, biochemical, immunochemical, or chemical means. For example, useful labels include radioactive substances (including, ³²P, ³⁵S, ³H, ¹²⁵I), fluorescent dyes (including, 5-bromodesoxyuridin, fluorescein, acetylaminofluorene, digoxigenin) or biotin. Preferably, polynucleotides are labeled at their 3′ and 5′ ends. A label can also be used to capture the primer, so as to facilitate the immobilization of either the primer or a primer extension product, such as amplified DNA, on a solid support. A capture label is attached to the primers or probes and can be a specific binding member which forms a binding pair with the solid's phase reagent's specific binding member (e.g. biotin and streptavidin). Therefore depending upon the type of label carried by a polynucleotide or a probe, it may be employed to capture or to detect the target DNA. Further, it will be understood that the polynucleotides, primers or probes provided herein, may, themselves, serve as the capture label.

The probes of the present invention are useful for a number of purposes. They can be notably used in Southern hybridization to genomic DNA. The probes can also be used to detect PCR amplification products. They may also be used to detect mismatches in the EFG gene or mRNA using other techniques.

Any of the polynucleotides, primers and probes of the present invention can be conveniently immobilized on a solid support. Solid supports are known to those skilled in the art. A solid support, as used herein, refers to any material which is insoluble, or can be made insoluble by a subsequent reaction. The solid support can be chosen for its intrinsic ability to attract and immobilize the capture reagent. Alternatively, the solid phase can retain an additional receptor which has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, duracytes and other configurations known to those of ordinary skill in the art. The polynucleotides of the invention can be attached to or immobilized on a solid support individually or in groups of at least 2, 5, 8, 10, 12, 15, 20, or 25 distinct polynucleotides of the invention to a single solid support. In addition, polynucleotides other than those of the invention may be attached to the same solid support as one or more polynucleotides of the invention.

Consequently, the invention also comprises a method for detecting the presence of a nucleic acid comprising a nucleotide sequence of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168, a fragment or a variant thereof and a complementary sequence thereto in a sample, said method comprising the following steps of:

a) bringing into contact a nucleic acid probe or a plurality of nucleic acid probes which can hybridize with a nucleotide sequence included in a nucleic acid sequence of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168, a fragment or a variant thereof and a complementary sequence thereto and the sample to be assayed; and

b) detecting the hybrid complex formed between the probe and a nucleic acid in the sample.

Any polynucleotide provided herein may be attached in overlapping areas or at random locations on a solid support. Alternatively the polynucleotides of the invention may be attached in an ordered array wherein each polynucleotide is attached to a distinct region of the solid support which does not overlap with the attachment site of any other polynucleotide. Preferably, such an ordered array of polynucleotides is designed to be “addressable” where the distinct locations are recorded and can be accessed as part of an assay procedure. Addressable polynucleotide arrays typically comprise a plurality of different oligonucleotide probes that are coupled to a surface of a substrate in different known locations. The knowledge of the precise location of each polynucleotides location makes these “addressable” arrays particularly useful in hybridization assays. Any addressable array technology known in the art can be employed with the polynucleotides of the invention. One particular embodiment of these polynucleotide arrays is known as the Genechips, and has been generally described in U.S. Pat. NO. 5,143,854; PCT publications WO 90/15070 and 92/10092.

### II. EFG Polypeptides and Anti-EFG Antibodies

One aspect of the invention pertains to isolated EFG proteins, and biologically active portions thereof, as well as polypeptide fragments suitable for use as immunogens to raise fungus, preferably Aspergillus, most preferably A. fumigatus, anti-EFG antibodies. In one embodiment, native EFG proteins can be isolated from cells or tissue sources by an appropriate purification scheme using standard protein purification techniques. In another embodiment, EFG proteins are produced by recombinant DNA techniques. Alternative to recombinant expression, a EFG protein or polypeptide can be synthesized chemically using standard peptide synthesis techniques.

An “isolated” or “purified” protein or biologically active portion thereof is substantially free of cellular material or other contaminating proteins from the cell or tissue source from which the EFG protein is derived, or substantially free from chemical precursors or other chemicals when chemically synthesized. The language “substantially free of cellular material” includes preparations of EFG protein in which the protein is separated from cellular components of the cells from which it is isolated or recombinantly produced. In one embodiment, the language “substantially free of cellular material” includes preparations of EFG protein having less than about 30% (by dry weight) of non-EFG protein (also referred to herein as a “contaminating protein”), more preferably less than about 20% of non-EFG protein, still more preferably less than about 10% of non-EFG protein, and most preferably less than about 5% non-EFG protein. When the EFG protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The term “polypeptide” refers to a polymer of amino acids without regard to the length of the polymer; thus, peptides, oligopeptides, and proteins are included within the definition of polypeptide. This term also does not specify or exclude post-expression modifications of polypeptides, for example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

The term “recombinant polypeptide” is used herein to refer to polypeptides that have been artificially designed and which comprise at least two polypeptide sequences that are not found as contiguous polypeptide sequences in their initial natural environment, or to refer to polypeptides which have been expressed from a recombinant polynucleotide.

Biologically active portions of a EFG protein include peptides comprising amino acid sequences sufficiently homologous to or derived from an amino acid sequence of the EFG protein having a sequence of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170, which include less amino acids than the full length EFG proteins, and exhibit at least one activity of a EFG protein. Typically, biologically active portions comprise a domain or motif with at least one activity of the EFG protein. A biologically active portion of a EFG protein can be a polypeptide which is, for example at least 15, 25, 50, 100, 150, 200, 300, 400, 500, or more amino acids in length. The upper length just mentioned is of course to be adapted according to the size of the protein.

In a preferred embodiment, the EFG protein comprises, consists essentially of, or consists of the amino acid sequence of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170. The invention also concerns the polypeptide encoded by a nucleotide sequence selected from the group consisting of SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168, a complementary sequence thereof or a fragment thereto. The present invention embodies isolated, purified, and recombinant polypeptides comprising a contiguous span of at least 6 amino acids, preferably at least 8 to 10 amino acids, more preferably at least 12, 15, 20, 25, 30, 40, 50, 100, 200, 300, 400, 500, 600 or 650 amino acids in length (upper length defined as mentioned above).

In other embodiments, the EFG protein is substantially homologous to SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170, and retains the functional activity (at least 50, 60, 80, 90, 99%) of a protein of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170 yet differs in amino acid sequence due to natural allelic variation or mutagenesis, as described in detail in subsection I above. Accordingly, in another embodiment, the EFG protein is a protein which comprises an amino acid sequence at least about 60% homologous to an amino acid sequence of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170, and retains the functional activity of an EFG proteins of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170, respectively. Preferably, the protein is at least about 30%, 40%, 50%, 60%, 70%, 80%, 85%, 90%, 92%, 95%, 97%, 98%, 99% or 99.8% homologous to a sequence of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90% or 95% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “identity” is equivalent to amino acid or nucleic acid “homology”).

The comparison of sequences and determination of percent homology between two sequences can be accomplished using a mathematical algorithm, preferably the alignment method of Needleman and Wush, J. Mol. Biol., 1970, no 48, p 443, using the GAP GCC package (Devereux et al., Nucl. Acid. Res., 1984, vol 12, p 387). An other non-limiting example of a mathematical algorithim utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to EFG nucleic acid molecules of the invention. BLAST protein searches can be performed with the)(BLAST program, score=50, wordlength=3 to obtain amino acid sequences homologous to EFG protein molecules of the invention. To obtain gapped alignments for comparison purposes, Gapped BLAST can be utilized as described in Altschul et al., (1997) Nucleic Acids Research 25(17):3389-3402. When utilizing

BLAST and Gapped BLAST programs, the default parameters of the respective programs (e.g., XBLAST and NBLAST) can be used. See http://www.ncbi.nlm.nih.gov. Another preferred, non-limiting example of a mathematical algorithim utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The invention also provides EFG chimeric or fusion proteins. As used herein, a EFG “chimeric protein” or “fusion protein” comprises a EFG polypeptide operatively linked, preferably fused in frame, to a non-EFG polypeptide. In a preferred embodiment, a EFG fusion protein comprises at least one biologically active portion of a EFG protein. In another preferred embodiment, a EFG fusion protein comprises at least two biologically active portions of a EFG protein. For example, in one embodiment, the fusion protein is a GST-EFG fusion protein in which the EFG sequences are fused to the C-terminus of the GST sequences. Such fusion proteins can facilitate the purification of recombinant EFG. In another embodiment, the fusion protein is a EFG protein containing a heterologous signal sequence at its N-terminus, such as for example to allow for a desired cellular localization in a certain host cell.

The EFG fusion proteins of the invention can be incorporated into pharmaceutical compositions and administered to a subject in vivo. Moreover, the EFG-fusion proteins of the invention can be used as immunogens to produce anti-EFG antibodies in a subject, to purify EFG ligands and in screening assays to identify molecules which inhibit the interaction of EFG with a EFG target molecule.

The present invention also pertains to variants of the EFG proteins which function as either EFG mimetics or as EFG inhibitors. Variants of the EFG proteins can be generated by mutagenesis, e.g., discrete point mutation or truncation of a EFG protein. An agonist of the EFG proteins can retain substantially the same, or a subset, of the biological activities of the naturally occurring form of a EFG protein. An antagonist of a EFG protein can inhibit one or more of the activities of the naturally occurring form of the EFG protein by, for example, competitively inhibiting the EFG activity of a EFG protein. Thus, specific biological effects can be elicited by treatment with a variant of limited function.

In a preferred embodiment, variants of a EFG protein which function as EFG antagonists can be identified by screening combinatorial libraries of mutants, e.g., truncation mutants, of a EFG protein for EFG protein antagonist activity. In one embodiment, a variegated library of EFG variants is generated by combinatorial mutagenesis at the nucleic acid level and is encoded by a variegated gene library. A variegated library of EFG variants can be produced by, for example, enzymatically ligating a mixture of synthetic oligonucleotides into gene sequences such that a degenerate set of potential EFG sequences is expressible as individual polypeptides, or alternatively, as a set of larger fusion proteins (e.g., for phage display) containing the set of EFG sequences therein. There are a variety of methods which can be used to produce libraries of potential EFG variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential EFG sequences.

In addition, libraries of fragments of a EFG protein coding sequence can be used to generate a variegated population of EFG fragments for screening and subsequent selection of variants of a EFG protein. In one embodiment, a library of coding sequence fragments can be generated by treating a double stranded PCR fragment of a EFG coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the EFG protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of EFG proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected.

In one embodiment, cell based assays can be exploited to analyze a variegated EFG library.

Modified EFG proteins can be used for such purposes as enhancing therapeutic or prophylactic efficacy, or stability (e.g., ex vivo shelf life and resistance to proteolytic degradation in vivo). Such modified peptides, when designed to retain at least one activity of the naturally occurring form of the protein, are considered functional equivalents of the EFG protein described in more detail herein. Such modified peptide can be produced, for instance, by amino acid substitution, deletion, or addition.

Whether a change in the amino acid sequence of a peptide results in a functional EFG homolog (e.g. functional in the sense that it acts to mimic or antagonize the wild-type form) can be readily determined by assessing the ability of the variant peptide to produce a response in cells in a fashion similar to the wild-type EFG protein or competitively inhibit such a response. Peptides in which more than one replacement has taken place can readily be tested in the same manner.

A wide range of techniques are known in the art for screening gene products of combinatorial libraries made by point mutations, as well as for screening cDNA libraries for gene products having a certain property. Such techniques will be generally adaptable for rapid screening of the gene libraries generated by the combinatorial mutagenesis of EFG proteins. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

The invention also provides for identification and reduction to functional minimal size of the EFG domains of the subject EFG proteins to generate mimetics, e.g. peptide or non-peptide agents, which are able to disrupt binding of a polypeptide of the present invention with a EFG target protein. Thus, such mutagenic techniques as described above are also useful to map the determinants of EFG proteins which participate in protein-protein interactions involved in, for example, binding to a EFG target protein. To illustrate, the critical residues of a EFG protein which are involved in molecular recognition of the EFG target can be determined and used to generate EFG target-13P-derived peptidomimetics that competitively inhibit binding of the EFG to the EFG target. By employing, for example, scanning mutagenesis to map tile amino acid residues of a particular EFG protein involved in binding a EFG target, peptidomimetic compounds can be generated which mimic those residues in binding to a EFG target, and which, by inhibiting binding of the EFG protein to the EFG target protein, can interfere with the function of a EFG target in transcriptional regulation of one or more genes. For instance, non hydrolyzable peptide analogs of such residues can be generated using retro-inverse peptides (e.g., see U.S. Pat. Nos. 5,116,947 and 5,219,089; and Pallai et al. (1983) Int J Pept Protein Res 21:84-92), benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides.—Chemistry and Biology, G. R. Marshall ed., ESCOM Publisher: Leiden, Netherlands, 1988).

An isolated EFG protein, or a portion or fragment thereof, can be used as an immunogen to generate antibodies that bind EFG using standard techniques for polyclonal and monoclonal antibody preparation. Even if they are internal fungus protein, EFG proteins may induce an immunitary response in contact with the host organism. A full-length EFG protein can be used or, alternatively, the invention provides antigenic peptide fragments of EFG for use as immunogens. The antigenic peptide of EFG comprises at least 8 amino acid residues of an amino acid sequence of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170, and encompasses an epitope of EFG such that an antibody raised against the peptide forms a specific immune complex with EFG. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of EFG that are located on the surface of the protein, e.g., hydrophilic regions.

A EFG immunogen typically is used to prepare antibodies by immunizing a suitable subject, (e.g., rabbit, goat, mouse or other mammal) with the immunogen. An appropriate immunogenic preparation can contain, for example, recombinantly expressed EFG protein or a chemically synthesized EFG polypeptide. The preparation can further include an adjuvant, such as Freund's complete or incomplete adjuvant, or similar immunostimulatory agent. Immunization of a suitable subject with an immunogenic EFG preparation induces a polyclonal anti-EFG antibody response.

Accordingly, another aspect of the invention pertains to anti-EFG antibodies. The term “antibody” as used herein refers to immunoglobulin molecules and immunologically active portions of immunoglobulin molecules, i.e., molecules that contain an antigen binding site which specifically binds (immunoreacts with) an antigen, such as EFG polypeptides. Examples of immunologically active portions of immunoglobulin molecules include F(ab) and F(ab').sub.2 fragments which can be generated by treating the antibody with an enzyme such as pepsin. The invention provides polyclonal and monoclonal antibodies that bind EFG polypeptides. The term “monoclonal antibody” or “monoclonal antibody composition”, as used herein, refers to a population of antibody molecules that contain only one species of an antigen binding site capable of immunoreacting with a particular epitope of EFG polypeptides. A monoclonal antibody composition thus typically displays a single binding affinity for a particular EFG protein with which it immunoreacts.

The invention concerns antibody compositions, either polyclonal or monoclonal, capable of selectively binding, or selectively bind to an epitope-containing a polypeptide comprising a contiguous span of at least 6 amino acids, preferably at least 8 to 10 amino acids, more preferably at least 12, 15, 20, 25, 30, 40, 50, or 100 amino acids of a sequence of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170 (upper length according to the total length of each EFG protein). The invention also concerns a purified or isolated antibody capable of specifically binding to a mutated EFG protein or to a fragment or variant thereof comprising an epitope of the mutated EFG protein

Polyclonal anti-EFG antibodies can be prepared as described above by immunizing a suitable subject with a EFG immunogen. The anti-EFG antibody titer in the immunized subject can be monitored over time by standard techniques, such as with an enzyme linked immunosorbent assay (ELISA) using immobilized EFG proteins. If desired, the antibody molecules directed against EFG proteins can be isolated from the mammal (e.g., from the blood) and further purified by well known techniques, such as protein A chromatography to obtain the IgG fraction. At an appropriate time after immunization, e.g., when the anti-EFG antibody titers are highest, antibody-producing cells can be obtained from the subject and used to prepare monoclonal antibodies by standard techniques.

Any of the many well known protocols used for fusing lymphocytes and immortalized cell lines can be applied for the purpose of generating an anti-EFG monoclonal antibody (see, e.g., G. Galfre et al. (1977) Nature 266:55052; Gefter et al. Somatic Cell Genet., cited supra; Lerner, Yale J. Biol. Med, cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC.

Alternative to preparing monoclonal antibody-secreting hybridomas, a monoclonal anti-EFG antibody can be identified and isolated by screening a recombinant combinatorial immunoglobulin library (e.g., an antibody phage display library) with EFG to thereby isolate immunoglobulin library members that bind EFG genes. Kits for generating and screening phage display libraries are commercially available (e.g., the Pharmacia Recombinant Phage Antibody System, Catalog NO. 27-9400-01; and the Stratagene SurfZAP.™. Phage Display Kit, Catalog NO. 240612).

Additionally, recombinant anti-EFG antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

An anti-EFG antibody (e.g., monoclonal antibody) can be used to isolate EFG by standard techniques, such as affinity chromatography or immunoprecipitation. An anti-EFG antibody can facilitate the purification of natural EFG from cells and of recombinantly produced EFG expressed in host cells. Moreover, an anti-EFG antibody can be used to detect EFG protein (e.g., in a cellular lysate or cell supernatant) in order to evaluate the abundance and pattern of expression of the EFG protein. Anti-EFG antibodies can be used diagnostically to monitor protein levels in tissue as part of a clinical testing procedure, e.g., to, for example, determine the efficacy of a given treatment regimen.

### III. Recombinant Expression Vectors and Host Cells.

Another aspect of the invention pertains to vectors, preferably expression vectors, containing a nucleic acid encoding a EFG protein (or a portion thereof). Vectors may have particular use in the preparation of a recombinant protein of the invention.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a EFG nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990), the disclosure of which is incorporated herein by reference in its entirety. Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., EFG proteins, mutant forms of EFG proteins, fusion proteins, or fragments of any of the preceding proteins, etc.).

The recombinant expression vectors of the invention can be designed for expression of EFG proteins in prokaryotic or eukaryotic cells. For example, EFG proteins can be expressed in bacterial cells such as E. coli, insect cells (using baculovirus expression vectors) yeast cells, or mammalian cells. Suitable host cells are discussed further in Goeddel, Gene Expression Technology: Methods in Enzymology 185, Academic Press, San Diego, Calif. (1990), the disclosure of which is incorporated herein by reference in its entirety. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.), the disclosures of which are incorporated herein by reference in their entireties, which fuse glutathione S-transferase (GST), maltose E binding protein, or protein A, respectively, to the target recombinant protein.

Purified fusion proteins can be utilized in EFG activity assays, (e.g., direct assays or competitive assays, or to generate antibodies specific for EFG proteins, for example.

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to EFG mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1 (1) 1986, the disclosure of which is incorporated herein by reference in its entirety.

Of course, in the present invention, antisense vectors are particularly useful for inhibiting

EFG genes expression, most preferably A. fumigatus EFG genes.

Antisense constructs may be designed to bind to the promoter and other control regions, exons, introns or even exon-intron boundaries of a gene. Antisense RNA constructs, or DNA encoding such antisense RNAs, may be employed to inhibit gene transcription or translation or both within a host cell, either in vitro or in vivo, such as within a host animal, including a human subject.

Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that oligonucleotides of 8, 9, 10, 11, 12, 13, 14, 15, 16, 17, 18, 19, 20 or more base pairs will be used. One can readily determine whether a given antisense nucleic acid is effective at targeting of the corresponding host cell gene simply by testing the constructs in vitro to determine whether the endogenous gene's function is affected or whether the expression of related genes having complementary sequences is affected.

Another aspect of the invention pertains to host cells into which a recombinant expression vector of the invention has been introduced. The terms “host cell” and “recombinant host cell” are used interchangeably herein. It is understood that such term refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a EFG protein can be expressed in bacterial cells such as E. coli, insect cells, yeast or mammalian cells (such as Chinese hamster ovary cells (CHO) or COS cells or human cells).

Vector DNA can be introduced into prokaryotic or eukaryotic cells via conventional transformation or transfection techniques, that can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989, the disclosure of which is incorporated herein by reference in its entirety), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest.

A host cell of the invention, such as a prokaryotic or eukaryotic host cell in culture, can be used to produce (i.e., express) a EFG protein. Accordingly, the invention further provides methods for producing a EFG protein using the host cells of the invention. In one embodiment, the method comprises culturing the host cell of invention (into which a recombinant expression vector encoding a EFG protein has been introduced) in a suitable medium such that a EFG protein is produced. In another embodiment, the method further comprises isolating a EFG protein from the medium or the host cell.

In another embodiment, the invention encompasses providing a cell capable of expressing a EFG protein, culturing said cell in a suitable medium such that a EFG protein is produced, and isolating or purifying the EFG protein from the medium or cell.

In certain indications, it may be desirable to activate transcription at specific times after administration of the gene therapy vector. This may be done with such promoters as those that are hormone or cytokine regulatable.

### IV. Drug Screening Assays.

The invention provides a method (also referred to herein as a “screening assay”) for identifying inhibitors, i.e., candidate or test compounds or agents (e.g., preferably small molecules, but also peptides, peptidomimetics or other drugs) which bind to EFG proteins, have an inhibitory effect on, for example, EFG expression or preferably EFG activity, or have an inhibitory effect on, for example, the activity of an EFG target molecule. Assays may be cell based or non-cell based assays. Drug screening assays may be binding assays or more preferentially functional assays.

In preferred embodiments, an assay is a cell-based assay in which a cell which expresses a EFG protein or biologically active portion thereof is contacted with a test compound and the ability of the test compound to inhibit EFG activity determined. Determining the ability of the test compound to inhibit EFG activity can be accomplished by monitoring the bioactivity of the EFG protein or biologically active portion thereof.

The invention further encompasses compounds capable of inhibiting EFG activity. Inhibiting EFG activity refers to the inhibition of EFG gene expression such that fungus growth is inhibited. Preferably, a EFG inhibitor is a selective EFG inhibitor.

In a preferred embodiment, an inhibitor is capable of inhibiting EFG activity of at least one EFG protein of SEQ ID NO 3, 6, 9, 12, 15, 18, 21, 24, 27, 30, 33, 36, 39, 42, 45, 48, 51, 54, 57, 60, 94, 98, 102, 106, 110, 114, 118, 122, 126, 130, 134, 138, 142, 146, 150, 154, 158, 162, 166, 170, or a fragment or a variant thereof as previously described. Compounds will be assayed for the activities indicated in Table 1 and Table ibis.

For instance, the following standard enzymatic tests are appropriated : ATP dependant RNA helicase, guanylate kinase, RNA synthetase, SAM decarboxylase, protein kinase, ferro-chelatase.

Assays are made by using compounds already known to have an effect on the activity tested. For instance compounds known to inhibit protein kinase activity will be tested.

In one embodiment, the invention provides assays for screening candidate or test compounds which are target molecules of a EFG protein or polypeptide or biologically active portion thereof. In another embodiment, the invention provides assays for screening candidate or test compounds which bind to or modulate the activity of a EFG protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is used with peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. NO. 5,223,409), spores (Ladner U.S. Pat. NO. '409), plasmids (Cull et al. (1992) Proc Natl Acad Sci USA 89:1865-1869) or on phage (Scott and Smith (1990) Science 249:386-390); (Devin (1990) Science 249:404-406); (Cwirla et al. (1990) Proc. Natl. Acad. Sci. 87:6378-6382); (Felici (1991) J. Mol. Biol. 222:301-310); (Ladner supra.).

Determining the ability of the test compound to inhibit EFG activity can also be accomplished, for example, by coupling the EFG protein or biologically active portion thereof with a radioisotope or enzymatic label such that binding of the EFG protein or biologically active portion thereof to its cognate target molecule can be determined by detecting the labeled EFG protein or biologically active portion thereof in a complex. For example, compounds (e.g., EFG protein or biologically active portion thereof) can be labeled with .sup.125 I , .sup.35 S, .sup.14 C, or .sup.3H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., EFG protein or biologically active portion thereof) to interact with its cognate target molecule without the labeling of any of the interactants. For example, a microphysiometer can be used to detect the interaction of a compound with its cognate target molecule without the labeling of either the compound or the receptor. McConnell, H. M. et al. (1992) Science 257:1906-1912. A microphysiometer such as a cytosensor is an analytical instrument that measures the rate at which a cell acidifies its environment using a light-addressable potentiometric sensor (LAPS). Changes in this acidification rate can be used as an indicator of the interaction between compound and receptor.

In a preferred embodiment, the assay comprises contacting a cell which expresses a EFG protein or biologically active portion thereof, with a target molecule to form an assay mixture, contacting the assay mixture with a test compound, and determining the ability of the test compound to inhibit the activity of the EFG protein or biologically active portion thereof, wherein determining the ability of the test compound to inhibit the activity of the EFG protein or biologically active portion thereof, comprises determining the ability of the test compound to inhibit a biological activity of the EFG expressing cell (e.g., determining the ability of the test compound to inhibit transduction or protein: protein interactions).

In another preferred embodiment, the assay comprises contacting a cell which is responsive to a EFG protein or biologically active portion thereof, with a EFG protein or biologically-active portion thereof, to form an assay mixture, contacting the assay mixture with a test compound, and determining the ability of the test compound to modulate the activity of the EFG protein or biologically active portion thereof, wherein determining the ability of the test compound to modulate the activity of the EFG protein or biologically active portion thereof comprises determining the ability of the test compound to modulate a biological activity of the EFG gene-responsive cell (e.g., determining the ability of the test compound to modulate signal transduction or protein:protein interactions).

In another embodiment, an assay is a cell-based assay comprising contacting a cell expressing a EFG target molecule with a test compound and determining the ability of the test compound to modulate (e.g. stimulate or inhibit) the activity of the EFG target molecule. Determining the ability of the test compound to modulate the activity of a EFG target molecule can be accomplished, for example, by determining the ability of the EFG protein to bind to or interact with the EFG target molecule.

Determining the ability of the EFG protein to bind to or interact with a EFG target molecule can be accomplished by one of the methods described above for determining direct binding. In a preferred embodiment, determining the ability of the EFG protein to bind to or interact with a EFG target molecule can be accomplished by determining the activity of the target molecule. For example, the activity of the target molecule can be determined by detecting induction of a cellular second messenger of the target, detecting catalytic/enzymatic activity of the target an appropriate substrate, detecting the induction of a reporter gene (comprising a target-responsive regulatory element operatively linked to a nucleic acid encoding a detectable marker, e.g., luciferase), or detecting a target-regulated cellular response, for example, signal transduction or protein:protein interactions.

In yet another embodiment, an assay of the present invention is a cell-free assay in which a EFG protein or biologically active portion thereof is contacted with a test compound and the ability of the test compound to bind to the EFG protein or biologically active portion thereof is determined. Binding of the test compound to the EFG protein can be determined either directly or indirectly as described above. In a preferred embodiment, the assay includes contacting the EFG protein or biologically active portion thereof with a known compound which binds EFG (e.g., a EFG target molecule) to form an assay mixture, contacting the assay mixture with a test compound, and determining the ability of the test compound to interact with a EFG protein, wherein determining the ability of the test compound to interact with a EFG protein comprises determining the ability of the test compound to preferentially bind to EFG or biologically active portion thereof as compared to the known compound.

In another embodiment, the assay is a cell-free assay in which a EFG protein or biologically active portion thereof is contacted with a test compound and the ability of the test compound to modulate (preferably inhibit) the activity of the EFG protein or biologically active portion thereof is determined Determining the ability of the test compound to modulate the activity of a EFG protein can be accomplished, for example, by determining the ability of the EFG protein to bind to a EFG target molecule by one of the methods described above for determining direct binding. Determining the ability of the EFG protein to bind to a EFG target molecule can also be accomplished using a technology such as real-time Biomolecular Interaction Analysis (BIA). Sjolander, S, and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of a EFG protein can be accomplished by determining the ability of the EFG protein to further modulate the activity of a downstream effector (e.g., a growth factor mediated signal transduction pathway component) of a EFG target molecule. For example, the activity of the effector molecule on an appropriate target can be determined or the binding of the effector to an appropriate target can be determined as previously described.

In yet another embodiment, the cell-free assay involves contacting a EFG protein or biologically active portion thereof with a known compound which binds the EFG protein to form an assay mixture, contacting the assay mixture with a test compound, and determining the ability of the test compound to interact with the EFG protein, wherein determining the ability of the test compound to interact with the EFG protein comprises determining the ability of the EFG protein to preferentially bind to or modulate the activity of a EFG target molecule.

The cell-free assays of the present invention are amenable to use of both soluble and/or membrane-bound forms of isolated proteins (e.g. EFG proteins or biologically active portions thereof or molecules to which EFG targets bind). In the case of cell-free assays in which a membrane-bound form an isolated protein is used it may be desirable to utilize a solubilizing agent such that the membrane-bound form of the isolated protein is maintained in solution.

In more than one embodiment of the above assay methods of the present invention, it may be desirable to immobilize either EFG or its target molecule to facilitate separation of complexed from uncomplexed forms of one or both of the proteins, as well as to accommodate automation of the assay. Binding of a test compound to a EFG protein, or interaction of a EFG protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/EFG fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or EFG protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of EFG binding or activity determined using standard techniques.

Other techniques for immobilizing proteins on matrices can also be used in the screening assays of the invention. For example, either a EFG protein or a EFG target molecule can be immobilized utilizing conjugation of biotin and streptavidin. Biotinylated EFG protein or target molecules can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with EFG protein or target molecules but which do not interfere with binding of the EFG protein to its target molecule can be derivatized to the wells of the plate, and unbound target or EFG protein trapped in the wells by antibody conjugation. Methods for detecting such complexes, in addition to those described above for the GST-immobilized complexes, include immunodetection of complexes using antibodies reactive with the EFG protein or target molecule, as well as enzyme-linked assays which rely on detecting an enzymatic activity associated with the EFG protein or target molecule.

In another embodiment, modulators of EFG expression are identified in a method wherein a cell is contacted with a candidate compound and the expression of EFG mRNA or protein in the cell is determined. The level of expression of EFG mRNA or protein in the presence of the candidate compound is compared to the level of expression of EFG mRNA or protein in the absence of the candidate compound. The candidate compound can then be identified as a modulator of EFG expression based on this comparison. For example, when expression of EFG mRNA or protein is greater (statistically significantly greater) in the presence of the candidate compound than in its absence, the candidate compound is identified as a stimulator of EFG mRNA or protein expression. Alternatively, when expression of EFG mRNA or protein is less (statistically significantly less) in the presence of the candidate compound than in its absence, the candidate compound is identified as an inhibitor of EFG mRNA or protein expression. The level of EFG mRNA or protein expression in the cells can be determined by methods described herein for detecting EFG mRNA or protein.

In yet another aspect of the invention, the EFG proteins can be used as “bait proteins” in a two-hybrid assay or three-hybrid assay (see, e.g., U.S. Pat. NO. 5,283,317; Zervos et al. (1993) Cell 72:223-232).

This invention further pertains to novel agents identified by the above-described screening assays and to processes for producing such agents by use of these assays. Accordingly, in one embodiment, the present invention includes a compound or agent obtainable by a method comprising the steps of any one of the aformentioned screening assays (e.g., cell-based assays or cell-free assays). For example, in one embodiment, the invention includes a compound or agent obtainable by a method comprising contacting a cell which expresses a EFG target molecule with a test compound and the determining the ability of the test compound to bind to, or modulate the activity of, the EFG target molecule. In another embodiment, the invention includes a compound or agent obtainable by a method comprising contacting a cell which expresses a EFG target molecule with a EFG protein or biologically-active portion thereof, to form an assay mixture, contacting the assay mixture with a test compound, and determining the ability of the test compound to interact with, or modulate the activity of, the EFG target molecule. In another embodiment, the invention includes a compound or agent obtainable by a method comprising contacting a EFG protein or biologically active portion thereof with a test compound and determining the ability of the test compound to bind to, or modulate (e.g., stimulate or inhibit) the activity of, the EFG protein or biologically active portion thereof. In yet another embodiment, the present invention included a compound or agent obtainable by a method comprising contacting a EFG protein or biologically active portion thereof with a known compound which binds the EFG protein to form an assay mixture, contacting the assay mixture with a test compound, and determining the ability of the test compound to interact with, or modulate the activity of the EFG protein.

Accordingly, it is within the scope of this invention to further use an agent identified as described herein in an appropriate animal model. For example, an agent identified as described herein (e.g., a EFG modulating agent, an antisense EFG nucleic acid molecule, a EFG-specific antibody, or a EFG-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

The present invention also pertains to uses of novel agents identified by the above-described screening assays for diagnoses, prognoses, and treatments as described herein. Accordingly, it is within the scope of the present invention to use such agents in the design, formulation, synthesis, manufacture, and/or production of a drug or pharmaceutical composition for use in diagnosis, prognosis, or treatment, as described herein. For example, in one embodiment, the present invention includes a method of synthesizing or producing a drug or pharmaceutical composition by reference to the structure and/or properties of a compound obtainable by one of the above-described screening assays. For example, a drug or pharmaceutical composition can be synthesized based on the structure and/or properties of a compound obtained by a method in which a cell which expresses a EFG target molecule is contacted with a test compound and the ability of the test compound to bind to, or modulate the activity of, the EFG target molecule is determined. In another exemplary embodiment, the present invention includes a method of synthesizing or producing a drug or pharmaceutical composition based on the structure and/or properties of a compound obtainable by a method in which a EFG protein or biologically active portion thereof is contacted with a test compound and the ability of the test compound to bind to, or inhibit the activity of, the EFG protein or biologically active portion thereof is determined

### V. Methods of Treatment.

EFG inhibitors identified according to the methods in the section titled “Drug Screening Assays” can be further tested for their ability to ameliorate or prevent the pathologies associated to Aspergillus fungus, and more particularly to A. fumigatus, namely invasive pulmonary aspergillosis.

An “individual” treated by the methods of this invention is a vertebrate, particularly a mammal (including model animals of human disease, farm animals, sport animals, and pets), and typically a human.

“Treatment” refers to clinical intervention in an attempt to alter the natural course of the individual being treated, and may be performed either for prophylaxis or during the course of clinical pathology. Desirable effects include preventing occurrence or recurrence of disease, alleviation of symptoms, diminishment of any direct or indirect pathological consequences of the disease, such as hyperresponsiveness, inflammation, or necrosis, lowering the rate of disease progression, amelioration or palliation of the disease state, and remission or improved prognosis. The “pathology” associated with a disease condition is anything that compromises the well-being, normal physiology, or quality of life of the affected individual.

Treatment is performed by administering an effective amount of a EFG inhibitor. An “effective amount” is an amount sufficient to effect a beneficial or desired clinical result, and can be administered in one or more doses.

The criteria for assessing response to therapeutic modalities employing the compositions of this invention are dictated by the specific condition, measured according to standard medical procedures appropriate for the condition.

### VI. Pharmaceutical Compositions.

Compounds capable of inhibiting EFG activity, preferably small molecules but also including peptides, EFG antisens nucleic acid molecules, EFG proteins inhibitors, and anti-EFG antibodies (also referred to herein as “active compounds”) of the invention can be incorporated into pharmaceutical compositions suitable for administration. Such compositions typically comprise a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL.TM. (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Where the active compound is a protein, peptide or anti-EFG antibody, sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. For administration by inhalation, the compounds are delivered in the form of an aerosol spray from pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Most preferably, active compound is delivered to a subject by intravenous injection.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art, for example, as described in U.S. Pat. NO. 4,522,811, or are commercially available.

It is especially advantageous to formulate oral or preferably parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

### VII. Diagnostic and Prognostic Uses

The nucleic acid molecules, proteins, protein homologues, and antibodies described herein can be used in one or more of the following methods: diagnostic assays, prognostic assays, monitoring clinical trials, and pharmacogenetics; and in drug screening and methods of treatment (e.g., therapeutic and prophylactic) as further described herein.

The invention provides diagnostic and prognositc assays for detecting EFG members, as further described. Also provided are diagnostic and prognostic assays for detecting interactions between EFG members and EFG target molecules.

The isolated nucleic acid molecules of the invention can be used, for example, to detect EFG mRNA (e.g., in a biological sample) or a genetic alteration in a EFG gene, and to modulate a EFG activity, as described further below. The EFG proteins can be used to screen for drugs or compounds which modulate, preferably inhibit EFG activity.

Accordingly one embodiment of the present invention involves a method of use (e.g., a diagnostic assay, prognostic assay, or a prophylactic/therapeutic method of treatment) wherein a molecule of the present invention (e.g., a EFG protein, EFG nucleic acid, or most preferably a EFG inhibitor or activator) is used.

### VIII. Fungicidal Compositions.

The invention also deals with fungicidal incorporating at least one compound capable to inhibit fungal growth, by inhibiting EFG genes, in particular in diseases of plants due to phytopathogenic fungi, namely Botrytis cinerea, Mycosphaerella graminicola, Stagnospora nodorum, Blumeria graminis, Colleotrichum lindemuthianum, Puccinia graminis, Leptosphaeria maculans, Fusarium oxysporum, Fusarium graminearum, Venturia inaequalis, most preferably fungi of the genus Magnaporthe, even most preferably Magnaporthe grisea.

The invention thus also provides a method of combating fungi at a locus infested or liable to be infested therewith, which comprises applying to the locus the compound of the invention.

The invention also provides an agricultural composition comprising the compound of the invention in admixture with an agriculturally acceptable diluent or carrier.

The composition can comprise one or more additional active ingredients, for example compounds known to possess plant-growth regulant, herbicidal, fungicidal (such as metalaxyl, oxadixyl, ofurace, benalaxyl and furalaxyl; cymoxanil; mancozeb; chlorothalonil; folpet; captan; famoxadone; fenamidone; spiroxamine; fluazinam; dimethomorph; strobilurins, such as kresoxim-methyl, azoxystrobin and trifloxystrobin, pyrimethanil, cyprodinil; mepanipyrim; and iprodione), insecticidal or acaricidal properties.

The composition of the invention may include for example a dispersing agent, emulsifying agent or wetting agent. Usually they are in the form of an aqueous concentrate.

The concentration of the active ingredient in the composition of the present invention, as applied to plants is preferably within the range of 0.0001 to 1.0 percent by weight, especially 0.0001 to 0.01 percent by weight. In a primary composition, the amount of active ingredient can vary widely and can be, for example, from 5 to 95 percent by weight of the composition.

In the method of the invention, the compound is generally applied to seeds, plants or their habitat. Thus, the compound can be applied directly to the soil before, at or after drilling so that the presence of active compound in the soil can control the growth of fungi which may attack seeds. When the soil is treated directly the active compound can be applied in any manner which allows it to be intimately mixed with the soil such as by spraying, by broadcasting a solid form of granules, or by applying the active ingredient at the same time as drilling by inserting it in the same drill as the seeds. A suitable application rate is within the range of from 5 to 1000 g per hectare, more preferably from 10 to 500 g per hectare.

Alternatively, the active compound can be applied directly to the plant by, for example, spraying or dusting either at the time when the fungus has begun to appear on the plant or before the appearance of fungus as a protective measure. In both such cases the preferred mode of application is by foliar spraying. It is generally important to obtain good control of fungi in the early stages of plant growth as this is the time when the plant can be most severely damaged. The spray or dust can conveniently contain a pre- or post-emergence herbicide if this is thought necessary. Sometimes, it is practicable to treat the roots of a plant before or during planting, for example, by dipping the roots in a suitable liquid or solid composition. When the active compound is applied directly to the plant a suitable rate of application is from 0.025 to 5 kg per hectare, preferably from 0.05 to 1 kg per hectare.

Having generally described this invention, a further understanding can be obtained by reference to certain specific examples which are provided herein for purposes of illustration only, and are not intended to be limiting unless otherwise specified.

### Example 1

### A. fumigatus Strain Construction

Media and growth conditions were as follows. A. fumigatus strains were propagated at 37° C. on complete medium or minimal medium with 0.5 mM of various nitrogen sources (sodium glutamate, ammonium tartrate, sodium nitrate, sodium nitrite and hypoxanthine) (Cove 1966). Uridine and uracil were added at a concentration of 5 mM when appropriate. Liquid cultures used for DNA-mediated transformation and genomic DNA preparation were grown in YG (0.5% Yeast Extract, 2% glucose). DNA-mediated transformation was achieved either on protoplasts as described previously (d′Enfert 1996; Osmani et al. 1987) or by electroporation of intact conidia as described (Weidner et al. 1998).

A. fumigatus stable diploids appropriate for transposon mutagenesis were obtained using the following procedure. In summary, insertional mutagenesis (Weidner et a/0.1998) of strain CEA17 has led to the isolation of spore color mutants CEA82 and CEA85. White strain CEA88 and reddish strain CEA94 are chlorate resistant derivatives of CEA82 and CEA85 with uncharacterized mutations in a gene involved in the biosynthesis of the molybdene cofactor (cnx) and in the nitrate reductase gene (niaD), respectively. Strains CEA125 (w1, cnx1, pyrG1) and CEA129 (r7, niaD2, pyrG1) were obtained from strains CEA88 and CEA94 by growth on media containing 5-fluoro-orotic acid (1 mg/ml) which selects for pyrG mutants. Simultaneous growth of CEA125 and CEA129 on minimal medium with nitrate as sole nitrogen source yielded heterokaryons that produced grey-green spores similar to that of A. fumigatus haploid wild-type strains. This led to the isolation of the stable diploid strain CEA131 (w1/+, +/r7, cnx1/+, +/niaD2, pyrG1/pyrG1). A chlorate resistant derivative of CEA131 was identified that was unable to use nitrate as the sole nitrogen source and was defective at both niaD alleles. This strain is referred to as CEA153 (w1/+, +/r7, cnx1/+, niaD4/niaD2, pyrG1/pyrG1). On minimal medium containing nitrate, spontaneous reversion of the haploid strain CEA113 and the diploid strain CEA153 for nitrate utilization was not observed.

### Example 2

### In Vivo Transposon Mutagenesis in A. fumigatus

Plasmid pNIL160 has been described³⁰. A 2.2 kb BamHI fragment from ppyrG containing the A. nidulans pyrG gene was cloned at the NheI restriction site in impala160, yielding pNIpyr. NdeI-digested pNIpyr was introduced into genomic DNA of strains CEA113 and CEA153 by electroporation of intact conidia as described¹⁹ yielding the haploid strain CEA165 and the diploid strains CEA225, 226 and 227, respectively. impala160::pyrG transposition occurs on minimal medium containing nitrate supplemented with 0.02% Triton X-100 at 37° C. for 3 days. Genomic DNA preparation and Southern analysis techniques were essentially performed according to Sambrook et al. (1989) and Ausubel et al. (1992).

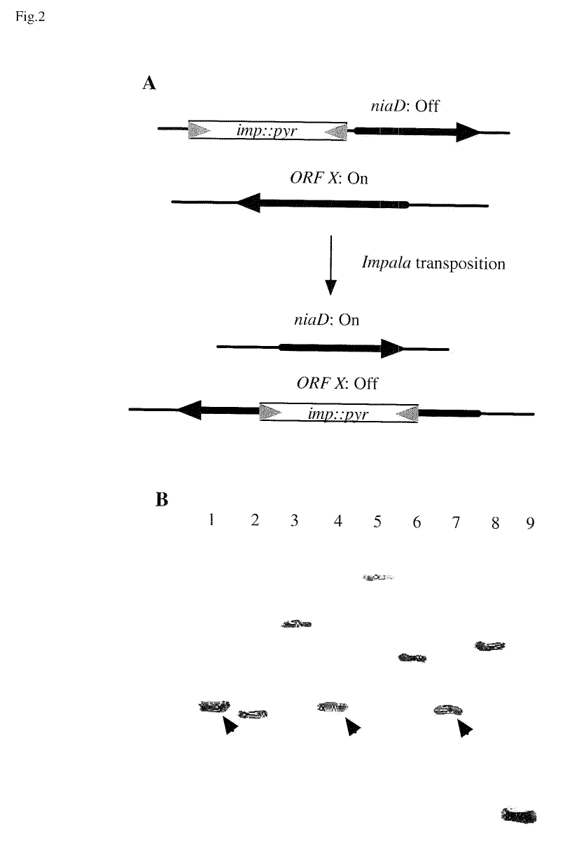

Plasmid pNIpyr, a derivative of pNIL160³⁰, carries the A. nidulans niaD gene encoding nitrate reductase with a copy of impala160 inserted 10 by upstream of the translation initiation codon of niaD and modified by the insertion of the A. nidulans pyrG gene between the 3′-end of the transposase-encoding gene and the 3′ inverted terminal repeat. pNIpyr was introduced in A. fumigatus strain CEA153, a stable pyrG, niaD diploid strain, heterozygous for spore color markers. Because of the insertion of impala160::pyrG into the niaD promoter, the niaD allele carried by pNIpyr is not functional. However, when diploid transformants were grown on selective minimal medium with nitrate as sole nitrogen source, pyrG⁺, NiaD⁺ revertants were observed at a frequency of 10⁻⁵-10⁻⁶. Southern analysis showed that these transformants have only one copy of impala160::pyrG integrated into their genome and that all the revertants resulted of impala transposition events from the niaD promoter to an apparent random site located elsewhere in the A. fumigatus genome (FIG. 2). Sequence analysis of the niaD promoter region in all revertants revealed a footprint of usually 5 by associated with impala excision. Characterization of integration targets by sequencing and comparison to public genomic sequences (Tables 1 and 1bis, and data not shown) revealed that the transposition of impala160::pyrG in A. fumigatus 1) occurs at a genomic TA dinucleotide which is duplicated during the integration process; 2) is apparently random without sequence preference (except the TA dinucleotide); and 3) is not associated with genomic rearrangements. All of these characteristics are typical for transposition of the Tcl-mariner family members and was previously observed for impala160 transposition events in F. oxysporum²⁸, A. nidulans²⁹ and M. grisea³⁰. Therefore, impala appears as the most suitable tool to generate random tagged mutation in A. fumigatus since insertional mutagenesis through DNA-mediated transformation results in various types of rearrangements of the transforming and genomic DNA.

### Example 3

### Parasexual Genetic Screening

Haploidization of A. fumigatus diploid strains was conducted on selective haploidization medium [complete medium containing 1.2 μg/ml benomyl (ALDRICH, 10 mg/ml in DMSO)] or on non-selective haploidization medium (selective haploidization medium plus uridine and uracil) for 5 days at 37° C. Haploid progenies are easily identified by the production of white and reddish-colored sectors after haploidization of grey-green diploid strains.

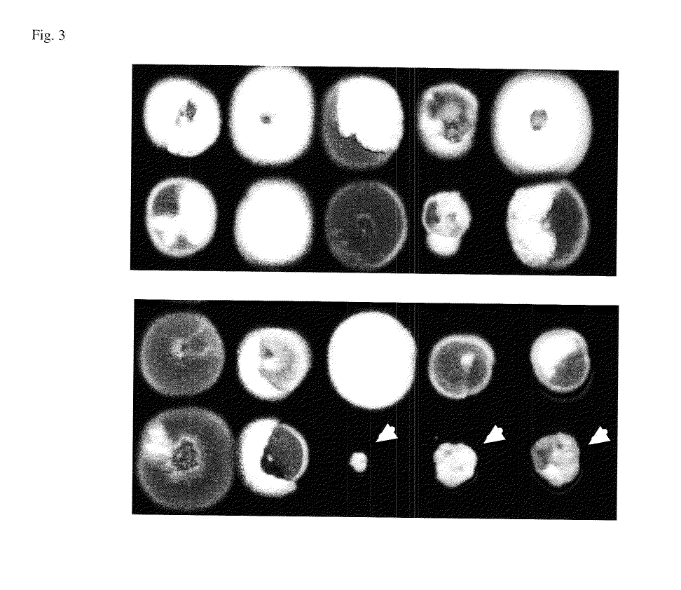

Three diploid transformants (namely A. fumigatus CEA225, CEA226 and CEA227) were used to generate a collection of random diploid heterozygous revertants. Haploidization of heterozygous strains was induced by the destabilizing reagent benomyl and results from mitotic chromosomal non-disjunction^{18,31}. Since each revertant has a single mutated chromosomal locus tagged by impala160::pyrG, parasexual genetics on selective and non-selective haploidization media permits to distinguish insertions that occur in non-essential versus essential chromosomal targets (FIG. 1). Strains CEA225, CEA226, CEA227 and 97% of 2,386 revertants showed no difference on selective and non-selective haploidization media after two independent tests, indicating that integration of pNIpyr into the genome of the parental transformants and integration of impala160::pyrG in the diploid revertants had not occurred into an essential chromosomal region. On selective haploidization medium, 73 revertants (3%) did not yield haploid conidia as evidenced by the absence of colored sectors (FIG. 3). Diploid strains of A. fumigatus are hypersensitive to benomyl and only haploid strains can grow at the benomyl concentration used³¹. However, the transient formation of aneuploids leads to the formation of a cal on selective haploidization medium that potentially over-grow haploid strains with morphological defects. To identify mutants which could have been selected in our screening because of the slow growth of haploid progenies rather than the lethality of the insertion, ca. 10⁶ haploid progenies obtained on non-selective haploidization medium were tested for the presence of impala160::pyrG by growth on selective medium. Twenty nine diploid revertants (29/2,386=1.2%) never yielded haploid pyrG⁺ progenies and were defined as carrying an integration of impala160::pyrG into a chromosomal locus essential for A. fumigatus growth.

### Example 4A

### Sequence Determination

Genomic sequences bordering impala160::pyrG are determined by an adaptation of a two-step PCR strategy developed by Chun et al.³⁶ and using transposon specific primers. More precisely concerning the two step PCR, first, ca. 100 ng of genomic DNA were amplified in 50 μl using oligonucleotides ppyrl and PCRall or ppyr3 and PCRall (4 μmol/μl final) and the following amplification protocol: a denaturation step at 94° C. for 3 min. followed by 5 cycles of the following steps: denaturation at 94° C. for 30 sec, annealing at 35° C. for 30 sec, extension at 72° C. for 1 min, and 30 cycles of the following steps: denaturation at 94° C. for 30 sec, annealing at 45° C. for 30 sec, extension at 72° C. for 1 min. A last elongation step was done at 72° C. for 3 min. Final concentrations for MgCl₂ and dNTPs were 3 mM and 0.2 mM, respectively. One microliter of the PCR reaction was subjected to a second amplification using similar reaction conditions and oligonucleotides ppyr2 and PCRa12N (if ppyr1 and PCRall had been used in the first reaction) or ppyr4 and PCRa12N (if ppyr3 and PCRall had been used in the first reaction). The following amplification protocol was used: 30 cycles of the following steps: denaturation at 94° C. for 30 sec, annealing at 60° C. for 30 sec, extension at 72° C. for 1 min. A last elongation step was done at 72° C. for 3 min. In some instances, oligonucleotides PCRa13, PCRa14, and PCRa15 were used in place of PCRall. PCR products were separated by electrophoresis on a 2% TBE-agarose gel and major PCR products were purified with the Qiaquick Gel Purification Kit (Qiagen, France) according to the supplier's instructions. Purified PCR products are sequenced using ppyr2 or ppyr4 as primers (ESGS, Evry, France). Nucleotide sequences obtained in this manner and trimmed for ppyrG sequences are compared using blastx or blastn (Altschul et al. 1990) to protein databases and to the preliminary sequence data of the A. fumigatus genome project which were obtained from The Institute for Genomic Research (TIGR) website (http://www.tigr.org).

More precisely concerning the transposon specific primers, two primers were used [primers Imp 1: ATGAAGGCGTAAGTTCCTTGC (SEQ ID NO. 61) and Imp2: GTGTGGAGGAAGAAAGAGC (SEQ ID NO. 62)]. Sequencing reactions were performed by ESGS (Evry, France) with the primer Imp2 directly on the major PCR product purified from agarose gel using the Qiaquick gel extraction kit (QIAGEN). After elimination of transposon sequences, genomic tags were compared to the A. fumigatus TIGR genomic data (www.tigr.org). Results present in this study were obtained from the sequence release of Nov. 14, 2001. At this time, the shotgun sequencing of A. fumigatus progressed to 6× sequence coverage (28.7 Mb in 1,578 assemblies of over 1,000 by in size). From TIGR data, specific primers were designed and used in standard PCR reactions and confirmed the absence of genomic rearrangements after transposon integration and the presence of a wild type chromosomal locus in each of the diploid revertants tested.

TABLE 2
Primer sequences

| | |
|---|---|
| ppyrl (5′end) | GGAAGACGGGCAGTTAGTCC |
| | (SEQ ID No. 63) |
| ppyr3 (3′end) | CCCAGGCTTTACACTTTATGC |
| | (SEQ ID No. 64) |
| PCRal 1 | GGCCACGCGTCGACTAGTAG(N)₁₀GATAT |
| | (SEQ ID No. 65) |
| PCRal 3 | GGCCACGCGTCGACTAGTAC(N)₁₀ACGTC |
| | (SEQ ID No. 66) |
| PCRal 4 | GGCCACGCGTCGACTAGTAC(N)₁₀TGGAC |
| | (SEQ ID No. 67) |
| PCRal 5 | GGCCACGCGTCGACTAGTAC(N)₁₀ACGTG |
| | (SEQ ID No. 68) |
| ppyr2N (5′end) | CGAAGTTGACGTTCAGTATGC |
| | (SEQ ID No. 69) |
| ppyr4N (3′end) | TGACCATGATTACGCCAAGC |
| | (SEQ ID No. 70) |
| PCRal 2N | GGCCACGCGTCGACTAGTAC |
| | (SEQ ID No. 71) |

Ppyr primers are specific of the ppyrG plasmid used for insertional mutagenesis. The pentamer at the 5′ end of the random primer (PCRal1, 3, 4 and 5) is expected to bind once in a kb. It is chosen according to the GC % of A. fumigatus (ca.50%) and does not occur in the region of ppyrG located between ppyr primers and the linearized plasmid end.

### Example 4B

### Characterization of A. fumigatus Essential Genes

Genomic sequences bordering impala160::pyrG were obtained for 21 of the 29 diploid strains mentioned above. Except for one strain (4-1-3), corresponding genomic regions were identified (Table 1 and Table 1bis) in the public preliminary sequence data of the A. fumigatus genome available at The Institute for Genomic Research (http://www.tigr.org). Similarity searches of the NCBI non-redundant sequence database performed using the BLASTx algorithm³² identified three main categories of insertional mutants (Tables 1 and 1bis). The first category includes 15 strains that have an insertion of impala160::pyrG into genes with homologues in other fungal species. The second category is composed of three strains with impala160::pyrG integration occurring into intergenic regions. The last category includes two strains with impala160::pyrG integration in genes without homologues in public databases and classified as A. fumigatus specific essential genes.

In nine of the fifteen strains of the first category, integration of impala160::pyrG occurs into homologues of genes demonstrated as essential for S. cerevisiae growth (Tables 1 and ibis). These genes are involved in a broad range of essential biological processes such as protein synthesis (YGL245W), protein maturation (WBP1) and protein transport (SRP101), nuclear architecture (NAR1), RNA processing (DBD10), nucleotide metabolism (GLIK1), chromatine structure (RSC9) and cell cycle control (CDC27). Four additional impala160::pyrG integrations occur into genes encoding homologues of non-essential S. cerevisiae proteins for which essentiality in A. fumigatus is not unexpected: ribosomal proteins RPL1 and RPL17 are duplicated in the yeast genome and therefore are not independently essential although the double mutation is lethal; a null mutation of MSW1 encoding the yeast tryptophanyl-tRNA synthetase localized in mitochondria leads to a slow growth phenotype; and S. cerevisiae gosl null mutant fail to germinate at 37° C., the temperature of the lethality screen.

Two genes encoding homologues of S. cerevisiae proteins that are not essential for yeast growth, namely Rim11p and Yfl034w (Tables 1 and 1bis), have been identified as essential for A. fumigatus growth. These proteins are conserved among lower eukaryotes but their role has not been precisely evaluated yet. Analysis of the Rim11p and its Schizosaccharomyces pombe counterpart (SKP1) suggest that these protein might be involved in spore formation and for mitosis, although they are not essential for viability in these two species. That the corresponding homologues are essential in A. fumigatus might be explained by their implication in additional essential pathways and for discrepencies in the contribution of similar biological pathways to the biology of A. fumigatus and other lower eukaryotes.

The second class of mutants includes three revertants in which transposon integration occurs in the vicinity (<200 bp) of the deduced translation initiation codon of three genes which are likely to be essential for A. fumigatus growth based to their homology to genes essential for S. cerevisiae growth: RPL14 (revertant 10-304) encodes an essential ribosomal protein and COX10 (revertant 10-175) and HEMI5 (revertant 11-4-9) are required for heme biosynthesis (Table 1). It is likely that impala integration prevents proper expression of these three genes but the inventors cannot exclude an additional effect on genes divergently transcribed from these intergenic regions.

### Example 5

### Impala Transposition Characteristics

The correlation between the parasexual genetics phenotype and the nature of the mutated genes supports the idea that genes genuinely essential for A. fumigatus growth can be identified among diploid heterozygous mutants. However, the proportion of impala160::pyrG integration in essential genes observed in this study (1.2%) is lower than expected. In S. cerevisiae, 17% of the 6,200 genes are essential¹³. Considering a similar frequency of essential genes in A. fumigatus, a genome size of 30 Mb with approximately 8000 genes, the inventors have estimated at 8 to 10% the frequency of heterozygous diploids that should have an essential phenotype after parasexual genetics. In an attempt to understand the observed lower frequency, the inventors have characterized insertions of impala160::pyrG in different contexts. In fourteen diploid revertants not classified as essential but that showed an altered growth pattern on selective haploidization medium only two revertants had an insertion of impala160::pyrG in ORFs greater than two kb and these were not similar to known proteins. Interestingly, five impala160::pyrG integration (36%) were located 5′ (<200 bp) of the deduced translation initiation codon of genes which have homologues in databases, including a tRNA seryl transferase essential in S. cerevisiae. In 18 random diploid strains with a non-essential phenotype only one transposon insertion occurred in a protein-coding region, corresponding to the homologue of the non-essential A. nidulans amdA gene, while five insertions (28%) were found 5′ (<300 bp) of start codons of homologues of non-essential S. cerevisiae genes (data not shown). Similar results have been obtained upon analysis of impala160::pyrG integrations in a haploid A. fumigatus strain (data not shown). These results suggest that impala160::pyrG has a tendency to insert preferentially into non-coding regions thus resulting in phenotypically silent mutations as already observed for other transposons³⁴. However, the inventors positive screening by parasexual analysis enrich for insertions that lie predominantly in ORFs.

In summary, through the analysis of 2,364 A. fumigatus diploid insertional mutants the inventors have been able to identify 21 previously uncharacterized essential genes (SEQ ID NO 1, 4, 7, 10, 13, 16, 19, 22, 25, 28, 31, 34, 37, 40, 43, 46, 49, 52, 55, 58, 92, 96, 100, 104, 108, 112, 116, 120, 124, 128, 132, 136, 140, 144, 148, 152, 156, 160, 164, 168) several of which could not have been predicted as essential for A. fumigatus growth based on the knowledge gained from studies in other fungal species. Despite an apparent not truly random distribution of the impala element, transposon mutagenesis is a promising tool for insertional mutagenesis in filamentous fungi. A large scale genomic approach is now underway for the systematic identification of essential A. fumigatus genes by automatisation of the strategy. This represent an important step for the definition of novel antifungal treatments. Interestingly, the inventors have observed that some heterozygous diploid strains with an integration of impala in genes necessary for efficient growth show reduced growth compared to a parental diploid strain (data not shown). In S. cerevisiae or C. albicans, several heterozygous diploids with a mutation in a gene essential for growth also show reduced growth or increased sensitivity to drugs targeting the corresponding gene product^{15,35}. This phenomenon, referred to as haploinsufficiency, can form the basis for the identification of antifungal components that target the product of the mutated gene^{15,35}. In order to achieve the results described the inventors used the following experimental protocols.

### Example 6

### cDNA Analysis

PCR were carried on DNA prepared from a A. fumigatus cDNA library in order to:

1) check for expression of a subset of genes identified by transposon;
2) confirm the location of postulated exon/intron splice sites; and
3) identify transcription start sites and polyadenylation sites.

The A. fumigatus cDNA library was obtained from M. Monod (CHUV, Lausanne, Switzerland). It was constructed by InVitrogen using cDNA prepared from A. fumigatus strain Y1090 and Lambda gT11 as the cloning vector. Following amplification, DNA of the library was prepared using the Qiagen Lambda Midi Kit.

PCR were performed on an aliquote of the prepared DNA using standard reaction conditions. PCR used universal primers (Gt11f1, Gt11f3, gt11Rev) corresponding to regions of Lambda flanking the cDNA cloning sites and primers specific to each of the candidate genes.

PCR primers used herein are listed in Table 3 below:

TABLE 3
Primer sequences

| Oligo- | | |
|---|---|---|
| nucleotide | 5′-3′ sequence | |
| 10.175.2 | GTTGGATCTTTGGGTTCTG | (SEQ ID No. 72) |
| 10.304.4 | CGCGAATCTGATGACATAGC | (SEQ ID No. 73) |
| 10.4.20.2 | CTCTTCGCTTCATCGTACCC | (SEQ ID No. 74) |
| 11.4.9.4 | ATTAGTCCATGCGAGCATCC | (SEQ ID No. 75) |
| 11.6.20.2 | GCCTGAGCCTAGTCCATCAC | (SEQ ID No. 76) |
| 2.1.1.1 | CTCGCAGGTCGATTTCACTC | (SEQ ID No. 77) |
| 2.1.1.2 | GGAGGAAACCTTGTCACCAC | (SEQ ID No. 78) |
| 2.1.1.5 | TACCGAGAAGGAGGTCATGG | (SEQ ID No. 79) |
| 2.1.1.6 | TCCAGTCAAGGTTGGTGATG | (SEQ ID No. 80) |
| 2.1.1.9 | CGAGACCATCCTACCTCAG | (SEQ ID No. 81) |
| 4.3.4.2 | ACACTCACCGCCTTAACCAC | (SEQ ID No. 82) |
| 5.3.11.2 | AGTGCCCTTCATTCAGTTCC | (SEQ ID No. 83) |
| 6.8.13.2 | GCGACTTTGAGGGAACTATCC | (SEQ ID No. 84) |
| 7.5.9.3 | CACCACCCACCTTATGAAGC | (SEQ ID No. 85) |
| 7.5.9.4 | ACCAGGAGAATCAGCGACAC | (SEQ ID No. 86) |
| 8.62.2 | GGGACGAAGAATACGAGCTG | (SEQ ID No. 87) |
| Gt1 1f1 | CTGAATATCGACGGTTTCC | (SEQ ID No. 88) |
| Gt1 1f3 | GCACATTGGCTGAATATCG | (SEQ ID No. 89) |
| Gt1 1Rev | TTGACACCAGACCAACTGGTA | (SEQ ID No. 90) |
| | ATG | |

The oligonucleotides that were used in the different PCR reactions are listed in Table 4 below. PCR products were gel purified using standard procedures and subjected to DNA sequencing using sequencing oligonucleotides as indicated in Table 4 below. Sequencing was performed at GenomeExpress (Grenoble, France) and Sequentia (Clermont-Ferrand, France).

TABLE 4
Oligonucleotides for PCR and sequencing

| | March 2002 | March 2003 | | | | |
|---|---|---|---|---|---|---|
| | ORF | ORF | | 5′ | | sequencing |
| Gene id | SEQ_ID | SEQ_ID | PCR | oligo. | 3′ oligo. | oligo. |
| CEA229_genomic | 59 | 97 | PCR1 | Gt11f3 | 8.62.2 | 8.62.2 |
| CEA232_genomic | 50 | 109 | PCR2 | Gt11f1 | 10.175.2 | 10.175 |
| CEA232_genomic | 50 | 109 | PCR3 | Gt11f1 | 10.175.2 | 10.175.2 |
| CEA234_genomic | 47 | 117 | PCR4 | Gt11f3 | 10.304.4 | 10.304.4 |
| CEA257_genomic | 17 | 133 | PCR5 | 2.1.1.1 | 2.1.1.2 | 2.1.1.2 |
| CEA257_genomic | 17 | 133 | PCR6 | 2.1.1.5 | 2.1.1.6 | 2.1.1.5 |
| CEA257_genomic | 17 | 133 | PCR7 | 2.1.1.9 | Gt11Rev | 2.1.1.9 |
| CEA257_genomic | 17 | 133 | PCR8 | Gt11f3 | 2.1.1.2 | 2.1.1.2 |
| CEA261_genomic | 29 | 149 | PCR9 | 7.5.9.3 | 7.5.9.4 | 7.5.9.3 |
| CEA261_genomic | 29 | 149 | PCR10 | 7.5.9.3 | Gt11Rev | 7.5.9.3 |
| CEA265_genomic | 53 | 165 | PCR11 | GT11f1 | 11.4.9.4 | 11.4.9.4 |
| CEA280_genomic | | 173 | PCR12 | Gt11f3 | 6.8.13.2 | 6.8.13.2 |
| CEA281.1_genomic/CEA281.2_genomic | | 177 and 181 | PCR13 | Gt11f3 | 5.3.11.2 | 5.3.11.2 |
| CEA282_genomic | | 185 and 189 | PCR14 | GT11f1 | 10.4.20.2 | 10.4.20.2 |
| CEA282_genomic | | 186 and 189 | PCR15 | GT11f3 | 10.4.20.2 | 10.4.20.2 |
| CEA283_genomic | | | PCR16 | GT11f1 | 11.6.20.2 | 11.6.20.2 |
| CEA284.1_genomic/CEA284.2_genomic | | 194 and 198 | PCR17 | Gt11f1 | 4.3.4.2 | 4.3.4.2 |

Using this approach, we could confirm that sequences of SEQ ID NO 95, 107, 115, 131, 147, 171 are expressed in A. fumigatus grown in standard culture medium. Furthermore, the following results could be obtained:

1. PCR1 located the polyadenylation site 189 by 3′ of the proposed stop codon in SEQ ID NO 95;

2. PCR3 located the transcription start of SEQ ID NO 107, 103 by 5′ of the proposed start codon and confirmed the location of the first intron;

3. PCR4 located the transcription start site of SEQ ID NO 115, 113 by upstream of the proposed start codon, identified an intron in the 5′-untranslated region from position −84 to position −12 relative to the proposed start codon and confirmed the proposed location of the first and second introns;

4. PCR5 and PCR8 confirmed the proposed location of the first intron in SEQ ID NO 131;

5. PCR6 confirmed the proposed location of the second and third introns in SEQ ID NO 131;

6. PCR 7 located the polyadenylation site 106 by 3′ of the proposed stop codon in SEQ ID NO 131;

7. PCR9 and PCR10 confirmed the proposed location of the third intron in SEQ ID NO 147 and located two alternative polyadenylation sites 143 by and 167 by 3′ of the proposed stop codon in SEQ ID NO 147.

### Example 7

### Comparison of A. fumigatus EFG Proteins with Proteins of Other Fungal Species

EFG proteins of SEQ ID 106 to SEQ ID 174 were systematically compared to the genome of A. nidulans using the TBLASTN algorithm, to the genome and gene set of Magnaporthe grisea using the TBLASTN algorithm and to the protein set of Neurospora crassa and Saccharomyces cerevisiae using the BLASTP algorithm.

A. nidulans sequence data were obtained from the Aspergillus Sequencing project at the Whitehead Institute for Genome Research (http://wwwgenome.wi.mit.edu/annotation/fungi/aspergillus/index.html). The first release referred to as the Monsanto release was used.

M. grisea sequence data were obtained from the Magnaporthe Sequencing Project [Ralph Dean, Fungal Genomics Laboratory at North Carolina State University (www.fungalgenomics.ncsu.edu), and Whitehead Institute/MIT Center for Genome Research (www-genome.wi.mit.edu); http://www enome.wi.mit.edu/annotation/fungi/magnaporthe/index.html]. Release 2.1 was used.

N. crassa sequence data were obtained from the Neurospora Sequencing Project [Whitehead Institute/MIT Center for Genome Research (www-genome.wi.mit.edu); http://www-genome.wi.mitedu/annotation/fungi/neurospordindex.html] Release 3 was used. S. cerevisiae sequence data were obtained from the Saccharomyces Genome Database (http://genome-www.stanford.edu/Saccharomyces/).

A. nidulans, M. grisea, N. crassa and S. cerevisiae closest homologues of the A. fumigatus EFG proteins were subsequently compared to the genome of A. fumigatus using the TBLASTN algorithm and the data available from the A. fumigatus genome project (http://tigrblast.tigr.org/ufmg/) in order to evaluate whether these proteins are orthologues of the A. fumigatus EFG proteins (BDBH=yes) or are only homologues of the A. fumigatus EFG proteins with an A. fumigatus orthologue that differs from the A. fumigatus EFG protein (BDBH=no).

Results presented in Table 5 below show that:
all EFG proteins have a orthologue in the genome of A. nidulans
two EFG proteins are specific to Aspergilli (SEQ ID 98 and SEQ ID 170)
one EFG protein is specific to filamentous ascomycetes (SEQ ID 174)
the remaining 18 EFG proteins have orthologues in all investigated species.
   Consequently targets might be identified that are specific to Aspergilli, to filamentous ascomycetes or that have a broad spectrum. This analysis is reinforced by the comparison that was made to human proteins, whose results are shown in Table 6 below. As expected, SEQ ID 98 and SEQ ID 174 do not have a homologue encoded by the human genome thus defining targets for antifungal drugs that would show limited side effects.

TABLE 5

| | | March | March | | | | | | |
|---|---|---|---|---|---|---|---|---|---|
| | | 2002 | 2003 | S. cerevisiae | | | | | |
| | | Amino | Amino | closest | Protein | | | | |
| Clone | Protein | acid | acid | homologue | length | Probability | | | Essential |
| Id | Id | SEQ_ID | SEQ_ID | (p < 0.01) | (aa) | (e value) | Similarity | BDBH | in S.c. |
| 10-80 | CEA231_prot | 3 | 106 | DBP10/YDL031w | 995 | e−166 | 55% on | yes | yes |
| | | | | | | | 949 aa | | |
| 10- | CEA233_prot | 6 | 114 | NAR1/YNL240c | 491 | 4E−59 | 48% on | yes | yes |
| 291 | | | | | | | 465 aa | | |
| 7-1- | CEA254_prot | 9 | 122 | GUK1/YDR454c | 187 | 2E−62 | 81% on | yes | yes |
| 19 | | | | | | | 182 aa | | |
| 10-3-7 | CEA255_prot | 12 | 126 | SRP101/YDR292c | 621 | e−102 | 62% on | yes | yes |
| | | | | | | | 431 aa | | |
| 2-6-4 | CEA256_prot | 15 | 130 | WBP1/YEL002c | 430 | 2E−37 | 46% on | yes | yes |
| | | | | | | | 432 aa | | |
| 2-1-1 | CEA257_prot | 18 | 134 | YGL245w | 724 | 0.0 | 67% on | yes | yes |
| | | | | | | | 622 aa | | |
| 2-10- | CEA258_prot | 21 | 138 | CDC27/YBL084c | 758 | 4E−71 | 63% on | yes | yes |
| 16 | | | | | | | 304 aa | | |
| 5-4- | CEA259_prot | 24 | 142 | RSC9/YML127w | 581 | 5E−29 | 45% on | yes | yes |
| 21 | | | | | | | 414 aa | | |
| 2-10- | CEA260_prot | 27 | 146 | SPE2/YOL052c | 396 | 1E−54 | 51% on | yes | yes |
| 21 | | | | | | | 462 aa | | |
| 7-5-9 | CEA261_prot | 30 | 150 | RPL17A/YKL180w | 136 | 5E−40 | 92% on | yes | no |
| | | | | RPL17B/YJL177w | | | 115 aa | | |
| 10-2- | CEA262_prot | 33 | 154 | RPL1A/YGL135w | 255 | 9E−86 | 86% on | yes | no |
| 18 | | | | RPL1B/YPL220w | | | 238 aa | | |
| 9-11 | CEA230_prot | 36 | 102 | MSW1/YDR268w | 379 | 2E−26 | 43% on | yes | no |
| | | | | | | | 154 aa | | |
| 4-3-3 | CEA263_prot | 39 | 158 | GOS1/YHL031c | 223 | 8E−31 | 60% on | yes | no |
| | | | | | | | 224 aa | | |
| 11-6- | CEA264_prot | 42 | 162 | RIM11/YMR139w | 370 | e−104 | 77% on | no | no |
| 11 | | | | | | | 323 aa | | |
| 8-47 | CEA228_prot | 45 | 94 | YFL034w | 1074 | 5E−42 | 60% on | no | no |
| | | | | | | | 248 aa | | |
| 10- | CEA234_prot | 48 | 118 | RPL14A/YHL001w | 138 | 1E−24 | 63% on | yes | no |
| 304 | | | | RPL14B/YKL006w | | | 130 aa | | |
| 10- | CEA232_prot | 51 | 110 | HEM15/YOR176w | 393 | e−112 | 71% on | yes | yes |
| 175 | | | | | | | 352 aa | | |
| 11-4-9 | CEA265_prot | 54 | 166 | COX10/YPL172c | 462 | 1E−45 | 43% on | yes | no |
| | | | | | | | 341 aa | | |
| 2-10- | CEA266_prot | 57 | 170 | no hit | | | | | |
| 18 | | | | found | | | | | |
| 8-62 | CEA229_prot | 60 | 98 | no hit | | | | | |
| | | | | found | | | | | |
| 6-8- | CEA280_prot | | 174 | no hit | | | | | |
| 13 | | | | found | | | | | |
| 5-3- | CEA281.1_prot | | 178 | PAC2/YER007w | 518 | 8.4e−14 | 50% | yes | no |
| 11 | | | | | | | fragmented | | |
| 5-3- | CEA281.2_prot | | 182 | no hit | | | | | |
| 11 | | | | found | | | | | |
| 10-4- | CEA282.1_prot | | 186 | PBP2/YBR233w | 413 | 2.1e−26 | 50% | yes | no |
| 20 | | | | | | | fragmented | | |
| 10-4- | CEA282.2_prot | | 190 | SEC3/YER008c | 1337 | 2.2e−05 | 44% on | no | yes |
| 20 | | | | | | | 277 aa | | |
| 4-3-4 | CEA284.1_prot | | 195 | ENA5/YDR038c | 1091 | 2.5e−260 | 65% | yes | no |
| | | | | | | | fragmeted | | |
| 4-3-4 | CEA284.2_prot | | 199 | no hit | | | | | |
| | | | | found | | | | | |

| | | | | | | N. crassa | |
|---|---|---|---|---|---|---|---|
| | | | | | | homologue | |
| | | | | M. grisea | | (e-value | |
| | | A. nidulans homologue | | homologue | | % Pos | |
| Clone | Protein | (% Pos length of alignment | | (e-value % Pos | | length of | |
| Id | Id | region in Af protein) | BDBH | length of match) | BDBH | match) | BDBH |
| 10-80 | CEA231_prot | ANI61C8821 (92% 347 #1-345); | yes | MG04179.1 (0.0 | yes | NCU07712.1 | yes |
| | | ANI61C3840 (69% | | 65% 943) | | (0.0 | |
| | | 166 #772-934 + 83% 55 | | | | 69% 958) | |
| | | #723-777); ANI61S3472 | | | | | |
| | | (83% 129 #471-599 + 92% | | | | | |
| | | 14 #600-613); ANI61S468 | | | | | |
| | | (57% 132 #418-547) | | | | | |
| 10- | CEA233_prot | ANI61C10656 (74% 313 #1-273 + | yes | CEA233_homol_Mgrisea | yes | NCU03204.1 | yes |
| 291 | | 77% 270 #307-574) | | (not in Mg | | (e−159 | |
| | | | | gene Db; contig | | 62% | |
| | | | | 2.226 1621 . . . 71) | | 613) | |
| 7-1- | CEA254_prot | ANI61C9151 (75% 132 #2-110 + | yes | MG06764.1 (6e−58 | yes | NCU06300.1 | yes |
| 19 | | 91% 88 #111-198) | | 77% 183) | | (1e−61 | |
| | | | | | | 78% | |
| | | | | | | 181) | |
| 10-3-7 | CEA255_prot | ANI61C10591 (77% 346 | yes | MG02663.1 (0.0 | yes | NCU00625.1 | yes |
| | | #168-513 + 86% 160 #502-658); | | 67% 654) | | (e−174 | |
| | | ANI61C6709 (80% | | | | 67% | |
| | | 149 #1-129) | | | | 620) | |
| 2-6-4 | CEA256_prot | ANI61C5302 (69% 520 #1-460) | yes | MG02821.1 (e−125 | yes | NCU00669.1 | yes |
| | | | | 66% 465) | | (e−128 | |
| | | | | | | 64% | |
| | | | | | | 469) | |
| 2-1-1 | CEA257_prot | ANI61C10340 (83% 624 | yes | MG05956.1 (0.0 | yes | NCU08894.1 | yes |
| | | #92-715); ANI61C6256 | | 69% 633) | | (0.0 | |
| | | (82% 62 #1-62) | | | | 72% 631) | |
| 2-10- | CEA258_prot | ANI61C8961 (84% 402 | yes | MG06292.1 (e−174 | yes | NCU00213.1 | yes |
| 16 | | #390-786 + 55% 229 #171-399 + | | 57% 821) | | 13.1 (e−173 | |
| | | 63% 53 #119-171); | | | | 57% | |
| | | ANI61C6854 (87% 114 #1-114) | | | | 820) | |
| 5-4- | CEA259_prot | ANI61C10567 (92% 382 #1-382); | yes | MG02493.1 (6e−61 | yes | NCU03892.1 | yes |
| 21 | | ANI61C1244 (81% | | 51% 470) | | (7e−66 | |
| | | 144 #425-568) | | | | 52% | |
| | | | | | | 478) | |
| 2-10- | CEA260_prot | ANI61C9610 (82% 247 #39-273 + | yes | MG10635.1 (e−143 | yes | NCU01083.1 | yes |
| 21 | | 80% 236 #264-491 + | | 67% 479) | | (e−163 | |
| | | 94% 36 #6-41) | | | | 71% | |
| | | | | | | 502) | |
| 7-5-9 | CEA261_prot | ANI61C1126 (96% 70 #50-119 + | yes | MG04114.1 (3e−57 | yes | NCU07014.1 | yes |
| | | 77% 69 #8-72) | | 96% 118) | | (3e−58 | |
| | | | | | | 86% | |
| | | | | | | 139) | |
| 10-2- | CEA262_prot | ANI61C3974 (96% 170 #43-212 + | yes | MG06919.1 (e−116 | yes | NCU01452.1 | yes |
| 18 | | 94% 50 #207-256) | | 93% 238) | | (e−114 | |
| | | | | | | 91% | |
| | | | | | | 241) | |
| 9-11 | CEA230_prot | ANI61C6741 (79% 150 | yes | MG00474.1 (6e−85 | yes | NCU00113.1 | yes |
| | | #249-398 + 84% 93 #53-145 + | | 64% 351) | | (e−101 | |
| | | 56% 140 #137-249) | | | | 67% | |
| | | | | | | 374) | |
| 4-3-3 | CEA263_prot | ANI61C8624 (93% 172 #56-227 + | yes | MG04454.1 (5e−71 | yes | NCU02706.1 | yes |
| | | 91% 38 #23-60) | | 76% 226) | | (7e−74 | |
| | | | | | | 78% | |
| | | | | | | 224) | |
| 11-6- | CEA264_prot | ANI61C8742 (74% 246 #16-217 + | yes | MG03972.1 (0.0 | yes | NCU04185.1 | yes |
| 11 | | 87% 33 #215-247 + | | 93% 394) | | (0.0 | |
| | | 93% 15 #1-15); | | | | 92% 394) | |
| | | ANI61C11836 (79% 108 | | | | | |
| | | #263-350 + 88% 44 #351-394) | | | | | |
| 8-47 | CEA228_prot | ANI61C7512 (75% 306 | yes | MG08873.1 (1e−69 | yes | NCU01672.1 | yes |
| | | #374-679); ANI61C7189 | | 73% 240) | | (2e−89 | |
| | | (49% 225 #1-217) | | | | 50% | |
| | | | | | | 631) | |
| 10- | CEA234_prot | ANI61C6709 (90% 90 #43-132 + | yes | MG02659.1 (3e−40 | yes | NCU00634.1 | yes |
| 304 | | 79% 55 #1-55) | | 70% 132) | | (5e−35 | |
| | | | | | | 38% | |
| | | | | | | 132) | |
| 10- | CEA232_prot | ANI61C8249 (94% 200 #79-278 + | yes | MG01513.1 (e−172 | yes | NCU08291.1 | yes |
| 175 | | 56% 132 #1-132) | | 78% 420) | | (e−175 | |
| | | | | | | 79% | |
| | | | | | | 422) | |
| 11-4-9 | CEA265_prot | ANI61C1412 (50% 301 #20-320) | yes | MG05944.1 (2e−79 | yes | NCU06141.1 | yes |
| | | | | 52% 351) | | (6e−81 | |
| | | | | | | 51% | |
| | | | | | | 351) | |
| 2-10- | CEA266_prot | ANI61C1220 (57% 305 | yes | MG00073.1 (9e−06 | no | NCU05588.1 | no |
| 18 | | #233-537) | | 39% 254) | | (2e−07 | |
| | | | | | | 37% | |
| | | | | | | 289) | |
| 8-62 | CEA229_prot | ANI61C3462 (72% 472 | yes | no hit found | ND | no hit | ND |
| | | #237-707 + 68% 59 #710-768); | | | | found | |
| | | ANI61C1809 (63% | | | | | |
| | | 179 #762-926); | | | | | |
| | | ANI61C9531 (80% 97 #7-103) | | | | | |
| 6-8- | CEA280_prot | ANI61C5802 (68% 182 #15-190) | yes | MG04487.1 (6e−11 | yes | NCU09996.1 | yes |
| 13 | | | | 45% 175) | | (1e−11 | |
| | | | | | | 46% | |
| | | | | | | 175) | |
| 5-3- | CEA281.1_prot | ANI61C7709 (76% 206 | yes | MG00378.1 (2e−95 | yes | NCU09139.1 | yes |
| 11 | | #280-482 + 74% 146 #60-205 + | | 51% 641) | | (2e−98 | |
| | | 96% 53 #6-58) | | | | 50% | |
| | | | | | | 634) | |
| 5-3- | CEA281.2_prot | ANI61C868 (72% 169 #47-211) | yes | MG07998.1 (2e−48 | yes | NCU04032.1 | yes |
| 11 | | | | 52% 300) | | (5e−42 | |
| | | | | | | 48% | |
| | | | | | | 355) | |
| 10-4- | CEA282.1_prot | ANI61C4164 (79% 205 #1-200 + | yes | MG00514.1 (e−125 | yes | NCU09237.1 | yes |
| 20 | | 82% 158 171-328); | | 60% 485) | | (e−131 | |
| | | ANI61C6581 (55% 142 | | | | 60% | |
| | | #323-464) | | | | 492) | |
| 10-4- | CEA282.2_prot | ANI61C11139 (67% 281 | yes | MG00515.1 (2e−41 | yes | NCU09238.1 | yes |
| 20 | | #64-344); ANI61C4164 | | 50% 315) | | (4e−36 | |
| | | (71% 60 #1-60) | | | | 49% | |
| | | | | | | 334) | |
| 4-3-4 | CEA284.1_prot | ANI61C1006 (88% 336 | yes | MG10730.1 (0.0 | yes | NCU05046.1 | yes |
| | | #429-760 + 86% 260 #167-426 + | | 72% 1072) | | (0.0 | |
| | | 78% 190 #1-169); | | | | 74% | |
| | | ANI61C1748 (84% 212 | | | | 1038) | |
| | | #851-1059 + 95% 73 #781-853) | | | | | |
| 4-3-4 | CEA284.2_prot | ANI61C8195 (39% 126 | yes | MG10932.1 (2e−08 | no | NCU00723.1 | no |
| | | #232-350); ANI61C3069 | | 40% 117) | | (3e−07 | |
| | | (63% 56 #35-90) | | | | 37% | |
| | | | | | | 106) | |

TABLE 6

| | | March | March | | |
|---|---|---|---|---|---|
| | | 2002 | 2003 | | |
| | | Amino | Amino | | |
| Clone | | acid | acid | Nearest human homologue at the | |
| Id | Protein Id | SEQ_ID | SEQ_ID | integratuion of site imp160::pyrG | Probability |
| 10-80 | CEA231_prot | 3 | 106 | ATP-dependent RNA helicase | e−123 |
| 10- | CEA233_prot | 6 | 114 | protein related to Narf | 4E−59 |
| 291 | | | | | |
| 7-1- | CEA254_prot | 9 | 122 | guanylate kinase 1 | 5E−52 |
| 19 | | | | | |
| 10-3-7 | CEA255_prot | 12 | 126 | signal recognition particle receptor | 1E−88 |
| 2-6-4 | CEA256_prot | 15 | 130 | dolichyl-diphosphooligosaccharide- | 4E−45 |
| | | | | protein glycosyltransferase | |
| 2-1-1 | CEA257_prot | 18 | 134 | glutamyl-prolyl tRNA synthetase | e−135 |
| 2-10- | CEA258_prot | 21 | 138 | cell division cycle protein 27 | 5E−81 |
| 16 | | | | | |
| 5-4- | CEA259_prot | 24 | 142 | zinc finger protein of the cerebellum 2 | 0.001 |
| 21 | | | | | |
| 2-10- | CEA260_prot | 27 | 146 | S-adenosylmethionine decarboxylase 1 | 5E−50 |
| 21 | | | | | |
| 7-5-9 | CEA261_prot | 30 | 150 | ribosomal protein S17; 40S ribosomal | 1E−36 |
| | | | | protein | |
| 10-2- | CEA262_prot | 33 | 154 | ribosomal protein S3a; 40S ribosomal | 1E−76 |
| 18 | | | | protein | |
| 9-11 | CEA230_prot | 36 | 102 | tryptophanyl tRNA synthetase 2 | 7E−27 |
| | | | | (mitochondrial) | |
| 4-3-3 | CEA263_prot | 39 | 158 | golgi SNAP receptor complex | 6E−21 |
| | | | | member 1; Golgi SNARE | |
| 11-6- | CEA264_prot | 42 | 162 | glycogen synthase kinase 3 beta | e−137 |
| 11 | | | | | |
| 8-47 | CEA228_prot | 45 | 94 | hypothetical protein | 6E−28 |
| 8-62 | CEA229_prot | 60 | 98 | no hit found | |
| 6-8- | CEA280_prot | | 174 | no hit found | |
| 13 | | | | | |
| 5-3- | CEA281.1_prot | | 178 | beta-tubulin cofactor E | 5E−29 |
| 11 | | | | | |
| 5-3- | CEA281.2_prot | | 182 | WW domain-containing binding | 0.020 |
| 11 | | | | protein 4; formin binding protein 21 | |
| 10-4- | CEA282.1_prot | | 186 | poly(rC) binding protein 1; | 1E−14 |
| 20 | | | | heterogenous nuclear | |
| | | | | ribonucleoprotein X | |
| 10-4- | CEA282.2_prot | | 190 | no hit found | |
| 20 | | | | | |
| 4-3-4 | CEA284.2_prot | | 199 | no hit found | |

| | | Protein | | | |
|---|---|---|---|---|---|
| Clone | | Length | | | Human |
| Id | Protein Id | (aa) | Identitity (%) | Similitary (%) | protein Ref |
| 10-80 | CEA231_prot | 882 | 261/594 (43%) | 353/594 | NP_076977.2 |
| | | | | (58%) | |
| 10- | CEA233_prot | 476 | 168/474 (35%) | 216/474 | NP_071938.1 |
| 291 | | | | (45%) | |
| 7-1- | CEA254_prot | 197 | 98/179 (54%) | 131/179 | NP_000849.1 |
| 19 | | | | (72%) | |
| 10-3-7 | CEA255_prot | 638 | 232/668 (34%) | 334/668 | NP_003130.1 |
| | | | | (49%) | |
| 2-6-4 | CEA256_prot | 456 | 124/423 (29%) | 213/423 | NP_005207.2 |
| | | | | (50%) | |
| 2-1-1 | CEA257_prot | 1440 | 260/614 (42%) | 372/614 | NP_004437.1 |
| | | | | (60%) | |
| 2-10- | CEA258_prot | 824 | 168/465 (36%) | 257/465 | NP_001247.2 |
| 16 | | | | (55%) | |
| 5-4- | CEA259_prot | 532 | 28/102 (27%) | 37/102 | NP_009060.2 |
| 21 | | | | (35%) | |
| 2-10- | CEA260_prot | 334 | 140/447 (31%) | 215/447 | NP_001625.1 |
| 21 | | | | (47%) | |
| 7-5-9 | CEA261_prot | 135 | 85/119 (71%) | 98/119 | NP_001012.1 |
| | | | | (81%) | |
| 10-2- | CEA262_prot | 264 | 151/246 (61%) | 190/246 | NP_000997.1 |
| 18 | | | | (76%) | |
| 9-11 | CEA230_prot | 360 | 64/183 (34%) | 104/183 | NP_056651.1 |
| | | | | (55%) | |
| 4-3-3 | CEA263_prot | 250 | 67/247 (27%) | 125/247 | NP_004862.1 |
| | | | | (50%) | |
| 11-6- | CEA264_prot | 433 | 241/352 (68%) | 281/352 | NP_002084.2 |
| 11 | | | | (79%) | |
| 8-47 | CEA228_prot | 421 | 69/188 (36%) | 100/188 | CAB39107.1 |
| | | | | (52%) | |
| 8-62 | CEA229_prot | | | | |
| 6-8- | CEA280_prot | | | | |
| 13 | | | | | |
| 5-3- | CEA281.1_prot | 527 | 75/199 (37%) | 108/199 | NP_003184.1 |
| 11 | | | 55/211 (26%) | (54%) | |
| | | | | 91/211 | |
| | | | | (43%) | |
| 5-3- | CEA281.2_prot | 376 | 22/64 (34%) | 36/64 | NP_009118.1 |
| 11 | | | | (56%) | |
| 10-4- | CEA282.1_prot | 356 | 59/194 (30%) | 96/194 | NP_006187.1 |
| 20 | | | | (49%) | |
| 10-4- | CEA282.2_prot | | | | |
| 20 | | | | | |
| 4-3-4 | CEA284.2_prot | | | | |

### REFERENCES

1. Latgé, J. P. Aspergillus fumigatus and aspergillosis. Clin Microbiol Rev 12, 310-350 (1999).
2. Latgé, J. The pathobiology of Aspergillus fumigatus. Trends Microbiol 9, 382-389 (2001).
3. Lin, S., Schranz, J. & Teutsch, S. Aspergillosis case-fatality rate: systematic review of the literature. Clin Infect Dis 32, 358-366. (2001).
4. McNeil, M. M. et al. Trends in mortality due to invasive mycotic diseases in the United States, 1980-1997. Clin Infect Dis 33, 641-647. (2001).
5. Georgopapadakou, N. H. Antifungals: mechanism of action and resistance, established and novel drugs. Curr Opin Microbiol 1, 547-557 (1998).
6. Tkacz, J. S. & DiDomenico, B. Antifungals: what's in the pipeline. Curr Opin Microbiol 4, 540-545. (2001).
7. Walsh, T. J. et al. New targets and delivery systems for antifungal therapy. Med Mycol 38, 335-347. (2000).
8. Groll, A. H., De Lucca, A. J. & Walsh, T. J. Emerging targets for the development of novel antifungal therapeutics. Trends Microbiol 6, 117-124 (1998).
9. Perfect, J. R. Fungal virulence genes as targets for antifungal chemotherapy. Antimicrob Agents Chemother 40, 1577-1583 (1996).
10. Brown, J. S. et al. Signature-tagged and directed mutagenesis identify PABA synthetase as essential for Aspergillus fumigatus pathogenicity. Mol Microbiol 36, 1371-1380. (2000).
11. Reich, K. A. The search for essential genes. Res Microbiol 151, 319-324 (2000).
12. Odds, F. C., Gow, N. A. & Brown, A. J. Fungal virulence studies come of age. Genome Biol 2 (2001).
13. Winzeler, E. A. et al. Functional characterization of the S. cerevisiae genome by gene deletion and parallel analysis. Science 285, 901-906 (1999).
14. Vidan, S. & Snyder, M. Large-scale mutagenesis: yeast genetics in the genome era. Curr Opin Biotechnol 12, 28-34. (2001).
15. De Backer, M. D. et al. An antisense-based functional genomics approach for identification of genes critical for growth of Candida albicans. Nat Biotechnol 19, 235-241. (2001).
16. Brookman, J. L. & Denning, D. W. Molecular genetics in Aspergillus fumigatus. Curr Opin Microbiol 3, 468-474. (2000).
17. Timberlake, W. E. in More Gene Manipulations in Fungi (eds. Bennett, J. W. & Lasure, L. L.) 51-85 (Academic Press Inc., Oxford, 1991).
18. Clutterbuck, A. J. Sexual and parasexual genetics of Aspergillus species. Biotechnology 23, 3-18 (1992).
19. Brown, J. S., Aufauvre-Brown, A. & Holden, D. W. Insertional mutagenesis of Aspergillus fumigatus. Mol Gen Genet. 259, 327-335 (1998).
20. d′Enfert, C., Weidner, G., Mol, P. C. & Brakhage, A. A. Transformation systems of Aspergillus fumigatus. New tools to investigate fungal virulence. Contrib Microbiol 2, 149-166 (1999).
21. Judson, N. & Mekalanos, J. J. Transposon-based approaches to identify essential bacterial genes. Trends Microbiol 8, 521-526. (2000).
22. Judson, N. & Mekalanos, J. J. TnAraOut, a transposon-based approach to identify and characterize essential bacterial genes. Nat Biotechnol 18, 740-745. (2000).
23. Kumar, A. & Snyder, M. Emerging technologies in yeast genomics. Nat Rev Genet. 2, 302-312. (2001).
24. Ross-Macdonald, P. et al. Large-scale analysis of the yeast genome by transposon tagging and gene disruption. Nature 402, 413-418 (1999).
25. Hamer, L. et al. Gene discovery and gene function assignment in filamentous fungi. Proc Nati Acad Sci USA 98, 5110-5115. (2001).
26. Brown, J. S. & Holden, D. W. Insertional mutagenesis of pathogenic fungi. Curr Opin Microbiol 1, 390-394 (1998).
27. Langin, T., Capy, P. & Daboussi, M. J. The transposable element impala, a fungal member of the Tcl-mariner superfamily. Mol Gen Genet. 246, 19-28 (1995).
28. Hua-Van, A., Pamphile, J. A., Langin, T. & Daboussi, M. J. Transposition of autonomous and engineered impala transposons in Fusarium oxysporum and a related species. Mol Gen Genet. 264, 724-731. (2001).
29. Li Destri Nicosia, M. G. et al. Heterologous transposition in Aspergillus nidulans. Mol Microbiol 39, 1330-1344. (2001).
30. Villalba, F., Lebrun, M. H., Hua-Van, A., Daboussi, M. J. & Grosjean-Cournoyer, M. C. Transposon impala, a novel tool for gene tagging in the rice blast fungus Magnaporthe grisea. Mol Plant Microbe Interact 14, 308-315. (2001).
31. Hastie, A. C. Benlate-induced Instability of Aspergillus diploids. Nature 226, 771 (1970).
32. Altschul, S. F. et al. Gapped BLAST and PSI-BLAST: a new generation of protein database search programs. Nucleic Acids Res 25, 3389-3402. (1997).
33. Akerley, B. J. et al. A genome-scale analysis for identification of genes required for growth or survival of Haemophilusinfluenzae. Proc Natl Acad Sci USA 99, 966-971. (2002).
34. Craig, N. L. Target site selection in transposition. Annu Rev Biochem 66, 437-474 (1997).
35. Giaever, G. et al. Genomic profiling of drug sensitivities via induced haploinsufficiency. Nat Genet. 21, 278-283 (1999).
36. Chun, K. T., Edenberg, H. J., Kelley, M. R. & Goebl, M. G. Rapid amplification of uncharacterized transposon-tagged DNA sequences from genomic DNA. Yeast 13, 233-240 (1997).
37. Bouchara, J. P. et al. The search for virulence determinants in Aspergillus fumigatus; Trends Microbiol. 3(8), 327-330 (1995).
38. Langfelder, K. et al. Identification of a polyketide Synthase Genes (pKsP) of Aspergillus fumigatus involved in conidial pigment biosynthesis and virulence. Med Microbiol Immunol (Berl.) 187(2), 79-89 (1998).
39. Tsai, H. F. et al. A developmentally regulated gene cluster involved in conidial pigment biosynthesis in Aspergillus fumigatus. J. Bacteriol 181(20), 6469-6477 (1999).

1. A nucleic acid encoding an Essential For Growth (EFG) polypeptide selected from the group consisting of:
(i) a nucleic acid molecule encoding a polypeptide comprising the amino acid sequence depicted in one of SEQ ID NO 9 and 122;
(ii) a nucleic acid molecule comprising the nucleic acid sequence as depicted in one of SEQ ID NO 7 and 120;
(iii) a nucleic sequence having at least 80, 85, 90, 95, 98, 99% identity with a sequence of SEQ ID NO 7 or 120;
(iv) a nucleic acid molecule which hybridizes under stringent conditions to:
(a) a nucleic acid as defined in (i), (ii) and (iii), or
(b) a complementary strand of (a); or
(v) a nucleic acid the sequence of which is degenerate as a result of the genetic code to the sequence of a nucleic acid as defined in (i), (ii), (iii) and (iv).

2. An isolated nucleic acid, said nucleic acid comprising a nucleotide sequence encoding:
i) an Essential For Growth (EFG) polypeptide comprising an amino acid sequence having at least 80% identity to a sequence of SEQ ID NO 9 or 122; or
ii) a biologically active fragment of said polypeptide.

3. An isolated nucleic acid, said nucleic acid comprising a nucleotide sequence encoding:
i) an Essential For Growth (EFG) polypeptide comprising an amino acid sequence which is orthologous to a sequence of SEQ ID NO 3 or 122; or
ii) a biologically active fragment of said polypeptide.

4. The nucleic acid sequence of claim 1 encoding a polypeptide of A. fumigatus exhibiting a biological function associated to fungal growth, said nucleic acid comprising a sequence of SEQ ID NO 8 or 121.

5. The nucleic acid sequence of claim 4, wherein said biological function associated to fungal growth nucleotide metabolism.

6. The nucleic acid of claim 1, wherein said nucleic acid is operably linked to a promoter.

7. An expression cassette comprising the nucleic acid of claim 6.

8. A host cell comprising the expression cassette of claim 7.

9. A biologically active polypeptide encoded by the nucleic acid according to claim 1.

10. The polypeptide according to claim 9 or a biologically active fragment thereof, said polypeptide comprising an amino acid sequence of at least 80% amino acid sequence identity to a sequence of SEQ ID NO 9 or 122.

11. A method of identifying a candidate inhibitor of an Essential For Growth (EFG) polypeptide, said method comprising
a) contacting an EFG polypeptide according to claim 9 with a test compound; b) determining whether said compound selectively binds to said polypeptide, said binding indicating that said compound is a candidate inhibitor.

12. (canceled)

13. A method for detecting the presence of a nucleic acid comprising a nucleotide sequence of SEQ ID NO 7 or 120, a fragment or a variant thereof and a complementary sequence thereto in a sample, said method comprising the following steps of:
a) bringing into contact a nucleic acid probe or a plurality of nucleic acid probes which can hybridize with a nucleotide sequence included in a nucleic acid sequence of SEQ ID NO 7 or 120, a fragment or a variant thereof and a complementary sequence thereto and the sample to he assayed; and
b) detecting the hybrid complex fanned between the probe and a nucleic acid in the sample.

14-26. (canceled)

27. A composition capable of inhibiting haploid fungal growth, wherein said composition comprises at least one compound capable of inhibiting the expression of at least one Essential For Growth (EFG) gene as defined in of claim 1.

28. The composition of claim 13, which is a pharmaceutical composition.

29. The composition of claim 13 or 14, which is a fungicidal composition.
